(19) United States
(12) Patent Application Publication
(10) Pub. No.: US 2005/0113379 A1
(43) Pub. Date: May 26, 2005

(54) Heteroaryl fused pyridines, pyrazines and pyrimidines as CRF1 receptor ligands

(76) Inventors: Ping Ge, Durham, CT (US); Raymond F. Horvath, Guilford, CT (US); Lu Yan Zhang, Branford, CT (US); Yasuchika Yamaguchi, Guilford, CT (US); Bernd Kaiser, Wallingford, CT (US); Xuechun Zhang, Branford, CT (US); Suoming Zhang, Madison, CT (US); He Zhao, Branford, CT (US); Stanly John, Basking Ridge, NJ (US); Neil Moorcroft, Bloomsbury, NJ (US); Greg Shutske, Pittstown, NJ (US)

Correspondence Address: EDWARDS & ANGELL, LLP, P.O. BOX 55874, BOSTON, MA, 02205, US

(21) Appl. No.: 10/933,834

(22) Filed: Sep. 3, 2004

Related U.S. Application Data
(60) Provisional application No. 60/500,414, filed on Sep. 5, 2003.

(51) Int. Cl. A61K 31/519; A61K 31/522; C07D 498/02; C07D 491/02; C07D 473/02

(52) U.S. Cl. 514/249; 514/260.1; 514/262.1; 514/263.3; 544/255; 544/262; 544/265; 544/350

(57) ABSTRACT

Substituted heteroaryl fused pyridine, pyrazine, and pyrimidine compounds that act as selective modulators of CRF 1 receptors are provided. These compounds are useful in the treatment of a number of CNS and periphereal disorders, particularly stress, anxiety, depression, cardiovascular disorders, and eating disorders. Methods of treatment of such disorders and well as packaged pharmaceutical compositions are also provided. Compounds of the invention are also useful as probes for the localization of CRF receptors and as standards in assays for CRF receptor binding. Methods of using the compounds in receptor localization studies are given.

This application claims priority from U.S. Provisional Application Ser. No. 60/500,414 filed on Sep. 5, 2003.

### FIELD OF THE INVENTION

The present invention relates to novel substituted heteroaryl fused pyridine, pyrazine, and pyrimidine compounds that bind with high selectivity and/or high affinity to CRF receptors (Corticotropin Releasing Factor Receptors). This invention also relates to pharmaceutical compositions comprising such compounds and to the use of such compounds in treatment of psychiatric disorders and neurological diseases, including major depression, anxiety-related disorders, post-traumatic stress disorder, supranuclear palsy and feeding disorders, as well as treatment of immunological, cardiovascular or heart-related diseases and colonic hypersensitivity associated with psychopathological disturbance and stress. Additionally this invention relates to the use such compounds as probes for the localization of CRF receptors in cells and tissues. Preferred CRF receptors are CRF1 receptors.

### BACKGROUND OF THE INVENTION

Corticotropin releasing factor (CRF), a 41 amino acid peptide, is the primary physiological regulator of proopiomelanocortin (POMC) derived peptide secretion from the anterior pituitary gland. In addition to its endocrine role at the pituitary gland, immunohistochemical localization of CRF has demonstrated that the hormone has a broad extrahypothalamic distribution in the central nervous system and produces a wide spectrum of autonomic, electrophysiological and behavioral effects consistent with a neurotransmitter or neuromodulator role in brain. There is also evidence that CRF plays a significant role in integrating the response of the immune system to physiological, psychological, and immunological stressors.

Clinical data provide evidence that CRF has a role in psychiatric disorders and neurological diseases including depression, anxiety-related disorders and feeding disorders. A role for CRF has also been postulated in the etiology and pathophysiology of Alzheimer's disease, Parkinson's disease, Huntington's disease, progressive supranuclear palsy and amyotrophic lateral sclerosis as they relate to the dysfunction of CRF neurons in the central nervous system.

In affective disorder, or major depression, the concentration of CRF is significantly increased in the cerebral spinal fluid (CSF) of drug-free individuals. Furthermore, the density of CRF receptors is significantly decreased in the frontal cortex of suicide victims, consistent with a hypersecretion of CRF. In addition, there is a blunted adrenocorticotropin (ACTH) response to CRF (i.v. administered) observed in depressed patients. Preclinical studies in rats and non-human primates provide additional support for the hypothesis that hypersecretion of CRF may be involved in the symptoms seen in human depression. There is also preliminary evidence that tricyclic antidepressants can alter CRF levels and thus modulate the numbers of CRF receptors in brain.

CRF has also been implicated in the etiology of anxiety-related disorders. CRF produces anxiogenic effects in animals and interactions between benzodiazepine/non-benzodiazepine anxiolytics and CRF have been demonstrated in a variety of behavioral anxiety models. Preliminary studies using the putative CRF receptor antagonist ac-helical ovine CRF (9-41) in a variety of behavioral paradigms demonstrate that the antagonist produces “anxiolytic-like” effects that are qualitatively similar to the benzodiazepines. Neurochemical, endocrine and receptor binding studies have all demonstrated interactions between CRF and benzodiazepine anxiolytics providing further evidence for the involvement of CRF in these disorders. Chlordiazepoxide attenuates the “anxiogenic” effects of CRF in both the conflict test and in the acoustic startle test in rats. The benzodiazepine receptor antagonist Ro 15-1788, which was without behavioral activity alone in the operant conflict test, reversed the effects of CRF in a dose-dependent manner, while the benzodiazepine inverse agonist FG 7142 enhanced the actions of CRF.

CRF has also been implicated in the pathogeneisis of certain immunological, cardiovascular or heart-related diseases such as hypertension, tachycardia and congestive heart failure, stroke and osteoporosis, as well as in premature birth, psychosocial dwarfism, stress-induced fever, ulcer, diarrhea, post-operative ileus and colonic hypersensitivity associated with psychopathological disturbance and stress.

The mechanisms and sites of action through which conventional anxiolytics and antidepressants produce their therapeutic effects remain to be fully elucidated. It has been hypothesized however, that they are involved in the suppression of CRF hypersecretion that is observed in these disorders. Of particular interest are that preliminary studies examining the effects of a CRF receptor antagonist peptide (α-helical CRF₉₋₄₁) in a variety of behavioral paradigms have demonstrated that the CRF antagonist produces “anxiolytic-like” effects qualitatively similar to the benzodiazepines.

### SUMMARY OF THE INVENTION

The invention provides novel compounds of Formula I (shown below), and pharmaceutical compositions comprising compounds of Formula I and at least one pharmaceutically acceptable carrier or excipient. Such compounds bind to cell surface receptors, preferably G-coupled protein receptors, especially CRF receptors (including CRF1 and CRF2 receptors) and most preferably CRF 1 receptors. Preferred compounds of the invention exhibit high affinity for CRF receptors, preferably CRF 1 receptors. Additionally, preferred compounds of the invention also exhibit high specificity for CRF receptors (i.e., they exhibit high selectivity compared to their binding to non-CRF receptors). Preferably they exhibit high specificity for CRF 1 receptors.

Thus, in certain aspects, the invention provides compounds of Formula I-a 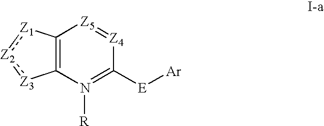
and the pharmaceutically acceptable salts thereof, wherein:
E is a single bond, O, S(O)ₘ, NR₁₀ or CR₁₀OR₁₁;
R₁₀ and R₁₁ are independently hydrogen or C₁-C₄ alkyl;
m is 0, 1, or 2;
Ar is chosen from:
phenyl which is mono-, di-, or tri-substituted, 1-naphthyl and 2-naphthyl, each of which is optionally mono-, di-, or tri-substituted, and optionally mono-, di-, or tri-substituted heteroaryl, said heteroaryl having from 1 to 3 rings, 5 to 7 ring members in each ring and, in at least one of said rings, from 1 to about 3 heteroatoms selected from the group consisting of N, O, and S;
R is oxygen or absent;
   the group: 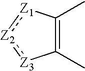
   represents a saturated, unsaturated or aromatic 5-membered ring system containing 0 or 1 heteroatoms, wherein:
Z₁ is CR₁ or CR₁R₁′;
Z₂ is nitrogen, oxygen, sulfur, CR₂, CR₂R₂′ or NR₂″,
Z₃ is nitrogen, oxygen, sulfur, sulfoxide, sulfone, CR₃, or CR₃R₃′;
R₁ is chosen from hydrogen, halogen, hydroxy, cyano, amino, optionally substituted alkyl, optionally substituted alkenyl, optionally substituted alkynyl, optionally substituted alkoxy, optionally substituted mono or dialkylamino, optionally substituted cycloalkyl, optionally substituted (cycloalkyl)alkyl, optionally substituted alkylthio, optionally substituted alkylsulfinyl, optionally substituted alkylsulfonyl, optionally substituted mono- or dialkylcarboxamide, optionally substituted carbocyclic aryl, optionally substituted heterocycle and optionally substituted heteroaryl, said optionally substituted heterocycle or heteroaryl having from 1 to 3 rings, 5 to 7 ring members in each ring and, in at least one of said rings, from 1 to about 3 heteroatoms selected from the group consisting of N, O, and S;
R₂ and R₃ are independently chosen from hydrogen, halogen, hydroxy, amino, cyano, nitro, alkyl, haloalkyl, alkoxy, aminoalkyl, hydroxyalkyl and mono and dialkylamino, wherein when R₁ or R₁″ is optionally substituted alkyl, then R₃ is optionally substituted C₁₋₃alkyl;
R₁′, R₂′ and R₃′ are independently chosen from hydrogen, halogen, alkyl, haloalkyl, and aminoalkyl;
R₂″ is chosen from hydrogen, optionally substituted alkyl, optionally substituted haloalkyl, and optionally substituted aminoalkyl;
Z₄ is NR or CR₄;
Z₅ is NR or CR₅;
R₄ and R₅ are independently chosen from hydrogen, halogen, hydroxy, amino, cyano, nitro, optionally substituted alkyl, optionally substituted alkenyl, optionally substituted alkynyl, optionally substituted alkoxy, optionally substituted mono or dialkylamino, optionally substituted (cycloalkyl)alkyl, optionally substituted alkylthio, optionally substituted alkylsulfinyl, optionally substituted alkylsulfonyl, optionally substituted mono- or dialkylcarboxamide, optionally substituted carbocyclic aryl, and optionally substituted heteroaryl, said optionally substituted heteroaryl having from 1 to 3 rings, 5 to 7 ring members in each ring and, in at least one of said rings, from 1 to about 3 heteroatoms selected from the group consisting of N, O, and S.

In certain other aspects, the invention provides compounds of Formula I-b 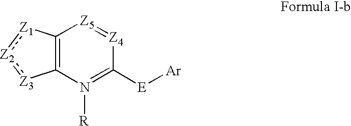
or a pharmaceutically acceptable salt thereof, wherein:
E is a single bond, O, S(O)ₘ, NR₁₀ or CR₁₀R₁₁;
R₁₀ and R₁₁ are independently hydrogen or C₁-C₄ alkyl;
m is 0, 1, or 2;
R is oxygen or absent;
Ar is chosen from:
phenyl which is mono-, di-, or tri-substituted, 1-naphthyl and 2-naphthyl, each of which is optionally mono-, di-, or tri-substituted, and optionally mono-, di-, or tri-substituted heteroaryl, said heteroaryl having from 1 to 3 rings, 5 to 7 ring members in each ring and, in at least one of said rings, from 1 to about 3 heteroatoms selected from the group consisting of N, O, and S;
   the group: 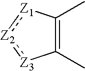
   represents a saturated, unsaturated or aromatic ring system comprising 0 or 1 heteroatoms, wherein:
Z₁ is CR₁, CR₁R₁′ or NR₁″;
Z₂ is CR₂ or CR₂R₂′;
Z₃ is CR₃, CR₃R₃′, or NR₃″;
R₁ and R₁″ are chosen from hydrogen, C₁-C₁₀alkyl, C₂-C₁₀alkenyl, C₂-C₁₀alkynyl, C₃-C₇cycloalkyl, (benzo)C₃-C₇cycloalkyl, (C₃-C₇cycloalkyl)C₁-C₄alkyl, C₃₋₉ heterocycloalkyl, (C₃₋₉heterocycloalkyl)C₁-C₄alkyl, (benzo)C₃₋₉heterocycloalkyl, ((benzo)C₃₋₉heterocycloalkyl)C₁-C₄alkyl and halo(C₁-C₆)alkyl, each of which is substituted with 0 or more substituents independently chosen from halogen, hydroxy, amino, oxo, cyano, C₁-C₆alkyl, C₁-C₆alkoxy, haloC₁-C₆alkoxy, C₁-C₆alkanoyl, C₁-C₆alkanoyloxy, C₁-C₆alkoxycarbonyl, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, C₁-C₆alkylsulfonamide, C₁-C₆alkylsulfonyl, C₁-C₆alkylsulfonyloxy, C₁-C₆ hydroxyalkyl, C₁-C₆alkoxyC₁-C₆alkyl, C₁-C₆ haloalkoxy, 5 to 7 membered heteroaryl, 5 to 7 membered heterocycloalkyl, mono- and di-(C₁-C₆)alkylamino, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, mono- and di-(C₁-C₆)alkylcarbamoyl, —XR_{C} and X-Z, with the proviso that R₁ and R₁″ is not aryl or heteroaryl substituted alkyl;
R₂ is chosen from hydrogen, halogen, hydroxy, amino, cyano, nitro, C₁-C₃alkyl, halo(C₁-C₃)alkyl, C₁-C₃alkoxy, amino(C₁-C₃)alkyl, and mono and di(C₁-C₆)alkylamino;
R₃ is chosen from hydrogen, hydroxy, amino, halogen, cyano, nitro, C₁-C₃alkyl, halo(C₁-C₃)alkyl, C₁-C₃alkoxy, amino(C₁-C₃)alkyl, hydroxy(C₁-C₃)alkyl, cyano(C₁-C₃)alkyl, and mono and di(C₁-C₃)alkylamino;
R₃″ is chosen from hydrogen, hydroxy, amino, C₁-C₃alkyl, halo(C₁-C₃)alkyl, C₁-C₃alkoxy, amino(C₁-C₃)alkyl, hydroxy(C₁-C₃)alkyl, cyano(C₁-C₃)alkyl, and mono and di(C₁-C₃)alkylamino;
R₁′, R₂′ and R₃′ are independently chosen from hydrogen, halogen, C₁-C₆alkyl, halo(C₁-C₆)alkyl, and amino(C₁-C₆)alkyl;
Z₄ is NR or CR₄;
Z₅ is NR or CR₅;
R₄ and R₅ are independently chosen from hydrogen, halogen, cyano, nitro, amino, mono or di(C₁-C₆carbhydryl)amino, C₁-C₆carbhydryl, (C₃-C₇cyclocarbhydryl)C₀-C₄carbhydryl, —O(C₃-C₇cyclocarbhydryl), halo(C₁-C₆)carbhydryl, —O(halo(C₁-C₆)carbhydryl), —O(C₁-C₆carbhydryl), S(O)ₙ(C₁-C₆carbhydryl), and 4 to 7 membered heterocycloalkyl,
   where each carbhydryl is independently straight, branched, or cyclic, contains zero or 1 or more double or triple bonds, and is optionally substituted with one or more substituents independently chosen from halogen, hydroxy, amino, oxo, cyano, C₁-C₄alkoxy, and mono- and di(C₁-C₄)alkylamino,
   and
   where each C₃-C₇carbhydryl heterocycloalkyl is optionally substituted by one or more substituents independently chosen from halogen, amino, hydroxy, oxo, cyano, C₁-C₄alkoxy, and mono- and di(C₁-C₄)alkylamino; or
R₅, taken in combination with R₁ or R₁″, forms a 5-9 membered heterocycle;
R_{A} is independently selected at each occurrence from halogen, cyano, nitro, halo(C₁-C₆)alkyl, halo(C₁-C₆)alkoxy, hydroxy, amino, C₁-C₆alkyl substituted with 0-2 R_{B}, C₂-C₆alkenyl substituted with 0-2 R_{B}, C₂-C₆alkynyl substituted with 0-2 R_{B}, C₃-C₇cycloalkyl substituted with 0-2 R_{B}, (C₃-C₇cycloalkyl)C₁-C₄alkyl substituted with 0-2 R_{B}, C₁-C₆alkoxy substituted with 0-2 R_{B}, —NH(C₁-C₆alkyl) substituted with 0-2 R_{B}, —N(C₁-C₆alkyl)(C₁-C₆alkyl) each C₁-C₆alkyl independently substituted with 0-2 R_{B}, —XR_{C}, and Y;
R_{B} is independently selected at each occurrence from the group consisting of halogen, hydroxy, cyano, amino, C₁-C₄alkyl, —O(C₁-C₄alkyl), —NH(C₁-C₄alkyl), —N(C₁-C₄alkyl)(C₁-C₄alkyl), —S(O)ₙ(alkyl), halo(C₁-C₄)alkyl, halo(C₁-C₄)alkoxy, CO(C₁-C₄alkyl), CONH(C₁-C₄alkyl), CON(C₁-C₄alkyl)(C₁-C₄alkyl), —XR_{C}, and Y;
R_{C} and R_{D}, which may be the same or different, are independently selected at each occurrence from:
   hydrogen, and
   straight, branched, or cyclic alkyl groups, including (cycloalkyl)alkyl groups consisting of 1 to 8 carbon atoms, which straight, branched, or cyclic alkyl groups contain zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
X is independently selected at each occurrence from the group consisting of —O—, —C(═O)O—, —S(O)ₙ—, —NH—, —NR_{D}—, —C(═O)NH—, —C(═O)NR_{D}—, —S(O)ₙNH—, —S(O)ₙNR_{D}—, —OC(═S)S—, —NHC(═O)—, —NR_{D}C(═O)—, —NHS(O)ₙ—, —OSiH₂—, —OSiH(C₁-C₄alkyl)-, —OSi(C₁-C₄alkyl)(C₁-C₄alkyl)-, and —NR_{D}S(O)ₙ—;
Y and Z are independently selected at each occurrence from: 3- to 7-membered carbocyclic or heterocyclic groups which are saturated, unsaturated, or aromatic, which may be further substituted with one or more substituents independently selected from halogen, oxo, hydroxy, amino, cyano, C₁-C₄alkyl, —O(C₁-C₄alkyl), —NH(C₁-C₄alkyl), —N(C₁-C₄alkyl)(C₁-C₄alkyl), —C(O)(C₁-C₄alkyl), and —S(O)ₙ(alkyl), wherein said 3- to 7-memberered heterocyclic groups contain one or more heteroatom(s) independently selected from N, O, and S, with the point of attachment being either carbon or nitrogen; and
n is independently selected at each occurrence from 0, 1, and 2.

Certain preferred compounds of Formula I-a or Formula I-b include those in which at least one of Z₄ and Z₅ is not NR. Certain other preferred compounds of Formula I-a or Formula I-b include those in which Z₄ is selected from N and CR₄ and Z₅ is selected from N and CR₅.

Certain preferred compounds of Formula I-b include those compounds in which
Ar is chosen from phenyl which is mono-, di-, or tri-substituted with R_{A}, and 1-naphthyl, 2-naphthyl, pyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A}; and
R₁ and R₁″ are chosen from C₁-C₁₀alkyl, C₂-C₁₀alkenyl, C₂-C₁₀alkynyl, C₃-C₇cycloalkyl, (C₃-C₇cycloalkyl)C₁-C₄alkyl, (benzo)C₃-C₇cycloalkyl, (benzo)C₃₋₉ heterocycloalkyl, ((benzo)C₃₋₉ heterocycloalkyl)C₁-C₄alkyl, and halo(C₁-C₆)alkyl, each of which is substituted with 0, 1, 2, or 3 substituents independently chosen from halogen, hydroxy, amino, oxo, cyano, C₁-C₆alkyl, C₁-C₆alkoxy, haloC₁-C₆alkoxy,C₁-C₆alkanoyl, C₁-C₆alkanoyloxy, C₁-C₆alkoxycarbonyl, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, C₁-C₆alkylsulfonamide, C₁-C₆alkylsulfonyl, C₁-C₆alkylsulfonyloxy, C₁-C₆ hydroxyalkyl, C₁-C₆alkoxyC₁-C₆alkyl, C₁-C₆ haloalkoxy, 5 to 7 membered heteroaryl, 5 to 7 membered heterocycloalkyl, mono- and di-(C₁-C₆)alkylamino, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, mono- and di-(C₁-C₆)alkylcarbamoyl, —XR_{C} and X-Z.

As used herein the term “Formula I” is generally intended to refer to compounds of either Formula I-a or Formula I-b and subformulae thereof.

The invention further comprises methods of treating patients suffering from certain disorders with a therapeutically effective amount of at least one compound of the invention. These disorders include CNS disorders, particularly affective disorders, anxiety disorders, stress-related disorders, eating disorders and substance abuse. The patient suffering from these disorders may be a human or other animal (preferably a mammal), such as a domesticated companion animal (pet) or a livestock animal. Preferred compounds of the invention for such therapeutic purposes are those that antagonize the binding of CRF to CRF receptors (preferably CRF1, or less preferably CRF2 receptors). The ability of compounds to act as antagonists can be measured as an IC₅₀ value as described below.

According to yet another aspect, the present invention provides pharmaceutical compositions comprising compounds of Formula I or the pharmaceutically acceptable salts (by which term is also encompassed pharmaceutically acceptable solvates) thereof, which compositions are useful for the treatment of the above-recited disorders. The invention further provides methods of treating patients suffering from any of the above-recited disorders with an effective amount of a compound or composition of the invention.

Additionally this invention relates to the use of the compounds of the invention (particularly labeled compounds of this invention) as probes for the localization of receptors in cells and tissues and as standards and reagents for use in determining the receptor-binding characteristics of test compounds.

Preferred heteroaryl fused pyridine, pyrazine, and pyrimidine compounds of the invention exhibit good activity, i.e., a half-maximal inhibitory concentration (IC₅₀) of less than 1 millimolar, in a standard in vitro CRF receptor binding assay such as the assay provided in Example 51, which follows. Particularly preferred substituted heteroaryl fused pyridine, pyrazine, and pyrimidine compounds of the invention exhibit an IC₅₀of about 1 micromolar or less, still more preferably an IC₅₀ of about 100 nanomolar or less even more preferably an IC₅₀ of about 10 nanomolar or less. Certain particularly preferred compounds of the invention will exhibit an IC₅₀ of 1 nanomolar or less in such a defined standard in vitro CRF receptor binding assay.

### DETAILED DESCRIPTION OF THE INVENTION

In addition to compounds of Formula I-a, described above, the invention is further directed to compounds and pharmaceutically acceptable salts of Formula I wherein:
R is oxygen or absent;
Ar is chosen from:
phenyl which is mono-, di-, or tri-substituted with R_{A}, and 1-naphthyl, 2-naphthyl, pyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
   the group: 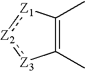
   represents a saturated, unsaturated or aromatic ring system comprising 0 or 1 heteroatoms, wherein:
Z₁ is CR₁ or CR₁R₁′;
Z₂ is nitrogen, oxygen, sulfur, CR₂, CR₂R₂′, or NR₂″,
Z₃ is nitrogen, oxygen, sulfur, sulfoxide, sulfone, CR₃, or CR₃R₃′;
R₁ is chosen from
i) halogen, hydroxy, cyano, amino, C₁-C₁₀alkyl, —O(C₁-C₆alkyl), mono or di(C₁-C₆alkyl)amino, (C₃-C₇cycloalkyl)C₁-C₄alkyl, halo(C₁-C₆)alkyl, —O(halo(C₁-C₆)alkyl) and S(O)ₙ(C₁-C₆alkyl), —O(C₃-C₇cycloalkyl)C₁-C₄alkyl, and S(O)ₙ(C₁-C₆alkyl),
   where each alkyl is independently straight, branched, or cyclic, contains zero or 1 or more double or triple bonds, and is optionally substituted with one or more substituents independently chosen from halogen, hydroxy, amino, oxo, cyano, C₁₋C₁-C₄alkoxy, and mono- or di(C₁-C₄)alkylamino,
   and
   where each C₃-C₇cycloalkyl is optionally substituted by one or more substituents independently chosen from halogen, amino, hydroxy, oxo, cyano, C₁-C₄alkoxy, and mono- or di(C₁-C₄)alkylamino, and
ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₂ and R₃ are independently chosen from hydrogen, halogen, hydroxy, amino, cyano, nitro, C₁-C₃alkyl, halo(C₁-C₃)alkyl, C₁-C₃alkoxy, amino(C₁-C₃)alkyl, and mono and di(C₁-C₆)alkylamino;
R₁′, R₂′ and R₃′ are independently chosen from hydrogen, halogen, C₁-C₆alkyl, halo(C₁-C₆)alkyl, and amino(C₁-C₆)alkyl;
R₂″ is chosen from hydrogen, C₁-C₆alkyl, halo(C₁-C₆)alkyl, and amino(C₁-C₆)alkyl;
Z₄ is NR or CR₄;
Z₅ is NR or CR₅;
R₄ and R₅ are independently chosen from hydrogen, halogen, cyano, nitro, amino, mono or di(C₁-C₆carbhydryl)amino, C₁-C₆carbhydryl, (C₃-C₇cyclocarbhydryl)C₀-C₄carbhydryl, —O(C₃-C₇cyclocarbhydryl), halo(C₁-C₆)carbhydryl, —O(halo(C₁-C₆)carbhydryl), —O(C₁-C₆carbhydryl), and S(O)ₙ(C₁-C₆carbhydryl),
   where each carbhydryl is independently straight, branched, or cyclic, contains zero or 1 or more double or triple bonds, and is optionally substituted with one or more substituents independently chosen from halogen, hydroxy, amino, oxo, cyano, C₁-C₄alkoxy, and mono- and di(C₁-C₄)alkylamino,
   and
   where each C₃-C₇carbhydryl is optionally substituted by one or more substituents independently chosen from halogen, amino, hydroxy, oxo, cyano, C₁-C₄alkoxy, and mono- and di(C₁-C₄)alkylamino;
R_{A} is independently selected at each occurrence from halogen, cyano, nitro, halo(C₁-C₆)alkyl, halo(C₁-C₆)alkoxy, hydroxy, amino, C₁-C₆alkyl substituted with 0-2 R_{B}, C₂-C₆alkenyl substituted with 0-2 R_{B}, C₂-C₆alkynyl substituted with 0-2 R_{B}, C₃-C₇cycloalkyl substituted with 0-2 R_{B}, (C₃-C₇cycloalkyl)C₁-C₄alkyl substituted with 0-2 R_{B}, C₁-C₆alkoxy substituted with 0-2 R_{B}, —NH(C₁-C₆alkyl) substituted with 0-2 R_{B}, —N(C₁-C₆alkyl)(C₁-C₆alkyl) each C₁-C₆alkyl independently substituted with 0-2 R_{B}, —XR_{C}, and Y;
R_{B} is independently selected at each occurrence from the group consisting of halogen, hydroxy, cyano, amino, C₁-C₄alkyl, —O(C₁-C₄alkyl), —NH(C₁-C₄alkyl), —N(C₁-C₄alkyl)(C₁-C₄alkyl), —S(O)ₙ(alkyl), halo(C₁-C₄)alkyl, halo(C₁-C₄)alkoxy, CO(C₁-C₄alkyl), CONH(C₁-C₄alkyl), CON(C₁-C₄alkyl)(C₁-C₄alkyl), —XR_{C}, and Y;
R_{C} and R_{D}, which may be the same or different, are independently selected at each occurrence from:
   hydrogen, and straight, branched, or cyclic alkyl groups, including (cycloalkyl)alkyl groups consisting of 1 to 8 carbon atoms, which straight, branched, or cyclic alkyl groups contain zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
X is independently selected at each occurrence from the group consisting of —CH₂—, —CHR_{D}—, —O—, —C(═O)—, —C(═O)O—, —S(O)ₙ—, —NH—, —NR_{D}—, —C(═O)NH—, —C(═O)NR_{D}—, —S(O)ₙNH—, —S(O)ₙNR_{D}—, —OC(═S)S—, —NHC(═O)—, —NR_{D}C(═O)—, —NHS(O)ₙ—, —OSiH₂—, —OSiH(C₁-C₄alkyl)-, —OSi(C₁-C₄alkyl)(C₁-C₄alkyl)-, and —NR_{D}S(O)ₙ—;
Y and Z are independently selected at each occurrence from: 3- to 7-membered carbocyclic or heterocyclic groups which are saturated, unsaturated, or aromatic, which may be further substituted with one or more substituents independently selected from halogen, oxo, hydroxy, amino, cyano, C₁-C₄alkyl, —O(C₁-C₄alkyl), —NH(C₁-C₄alkyl), —N(C₁-C₄alkyl)(C₁-C₄alkyl), and —S(O)ₙ(alkyl),
   wherein said 3- to 7-memberered heterocyclic groups contain one or more heteroatom(s) independently selected from N, O, and S, with the point of attachment being either carbon or nitrogen; and
n is independently selected at each occurrence from 0, 1, and 2. Such compounds will be referred to as compounds of Formula I-c.

Certain preferred compounds of Formula I-c include those in which at least one of Z₄ and Z₅ is not NR. Certain other preferred compounds of Formula I-c include those in which Z₄ is selected from N and CR₄ and Z₅ is selected from N and CR₅.

Particular embodiments of the invention include compounds having the following Formula:

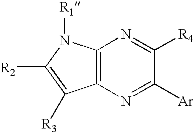

Formula II

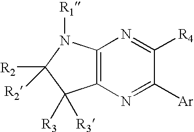

Formula III

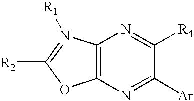

Formula IV

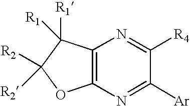

Formula V

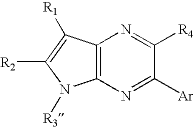

Formula VI

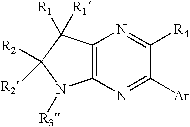

Formula VII

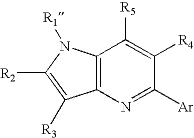

Formula VIII

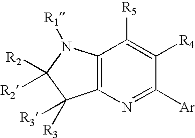

Formula IX

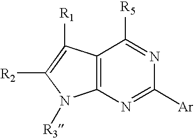

Formula X

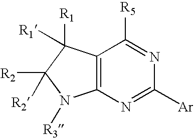

Formula XI

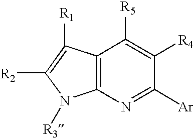

Formula XII

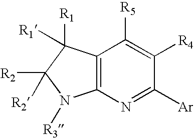

Formula XIII

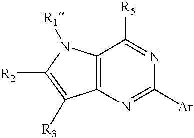

Formula XIV

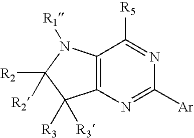

Formula XV

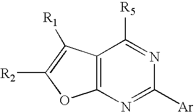

Formula XVI

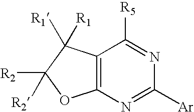

Formula XVII

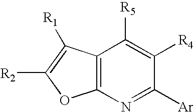

Formula XIII

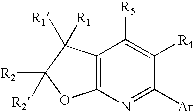

Formula XIX

For each of the compounds and salts of Formula II-Formula XIX, R₁, R₁′, R₁″, R₂, R₂′, R₂″, R₃, R₃′, R₃″, R₄, R₅, and Ar are as defined above for Formula 1, or preferably are as defined above for Formula I-a, I-b, or I-c.

More prefereably
R₁, R₁′, and R₁″ are as defined for Formula I-a, I-b, or I-c;
R₂′ and R₃′ are hydrogen;
R₂ (or R₂″) is selected from hydrogen, methyl, and ethyl;
R₃ (or R₃″) is selected from hydrogen, and C₁-C₆alkyl (or more preferably R₃ or R₃″ is C₁-C₃alkyl when Z₁ is NR₁″ or when Z₃ is NR₃″;
R₄ and R₅ are independently selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula II-Formula XX, above, is substituted.

In certain preferred compounds of Formula I (e.g., I-a and I-b) and various subformulae thereof which comprise a R₁ or R₁″ group, the R₁ or R₁″ residue is selected from C₁-C₁₀alkyl and (C₃-C₇cycloalkyl)C₀-C₄alkyl, each of which is substituted with 0 or more substituents independently chosen from halogen, hydroxy, amino, oxo, cyano, C₁-C₄alkoxy, and mono- and di-(C₁-C₄)alkylamino.

In certain other preferred compounds of Formula I (e.g., I-a and I-b) and various subformulae thereof which comprise a R₁ or R₁″ group, the R₁ or R₁″ residue is selected from C₃₋₉ heterocycloalkyl and (C₃₋₉ heterocycloalkyl)C₁₋₄alkyl, each of which is substituted with 0-4 substitutents selected from halogen, amino, hydroxy, nitro, cyano, C₁-C₆alkyl, C₁-C₆alkoxy, C₁-C₆ hydroxyalkyl, C₁-C₆alkoxyC₁-C₆alkyl, (C₁-C₆)haloalkyl, (C₁-C₆)haloalkoxy, mono- and di-(C₁-C₆)alkylamino, —XR_{C}. Certain preferred C₃₋₉ heterocycloalkyl and (C₃₋₉heterocycloalkyl)C₁₋₄alkyl groups include those chosen from tetrahydrofuranyl, tetrahydropyranyl, morpholinyl, pyrrolidinyl, piperidinyl, piperazinyl [2.2.1]-azabicyclic rings, [2.2.2]-azabicyclic rings, [3.3.1]-azabicyclic rings, quinuclidinyl, azetidinyl, azetidinonyl, oxindolyl, dihydroimidazolyl, and pyrrolidinonyl, each of which is substituted with from 0 to 2 substituents independently chosen from: (i) halogen, hydroxy, amino, cyano, or (ii) C₁-C₄alkyl, C₁-C₄alkoxy, and mono- and di-(C₁-C₄)alkylamino, each of which is substituted with 0 or 1 substituents selected from halogen, hydroxy, amino, C₁₋₂alkoxy, or C₃₋₉ heterocycloalkyl.

Certain other preferred compounds of Formula I (e.g., I-a or I-b) and compounds of Formulae II-XIX include those compounds in which R₁ or R₁″ is selected from 3-pentyl, 2-butyl, 1-methoxy-but-2-yl, 1-dimethylamino-but-2-yl, 3-(thiazol-2-yl)-1H-pyrazol-1-yl, and groups of formula:

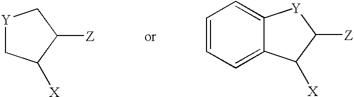

wherein X is the point of attachment to the nitrogen of the imidazo ring,

Y is selected from CH₂, O, S, S(O), SO₂, NC₁-C₈alkyl (including linear and branched alkyl groups), NC₁-C₆ haloalkyl, NC₃-C₈cycloalkyl, NC(O)C₁-C₈alkyl (including linear and branched alkyl groups), NC(O)C₁-C₆ haloalkyl, NC(O)C₃-C₈cycloalkyl, N-benzoyl, N-benzyl, NCOOC₁-C₈alkyl (including linear and branched alkyl groups), NCOOC₁-C₆ haloalkyl, NCOOC₃-C₈cycloalkyl, and

Z is selected from hydrogen, hydroxy, amino, NC₁-C₈alkyl (including linear and branched alkyl groups), NHC₁-C₆ haloalkyl, NHC₃-C₈cycloalkyl, NHC(O)C₁-C₈alkyl (including linear and branched alkyl groups), NHC(O)C₁-C₆ haloalkyl, NHC(O)C₃-C₈cycloalkyl, NH-benzoyl, NH-benzyl, NHCOOC₁-C₈alkyl (including linear and branched alkyl groups), NHCOOC₁-C₆ haloalkyl, NHCOOC₃-C₈cycloalkyl, C₁-C₈alkoxy (including linear and branched alkoxy groups), C₁-C₆ haloalkoxy, C₃-C₈cycloalkoxy, OC(O)C₁-C₈alkyl (including linear and branched alkyl groups), OC(O)C₁-C₆ haloalkyl, OC(O)C₃-C₈cycloalkyl, benzoyloxy, benzyloxy, OCONHC₁-C₈alkyl (including linear and branched alkyl groups), OCONHC₁-C₆ haloalkyl, OCONHC₃-C₈cycloalkyl, C₁-C₈alkylthio (including linear and branched alkyl groups), C₁-C₆ haloalkylthio, C₃-C₈cycloalkylthio, S(O)C₁-C₈alkyl (including linear and branched alkyl groups), S(O)C₁-C₆ haloalkyl, S(O)C₃-C₈cycloalkyl, SO₂C₁-C₈alkyl (including linear and branched alkyl groups), SO₂C₁-C₆ haloalkyl, SO₂C₃-C₈cycloalkyl.

In yet other aspects, preferred compounds of Formula I (e.g., I-a or I-b) and compounds of Formulae II-XIX include those compounds in which R₁ or R₁″ is selected from 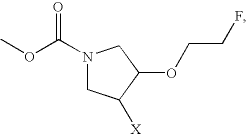
or more preferably a group of formula 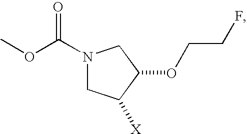
wherein X is the point of attachment to the nitrogen of the imidazo ring.

Particularly preferred R₁ groups are shown in the R₂2-Matrix and particularly preferred R₁″ groups are shown in the R₁2-Matrix, both in Example 1, which follows.

Other preferred R₁ groups include groups of the formula 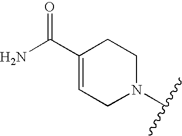
and groups of the formula 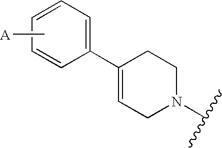
where A represents up to three groups independently chosen from hydrogen, halogen, alkyl, and alkoxy.

Another embodiment of the invention is directed to compounds of Formula XX 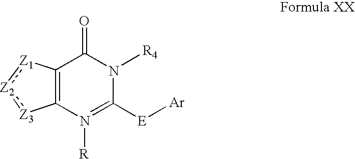
or a pharmaceutically acceptable salt thereof, wherein:
E is a single bond, O, S(O)ₘ, NR₁₀ or CR₁₀R₁₁;
R₁₀ and R₁ are independently hydrogen or C₁-C₄ alkyl;
m is 0, 1, or 2;
Ar is chosen from:
phenyl which is mono-, di-, or tri-substituted, 1-naphthyl and 2-naphthyl, each of which is optionally mono-, di-, or tri-substituted, and optionally mono-, di-, or tri-substituted heteroaryl, said heteroaryl having from 1 to 3 rings, 5 to 7 ring members in each ring and, in at least one of said rings, from 1 to about 3 heteroatoms selected from the group consisting of N, O, and S;
R is oxygen or absent;
   the group: 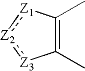
   represents a saturated, unsaturated or aromatic 5-membered ring system containing 0 or 1 heteroatoms, wherein:
Z₁ is CR₁, CR₁R₁′, or NR₁″;
Z₂ is nitrogen, oxygen, sulfur, CR₂, CR₂R₂′ or NR₂″,
Z₃ is nitrogen, oxygen, sulfur, sulfoxide, sulfone, CR₃, CR₃R₃′, or NR₃″;
R₁ is chosen from halogen, hydroxy, cyano, amino, optionally substituted alkyl, optionally substituted alkenyl, optionally substituted alkynyl, optionally substituted alkoxy, optionally substituted mono or dialkylamino, optionally substituted (cycloalkyl)alkyl, optionally substituted cycloalkyl, optionally substituted heterocycloalkyl, optionally substituted alkylthio, optionally substituted alkylsulfinyl, optionally substituted alkylsulfonyl, optionally substituted mono- or dialkylcarboxamide, optionally substituted carbocyclic aryl, and optionally substituted heteroaryl, said optionally substituted heteroaryl having from 1 to 3 rings, 5 to 7 ring members in each ring and, in at least one of said rings, from 1 to about 3 heteroatoms selected from the group consisting of N, O, and S;
R₁″ is chosen from optionally substituted alkyl, optionally substituted alkenyl, optionally substituted alkynyl, optionally substituted (cycloalkyl)alkyl, optionally substituted cycloalkyl, optionally substituted heterocycloalkyl, optionally substituted (heterocycloalkyl)alkyl, optionally substituted carbocyclic aryl, and optionally substituted heteroaryl, said optionally substituted heteroaryl having from 1 to 3 rings, 5 to 7 ring members in each ring and, in at least one of said rings, from 1 to about 3 heteroatoms selected from the group consisting of N, O, and S;
R₂ and R₃ are independently chosen from hydrogen, halogen, hydroxy, amino, cyano, nitro, alkyl, haloalkyl, alkoxy, aminoalkyl, and mono and dialkylamino;
R₁′, R₂′ and R₃′ are independently chosen from hydrogen, halogen, alkyl, haloalkyl, and aminoalkyl;
R₂″ and R₃″ are independently chosen from hydrogen, alkyl, haloalkyl, and aminoalkyl; and
R₄ is hydrogen, alkyl, aminoalkyl, and haloalkyl

Certain other preferred compounds and pharmaceutically acceptable salts of the invention include those compounds of Formula XX: 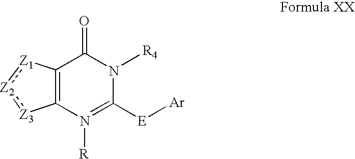
or a pharmaceutically acceptable salt thereof, wherein:
E is a single bond, O, S(O)ₘ, N R₁₀ or CR₁₀R₁₁;
R₁₀ and R₁ are independently hydrogen or C₁-C₄ alkyl;
m is 0, 1, or 2;
R is oxygen or absent;
Ar is chosen from:
phenyl which is mono-, di-, or tri-substituted, 1-naphthyl and 2-naphthyl, each of which is optionally mono-, di-, or tri-substituted, and optionally mono-, di-, or tri-substituted heteroaryl, said heteroaryl having from 1 to 3 rings, 5 to 7 ring members in each ring and, in at least one of said rings, from 1 to about 3 heteroatoms selected from the group consisting of N, O, and S;
   the group: 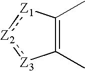
   represents a saturated, unsaturated or aromatic ring system comprising 0 or 1 heteroatoms, wherein:
Z₁ is CR₁, CR₁R₁′, or NR₁″;
Z₂ is nitrogen, oxygen, sulfur, CR₂, CR₂R₂′ or NR₂″,
Z₃ is nitrogen, oxygen, sulfur, sulfoxide, sulfone, CR₃, CR₃R₃′ or NR₃″;
R₁is chosen from
i) halogen, hydroxy, cyano, amino, C₁-C₁₀carbhydryl, —O(C₁-C₆carbhydryl), mono or di(C₁-C₆carbhydryl)amino, (C₃-C₇cyclocarbhydryl)C₁-C₄carbhydryl, halo(C₁-C₆)carbhydryl, —O(halo(C₁-C₆)carbhydryl) and S(O)ₙ(C₁-C₆carbhydryl), —O(C₃-C₇cyclocarbhydryl)C₁-C₄carbhydryl, C₃₋₉ heterocycloalkyl, (C₃₋₉ heterocycloalkyl)C₁-C₄alkyl, and S(O)ₙ(C₁-C₆carbhydryl),
   where each carbhydryl is independently straight, branched, or cyclic, contains zero or 1 or more double or triple bonds,
   where each heterocycloalkyl has 1 or 2 ring heteroatoms selected from N, O, or S and
the point of attachment is carbon or nitrogen; and
   where each carbhydryl, heterocycloalkyl, or cyclocarbhydryl is optionally substituted by one or more substituents independently chosen from halogen, hydroxy, amino, oxo, cyano, C₁-C₆alkyl, C₁-C₆alkoxy, haloC₁-C₆alkoxy,C₁-C₆alkanoyl, C₁-C₆alkanoyloxy, C₁-C₆alkoxycarbonyl, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, C₁-C₆alkylsulfonamide, C₁-C₆alkylsulfonyl, C₁-C₆alkylsulfonyloxy, C₁-C₆ hydroxyalkyl, C₁-C₆alkoxyC₁-C₆alkyl, C₁-C₆ haloalkoxy, 5 to 7 membered heteroaryl, 5 to 7 membered heterocycloalkyl, mono- and di-(C₁-C₆)alkylamino, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, mono- and di-(C₁-C₆)alkylcarbamoyl, —XR_{C} and X-Z, and
ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₁″ is chosen from C₁-C₁₀alkyl, C₂-C₁₀alkenyl, C₂-C₁₀alkynyl, C₃-C₇cycloalkyl, (C₃-C₇cycloalkyl)C₁-C₄alkyl, C₃₋₉ heterocycloalkyl, (C₃₋₉ heterocycloalkyl)C₁-C₄alkyl and halo(C₁-C₆)alkyl, each of which is substituted with 0 or more substituents independently chosen from halogen, hydroxy, amino, oxo, cyano, C₁-C₆alkyl, C₁-C₆alkoxy, haloC₁-C₆alkoxy,C₁-C₆alkanoyl, C₁-C₆alkanoyloxy, C₁-C₆alkoxycarbonyl, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, C₁-C₆alkylsulfonamide, C₁-C₆alkylsulfonyl, C₁-C₆alkylsulfonyloxy, C₁-C₆ hydroxyalkyl, C₁-C₆alkoxyC₁-C₆alkyl, C₁-C₆ haloalkoxy, 5 to 7 membered heteroaryl, 5 to 7 membered heterocycloalkyl, mono- and di-(C₁-C₆)alkylamino, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, mono- and di-(C₁-C₆)alkylcarbamoyl, —XR_{C} and X-Z;
R₂ and R₃ are independently chosen from hydrogen, halogen, hydroxy, amino, cyano, nitro, C₁-C₆alkyl, halo(C₁-C₆)alkyl, C₁-C₆alkoxy, amino(C₁-C₆)alkyl, and mono and di(C₁-C₆)alkylamino;
R₂′ and R₃′ are independently chosen from hydrogen, halogen, C₁-C₆alkyl, halo(C₁-C₆)alkyl, and amino(C₁-C₆)alkyl;
R₂″ and R₃″ are independently chosen from hydrogen, C₁-C₆alkyl, halo(C₁-C₆)alkyl, and amino(C₁-C₆)alkyl;
R₄ is hydrogen, C₁-C₆alkyl, C₁-C₆aminoalkyl, and C₁-C₆ haloalkyl
R_{A} is independently selected at each occurrence from halogen, cyano, nitro, halo(C₁-C₆)alkyl, halo(C₁-C₆)alkoxy, hydroxy, amino, C₁-C₆alkyl substituted with 0-2 R_{B}, C₂-C₆alkenyl substituted with 0-2 R_{B}, C₂-C₆alkynyl substituted with 0-2 R_{B}, C₃-C₇cycloalkyl substituted with 0-2 R_{B}, (C₃-C₇cycloalkyl)C₁-C₄alkyl substituted with 0-2 R_{B}, C₁-C₆alkoxy substituted with 0-2 R_{B}, —NH(C₁-C₆alkyl) substituted with 0-2 R_{B}, —N(C₁-C₆alkyl)(C₁-C₆alkyl) each C₁-C₆alkyl independently substituted with 0-2 R_{B}, —XR_{C}, and Y;
R_{B} is independently selected at each occurrence from the group consisting of halogen, hydroxy, cyano, amino, C₁-C₄alkyl, —O(C₁-C₄alkyl), —NH(C₁-C₄alkyl), —N(C₁-C₄alkyl)(C₁-C₄alkyl), —S(O)ₙ(alkyl), halo(C₁-C₄)alkyl, halo(C₁-C₄)alkoxy, CO(C₁-C₄alkyl), CONH(C₁-C₄alkyl), CON(C₁-C₄alkyl)(C₁-C₄alkyl), —XR_{C}, and Y;
R_{C} and R_{D}, which may be the same or different, are independently selected at each occurrence from:
   hydrogen, and
   straight, branched, or cyclic alkyl groups, including (cycloalkyl)alkyl groups consisting of 1 to 8 carbon atoms, which straight, branched, or cyclic alkyl groups contain zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
X is independently selected at each occurrence from the group consisting of —CH₂—, —CHR_{D}—, —O—, —C(═O)—, —C(═O)O—, —S(O)ₙ—, —NH—, —NR_{D}—, —C(═O)NH—, —C(═O)NR_{D}—, —S(O)ₙNH—, —S(O)ₙNR_{D}—, —OC(═S)S—, —NHC(═O)—, —NR_{D}C(═O)—, —NH S(O)ₙ—, —OSiH₂—, —OSiH(C₁-C₄alkyl)-, —OSi(C₁-C₄alkyl)(C₁-C₄alkyl)-, and —NR_{D}S(O)ₙ—;
Y and Z are independently selected at each occurrence from: 3- to 7-membered carbocyclic or heterocyclic groups which are saturated, unsaturated, or aromatic, which may be further substituted with one or more substituents independently selected from halogen, oxo, hydroxy, amino, cyano, C₁-C₄alkyl, —O(C₁-C₄alkyl), —C(O)(C₁-C₄alkyl), —NH(C₁-C₄alkyl), —N(C₁-C₄alkyl)(C₁-C₄alkyl), and —S(O)ₙ(alkyl),
   wherein said 3- to 7-memberered heterocyclic groups contain one or more heteroatom(s) independently selected from N, O, and S, with the point of attachment being either carbon or nitrogen; and
n is independently selected at each occurrence from 0, 1, and 2.

Preferred compounds and pharmaceutically acceptable salts of Formula XX are those for which:
Ar is chosen from:
phenyl which is mono-, di-, or tri-substituted with R_{A}, and 1-naphthyl, 2-naphthyl, pyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
   the group: 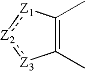
   represents a saturated, unsaturated or aromatic ring system comprising 0 or 1 heteroatoms, wherein:
Z₁ is CR₁, CR₁R₁′or NR₁″;
Z₂ is nitrogen, oxygen, sulfur, CR₂, CR₂R₂′ or NR₂″,
Z₃ is nitrogen, oxygen, sulfur, sulfoxide, sulfone, CR₃, CR₃R₃′ or NR₃″;
R₁ is chosen from
i) halogen, hydroxy, cyano, amino, C₁-C₁₀carbhydryl, —O(C₁-C₆ carbhydryl), mono or di(C₁-C₆carbhydryl)amino, (C₃-C₇cycloalkyl)C₁-C₄carbhydryl, halo(C, C₆)carbhydryl, —O(halo(C₁-C₆)carbhydryl) and S(O)ₙ(C₁-C₆carbhydryl), —O(C₃-C₇cycloalkyl)C₁-C₄carbhydryl, and S(O)ₙ(C₁-C₆carbhydryl),
   where each carbhydryl is independently straight, branched, or cyclic, contains zero or 1 or more double or triple bonds, and is optionally substituted with one or more substituents independently chosen from halogen, hydroxy, amino, oxo, cyano, C, C₁-C₄alkoxy, and mono- or di(C₁-C₄)alkylamino,
   and
   where each C₃-C₇cycloalkyl is optionally substituted by one or more substituents independently chosen from halogen, amino, hydroxy, oxo, cyano, C₁-C₄alkoxy, and mono- or di(C₁-C₄)alkylamino,
and
ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₁″ is chosen from C₁-C₁₀carbhydryl, (C₃-C₇cycloalkyl)C₁-C₄carbhydryl, halo(C₁-C₆)carbhydryl,
   where each carbhydryl is independently straight, branched, or cyclic, contains zero or 1 or more double or triple bonds, and is optionally substituted with one or more substituents independently chosen from halogen, hydroxy, amino, oxo, cyano, C₁-C₄alkoxy, and mono- or di(C₁-C₄)alkylamino,
   and
   where each C₃-C₇cycloalkyl is optionally substituted by one or more substituents independently chosen from halogen, amino, hydroxy, oxo, cyano, C₁-C₄alkoxy, and mono- or di(C₁-C₄)alkylamino,
and
ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₂ and R₃ are independently chosen from hydrogen, halogen, hydroxy, amino, cyano, nitro, C₁-C₆alkyl, halo(C₁-C₆)alkyl, C₁-C₆alkoxy, amino(C₁-C₆)alkyl, and mono and di(C₁-C₆)alkylamino;
R₂′ and R₃′ are independently chosen from hydrogen, halogen, C₁-C₆alkyl, halo(C₁-C₆)alkyl, and amino(C₁-C₆)alkyl;
R₂″ and R₃″ are independently chosen from hydrogen, C₁-C₆alkyl, halo(C₁-C₆)alkyl, and amino(C₁-C₆)alkyl;
R₄ is hydrogen or C₁-C₆alkyl;
R_{A} is independently selected at each occurrence from halogen, cyano, nitro, halo(C₁-C₆)alkyl, halo(C₁-C₆)alkoxy, hydroxy, amino, C₁-C₆alkyl substituted with 0-2 R_{B}, C₂-C₆alkenyl substituted with 0-2 R_{B}, C₂-C₆alkynyl substituted with 0-2 R_{B}, C₃-C₇cycloalkyl substituted with 0-2 R_{B}, (C₃-C₇cycloalkyl)C₁-C₄alkyl substituted with 0-2 R_{B}, C₁-C₆alkoxy substituted with 0-2 R_{B}, —NH(C₁-C₆alkyl) substituted with 0-2 R_{B}, —N(C₁-C₆alkyl)(C₁-C₆alkyl) each C₁-C₆alkyl independently substituted with 0-2 R_{B}, —XR_{C}, and Y;
R_{B} is independently selected at each occurrence from the group consisting of halogen, hydroxy, cyano, amino, C₁-C₄alkyl, —O(C₁-C₄alkyl), —NH(C₁-C₄alkyl), —N(C₁-C₄alkyl)(C₁-C₄alkyl), —S(O)ₙ(alkyl), halo(C₁-C₄)alkyl, halo(C₁-C₄)alkoxy, CO(C₁-C₄alkyl), CONH(C₁-C₄alkyl), CON(C₁-C₄alkyl)(C₁-C₄alkyl), —XR_{C}, and Y;
R_{C} and R_{D}, which may be the same or different, are independently selected at each occurrence from:
   hydrogen, and
   straight, branched, or cyclic alkyl groups, including (cycloalkyl)alkyl groups consisting of 1 to 8 carbon atoms, which straight, branched, or cyclic alkyl groups contain zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
X is independently selected at each occurrence from the group consisting of —CH₂—, —CHR_{D}—, —O—, —C(═O)—, —C(═O)O—, —S(O)ₙ—, —NH—, —NR_{D}—, —C(═O)NH—, —C(═O)NR_{D}—, —S(O)ₙNH—, —S(O)ₙNR_{D}—, —OC(═S)S—, —NHC(═O)—, —NR_{D}C(═O)—, —NHS(O)ₙ—, —OSiH₂—, —OSiH(C₁-C₄alkyl)-, —OSi(C₁-C₄alkyl)(C₁-C₄alkyl)-, and —NR_{D}S(O)ₙ—;
Y and Z are independently selected at each occurrence from: 3- to 7-membered carbocyclic or heterocyclic groups which are saturated, unsaturated, or aromatic, which may be further substituted with one or more substituents independently selected from halogen, oxo, hydroxy, amino, cyano, C₁-C₄alkyl, —O(C₁-C₄alkyl), —NH(C₁-C₄alkyl), —N(C₁-C₄alkyl)(C₁-C₄alkyl), and —S(O)ₙ(alkyl),
   wherein said 3- to 7-memberered heterocyclic groups contain one or more heteroatom(s) independently selected from N, O, and S, with the point of attachment being either carbon or nitrogen; and
n is independently selected at each occurrence from 0, 1, and 2. Such compounds will be referred to as compounds of Formula XXA.

The invention is particularly directed to compounds and salts of the following Formula: 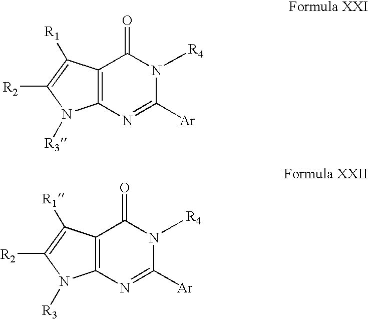
Preferred compounds and salts of Formula XXI and Formula XXII
R₁ or R₁″ are as defined for Formula XX, or preferably as defined for Formula XXA.
R₂ is selected from hydrogen, methyl, and ethyl;
R₃ is selected from hydrogen and C₁-C₆alkyl; and
Ar is selected from the group consisting of phenyl, pyridyl which is mono- di- or trisubstituted with substituents independently chosen from halogen, cyano, nitro, halo(C₁-C₆)alkyl, halo(C₁-C₆)alkoxy, hydroxy, amino, C₁-C₆alkyl, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XXI or XXII is substituted.

Compounds of the invention are useful in treating a variety of conditions including affective disorders, anxiety disorders, stress disorders, eating disorders, and drug addiction.

Affective disorders include all types of depression, bipolar disorder, cyclothymia, and dysthymia.

Anxiety disorders include generalized anxiety disorder, panic, phobias and obsessive-compulsive disorder.

Stress-related disorders include post-traumatic stress disorder, hemorrhagic stress, stress-induced psychotic episodes, psychosocial dwarfism, stress headaches, stress-induced immune systems disorders such as stress-induced fever, and stress-related sleep disorders.

Eating disorders include anorexia nervosa, bulimia nervosa, and obesity.

Modulators of the CRF receptors are also useful in the treatment (e.g., symptomatic treatment)of a variety of neurological disorders including supranuclear palsy, AIDS related dementias, multiinfarct dementia, neurodegenerative disorders such as Alzheimer's disease, Parkinson's disease, and Huntington's disease, head trauma, spinal cord trauma, ischemic neuronal damage, amyotrophic lateral sclerosis, disorders of pain perception such as fibromyalgia and epilepsy.

Additionally compounds of Formula I are useful as modulators of the CRF receptor in the treatment (e.g., symptomatic treatment) of a number of gastrointestinal, cardiovascular, hormonal, autoimmune and inflammatory conditions. Such conditions include irritable bowel syndrome, ulcers, Crohn's disease, spastic colon, diarrhea, post operative ilius and colonic hypersensitivity associated with psychopathological disturbances or stress, hypertension, tachycardia, congestive heart failure, infertility, euthyroid sick syndrome, inflammatory conditions effected by rheumatoid arthritis and osteoarthritis, pain, asthma, psoriasis and allergies.

Compounds of Formula I are also useful as modulators of the CRF1 receptor in the treatment of animal disorders associated with aberrant CRF levels. These conditions include porcine stress syndrome, bovine shipping fever, equine paroxysmal fibrillation, and dysfunctions induced by confinement in chickens, sheering stress in sheep or human-animal interaction related stress in dogs, psychosocial dwarfism and hypoglycemia.

Typical subjects to which compounds of the invention may be administered will be mammals, particularly primates, especially humans. For veterinary applications, a wide variety of subjects will be suitable, e.g. livestock such as cattle, sheep, goats, cows, swine and the like; poultry such as chickens, ducks, geese, turkeys, and the like; and other domesticated animals particularly pets such as dogs and cats. For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids (e.g., blood, plasma, serum, CSF, lymph, cellular interstitial fluid, aqueous humor, saliva, synovial fluid, feces, or urine) and cell and tissue samples of the above subjects will be suitable for use..

The CRF binding compounds provided by this invention and labeled derivatives thereof are also useful as standards and reagents in determining the ability of test compounds (e.g., a potential pharmaceutical) to bind to a CRF receptor.

Labeled derivatives the CRF antagonist compounds provided by this invention are also useful as radiotracers for positron emission tomography (PET) imaging or for single photon emission computerized tomography (SPECT).

More particularly compounds of the invention may be used for demonstrating the presence of CRF receptors in cell or tissue samples. This may be done by preparing a plurality of matched cell or tissue samples, at least one of which is prepared as an experiment sample and at least one of which is prepared as a control sample. The experimental sample is prepared by contacting (under conditions that permit binding of CRF to CRF receptors within cell and tissue samples) at least one of the matched cell or tissue samples that has not previously been contacted with any compound or salt of the invention with an experimental solution comprising the detectably-labeled preparation of the selected compound or salt at a first measured molar concentration. The control sample is prepared by in the same manner as the experimental sample and is incubated in a solution that contains the same ingredients as the experimental solution but that also contains an unlabelled preparation of the same compound or salt of the invention at a molar concentration that is greater than the first measured molar concentration.

The experimental and control samples are then washed to remove unbound detectably-labeled compound. The amount of detectably-labeled compound remaining bound to each sample is then measured and the amount of detectably-labeled compound in the experimental and control samples is compared. A comparison that indicates the detection of a greater amount of detectable label in the at least one washed experimental sample than is detected in any of the at least one washed control samples demonstrates the presence of CRF receptors in that experimental sample.

The detectably-labeled compound used in this procedure may be labeled with any detectable label, such as a radioactive label, a biological tag such as biotin (which can be detected by binding to detectably-labeled avidin), an enzyme (e.g., alkaline phosphatase, beta galactosidase, or a like enzyme that can be detected its activity in a calorimetric assay) or a directly or indirectly luminescent label. When tissue sections are used in this procedure and the detectably-labeled compound is radiolabeled, the bound, labeled compound may be detected autoradiographically to generate an autoradiogram. When autoradiography is used, the amount of detectable label in an experimental or control sample may be measured by viewing the autoradiograms and comparing the exposure density of the autoradiograms.

The present invention also pertains to methods of inhibiting the binding of CRF to CRF receptors (preferably CRF1 receptors) which methods involve contacting a solution containing a CRF antagonist compound of the invention with cells expressing CRF receptors, wherein the compound is present in the solution at a concentration sufficient to inhibit CRF binding to CRF receptors in vitro. This method includes inhibiting the binding of CRF to CRF receptors in vivo, e.g., in a patient given an amount of a compound of Formula I that would be sufficient to inhibit the binding of CRF to CRF receptors in vitro. In one embodiment, such methods are useful in treating physiological disorders associated with excess concentrations of CRF. The amount of a compound that would be sufficient to inhibit the binding of a CRF to the CRF receptor may be readily determined via a CRF receptor binding assay (see, e.g., Example 51), or from the EC₅₀ of a CRF receptor functional assay, such as a standard assay of CRF receptor mediated chemotaxis. The CRF receptors used to determine in vitro binding may be obtained from a variety of sources, for example from cells that naturally express CRF receptors, e.g. IMR₃₂ cells or from cells expressing cloned human CRF receptors.

The present invention also pertains to methods for altering the activity of CRF receptors, said method comprising exposing cells expressing such receptors to an effective amount of a compound of the invention, wherein the compound is present in the solution at a concentration sufficient to specifically alter the signal transduction activity in response to CRF in cells expressing CRF receptors in vitro, preferred cells for this purpose are those that express high levels of CRF receptors (i.e., equal to or greater than the number of CRF1 receptors per cell found in differentiated IMR-32 human neuroblastoma cells), with IMR-32 cells being particularly preferred for testing the concentration of a compound required to alter the activity of CRF1 receptors. This method includes altering the signal transduction activity of CRF receptors in vivo, e.g., in a patient given an amount of a compound of Formula I that would be sufficient to alter the signal transduction activity in response to CRF in cells expressing CRF receptors in vitro. The amount of a compound that would be sufficient to alter the signal transduction activity in response to CRF of CRF receptors may also be determined via an assay of CRF receptor mediated signal transduction, such as an assay wherein the binding of CRF to a cell surface CRF receptor effects a changes in reporter gene expression.

The present invention also pertains to packaged pharmaceutical compositions for treating disorders responsive to CRF receptor modulation, e.g., eating disorders, depression or stress. The packaged pharmaceutical compositions include a container holding a therapeutically effective amount of at least one CRF1 receptor modulator as described supra and instructions for using the treating disorder responsive to CRF 1 receptor modulation in the patient.

Chemical Description and Terminology

The compounds herein described may have one or more asymmetric centers or planes. Compounds of the present invention containing an asymmetrically substituted atom may be isolated in optically active or racemic forms. It is well known in the art how to prepare optically active forms, such as by resolution of racemic forms (racemates), by asymmetric synthesis, or by synthesis from optically active starting materials. Resolution of the racemates can be accomplished, for example, by conventional methods such as crystallization in the presence of a resolving agent, or chromatography, using, for example a chiral HPLC column. Many geometric isomers of olefins, C=N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral (enantiomeric and diastereomeric), and racemic forms, as well as all geometric isomeric forms of a structure are intended, unless the specific stereochemistry or isomeric form is specifically indicated.

When any variable occurs more than one time in any constituent or formula for a compound, its definition at each occurrence is independent of its definition at every other occurrence. Thus, for example, if a group is shown to be substituted with 0-2 R*, then said group may optionally be substituted with up to two R* groups and R* at each occurrence is selected independently from the definition of R*. Also, combinations of substituents and/or variables are permissible only if such combinations result in stable compounds.

Formula I includes, but is not limited to, compounds of Formula I, IA, and II-XXII. As indicated above, various substituents of the various formulae (compounds of Formula I, I, IA, and II-XXII) are “optionally substituted”, including Ar¹, Ar², R¹, R², and R³ of Formula I and subformulae thereof, and such substituents as recited in the sub-formulae such as Formula I and subformulae. The term “substituted,” as used herein, means that any one or more hydrogens on the designated atom or group is replaced with a selection from the indicated group of substituents, provided that the designated atom's normal valence is not exceeded, and that the substitution results in a stable compound. When a substituent is oxo (keto, i.e., ═O), then 2 hydrogens on an atom are replaced. The present invention is intended to include all isotopes (including radioisotopes) of atoms occurring in the present compounds.

When substituents such as Ar, R₁, R₂, R₃, R₄, and R₅ are further substituted, they may be so substituted at one or more available positions, typically 1 to 3 or 4 positions, by one or more suitable groups such as those disclosed herein. Suitable groups that may be present on a “substituted” Ar, R₁, R₂, R₃, R₄, and R₅ or other group include e.g., halogen; cyano; hydroxyl; nitro; azido; alkanoyl (such as a C₁-C₆ alkanoyl group such as acyl or the like); carboxamido; alkyl groups (including cycloalkyl groups, having 1 to about 8 carbon atoms, preferably 1, 2, 3, 4, 5, or 6 carbon atoms); alkenyl and alkynyl groups (including groups having one or more unsaturated linkages and from 2 to about 8, preferably 2, 3, 4, 5 or 6, carbon atoms); alkoxy groups having one or more oxygen linkages and from 1 to about 8, preferably 1, 2, 3, 4, 5 or 6 carbon atoms; aryloxy such as phenoxy; alkylthio groups including those having one or more thioether linkages and from 1 to about 8 carbon atoms, preferably 1, 2, 3, 4, 5 or 6 carbon atoms; alkylsulfinyl groups including those having one or more sulfinyl linkages and from 1 to about 8 carbon atoms, preferably 1, 2, 3, 4, 5, or 6 carbon atoms; alkylsulfonyl groups including those having one or more sulfonyl linkages and from 1 to about 8 carbon atoms, preferably 1, 2, 3, 4, 5, or 6 carbon atoms; aminoalkyl groups including groups having one or more N atoms and from 1 to about 8, preferably 1, 2, 3, 4, 5 or 6, carbon atoms; carbocyclic aryl having 6 or more carbons and one or more rings, (e.g., phenyl, biphenyl, naphthyl, or the like, each ring either substituted or unsubstituted aromatic); arylalkyl having 1 to 3 separate or fused rings and from 6 to about 18 ring carbon atoms, with benzyl being a preferred arylalkyl group; arylalkoxy having 1 to 3 separate or fused rings and from 6 to about 18 ring carbon atoms, with 0-benzyl being a preferred arylalkoxy group; or a saturated, unsaturated, or aromatic heterocyclic group having 1 to 3 separate or fused rings with 3 to about 8 members per ring and one or more N, O or S atoms, e.g. coumarinyl, quinolinyl, isoquinolinyl, quinazolinyl, pyridyl, pyrazinyl, pyrimidyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl, morpholinyl, piperazinyl, and pyrrolidinyl. Such heterocyclic groups may be further substituted, e.g. with hydroxy, alkyl, alkoxy, halogen and amino.

As used herein, “alkyl” is intended to include both branched and straight-chain saturated aliphatic hydrocarbon groups, having the specified number of carbon atoms. Examples of alkyl include, but are not limited to, methyl, ethyl, n-propyl, i-propyl, n-butyl, s-butyl, t-butyl, n-pentyl, and s-pentyl. Preferred alkyl groups are C₁-C₁₀ alkyl groups. Especially preferred alkyl groups are methyl, ethyl, propyl, butyl, and 3-pentyl. The term C, 4 alkyl as used herein includes alkyl groups consisting of 1 to 4 carbon atoms, which may contain a cyclopropyl moiety. Suitable examples are methyl, ethyl, and cyclopropylmethyl.

The term “carbhydryl” refers to both branched and straight-chain hydrocarbon groups, which are saturated or unsaturated. In other words, a carbhydryl group may be alkyl, alkenyl or alkynyl. The number of carbon atoms may be specified as indicated above.

“Cycloalkyl” is intended to include saturated ring groups, having the specified number of carbon atoms, such as cyclopropyl, cyclobutyl, cyclopentyl, or cyclohexyl. Cycloalkyl groups typically will have 3 to about 8 ring members.

In the term “(C₃-C₇cycloalkyl)C₁-C₄alkyl”, cycloalkyl, and alkyl are as defined above, and the point of attachment is on the alkyl group. This term encompasses, but is not limited to, cyclopropylmethyl, cyclohexylmethyl, and cyclohexylmethyl.

“Alkenyl” is intended to include hydrocarbon chains of either a straight or branched configuration comprising one or more unsaturated carbon-carbon bonds, which may occur in any stable point along the chain, such as ethenyl and propenyl. Alkenyl groups typically will have 2 to about 8 carbon atoms, more typically 2 to about 6 carbon atoms.

“Alkynyl” is intended to include hydrocarbon chains of either a straight or branched configuration comprising one or more carbon-carbon triple bonds, which may occur in any stable point along the chain, such as ethynyl and propynyl. Alkynyl groups typically will have 2 to about 8 carbon atoms, more typically 2 to about 6 carbon atoms. carbhydryl is independently straight, branched, or cyclic, contains zero or 1 or more double or triple bonds.

“Haloalkyl” is intended to include both branched and straight-chain saturated aliphatic hydrocarbon groups having the specified number of carbon atoms, substituted with 1 or more halogen atoms. Examples of haloalkyl include, but are not limited to, mono-, di-, or tri-fluoromethyl, mono-, di-, or tri-chloromethyl, mono-, di-, tri-, tetra-, or penta-fluoroethyl, and mono-, di-, tri-, tetra-, or penta-chloroethyl. Typical haloalkyl groups will have 1 to about 8 carbon atoms, more typically 1 to about 6 carbon atoms.

“Alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Alkoxy groups typically have 1 to about 8 carbon atoms, more typically 1 to about 6 carbon atoms.

“Halolkoxy” represents a haloalkyl group as defined above with the indicated number of carbon atoms attached through an oxygen bridge.

As used herein, the term “alkylthio” includes those groups having one or more thioether linkages and preferably from 1 to about 8 carbon atoms, more typically 1 to about 6 carbon atoms.

As used herein, the term “alkylsulfinyl” includes those groups having one or more sulfoxide (SO) linkage groups and typically from 1 to about 8 carbon atoms, more typically I to about 6 carbon atoms.

As used herein, the term “alkylsulfonyl” includes those groups having one or more sulfonyl (SO₂) linkage groups and typically from 1 to about 8 carbon atoms, more typically I to about 6 carbon atoms.

As used herein, the term “alkylamino” includes those groups having one or more primary, secondary and/or tertiary amine groups and typically from 1 to about 8 carbon atoms, more typically 1 to about 6 carbon atoms.

“Halo” or “halogen” as used herein refers to fluoro, chloro, bromo, or iodo; and “counter-ion” is used to represent a small, negatively charged species such as chloride, bromide, hydroxide, acetate, sulfate, and the like.

As used herein, “carbocyclic group” is intended to mean any stable 3- to 7-membered monocyclic or bicyclic or 7- to 13-membered bicyclic or tricyclic group, any of which may be saturated, partially unsaturated, or aromatic. In addition to those exemplified elsewhere herein, examples of such carbocycles include, but are not limited to, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, phenyl, naphthyl, indanyl, and tetrahydronaphthyl.

As used herein, the term “heterocyclic group” is intended to include saturated, partially unsaturated, or unsaturated (aromatic) groups having 1 to 3 (preferably fused) rings with 3 to about 8 members per ring at least one ring containing an atom selected from N, O or S. The nitrogen and sulfur heteroatoms may optionally be oxidized The term or “heterocycloalkyl” is used to refer to saturated heterocyclic groups having one or more non-carbon ring atoms (e.g., N, O, S, P, Si, or the like) and a specified number of carbon atoms. Thus, a C₃₋₉ heterocycloalkyl is a cyclic group having between 3 and 9 ring carbon atoms and at least one ring heteroatom.

The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. As used herein, the term “aromatic heterocyclic system” is intended to include any stable 5- to 7-membered monocyclic or 10- to 14-membered bicyclic heterocyclic aromatic ring system which comprises carbon atoms and from 1 to 4 heteroatoms independently selected from the group consisting of N, O and S. It is preferred that the total number of S and 0 atoms in the aromatic heterocycle is not more than 2, more preferably not more than 1.

Examples of heterocycles include, but are not limited to, those exemplified elsewhere herein and further include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; 1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

Preferred heterocyclic groups include, but are not limited to, pyridinyl, pyrimidinyl, furanyl, thienyl, pyrrolyl, pyrazolyl, pyrrolidinyl, morpholinyl, piperidinyl, piperazinyl, and imidazolyl. Also included are fused ring and spiro compounds containing, for example, the above heterocycles.

As used herein, the term “carbocyclic aryl” includes groups that contain 1 to 3 separate or fused rings and from 6 to about 18 ring atoms, without hetero atoms as ring members. Specifically preferred carbocyclic aryl groups include phenyl, and naphthyl including 1-napthyl and 2-naphthyl.

As used herein, “pharmaceutically acceptable salts” refer to derivatives of the disclosed compounds wherein the parent compound is modified by making non-toxic acid or base salts thereof, and further refers to pharmaceutically acceptable solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, malefic, hydroxymaleic, phenylacetic, glutamic, benzoic, salicylic, mesylic, sulfanilic, 2-acetoxybenzoic, fumaric, toluenesulfonic, methanesulfonic, ethane disulfonic, oxalic, isethionic, HOOC—(CH₂)n-COOH where n is 0-4, and the like. The pharmaceutically acceptable salts of the present invention can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts may be found, e.g., in Remington's Pharmaceutical Sciences, 17th ed., Mack Publishing Company, Easton, Pa., p. 1418 (1985).

“Prodrugs” are intended to include any compounds that become compounds of Formula I when administered to a mammalian subject, e.g., upon metabolic processing of the prodrug. Examples of prodrugs include, but are not limited to, acetate, formate and benzoate and like derivatives of functional groups (such as alcohol or amine groups) in the compounds of Formula I.

Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent. The term “therapeutically effective amount” of a compound of this invention means an amount effective, when administered to a human or non-human patient, to provide a therapeutic benefit such as an amelioration of symptoms, e.g., an amount effective to antagonize the effects of pathogenic levels of CRF or to treat the symptoms of stress disorders, affective disorder, anxiety or depression.

Pharmaceutical Preparations

The compounds of general Formula I may be administered orally, topically, transdermally, parenterally, by inhalation or spray or rectally or vaginally in dosage unit formulations containing conventional non-toxic pharmaceutically acceptable carriers, adjuvants and vehicles. The term parenteral as used herein includes subcutaneous, intravenous, intramuscular, intrathecal and like types of injection or infusion techniques. In addition, there is provided a pharmaceutical formulation comprising a compound of general Formula I and a pharmaceutically acceptable carrier. One or more compounds of general Formula I may be present in association with one or more non-toxic pharmaceutically acceptable carriers and/or diluents and/or adjuvants and if desired other active ingredients. The pharmaceutical compositions containing compounds of general Formula I may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be sterile injectable solution or suspension in a non-toxic parentally acceptable dilutent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of general Formula I may also be administered in the form of suppositories, e.g., for rectal administration of the drug. These compositions can be prepared by mixing the drug with a suitable non-irritating excipient that is solid at ordinary temperatures but liquid at body temperature and will therefore melt in the body to release the drug. Such materials include cocoa butter and polyethylene glycols.

Compounds of general Formula I may be administered parenterally in a sterile medium. The drug, depending on the vehicle and concentration used, can either be suspended or dissolved in the vehicle. Advantageously, one or more adjuvants such as preservatives, buffering agents, or local anesthetics can also be present in the vehicle.

Dosage levels of the order of from about 0.05 mg to about 100 mg per kilogram of body weight per day are useful in the treatment of the above-indicated conditions, preferred dosages range from about 0.1 to about 30 mg per kg and more preferably from about 0.5 to about 5 mg per kg per subject per day. The amount of active ingredient that may be combined with the carrier materials to produce a single dosage form will vary depending upon the host treated and the particular mode of administration. Dosage unit forms will generally contain between from about 0.1 mg to about 750 mg of an active ingredient.

Frequency of dosage may also vary depending on the compound used and the particular disease treated. However, for treatment of most CNS and gastrointestinal disorders, a dosage regimen of four times daily, preferably three times daily, more preferably two times daily and most preferably once daily is contemplated. For the treatment of stress and depression a dosage regimen of 1 or 2 times daily is particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination (i.e. other drugs being used to treat the patient) and the severity of the particular disease undergoing therapy.

Preferred compounds of the invention will have certain pharmacological properties. Such properties include, but are not limited to oral bioavailability, such that the preferred oral dosage forms discussed above can provide therapeutically effective levels of the compound in vivo. Penetration of the blood brain barrier is necessary for most compounds used to treat CNS disorders, while low brain levels of compounds used to treat periphereal disorders are generally preferred.

Assays may be used to predict these desirable pharmacological properties. Assays used to predict bioavailability include transport across human intestinal cell monolayers, including Caco-2 cell monolayers. Toxicity to cultured hepatocyctes may be used to predict compound toxicity, with non-toxic compounds being preferred. Penetration of the blood brain barrier of a compound in humans may be predicted from the brain levels of the compound in laboratory animals given the compound, e.g., intravenously.

Percentage of serum protein binding may be predicted from albumin binding assays. Examples of such assays are described in a review by Oravcová, et al. (Journal of Chromatography B (1996) volume 677, pages 1-27). Preferred compounds exhibit reversible serum protein binding. Preferably this binding is less than 99%, more preferably less than 95%, even more preferably less than 90%, and most preferably less than 80%.

Frequency of administration is generally inversely proportional to the in vivo half-life of a compound. In vivo half-lives of compounds may be predicted from in vitro assays of microsomal half-life as described by Kuhnz and Gieschen (Drug Metabolism and Disposition, (1998) volume 26, pages 1120-1127). Preferred half lives are those allowing for a preferred frequency of administration.

As discussed above, preferred compounds of the invention exhibit good activity in standard in vitro CRF receptor binding assays, preferably the assay as specified in Example 51, which follows. References herein to “standard in vitro receptor binding assay” are intended to refer to standard assay protocols such as that protocol defined in Example 51, which follows. Generally preferred compounds of the invention have an IC₅₀ (half-maximal inhibitory concentration) of about 1 micromolar or less, still more preferably and IC₅₀ of about 100 nanomolar or less even more preferably an IC₅₀ of about 10 nanomolar or less or even 1 nanomolar or less in such a defined standard in vitro CRF receptor binding assay as exemplified by Example 51 which follows.

### EXAMPLES

Preparation of Compounds

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include but are not limited to those methods described below. Each of the references cited below are hereby incorporated herein by reference. Preferred methods for the preparation of compounds of the present invention include, but are not limited to, those described in Scheme I. Those who are skilled in the art will recognize that the starting materials may be varied and additional steps employed to produce compounds encompassed by the present invention. 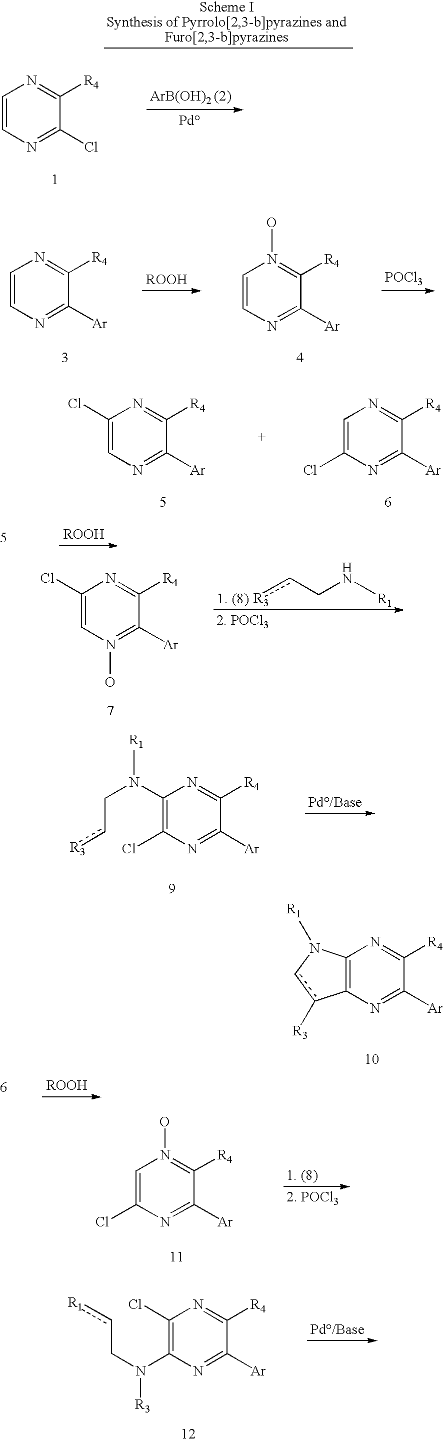 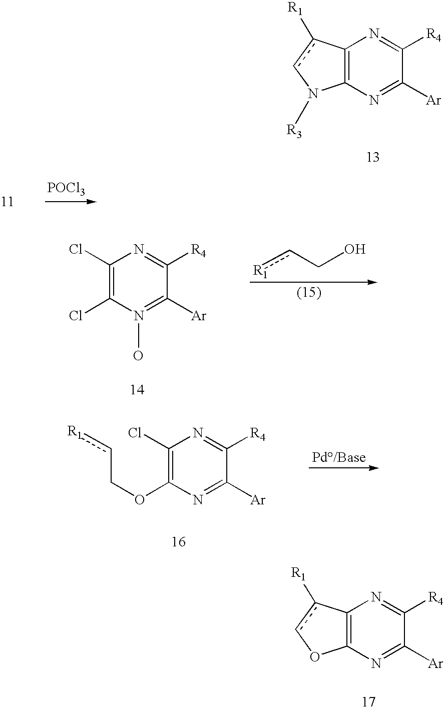 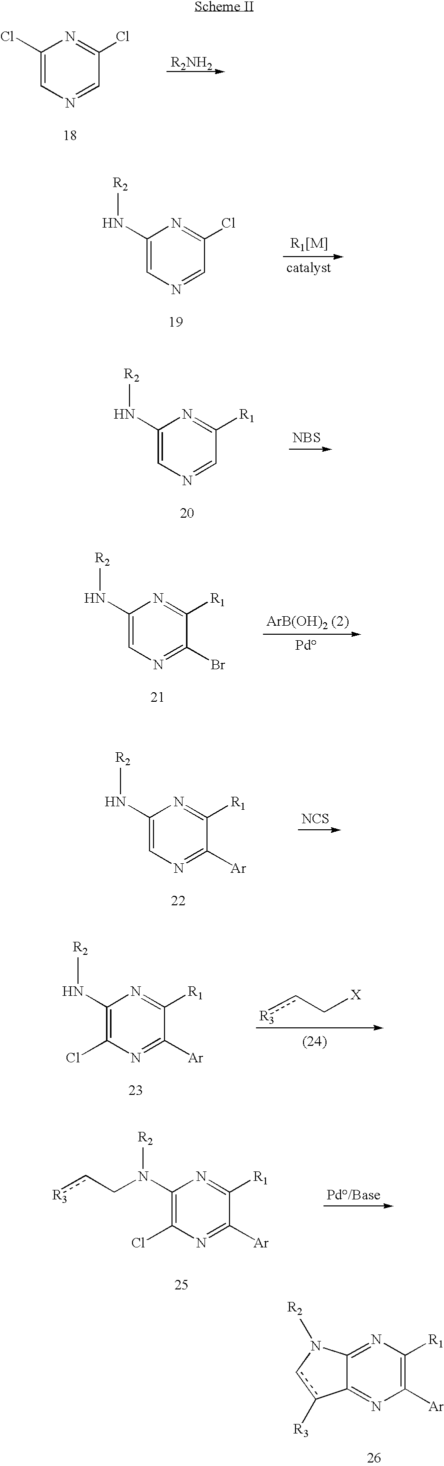 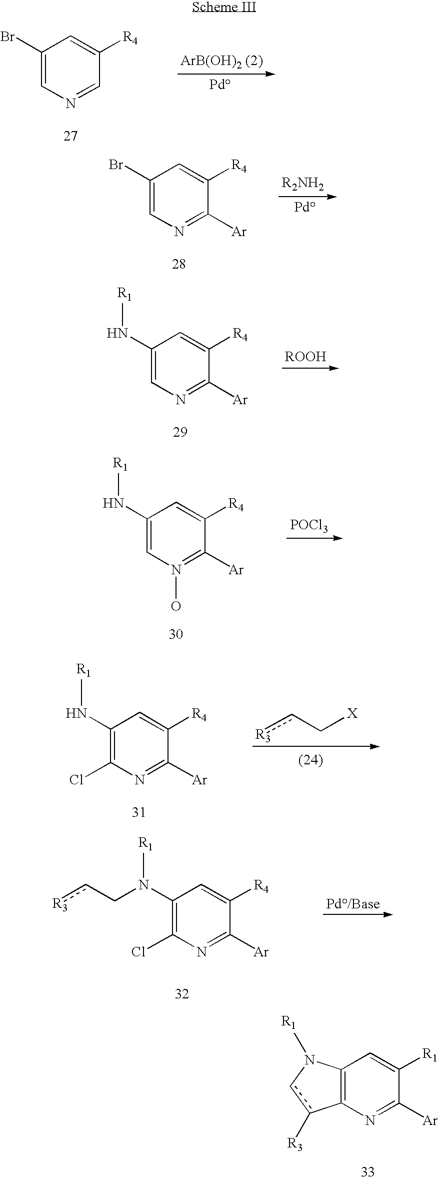 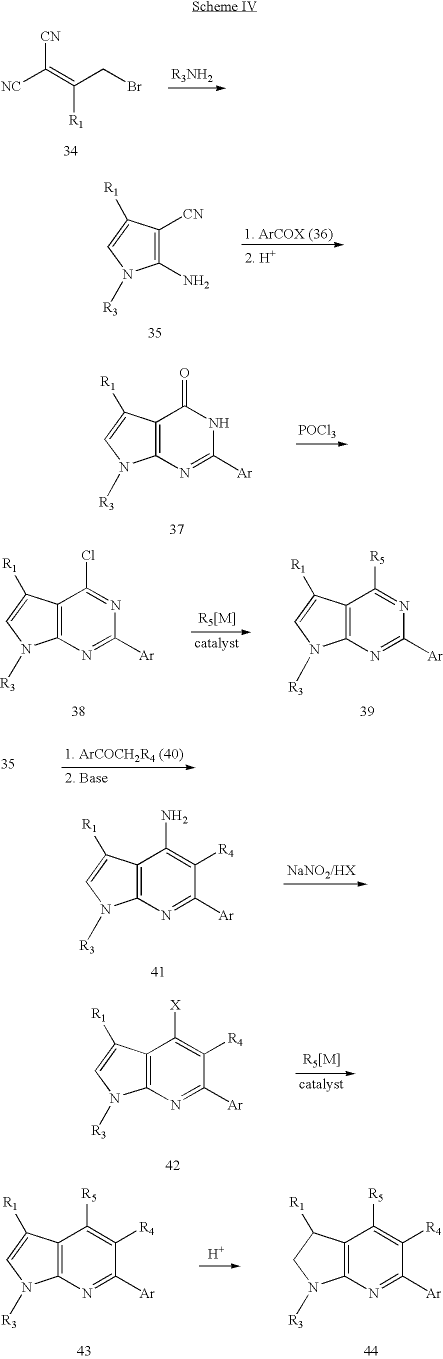 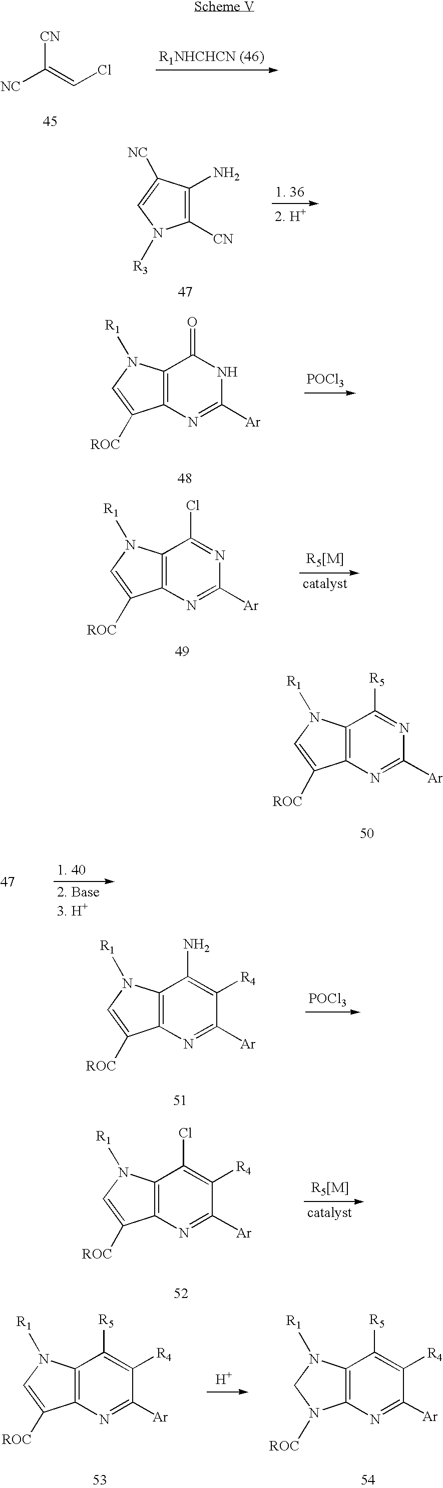 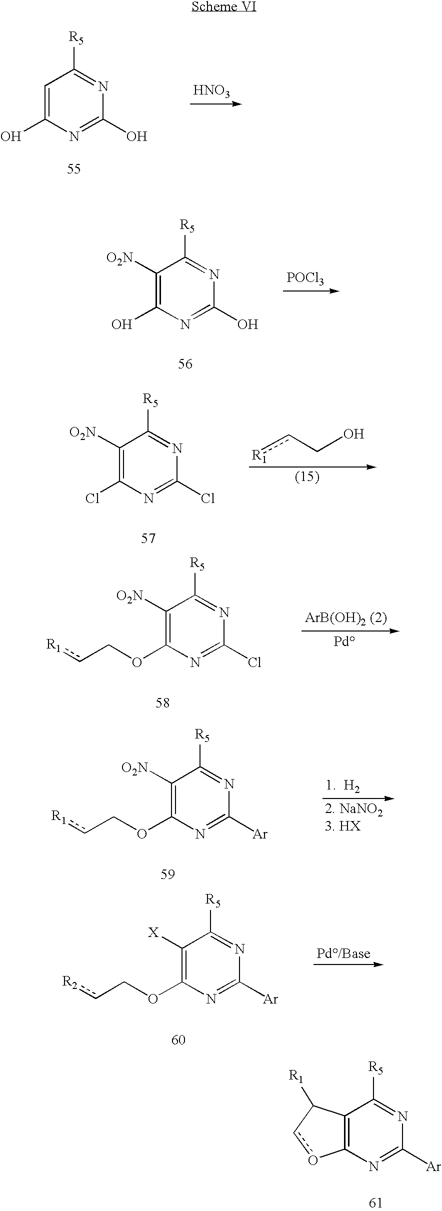 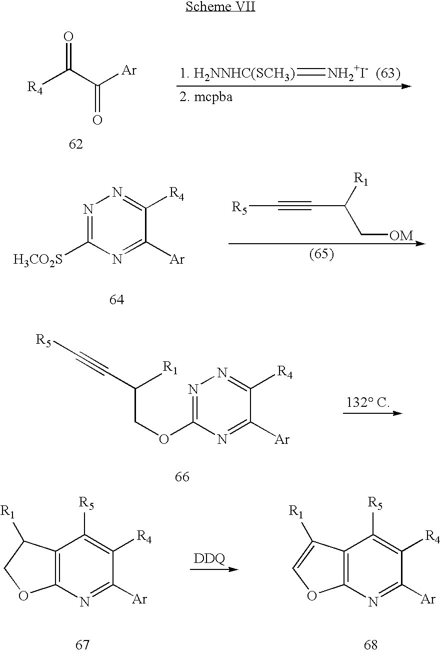
Exemplified Compounds of the Invention

The R₂1-Matrix, R₂2-Matrix, Het-Matrix, and Ar-Matrix tables below set forth a number of compounds of the invention which are prepared by the methods illustrated in Reaction Schemes I-VII shown above. Compounds are formed by combining any element from the R₂1 Matrix or R₂2-Matrix with any element from the Het-matrix to form an R₂1-Het or R₂2 moiety, and then combining this moiety with any element of the Ar-Matrix to form a compound of the invention. For example, the combination of element 101 from the R₂1-Matrix, with element 408 from the Het-matrix, gives the moiety 101408. This moiety is then combined with element 504 from the Ar-matrix, to form a compound of the invention, compound 101408504, which is 3-(2,4-Dimethoxy-phenyl)-2-ethyl-7-(1-ethyl-propyl)-furo[2,3-b]pyrazine.

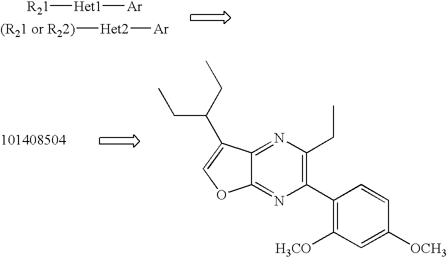

R₂1-Matrix

| 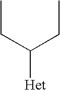 | 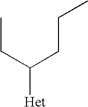 | 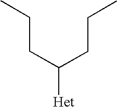 | 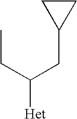 | 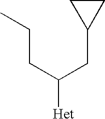 |
|---|---|---|---|---|
| 101 | 102 | 103 | 104 | 105 |
| 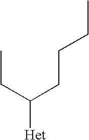 | 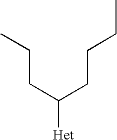 | 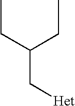 | 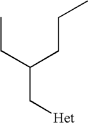 | 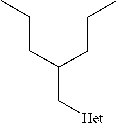 |
| 106 | 107 | 108 | 109 | 110 |
| 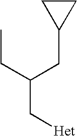 | 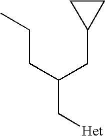 | 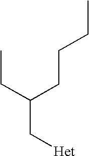 | 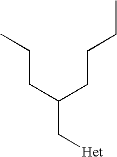 | 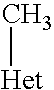 |
| 111 | 112 | 113 | 114 | 115 |
| 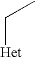 | 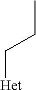 | 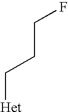 | 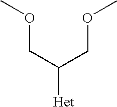 | 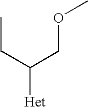 |
| 116 | 117 | 118 | 119 | 120 |
| 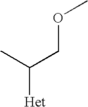 | 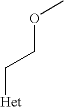 | 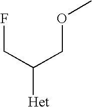 | 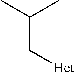 | 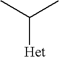 |
| 121 | 122 | 123 | 124 | 125 |
| 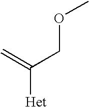 | 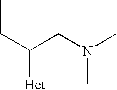 | 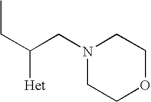 | 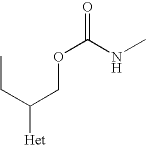 | 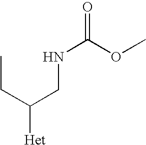 |
| 126 | 127 | 128 | 129 | 130 |
| 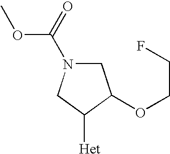 | 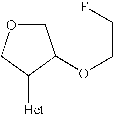 | 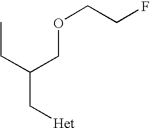 | 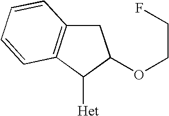 | 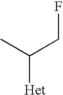 |
| 131 | 132 | 133 | 134 | 135 |
| 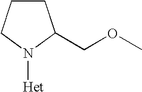 | 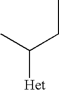 | 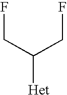 | 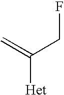 | 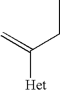 |
| 136 | 137 | 138 | 139 | 140 |

R₂2-Matrix

| | | | | |
|---|---|---|---|---|
| 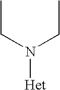 | 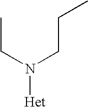 | 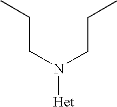 | 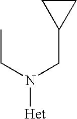 | 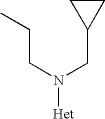 |
| 201 | 202 | 203 | 204 | 205 |
| 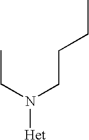 | 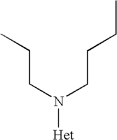 | 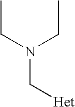 | 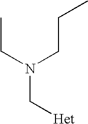 | 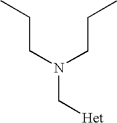 |
| 206 | 207 | 208 | 209 | 210 |
| 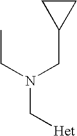 | 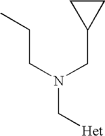 | 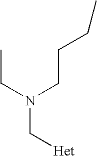 | 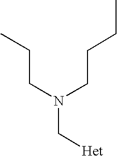 | 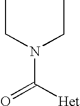 |
| 211 | 212 | 213 | 214 | 215 |
| 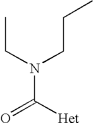 | 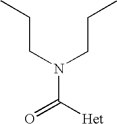 | 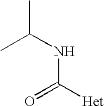 | 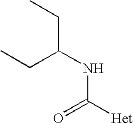 | 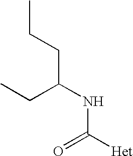 |
| 216 | 217 | 218 | 219 | 220 |
| 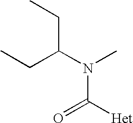 | 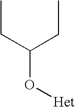 | 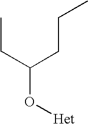 | 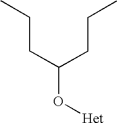 | 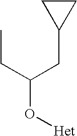 |
| 221 | 222 | 223 | 224 | 225 |
| 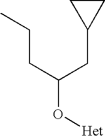 | 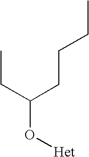 | 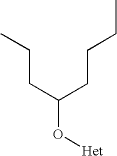 | 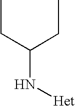 | 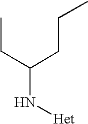 |
| 226 | 227 | 228 | 229 | 230 |
| 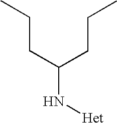 | 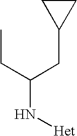 | 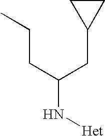 | 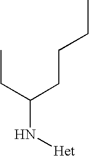 | 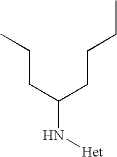 |
| 231 | 232 | 233 | 234 | 235 |
| 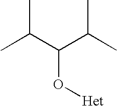 | 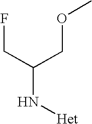 | 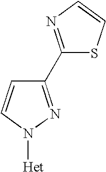 | 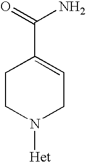 | 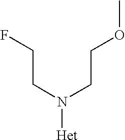 |
| 236 | 237 | 238 | 239 | 240 |

Het1-Matrix

| | | | |
|---|---|---|---|
| 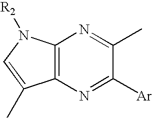 | 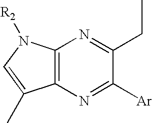 | 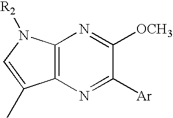 | 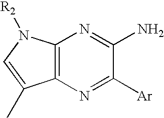 |
| 301 | 302 | 303 | 304 |
| 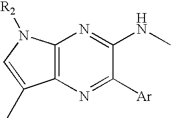 | 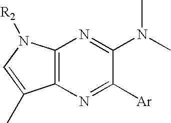 | 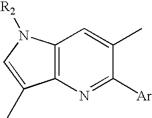 | 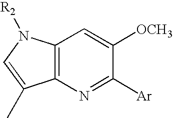 |
| 305 | 306 | 307 | 308 |
| 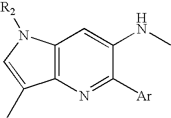 | 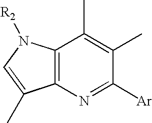 | 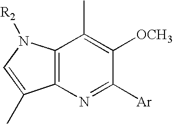 | 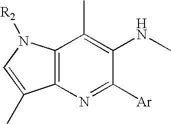 |
| 309 | 310 | 311 | 312 |
| 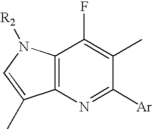 | 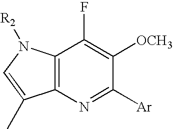 | 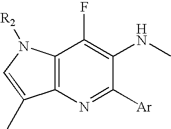 | 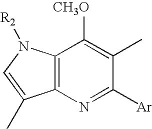 |
| 313 | 314 | 315 | 316 |
| 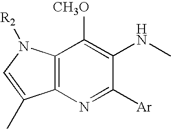 | 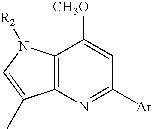 | 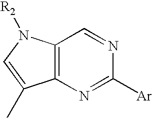 | 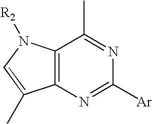 |
| 317 | 318 | 319 | 320 |
| 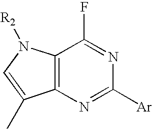 | 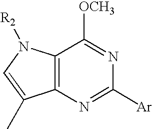 | 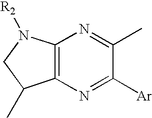 | 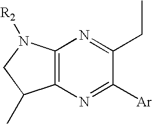 |
| 321 | 322 | 323 | 324 |
| 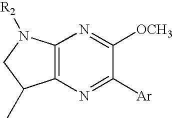 | 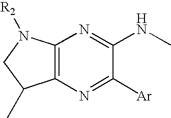 | 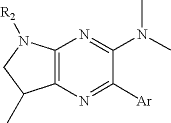 | 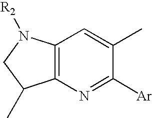 |
| 325 | 326 | 327 | 328 |
| 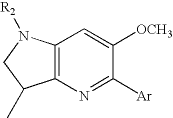 | 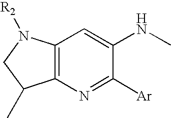 | 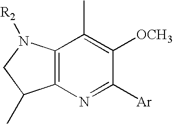 | 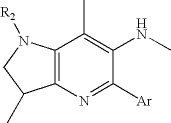 |
| 329 | 330 | 331 | 332 |
| 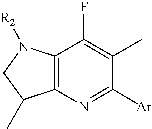 | 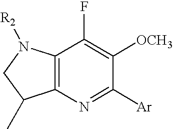 | 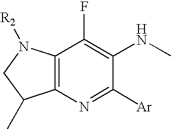 | 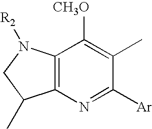 |
| 333 | 334 | 335 | 336 |
| 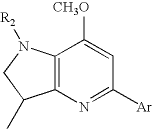 | 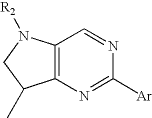 | 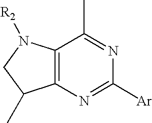 | 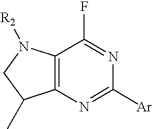 |
| 337 | 338 | 339 | 340 |
| 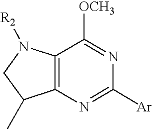 | 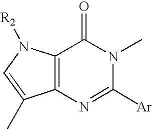 | 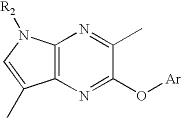 | 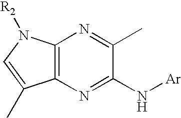 |
| 341 | 342 | 343 | 344 |
| 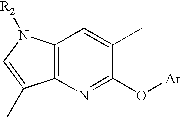 | 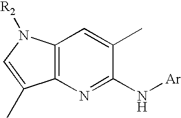 | 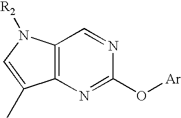 | 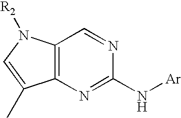 |
| 345 | 346 | 347 | 348 |

Het2-Matrix

| | | | |
|---|---|---|---|
| 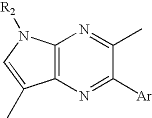 | 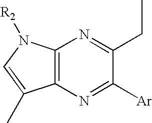 | 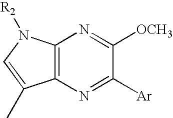 | 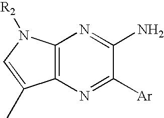 |
| 401 | 402 | 403 | 404 |
| 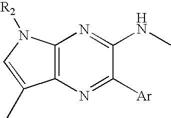 | 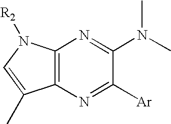 | 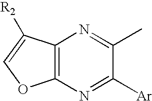 | 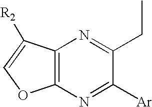 |
| 405 | 406 | 407 | 408 |
| 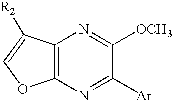 | 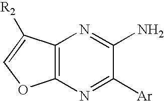 | 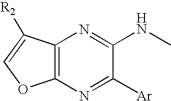 | 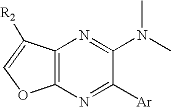 |
| 409 | 410 | 411 | 412 |
| 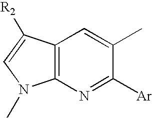 | 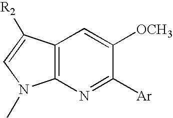 | 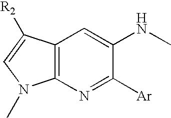 | 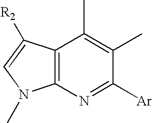 |
| 413 | 414 | 415 | 416 |
| 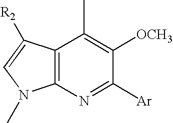 | 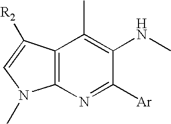 | 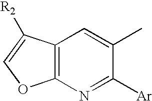 | 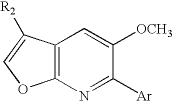 |
| 417 | 418 | 419 | 420 |
| 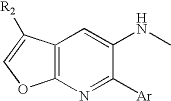 | 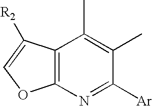 | 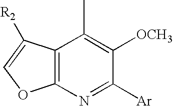 | 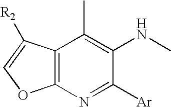 |
| 421 | 422 | 423 | 424 |
| 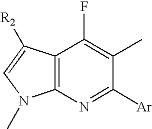 | 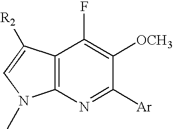 | 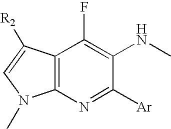 | 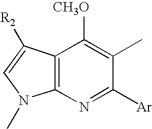 |
| 425 | 426 | 427 | 428 |
| 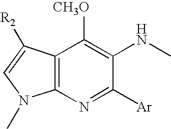 | 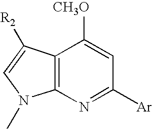 | 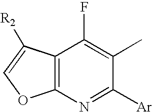 | 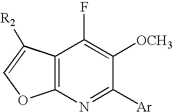 |
| 429 | 430 | 431 | 432 |
| 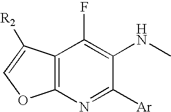 | 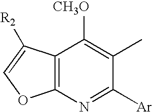 | 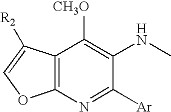 | 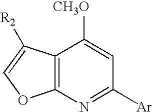 |
| 433 | 434 | 435 | 436 |
| 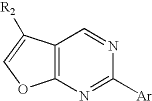 | 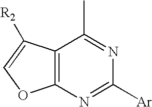 | 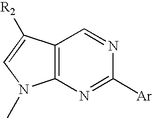 | 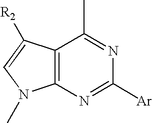 |
| 437 | 438 | 439 | 440 |
| 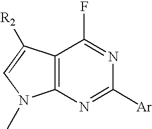 | 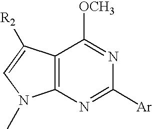 | 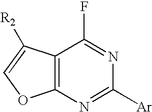 | 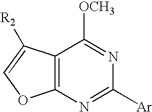 |
| 441 | 442 | 443 | 444 |
| 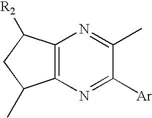 | 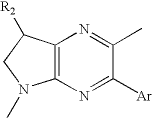 | 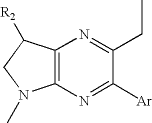 | 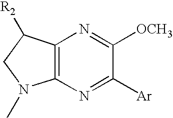 |
| 445 | 446 | 447 | 448 |
| 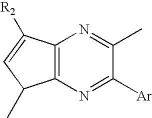 | 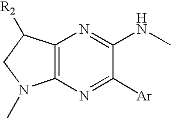 | 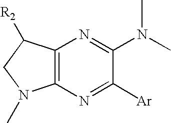 | 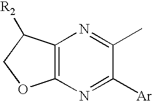 |
| 449 | 450 | 451 | 452 |
| 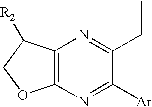 | 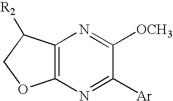 | 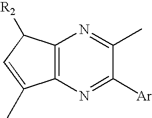 | 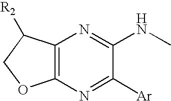 |
| 453 | 454 | 455 | 456 |
| 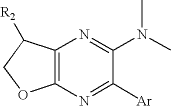 | 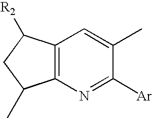 | 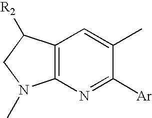 | 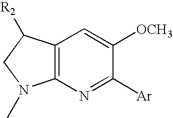 |
| 457 | 458 | 459 | 460 |
| 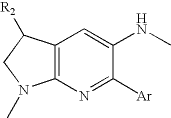 | 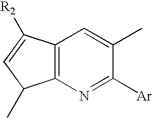 | 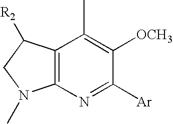 | 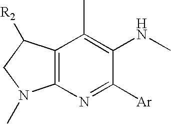 |
| 461 | 462 | 463 | 464 |
| 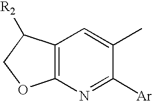 | 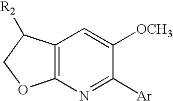 | 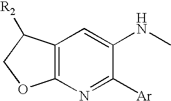 | 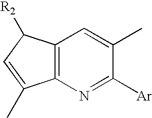 |
| 465 | 466 | 467 | 468 |
| 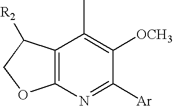 | 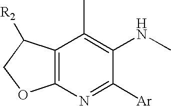 | 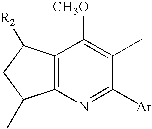 | 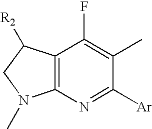 |
| 469 | 470 | 471 | 472 |
| 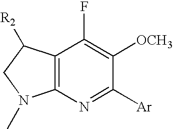 | 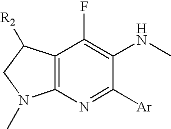 | 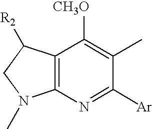 | 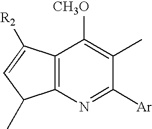 |
| 473 | 474 | 475 | 476 |
| 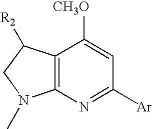 | 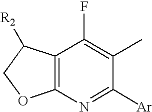 | 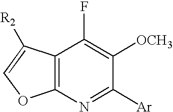 | 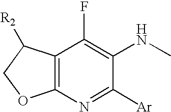 |
| 477 | 478 | 479 | 480 |
| 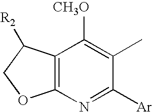 | 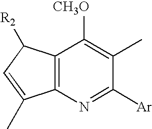 | 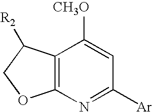 | 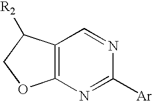 |
| 481 | 482 | 483 | 484 |
| 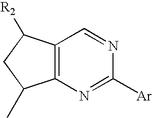 | 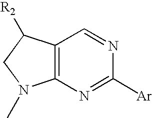 | 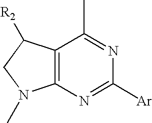 | 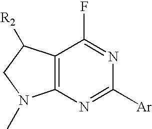 |
| 485 | 486 | 487 | 488 |
| 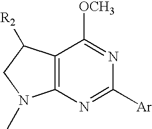 | 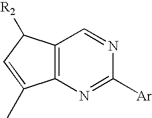 | 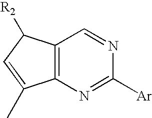 | 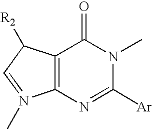 |
| 489 | 490 | 491 | 492 |
| 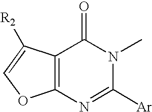 | 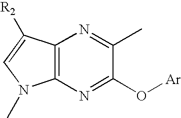 | 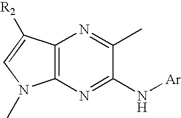 | 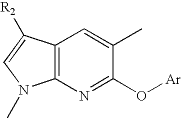 |
| 493 | 494 | 495 | 496 |
| 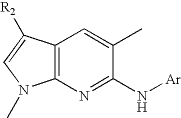 | 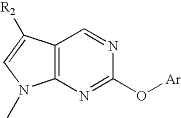 | 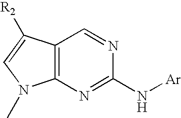 | 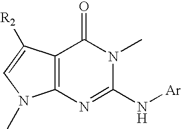 |
| 497 | 498 | 499 | 500 |

Ar-Matrix

| 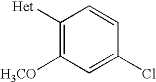 | 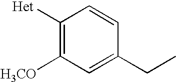 | 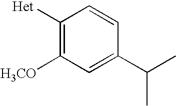 |
|---|---|---|
| 501. | 502. | 503. |
| 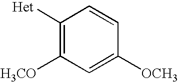 | 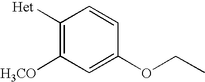 | 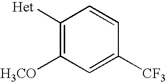 |
| 504. | 505. | 506. |
| 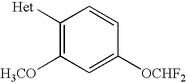 | 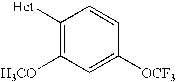 | 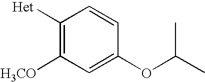 |
| 507. | 508. | 509. |
| 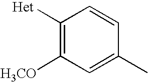 | 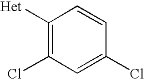 | 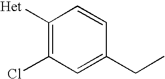 |
| 510. | 511. | 512. |
| 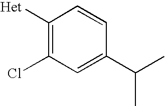 | 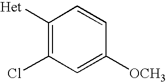 | 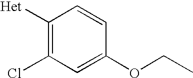 |
| 513. | 514. | 515. |
| 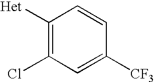 | 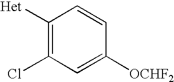 | 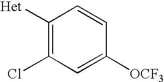 |
| 516. | 517. | 518. |
| 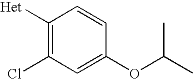 | 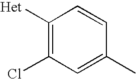 | 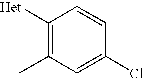 |
| 519. | 520. | 521. |
| 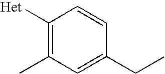 | 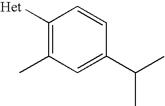 | 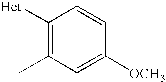 |
| 522. | 523. | 524. |
| 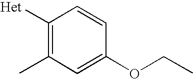 | 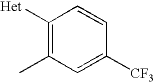 | 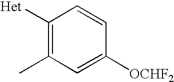 |
| 525. | 526. | 527. |
| 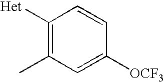 | 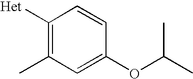 | 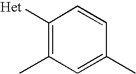 |
| 528. | 529. | 530. |
| 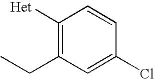 | 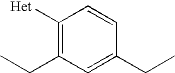 | 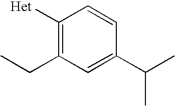 |
| 531. | 532. | 533. |
| 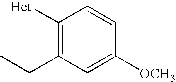 | 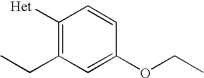 | 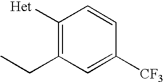 |
| 534. | 535. | 536. |
| 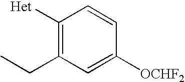 | 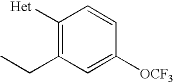 | 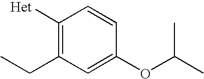 |
| 537. | 538. | 539. |
| 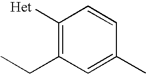 | 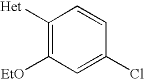 | 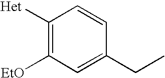 |
| 540. | 541. | 542. |
| 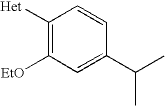 | 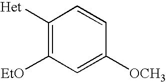 | 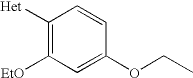 |
| 543. | 544. | 545. |
| 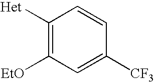 | 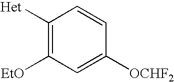 | 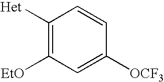 |
| 546. | 547. | 548. |
| 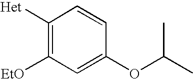 | 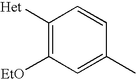 | 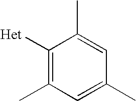 |
| 549. | 550. | 551. |
| 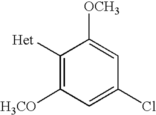 | 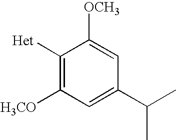 | 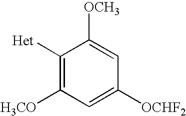 |
| 552. | 553. | 554. |
| 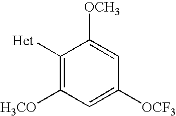 | 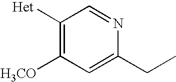 | 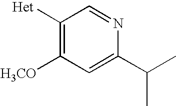 |
| 555. | 556. | 557. |
| 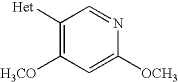 | 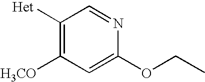 | 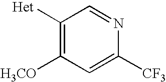 |
| 558. | 559. | 560. |
| 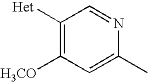 | 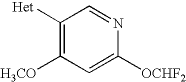 | 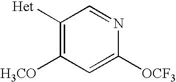 |
| 561. | 562. | 563. |
| 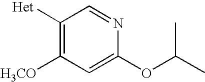 | 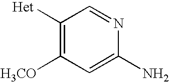 | 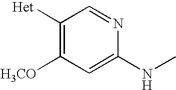 |
| 564. | 565. | 566. |
| 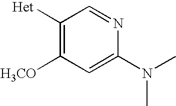 | 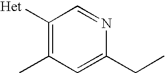 | 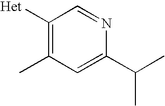 |
| 567. | 568. | 569. |
| 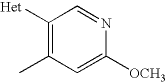 | 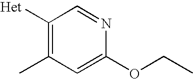 | 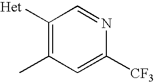 |
| 570. | 571. | 572. |
| 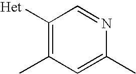 | 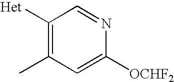 | 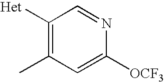 |
| 573. | 574. | 575. |
| 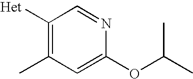 | 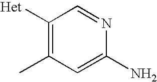 | 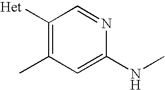 |
| 576. | 577. | 578. |
| 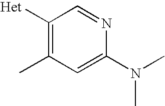 | 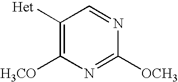 | 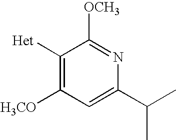 |
| 579. | 580. | 581. |
| 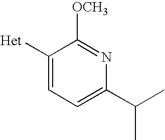 | 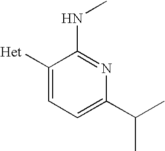 | 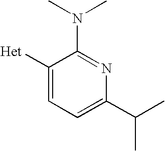 |
| 582. | 583. | 584. |
| 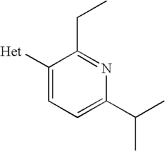 | 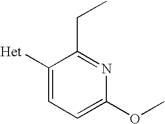 | 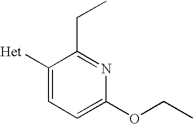 |
| 585. | 586. | 587. |
| 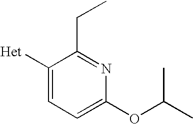 | 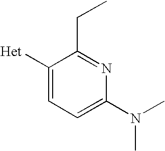 | 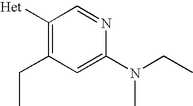 |
| 588. | 589. | 590. |
| 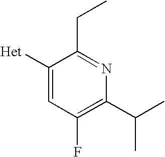 | 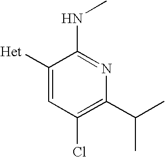 | 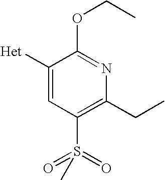 |
| 591. | 592. | 593. |
| 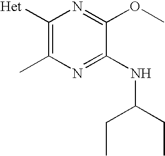 | 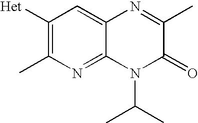 | 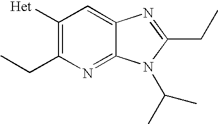 |
| 594. | 595. | 596. |

All compounds listed below are characterized at least by ¹H-NMR and LCMS. One of the following LCMS methods is used for the compounds shown below.

Method 1.

HPLC instrumentation: Analyses are performed using a Waters 600 series pump (Waters Corp.), a Waters 996 Diode Array detector and a Gilson 215 auto-sampler (Gilson Inc.). Data are acquired using MassLynx 4.0 software, with OpenLynx processing.

HPLC conditions: 4.6×50 mm, XTerra MS C18, 5 mm column (Waters Corp.); UV 10 spectra/sec, 220, 254 nm; flow rate 4.0 mL/min; injection volume 1-10 μl; Gradient conditions—Mobile phase A 95% Water, 5% Methanol with 0.05% Formic acid; Mobile phase B 95% methanol, 5% Water with 0.025% Formic acid;

| Gradient: | Time (min) | % B |
|---|---|---|
| | 0 | 5 |
| | 0.01 | 5 |
| | 1.0 | 100 |
| | 2.0 | 100 |
| | 2.1 | 5 |

MS instrumentation: LC-MS experiments are performed using a Waters ZMD 11 Mass Spectrometer.

MS conditions: Electrospray positive ionization; capillary voltage 3.5 kV; cone voltage 30V; desolvation and source temperature 250° C. and 100° C. respectively; mass range 120-800 with a scan time of 0.5 seconds and an inter scan delay of 0.1 min.

Method 2.

Flow Injection Condition:

A Perkin Elmer HPLC system (tow Series 200 micro LC pumps, pump A and pump B, with a Series 200 autosampler) is used to perform flow injection. Mobile phase is a combination of 85% methanol (pump B) with 15% of water (pump A). The flow rate is 1.0 mL/min; and the injection volume is 3 μL.

MS instrumentation: LC-MS experiments are performed using a Sciex 150MA Mass Spectrometer.

MS conditions: Ion source is Heated Nebulizer (atmosphere pressure chemical ionization). The mass range is 100-1000 amu. Both positive and negative modes are in place. For positive ion mode, Nebulizer current is 2.0 mA, and the temperature is 350° C. The Nebulizer gas is 10, and the Curtain gas is 12. The declustering potential is 30 V. The Focusing potential is 200 V, and the entrance potential is −10 V. For negative ion mode, Nebulizer current is −2.0 mA, and the temperature is 350° C. The Nebulizer gas is 10, and the Curtain gas is 12. The declustering potential is −30 V. The Focusing potential is −200 V, and the entrance potential is 10 V.

Method 3.

HPLC Instrumentation: HP1100 PUMP, HP1100 UV detector with 220 nm, HTS/PAL autosampler from Leap Technology, data acquired by Micromass Ma

HPLC conditions: Synergi 2U HYDRO-RP 20×4.0 mm column, flow rate 1.0 mL/min, injection volume 5 μL.

Gradient conditions: 0.1% formic acid in aqueous acetonitrile, 10-90% acetonitrile over 3 min, then 100% acetonitrile, end at 5 min.

MS instrumentation: Micromass LCT-TOF MS

MS conditions: Scan m/z 100-1200, capillary voltage 3000V, cone voltage 25V, desolvation 200° C. and source temperature 100° C.

### Example 1

### Preparation of Boronic Acid Intermediates

a. Synthesis of 2-(Dimethylamino)-4-methoxypyridin-5-boronic acid

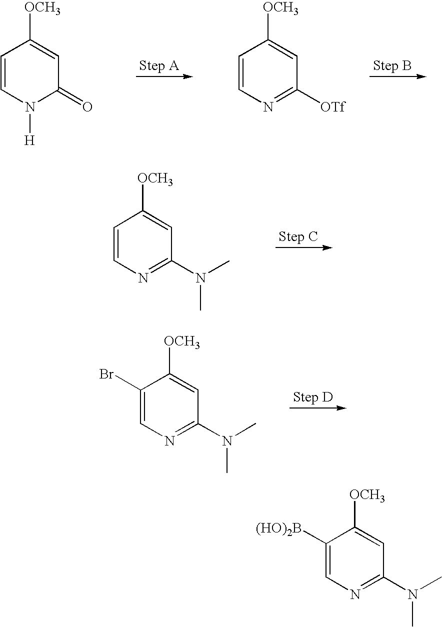

Step A

To a stirred solution of 4-methoxy-1H-pyridin-2-one (Walters and Shay, Tetrahedron Letters 36 (1995), 7575) in methylene chloride (30 mL) at 0° C. is added triflic anhydride (12.9 g) followed by triethylamine (8.4 g). The reaction mixture is stirred for 20 min and then allowed to warm to room temperature. The volatile components are evaporated under vacuum and then the residue is dissolved in EtOAc and washed consecutively with aqueous sodium bicarbonate, water and brine solution. The organic phase is separated, dried and evaporated under vacuum to give trifluoro-methanesulfonic acid 4-methoxy-pyridin-2-yl ester. It is used in the next step without further purification.

Step B

Trifluoro-methanesulfonic acid 4-methoxy-pyridin-2-yl ester (0.5 g) and dimethylamine (2.4 mL of 2M in THF) are dissolved in DMSO (7 mL) and warmed overnight at 40° C. EtOAc is added to the reaction mixture and it is washed with brine solution. The organic phase is separated, dried, and evaporated under vacuum. Silica gel purification gives (4-methoxypyridin-2-yl)dimethylamine. It is used in the next step without further purification.

Step C

N-Bromosuccinimide (1.75 g) is added portionwise to a solution of (4-methoxy-pyridin-2-yl)dimethylamine (1.5 g) at 0° C. in chloroform (30 mL). After 30 min water (4 mL) is added to the reaction mixture and it is extracted three times with methylene chloride. The combined organic phase is separated, dried and evaporated under vacuum. Silica gel purification gives (5-bromo-4-methoxy-pyridin-2-yl)dimethylamine. LCMS: Rt 1.20 min m/z 231.03 (M+H)⁺.

Step D

To a mixture of n-butyllithium (2.68 mL of 1.6M in hexanes) and toluene (7.4 mL) at −65° C. is added dropwise (5-bromo-4-methoxy-pyridin-2-yl)dimethylamine (0.9 g) in toluene (4 mL). The reaction mixture is stirred in the cold for 30 min and the THF (1.6 mL) is added and stirring is continued for a further 15 min. Triisopropylborate (1.5 g) is then added slowly and stirring is continued for 45 min. The reaction mixture is then allowed to warm to room temperature overnight and 1N HCl (10 mL) is added. The aqueous layer is separated and the organic phase is washed consecutively with 1N HCl and water. The combined aqueous phase was adjusted to pH7 with solid sodium bicarbonate and extracted with 1:1 EtOAc/THF. The organic phase is separated, dried and evaporated under vacuum to give 2-(dimethylamino)-4-methoxypyridin-5-boronic acid. LCMS: Rt 2.56 min m/z 197.12 (M+H)⁺
b. Synthesis of 2-(diethylamino)-4-ethylpyridin-5-boronic acid

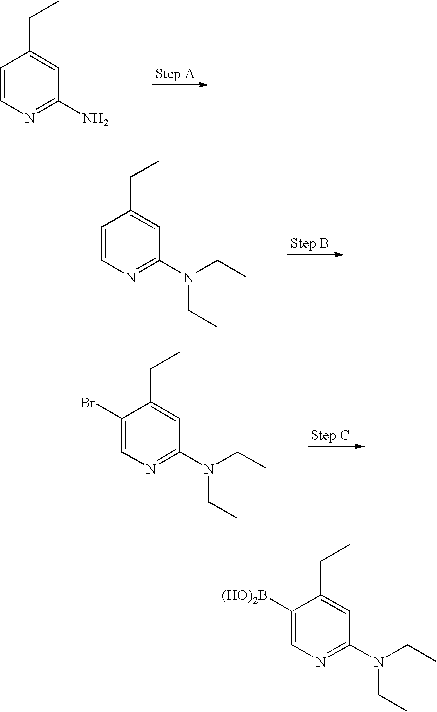

Step A

2-Amino-4-ethylpyridine (4.70 g) is dissolved in dichloromethane (80 mL). Addition of acetaldehyde (8.60 mL) and stirring for 10 min is followed by addition of sodium triacetoxyborohydride (24.6 g). After 1 h, the reaction is put into a mixture of water (300 mL) and sat. sodium bicarbonate (50 mL). Extraction with DCM (3×200 mL) and drying over magnesium sulfate yields a crude mixture that is used in step B without any further purification. LCMS: m/z 179.17 (M+H)⁺

Step B

The crude mixture from step A is dissolved in chloroform (150 mL) and cooled to 0° C. Addition of NBS (6.50 g, in three portions) is followed by stirring for 15 min. The light yellow solution is then put into a mixture of water (500 mL) and sat. sodium bicarbonate (100 mL). Extraction with DCM (3×150 mL) and drying over magnesium sulfate yields a crude mixture that is purified on silica gel. LCMS: m/z 257.10 (M+H)⁺

Step C

t-BuLi (50.1 mL, 1.7N in pentanes) is added to THF (200 mL) at −78° C. Slow addition of the purified material from step B (7.31 g, in 30 mL of THF) is followed by stirring for 15 min at −78° C. Upon LCMS check for unreacted bromide, triisopropyl borate (26.2 mL) is added and the reaction mixture is warmed to room temperature over night. The yellowish solution is then put into a mixture of water (1000 mL) and sat. sodium bicarbonate (100 mL). Extraction with DCM (3×300 mL) and drying over magnesium sulfate yields a crude material of good purity that can be used directly in palladium mediated couplings. LCMS: m/z 223.19 (M+H)⁺

2-(Dimethylamino)-4-ethylpyridin-5-boronic acid (MS m/z 195.09 (M+H)⁺) and 2-(ethyl-methyl-amino)-4-ethylpyridin-5-boronic acid (MS m/z 209.16 (M+H)⁺) are analogously prepared.
c. Synthesis of 2-isopropyl-6-methoxypyridine-3-boronic acid

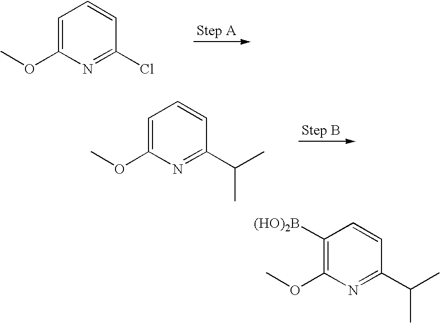

Step A

Following the procedure of Fürstner et al. (JACS 124 (2002) 13856), 2-chloro-6-methoxypyridine (10 g) is stirred at −30° C. in a mixture of THF (2300 mL) and NMP (335 mL). Fe(acac)₃ (14.8 g) is added, followed by isopropyl magnesium chloride (490 mL of 2M in THF). The reaction mixture is allowed to warm to 0° C. over 1 hour and then saturated aqueous ammonium chloride (1000 mL) is added. The aqueous phase is separated and the organic layer is washed two times with water (1000 mL). The organic layer is distilled under reduced pressure to give 2-isopropyl-6-methoxypyridine. LCMS: Rt 1.95 min m/z 152.12 (M+H)⁺

Step B

2-Isopropyl-6-methoxypyridine (191.4 g) and TMEDA (146.3 g) are dissolved in diethyl ether (1565 mL) and cooled to −60° C. n-BuLi (760 mL of 2M) is added over 10 min. and the reaction mixture is allowed to warm to room temperature over 3.5 hours. The reaction mixture is chilled again to −60° C., triisopropylborate (476.2 g) is added and stirring is continued for 24 hours. 3M HCl is then added (510 mL), followed by water (2500 mL). The aqueous phase is separated and the organic layer is washed three times with 5% aqueous NaCl (1500 mL). The four aqueous phases are sequentially extracted with diethyl ether (2000 mL) and the combined ether extracts are concentrated under vacuum to give 2-isopropyl-6-methoxypyridine-3-boronic acid. LCMS: Rt 2.80 min m/z 196.11 (M+H)⁺
d. Synthesis of 2-methoxy-4-trifluoromethoxyphenylboronic acid

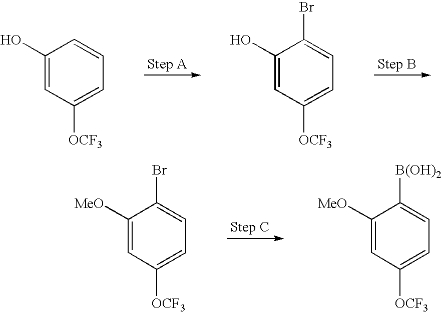

Step A

3-Trifluoromethoxyphenol (256.42 g) is dissolved in dichloromethane (2000 mL) and cooled to 5-10° C. under nitrogen. Bromine (241.6 g) is added dropwise over 2 hours, maintaining the temperature between 5-10° C. and then the cooling bath is removed. Water (1000 mL) is added and the mixture is stirred for 10 minutes and separated. More water is added to the organic phase (500 mL) followed by powdered sodium carbonate (10-12 g) until the pH is 10-11. The organic layer is separated again, dried and concentrated under vacuum. Distillation affords 2-bromo-5-trifluoromethoxyphenol, which is used in the next step without further purification.

Step B

To 2-bromo-5-trifluoromethoxyphenol (479 g) dissolved in toluene (2600 mL) at 1-10° C. is added a solution of sodium hydroxide (80 g) in water (400 mL). The reaction mixture is stirred for 20 min and then tetra-n-butylammonium bromide (24 g) is added. Dimethyl sulfate (239.3 g) is divided into four portions and one portion is added to the mixture every 30 min, maintaining the internal temperature around 12-15° C. The reaction mixture is stirred overnight at this temperature and then water (1000 mL) is added and the organic layer is separated. It is washed consecutively with water (600 mL) and brine (600 mL) and then dried and evaporated to give 3-trifluoromethoxyanisole, which is used in the next step without further purification.

Step C

n-Butyllithium (156 mL of 2.5 M solution in hexanes) is added under nitrogen to THF (800 mL) over a period of 5 min while maintaining the temperature between −77 and −67° C. 2-Methoxy-4-trifluoromethoxy bromobenzene (100 g) is added over a 10-min period while maintaining the temperature between −76.0 and −62° C. Trimethylborate (53.8 g) is added over 10 min at a temperature of −76.3 to −63.2° C. After 1 hour, 200 ml of 2 N hydrochloric acid (200 mL) is added to pH 1. The mixture is allowed to warm to room temperature and the organic phase is separated and concentrated under vacuum to give crude 2-methoxy-4-trifluoromethoxyphenylboronic acid. The solid is treated with boiling n-heptane to give 2-methoxy-4-trifluoromethoxyphenylboronic acid. ¹H-NMR (CDCl₃, 400 MHz) δ 7.89 (d, J=8.5 Hz, 1H), 6.90 (d, J=8.5 Hz, 1H), 6.75 (s, 1H), 6.13 (bs, 2H), 3.94 (s, 3H), Rt 2.87 min m/z 281.02 (M+HCOO)⁻.

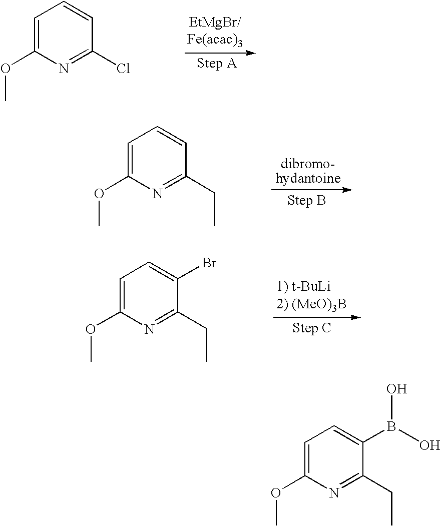

Step A

Commercially available 2-chloro-6-methoxypyridine is transformed into the ethyl compound as described for the corresponding 2-isopropyl-6-methoxypyridine.

Step B

The crude mixture (30.1 g) of step B is dissolved in THF (300 mL) and treated with 1,3-dibromo-5,5-dimethylhydantoine (1.0-1.2 eq, in portions). Once TLC control shows completed conversion of the starting material the addition of the hydantoine is stopped and the mixture is put into water (1 L). Extraction with DCM (3×300 mL), drying over magnesium sulfate, and purification on silica gel affords the bromide. LCMS: m/z 215.97 (M+H)⁺

Step C

Conversion of the bromide into the corresponding boronic acid follows the last step of the previously described synthesis of 2-diethylamino-4-ethyl-5-pyridine boronic acid with methyl borate being used as electrophile. The resulting crude material is of good purity and can be used directly in palladium mediated couplings. LCMS: m/z 182.05 (M+H)⁺

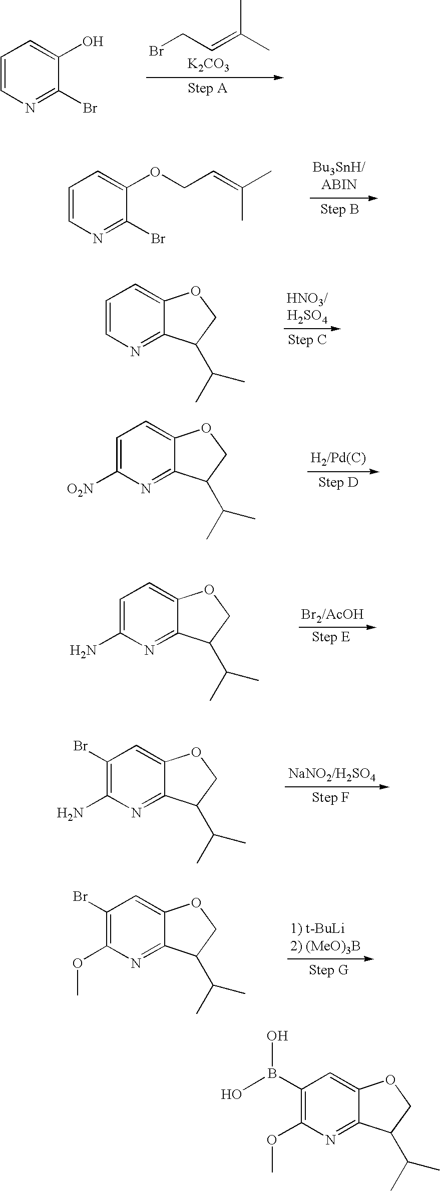

Step A

Commercially available 2-bromo-3-hydroxypyridine (9.41 g) and 3,3-dimethylallyl bromide (9.67 g) are dissolved in acetone (150 mL). After addition of potassium carbonate (17.9 g), the mixture is refluxed for 90 min before being put into water (300 mL). Extraction with DCM (4×200 mL), drying over magnesium sulfate and purification on silica gel affords the allyl ether. LCMS: m/z 241.98 (M+H)⁺

Step B

The ether of step A (960 mg), tributyltin hydride (1.28 g), and ABIN (218 mg) are dissolved in toluene (20 mL) and heated to 95° C. for 26 h. The resulting mixture is put into water (300 mL) and sat. sodium bicarbonate (30 mL). Extraction with DCM (3×100 mL), drying over magnesium sulfate, and purification on silica gel yields the bicyclus. LCMS: m/z 164.13 (M+H)⁺

Step C

The cyclic ether (524 mg) of step B is dissolved in conc. sulfuric acid (5 mL) and then cooled to 0° C. After slow addition of fuming nitric acid (1.25 mL), the reaction mixture is stirred for 2 h before being put onto 30 ml of ice. The resulting suspension is basified (ph=10) with 10N NaOH and subsequently extracted with DCM (3×100 mL). Drying over magnesium sulfate and purification on silica gel affords the desired nitro compound. LCMS: m/z 209.14 (M+H)⁺

Step D

The nitro compound (622 mg) of step C is dissolved in methanol (20 mL). Reduction is achieved by adding a catalytic amount of Pd/C (10%) and maintaining a hydrogen atmosphere (normal pressure) for 90 min. Filtration through celite (10 g) and concentration affords a crude mixture that is directly used in step E. LCMS: m/z 179.11 (M+H)⁺

Step E

The crude mixture of step D (459 mg) is dissolved in acetic acid (10 mL) and then cooled to 0° C. to yield a semi frozen mixture. Bromine (0.139 mL) is slowly added and the reaction is stirred for another 5 min before being put into sat. sodium bicarbonate (100 ml) and 1N sodium sulfite solution (20 mL). Extraction with DCM (3×100 mL), drying over magnesium sulfate, and purification on silica gel affords the bromide. LCMS: m/z 256.98 (M+H)⁺

Step F

The amino bromide (500 mg) of step E is dissolved in a solution of sulfuric acid in methanol (10 mL, 15% sulfuric acid) and then cooled to 0° C. After addition of sodium nitrite (268 mg), the solution is allowed to warm to rt over a period of 16 h. After being put into sat. sodium bicarbonate (100 mL), the aqueous layer is extracted with DCM (3×100 mL) and dried over magnesium sulfate. Purification on silica gel affords the methoxy bromide. LCMS: m/z 272.00 (M+H)⁺

Step G

Conversion of the bromide into the corresponding boronic acid follows the last step of the previously described synthesis of 2-diethylamino-4-ethyl-5-pyridine boronic acid with methyl borate being used as electrophile. The resulting crude material is of good purity and can be used directly in palladium mediated couplings. LCMS: m/z 238.04 (M+H)⁺
g. Synthesis of 2-ethoxy-6-ethyl-5-methanesulfonyl-3-(4,4,5,5-tetramethyl-[1,3,2]dioxaborolan-2-yl)-pyridine

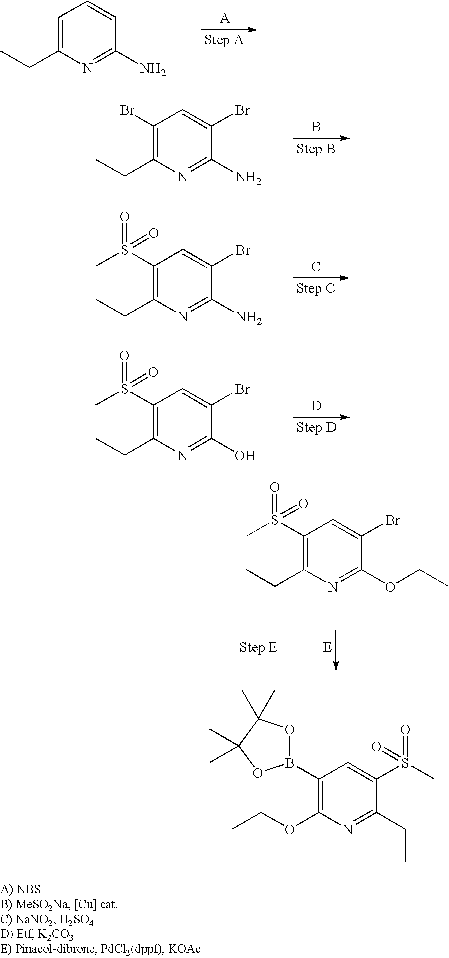

Step A

A mixture of 18.8 g of 6-ethyl-pyridin-2-ylamine in 400 ml CH₂Cl₂ is added NBS (55.32 g) portionwise at room temperature. After addition, the resulting mixture is stirred at room temperature for 10 min before it is washed with water and brine. The resulting organic phase is dried, evaporated and purified by column chromatography (Hexane/EtOAc=7/1) to give desired product 3,5-dibromo-6-ethyl-pyridin-2-ylamine. m/z 281.0 (M+H)⁺.

Step B

3,5-Dibromo-6-ethyl-pyridin-2-ylamine (37.5 g) is dissolved in anhydrous DMSO (300 ml) and the mixture is degassed with N₂ for 2 min followed by addition of sodium methylsulfonate (19.5 g), (CuOTf)₂.Ph.H (3.9 g) and trans-1,2-cyclohexane-diamine (3.06 g). After stirring at 110° C. for 20 hours, the resulting mixture is diluted with water, extracted with EtOAc (4×100 ml), washed with brine and dried over Na₂SO₄. After evaporation of solvent, the residue is purified by column chromatography (Hexane/EtOAc=1/1) to give desired product 3-bromo-6-ethyl-5-methanesulfonyl-pyridin-2-ylamine. m/z 281.2 (M+H)⁺.

Step C

3-Bromo-6-ethyl-5-methanesulfonyl-pyridin-2-ylamine (7.88 g) is dissolved in H₂SO₄-H₂O (ratio 1:6) (175 ml) and the mixture is cooled to 0° C. After adding the solution of NaNO₂ (4.1 g) in 15 ml H₂O dropwise (keep inner temperature below 5° C.), the mixture is stirred at 0° C. to room temperature for overnight. The desired product 3-bromo-6-ethyl-5-methanesulfonyl-pyridin-2-ol is collected by filtration following by washing with water (50 ml). This crude product is used for next step without further purification. m/z 280.0 (M+H)⁺.

Step D

A mixture of 3-bromo-6-ethyl-5-methanesulfonyl-pyridin-2-ol (15.84 g) and DMF (200 ml) is cooled to 0° C., and K₂CO₃ (11.71 g) is added, followed by ethyl iodide (11.3 ml). The resulting mixture is stirred at 0° C. to room temperature for overnight. After the reaction is complete, water is added and the resulting mixture is extracted with EtOAc (3×200 ml). The combined organic layers are washed with brine, dried over Na₂SO₄ and evaporated. The pure product 3-bromo-2-ethoxy-6-ethyl-5-methanesulfonyl-pyridine is obtained after column chromatography. m/z 282.1 (M+H-Et)⁺.

Step E

A mixture of 3-bromo-2-ethoxy-6-ethyl-5-methanesulfonyl-pyridine (400 mg) in DMSO (20 ml) is added bis(pinacolato)diboron (396 mg), KOAc (382 mg) and PdCl₂(dppf) (49 mg) and the resulting mixture is stirred at 90° C. for overnight. After the reaction is complete, the mixture is poured into water and extracted with ethyl acetate (3×40 ml). The combined organic layers are washed with brine, dried over Na₂SO₄. Flash column chromatography purification (Hexane/EtOAc=8/1) gives the pure product 2-ethoxy-6-ethyl-5-methanesulfonyl-3-(4,4,5,5-tetramethyl-[1,3,2]dioxaborolan-2-yl)-pyridine. m/z 356.3 (M+H)⁺.
h. Synthesis of 5-ethyl-3-isopropyl-6-(4,4,5,5-tetramethyl-[1,3,2]dioxaborolan-2-yl)-3H-imidazo[4,5-b]pyridine

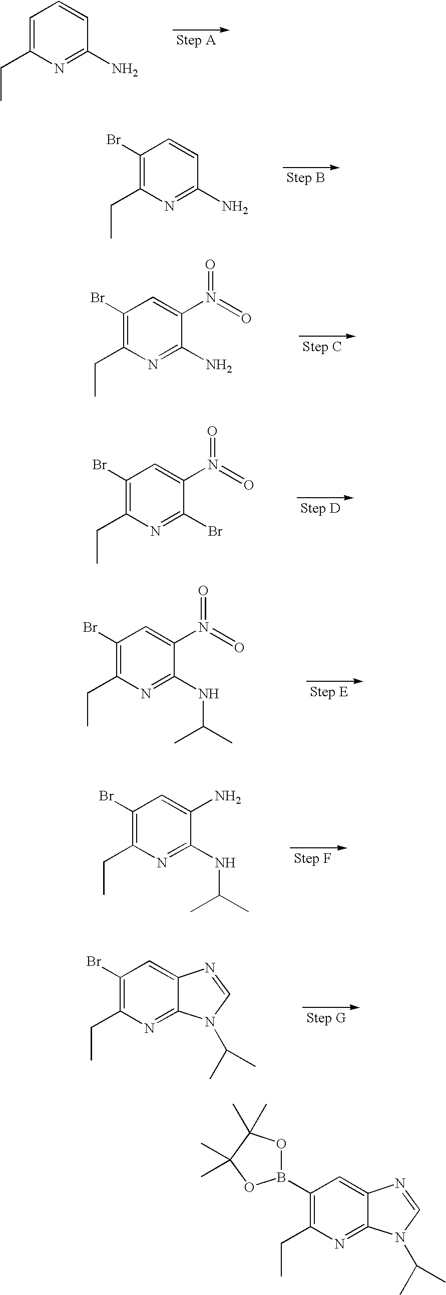

Step A

To a solution of 2-amino-6-ethyl-pyridine (50 g) in chloroform (250 mL) is added NBS (73 g) at 0° C. over 30 min. The mixture is stirred for additional 30 min and is directly purified by flash column chromatography on silica gel to give 5-bromo-6-ethyl-pyridin-2-ylamine as white solid. Rf (hexane:EtOAc=4:1)=0.34.

Step B

5-Bromo-6-ethyl-pyridin-2-ylamine (34 g) is added to CH₂SO₄ (110 mL) below 10° C. To the stirred mixture is added HNO₃ (8.2 mL) below 15° C. over 40 min. The mixture is stirred at 0° C. for 1 h, at RT for 1 h and finally at 50° C. for 1 h. The mixture is poured into ice-water and is basified by 50% NaOH. Yellow crystals are collected by filtration, washed with water and dried under reduced pressure to give 5-bromo-6-ethyl-3-nitro-pyridin-2-ylamine. Rf (hexane:EtOAc=4:1)=0.5.

Step C

To a stirred suspension of 5-bromo-6-ethyl-3-nitro-pyridin-2-ylamine (5 g) in AcOH (20 mL) is added 48% HBr (20 mL) below 10° C. Bromine (2.92 mL) is added to the mixture below 10° C. over 15 min. At 0° C., a solution of NaNO₂ in water (3.65 g, 15 mL) is added over 20 min below 15° C. The mixture is stirred at 0° C. for 30 min and at RT for 1 h. The mixture is cooled to 0° C., neutralized by 50% of NaOH, and extracted with DCM. The extract is dried over MgSO₄ and is concentrated under reduced pressure to give 2,5-dibromo-6-ethyl-3-nitro-pyridine as yellow oil. Rf (hexane:EtOAc=9:1)=0.7

Step D

To a stirred suspension of 2,5-dibromo-6-ethyl-3-nitro-pyridine (20 g) in EtOH (20 mL) is added a solution of isopropylamine (25 mL) in water (60 mL) at 0° C. The mixture is stirred at 0° C. for 10 min and at RT for 2 h. Red-yellow crystals formed are collected by filtration and are washed with water. The wet crystals are dissolved in DCM (250 mL). After drying over MgSO₄, the solvent is removed under reduced pressure to give (5-bromo-6-ethyl-3-nitro-pyridin-2-yl)-isopropyl-amine as red-yellow solid. Rf (hexane:EtOAc=9:1)=0.77

Step E

To a solution of (5-bromo-6-ethyl-3-nitro-pyridin-2-yl)-isopropyl-amine (1 g) in EtOH (4 mL) is added conc. HCl (0.05 mL), water (1 mL) and reduced iron (3 g) at RT. The mixture is refluxed for 90 min. The iron residue is removed by filtration and is washed with EtOH. The combined filtrates are concentrated under reduced pressure. To the residue is added water and the mixture is extracted with EtOAc. The combined extracts are washed with brine and dried over MgSO₄. The solvent is removed under reduced pressure to give 5-bromo-6-ethyl-N*2*-isopropyl-pyridine-2,3-diamine as gum. LCMS Rt 1.20 min, m/z 258.05/260.04 (M+H)⁺

Step F

5-Bromo-6-ethyl-N*2*-isopropyl-pyridine-2,3-diamine (1 g) is dissolved in diethoxymethylacetate (4 mL) and is heated at 120° C. for 90 min. After cooling to RT the mixture is directly purified by flash column chromatography on silica gel to give 6-bromo-5-ethyl-3-isopropyl-3H-imidazo[4,5-b]pyridine as colorless oil. Rf (hexane:EtOAc=2:1)=0.32

Step G

To a solution of 6-bromo-5-ethyl-3-isopropyl-3H-imidazo[4,5-b]pyridine (59 mg) in DMSO (2 mL) is added bis(pinacolate)diborane (69 mg), KOAc (65 mg) and PdCl₂(dppf)-DCM complex (9 mg) at RT. The mixture is stirred at 90° C. for 20 h to give 5-ethyl-3-isopropyl-6-(4,4,5,5-tetramethyl-[1,3,2]dioxaborolan-2-yl)-3H-imidazo[4,5-b]pyridine, which is able to be used for the coupling. LCMS Rt 1.66 min, m/z 316.22 (M+H)⁺
i. Synthesis of 6-Ethyl-4-isopropyl-2-methyl-7-(4,4,5, 5-tetramethyl-[1,3,2]dioxaborolan-2-yl)-4H-pyrido[2,3-b]pyrazin-3-one

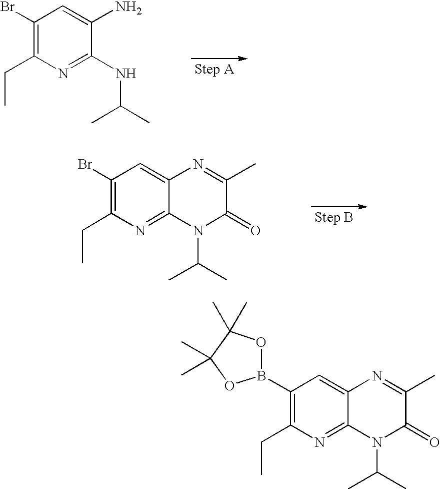

Step A

To a solution of 5-bromo-6-ethyl-N*2*-isopropyl-pyridine-2,3-diamine (2.38 g) in toluene (10 mL) is added ethyl pyruvate (2.05 mL) at RT. The mixture is refluxed for 18 h and is poured into water and is extracted with EtOAc. The extract is washed with brine and is dried over MgSO₄. After removal of the solvent under reduced pressure the residue is purified by flash column chromatography on silica gel to give 7-bromo-6-ethyl-4-isopropyl-2-methyl-4H-pyrido[2,3-b]pyrazin-3-one as white crystal. LCMS Rt 1.74 min, m/z 310.02/312.02 (M+H)⁺

Step B

To a solution of 7-bromo-6-ethyl-4-isopropyl-2-methyl-4H-pyrido[2,3-b]pyrazin-3-one (0.2 g) in DMSO (4 mL) is added bis(pinacolate)diborane (0.2 g), KOAc (0.19 g) and PdCl₂(dppf)-DCM complex (29 mg) at RT. The mixture is stirred at 90° C. for 20 h to give 6-ethyl-4-isopropyl-2-methyl-7-(4,4,5,5-tetramethyl-[1,3,2]dioxaborolan-2-yl)-4H-pyrido[2,3-b]pyrazin-3-one, which is able to be used for the coupling. LCMS Rt 1.80 min, m/z 358.22 (M+H)⁺

### Example 2

### Synthesis of 5-(1-ethyl-propyl)-2-(2-methoxy-4-trifluoromethoxy-phenyl)-3,7-dimethyl-5H-pyrrolo[2,3-b]pyrazine

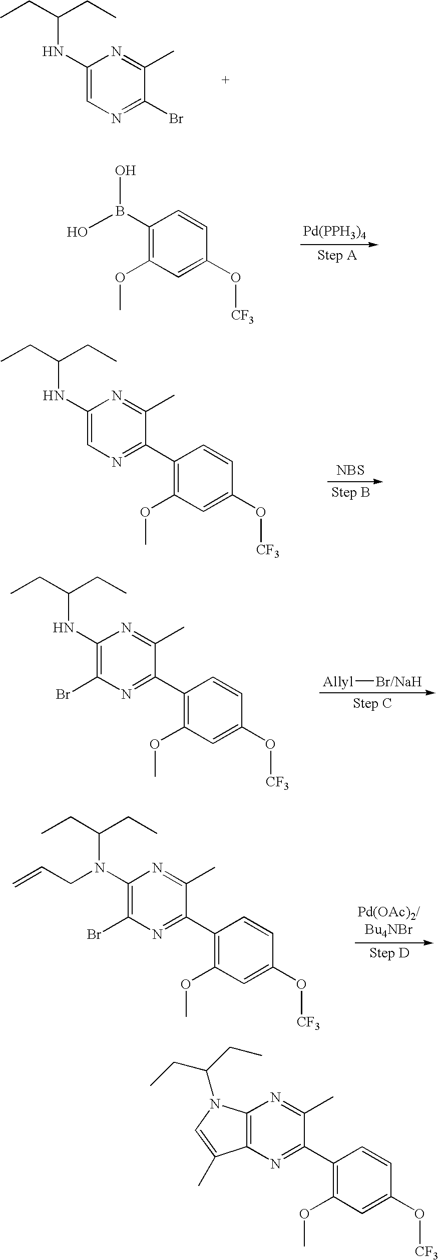

Step A

The previously described 2-bromo-3-methyl-5-isopentylaminopyrazine (870 mg) and the literature known 2-methoxy-4-trifluoromethoxyphenyl boronic acid (796 mg) are dissolved in DME (1 5 mL). After degassing, tetrakis(triphenylphosphine)palladium(0) (390 mg) is added. A second degassing is followed by addition of a 1N sodium carbonate solution (6.74 mL) whereupon the reaction is heated to 80° C. for 6 h. The yellowish mixture is then put into water (200 mL), extracted with DCM (3×100 mL), and dried over magnesium sulfate. Purification on silica gel affords the coupled product. LCMS: m/z 370.17 (M+H)⁺

Step B

The product (205 mg) of step A is dissolved in chloroform (10 mL) and NBS (99 mg) is added. After being stirred for 10 min, the yellowish mixture is put into water (100 mL), extracted with DCM (3×100 mL), and dried over magnesium sulfate. Purification on silica gel affords the bromide. LCMS: m/z 448.11 (M+H)⁺

Step C

The bromide (173 mg) of step B and allyl bromide (0.33 mL) are dissolved in DMF (5 mL). Sodium hydride (100 mg) is added and the reaction is stirred for 10 min at rt. The mixture is then put into water (100 mL) and extracted with ethyl ether (2×100 mL). The combined organic layers are washed with water (50 mL), dried over magnesium sulfate, and purified on silica gel to afford the allylated amino-compound. LCMS: m/z 488.11 (M+H)⁺

Step D

The allyl compound (138 mg) of step C, tetrabutylammonium bromide (91 mg), palladium acetate (6.4 mg), and potassium carbonate (117 mg) are dissolved in DMF (5 mL). After heating to 80° C. for 90 min, the mixture is worked-up according to step C. Final purification on silica gel affords the title compound. LCMS: m/z 408.21 (M+H)⁺

### Example 3

### Synthesis of {5-[3-chloro-5-(1-ethyl-propyl)-7-methyl-5H-pyrrolo[2,3-b]pyrazin-2-yl]-4-ethyl-pyridin-2-yl}-dimethyl-amine

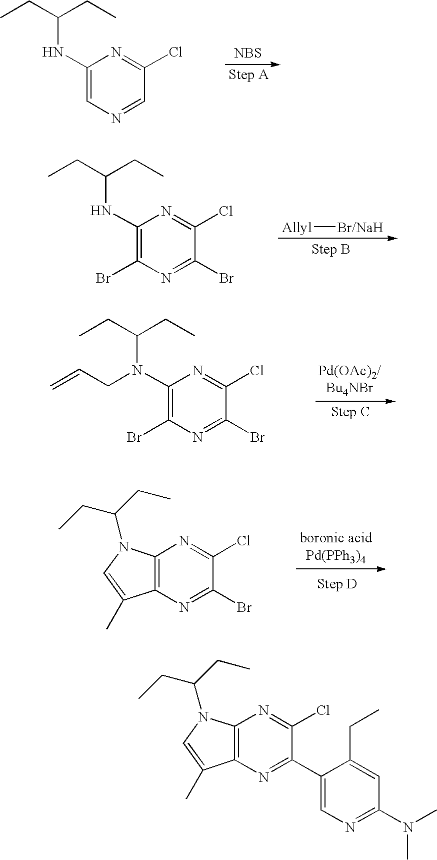

Step A

The previously described 2-chloro-6-isopentylaminopyrazine (25.1 g) is dissolved in chloroform (450 mL) and NBS (47.1 g) is added in portions. After being stirred for 30 min, the yellowish mixture is put into water (400 mL) and sat. sodium bicarbonate (100 mL), extracted with DCM (3×200 mL), and dried over magnesium sulfate. The crude material is carried on to step B without any further purification. Rf=0.57 in hex/ethyl acetate (10/1)

Step B

The crude material (28.37 g) of step A and allyl bromide (20.6 mL) are dissolved in DMF (200 mL). Sodium hydride (4.76 g) is added in portions and the reaction is stirred for 5 h at rt. The mixture is then put into water (500 mL) and extracted with ethyl acetate/hexane (1/20, 3×300 mL). The combined organic layers are dried over magnesium sulfate and purified on silica gel to afford the allylated product. LCMS: m/z 395.85 (M+H)⁺

Step C

The allyl compound (23.36 g) of step B, tetrabutylammonium bromide (19.00 g), palladium acetate (1.32 g), and potassium carbonate (24.8 g) are dissolved in DMF (200 mL). After heating to 80° C. for 20 min, the mixture is put into water (500 mL) and extracted with ethyl acetate/hexane (1/4, 3×300 mL). The combined organic layers are washed with water (100 mL), dried over magnesium sulfate, and purified on silica gel to afford the Heck-product. LCMS: m/z 316.01 (M+H)⁺

Step D

The Heck-product of step C (1.5 g) and the previously described 2-dimethylamino-4-ethyl-5-pyridine boronic acid (1.38 g) are dissolved in DME (30 mL). After degassing, tetrakis(triphenylphosphine)palladium(0) (550 mg) is added. A second degassing is followed by addition of a 1N sodium carbonate solution (9.5 mL) whereupon the reaction is heated to 80° C. for 16 h. The yellowish mixture is then put into water (200 mL), extracted with DCM (3×100 mL), and dried over magnesium sulfate. Purification on silica gel affords the title compound. LCMS: m/z 386.20 (M+H)⁺

### Example 4

### Synthesis of 3-chloro-5-(1-ethyl-propyl)-2-(3-isopropyl-5-methoxy-furo[3,2-b]pyridin-6-yl)-7-methyl-5H-pyrrolo[2,3-b]pyrazine

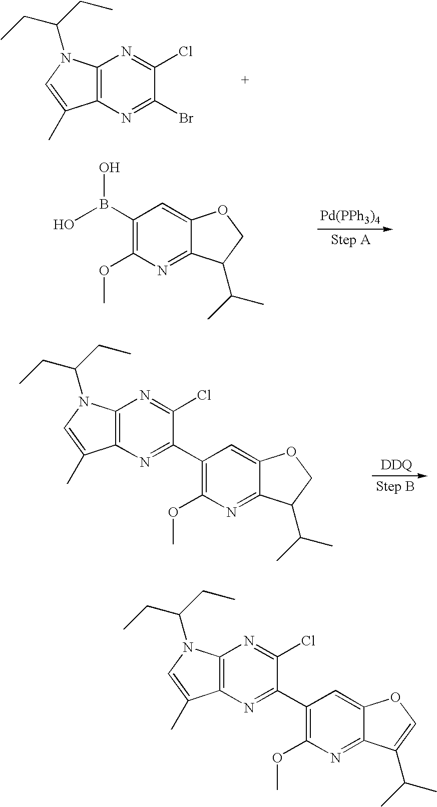

Step A

The previously described bromide (85 mg) and the also previously described pyridine boronic acid (64 mg) are dissolved in DME (3 mL). After degassing, tetrakis(triphenylphosphine)palladium(0) (31 mg) is added. A second degassing is followed by addition of a 1N sodium carbonate solution (0.54 mL) whereupon the reaction is heated to 80° C. for 16 h. The yellowish mixture is then put into water (100 mL), extracted with DCM (3×100 mL), and dried over magnesium sulfate. Purification on silica gel affords the coupled product. LCMS: m/z 429.08 (M+H)⁺

Step B

The Suzuki product (52 mg) of step A and DDQ (41 mg) are dissolved in benzene (5 mL) and heated to 80° C. for 3 h. The reaction mixture is then put into water (100 mL), extracted with DCM (3×100 mL), and dried over magnesium sulfate. Purification on silica gel affords the title compound. LCMS: m/z 427.12 (M+H)⁺

### Example 5

### Synthesis of (S)-3-chloro-2-(6-isopropyl-2-methoxy-pyridin-3-yl)-5-(2-methoxy-1-methyl-ethyl)-7-methyl-5H-pyrrolo[2,3-b]pyrazine and (S)-3-ethyl-2-(6-isopropyl-2-methoxy-pyridin-3-yl)-5-(2-methoxy-1-methyl-ethyl)-7-methyl-5H-pyrrolo[2,3-b]pyrazine

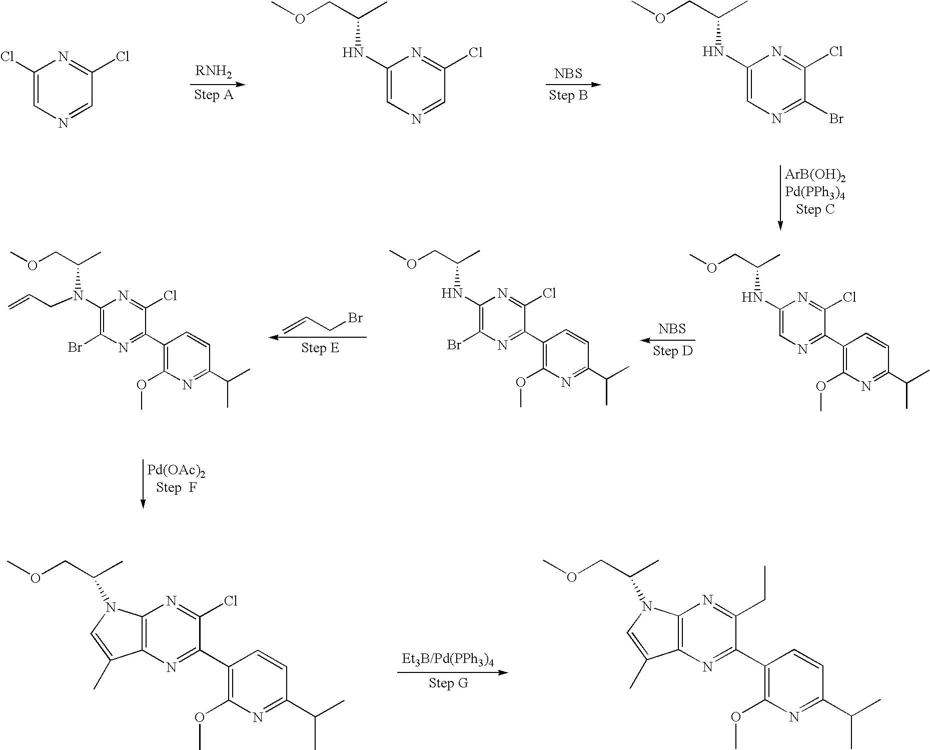

Step A

2,6-Dichloropyrazine (11.7 g), (S)-(+)-1-methoxy-2-propylamine (7 g) and Et₃N (15 mL) in EtOH (100 mL) are heated at 105° C. for 2 days. The mixture is evaporated and dissolved in EtOAc and washed with sat. NaHCO₃, H₂O and dried. Evaporation affords 2-chloro-6-[(S)-1-methoxy-2-propyl]aminopyrazine. LCMS: m/z 202.3 and 204.3 (M+H)⁺

Step B

2-Chloro-6-[(S)-1-methoxy-2-propyl]aminopyrazine (8.3 g) is dissolved in CHCl₃ (250 mL). Upon addition of NBS (7.33 g), the reaction mixture is stirred at 25° C. for 30 min. Subsequently, the crude mixture is evaporated, dissolved in EtOAc/hexane (1:4, 500 mL), washed with water and dried over sodium sulfate. Purification on silica gel affords 3-bromo-2-chloro-6-[(S)-1-methoxy-2-propyl]aminopyrazine. LCMS: m/z 280.2, 282.2 and 284.2 (M+H)⁺

Step C

3-Bromo-2-chloro-6-[(S)-1-methoxy-2-propyl]aminopyrazine (10.7 g) and 2-methoxy-6-isopropyl-3-pyridineboronic acid (9.7 g) are dissolved in DME (250 mL). After 10 min of degassing, tetrakis(triphenylphosphine)palladium(0) (2.2 g) is added, followed by 1 min of degassing. Upon addition of an aqueous 1N sodium carbonate solution (76 mL), the reaction mixture is heated at 90° C. for 12 h. Subsequently, the crude mixture is put into water (800 mL), extracted with EtOAc/hexane (1:1, 3×250 mL), and dried over sodium sulfate. Purification on silica gel affords 3-{2-chloro-6-[(S)-1-methoxy-2-propyl]aminopyrazin-3-yl}-2-methoxy-6-isopropylpyridine. LCMS: m/z 351.3 and 353.3 (M+H)⁺

Step D

3-{2-Chloro-6-[(S)-1-methoxy-2-propyl]aminopyrazin-3-yl}-2-methoxy-6-isopropylpyridine (4.85 g) is dissolved in CHCl₃ (60 mL). Upon addition of NBS (2.46 g), the reaction mixture is stirred at 25° C. for 30 min. Subsequently, the crude mixture is evaporated, dissolved in EtOAc/hexane (1:4, 250 mL), washed with water and dried over sodium sulfate. Purification on silica gel affords 3-{5-bromo-2-chloro-6-[(S)-1-methoxy-2-propyl]aminopyrazin-3-yl}-2-methoxy-6-isopropylpyridine. LCMS: m/z 429.2, 431.2 and 433.2 (M+H)⁺

Step E

3-{5-Bromo-2-chloro-6-[(S)-1-methoxy-2-propyl]aminopyrazin-3-yl}-2-methoxy-6-isopropylpyridine (4.3 g) is dissolved in DMSO (50 ml). Upon addition of NaH (60%, 0.8 g), the reaction mixture is stirred at 25° C. for 30 min before allyl bromide (1.7 mL) is added. The reaction mixture is stirred at 25° C. for 2 h. Subsequently, the crude mixture is put into water (250 mL), extracted with EtOAc/hexane (1:4, 2×250 mL), and dried over sodium sulfate. Purification on silica gel affords 3-{5-bromo-2-chloro-6-[(S)-N-allyl-1-methoxy-2-propyl]aminopyrazin-3-yl}-2-methoxy-6-isopropylpyridine. LCMS: m/z 469.3, 471.3 and 473.3 (M+H)⁺

Step F

3-{5-Bromo-2-chloro-6-[(S)-N-allyl-1-methoxy-2-propyl]aminopyrazin-3-yl}-2-methoxy-6-isopropylpyridine (4.6 g) is dissolved in DMF (80 mL). After 10 min of degassing, Pd(OAc)₂ (225 mg) is added, followed by 1 min of degassing. Upon addition of potassium carbonate (4.1 g) and Bn₄NBr (4.0 g), the reaction mixture is heated at 90° C. for 1 h. Subsequently, the crude mixture is put into water (500 mL), extracted with EtOAc/hexane (1:2, 3×150 mL), and dried over sodium sulfate. Purification on silica gel affords (S)-3-chloro-2-(6-isopropyl-2-methoxy-pyridin-3-yl)-5-(2-methoxy-1-methyl-ethyl)-7-methyl-5H-pyrrolo[2,3-b]pyrazine. LCMS: m/z 389.4 and 391.4 (M+H)⁺

Step G

(S)-3-Chloro-2-(6-isopropyl-2-methoxy-pyridin-3-yl)-5-(2-methoxy-1-methyl-ethyl)-7-methyl-5H-pyrrolo[2,3-b]pyrazine (400 mg) is dissolved in toluene (5 mL). After 10 min of degassing, tetrakis(triphenylphosphine)palladium(0) (35 mg) is added, followed by 1 min of degassing. Upon addition of triethylborane (1N in hexane, 3 mL) and aqueous 1N sodium carbonate solution (2 mL), the reaction mixture is heated at 110° C. for 36 h. Subsequently, the crude mixture is put into water (10 mL), extracted with EtOAc/hexane (1:3, 3×25 mL), and dried over sodium sulfate. Purification on silica gel affords (S)-3-ethyl-2-(6-isopropyl-2-methoxy-pyridin-3-yl)-5-(2-methoxy-1-methyl-ethyl)-7-methyl-5H-pyrrolo[2,3-b]pyrazine. LCMS: m/z 383.4 (M+H)⁺

### Example 6

### Synthesis of methanesulfonic acid 2-[(S)-2-(6-isopropyl-2-methoxy-pyridin-3-yl)-3,7-dimethyl-pyrrolo[2,3-b]pyrazin-5-yl]-butyl ester, 3-{2-[(S)-2-(6-isopropyl-2-methoxy-pyridin-3-yl)-3,7-dimethyl-pyrrolo[2,3-b]pyrazin-5-yl]-butyl}-oxazolidin-2-one, {2-[(S)-2-(6-isopropyl-2-methoxy-pyridin-3-yl)-3,7-dimethyl-pyrrolo[2,3-b]pyrazin-5-yl]-butyl}-methyl-amine, N-{2-[(S)-2-(6-isopropyl-2-methoxy-pyridin-3-yl)-3,7-dimethyl-pyrrolo[2,3-b]pyrazin-5-yl]-butyl}-N-methyl-acetamide, N-{2-[(S)-2-(6-isopropyl-2-methoxy-pyridin-3-yl)-3,7-dimethyl-pyrrolo[2,3-b]pyrazin-5-yl]-butyl}-N-methyl-methanesulfonamide, {2-[(S)-2-(6-isopropyl-2-methoxy-pyridin-3-yl)-3,7-dimethyl-pyrrolo[2,3-b]pyrazin-5-yl]-butyl}-methyl-carbamic acid methyl ester and (S)-2-(6-isopropyl-2-methoxy-pyridin-3-yl)-5-(1-methoxymethyl-ropyl)-3,7-dimethyl-5H-pyrrolo[2,3-b]pyrazine

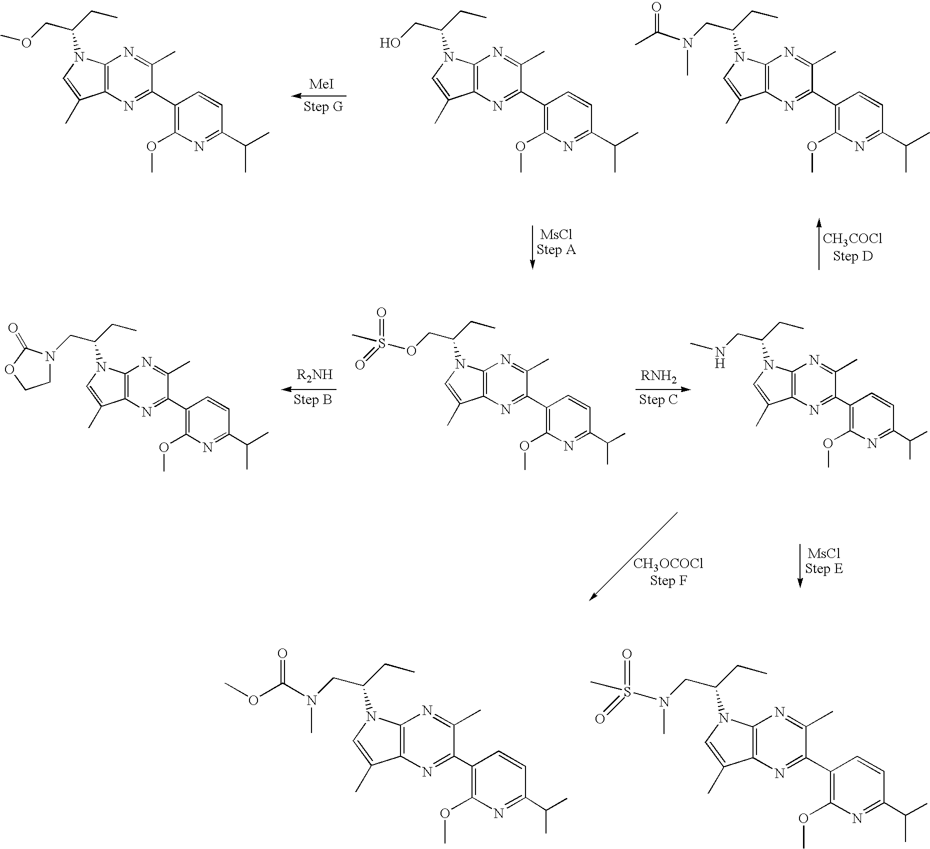

Step A

2-[(S)-2-(6-Isopropyl-2-methoxy-pyridin-3-yl)-3,7-dimethyl-pyrrolo[2,3-b]pyrazin-5-yl]-butan-1-ol (275 mg) is dissolved in CH₂Cl₂ (6 mL). MsCl (0.07 mL) and Et₃N (0.16 mL) are added at r.t. and the mixture is stirred for 1 h. The mixture is evaporated and dissolved in EtOAc/hexane (1:1) and washed with sat. NaHCO₃, H₂O and dried. Evaporation affords the methanesulfonate. LCMS: m/z 447.1 (M+H)⁺

Step B

2-Oxazolidone (26 mg) is dissolved in DMF (3 mL). NaH (12 mg, 60%) is added at r.t. and the mixture is stirred for 10 min at 85° C. Upon addition of the methanesulfonate from step A (35 mg), the reaction mixture is heated at 85° C. for 3 h. Subsequently, the mixture is poured into H₂O and extracted with EtOAc. Evaporation and purification on silica gel affords 3-{2-[(S)-2-(6-isopropyl-2-methoxy-pyridin-3-yl)-3,7-dimethyl-pyrrolo[2,3-b]pyrazin-5-yl]-butyl}-oxazolidin-2-one. LCMS: m/z 438.4 (M+H)⁺

Step C

The above methanesulfonate (120 mg) from step A, LiI (150 mg) and methylamine (7M in NMP, 2 mL) are heated at 90° C. for 4 h. Subsequently, the crude mixture is put into water (10 mL), extracted with EtOAc (2×15 mL), and dried over sodium sulfate. Purification on silica gel affords {2-[(S)-2-(6-isopropyl-2-methoxy-pyridin-3-yl)-3,7-dimethyl-pyrrolo[2,3-b]pyrazin-5-yl]-butyl}-methyl-amine. LCMS: m/z 383.3 (M+H)⁺

Step D

{2-[(S)-2-(6-Isopropyl-2-methoxy-pyridin-3-yl)-3,7-dimethyl-pyrrolo[2,3-b]pyrazin-5-yl]-butyl}-methyl-amine (30 mg) is dissolved in CH₂Cl₂ (1 mL). Acetyl chloride (0.017 mL) and Et₃N (0.033 mL) are added. The resulting reaction mixture is stirred at r.t. for 30 min. Subsequently, the crude mixture is put into water (2 mL), extracted with EtOAc (2×5 mL), washed with sat. NaHCO₃ (2 mL) and dried over sodium sulfate. Purification on silica gel affords N-{2-[(S)-2-(6-isopropyl-2-methoxy-pyridin-3-yl)-3,7-dimethyl-pyrrolo[2,3-b]pyrazin-5-yl]-butyl}-N-methyl-acetamide. LCMS: m/z 424.5 (M+H)⁺

Step E

{2-[(S)-2-(6-Isopropyl-2-methoxy-pyridin-3-yl)-3,7-dimethyl-pyrrolo[2,3-b]pyrazin-5-yl]-butyl}-methyl-amine (20 mg) is dissolved in CH₂Cl₂ (1 mL). Methanesulfonyl chloride (0.008 mL) and Et₃N (0.021 mL) are added. The resulting reaction mixture is stirred at r.t. for 30 min. Subsequently, the crude mixture is put into water (2 mL), extracted with EtOAc (2×5 mL), washed with sat. NaHCO₃ (2 mL) and dried over sodium sulfate. Purification on silica gel affords N-{2-[(S)-2-(6-isopropyl-2-methoxy-pyridin-3-yl)-3,7-dimethyl-pyrrolo[2,3-b]pyrazin-5-yl]-butyl}-N-methyl-methanesulfonamide. LCMS: m/z 460.3 (M+H)⁺

Step F

{2-[(S)-2-(6-Isopropyl-2-methoxy-pyridin-3-yl)-3,7-dimethyl-pyrrolo[2,3-b]pyrazin-5-yl]-butyl}-methylamine (20 mg) is dissolved in CH₂Cl₂ (1 mL). Methyl chloroformate (0.012 mL) and Et₃N (0.013 mL) are added. The resulting reaction mixture is stirred at r.t. for 30 min. Subsequently, the crude mixture is put into water (2 mL), extracted with EtOAc (2×5 mL), washed with sat. NaHCO₃ (2 mL) and dried over sodium sulfate. Purification on silica gel affords {2-[(S)-2-(6-isopropyl-2-methoxy-pyridin-3-yl)-3,7-dimethyl-pyrrolo[2,3-b]pyrazin-5-yl]-butyl}-methyl-carbamic acid methyl ester. LCMS: m/z 440.4 (M+H)⁺

Step G

2-[(S)-2-(6-Isopropyl-2-methoxy-pyridin-3-yl)-3,7-dimethyl-pyrrolo[2,3-b]pyrazin-5-yl]-butan-1-ol (40 mg) is dissolved in DMF (1 mL). NaH (60%, 7 mg) is added, followed by CH₃I (0.02 mL) at r.t. and the mixture is stirred for 1 h. The mixture is evaporated and dissolved in EtOAc/hexane (1:1) and washed with sat. NaHCO₃, H₂O and dried. Evaporation affords (S)-2-(6-isopropyl-2-methoxy-pyridin-3-yl)-5-(1-methoxymethyl-ropyl)-3,7-dimethyl-5H-pyrrolo[2,3-b]pyrazine. LCMS: m/z 383.2 (M+H)⁺

### Example 7

### Synthesis of (R)-2-(6-isopropyl-2-methoxy-pyridin-3-yl)-3,7-dimethyl-5-(1-morpholin-4-ylmethyl-propyl)-5H-pyrrolo[2,3-b]pyrazine, diethyl-{2-[(R)-2-(6-isopropyl-2-methoxy-pyridin-3-yl)-3,7-dimethyl-pyrrolo[2,3-b]pyrazin-5-yl]-butyl}-amine, (R)-2-(6-isopropyl-2-methoxy-pyridin-3-yl)-5-(1-methoxymethyl-propyl)-3,7-dimethyl-5H-pyrrolo[2,3-b]pyrazine, acetic acid 2-[(R)-2-(6-isopropyl-2-methoxy-pyridin-3-yl)-3,7-dimethyl-pyrrolo[2,3-b]pyrazin-5-yl]-butyl ester, and dimethylcarbamic acid 2-[(R)-2-(6-isopropyl-2-methoxy-pyridin-3-yl)-3,7-dimethyl-pyrrolo[2,3-b]pyrazin-5-yl]-butyl ester

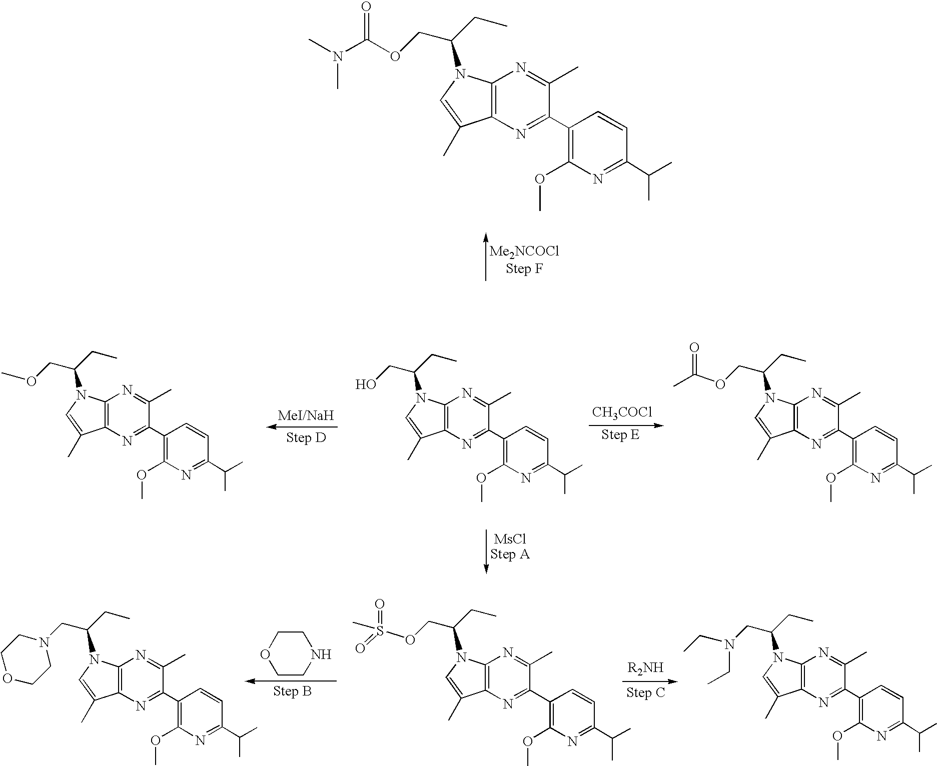

Step A

2-[(R)-2-(6-Isopropyl-2-methoxy-pyridin-3-yl)-3,7-dimethyl-pyrrolo[2,3-b]pyrazin-5-yl]-butan-1-ol (275 mg) is dissolved in CH₂Cl₂ (6 mL). MsCl (0.07 mL) and Et₃N (0.16 mL) are added at r.t. and the mixture is stirred for 1 h. The mixture is evaporated and dissolved in EtOAc/hexane (1:1) and washed with sat. NaHCO₃, H₂O and dried. Evaporation affords the methanesulfonate. LCMS: m/z 447.1 (M+H)⁺

Step B

The above methanesulfonate (95 mg), LiI (50 mg) and morpholine (0.35 mL) are heated at 90° C. for 4 h. Subsequently, the crude mixture is put into water (10 mL), extracted with EtOAc (2×15 mL), and dried over sodium sulfate. Purification on silica gel affords (R)-2-(6-isopropyl-2-methoxy-pyridin-3-yl)-3,7-dimethyl-5-(1-morpholin-4-ylmethyl-propyl)-5H-pyrrolo[2,3-b]pyrazine. LCMS: m/z 438.5 (M+H)⁺

Step C

The above methanesulfonate (115 mg), LiI (50 mg) and diethylamine (0.5 mL) in CH₃CN (3 mL) are heated at 90° C. for 4 h. Subsequently, the crude mixture is put into water (10 mL), extracted with EtOAc (2×15 mL), and dried over sodium sulfate. Purification on silica gel affords diethyl-{2-[(R)-2-(6-isopropyl-2-methoxy-pyridin-3-yl)-3,7-dimethyl-pyrrolo[2,3-b]pyrazin-5-yl]-butyl}-amine. LCMS: m/z 424.14 (M+H)⁺

Step D

2-[(R)-2-(6-sopropyl-2-methoxy-pyridin-3-yl)-3,7-dimethyl-pyrrolo[2,3-b]pyrazin-5-yl]-butan-1-ol (185 mg) is dissolved in DMF (2 mL). NaH (60%, 40 mg) is added, followed by CH₃I (0.1 mL) at r.t. and the resulting mixture is stirred for 1 h. The mixture is evaporated and dissolved in EtOAc/hexane (1:1) and washed with sat. NaHCO₃, H₂O and dried. Evaporation affords (R)-2-(6-isopropyl-2-methoxy-pyridin-3-yl)-5-(1-methoxymethyl-propyl)-3,7-dimethyl-5H-pyrrolo[2,3-b]pyrazine. LCMS: m/z 383.2 (M+H)⁺

Step E

2-[(R)-2-(6-Isopropyl-2-methoxy-pyridin-3-yl)-3,7-dimethyl-pyrrolo[2,3-b]pyrazin-5-yl]-butan-1-ol (37 mg) is dissolved in CH₂Cl₂ (1 mL). Acetyl chloride (0.015 mL) and Et₃N (0.028 mL) are added. The resulting reaction mixture is stirred at r.t. for 30 min. Subsequently, the crude mixture is put into water (2 mL), extracted with EtOAc (2×5 mL), washed with sat. NaHCO₃ (2 mL) and dried over sodium sulfate. Purification on silica gel affords acetic acid 2-[(R)-2-(6-isopropyl-2-methoxy-pyridin-3-yl)-3,7-dimethyl-pyrrolo[2,3-b]pyrazin-5-yl]-butyl ester. LCMS: m/z 411.4 (M+H)⁺

Step F

2-[(R)-2-(6-Isopropyl-2-methoxy-pyridin-3-yl)-3,7-dimethyl-pyrrolo[2,3-b]pyrazin-5-yl]-butan-1-ol (70 mg) is dissolved in CH₂Cl₂ (2 mL). Dimethylcarbamyl chloride (0.08 mL) and pyridine (0.2 mL) are added. The resulting reaction mixture is stirred at 75° C. overnight. Subsequently, the crude mixture is put into water (2 mL), extracted with EtOAc (2×10 mL), washed with sat. NaHCO₃ (4 mL) and dried over sodium sulfate. Purification on silica gel affords dimethylcarbamic acid 2-[(R)-2-(6-isopropyl-2-methoxy-pyridin-3-yl)-3,7-dimethyl-pyrrolo[2,3-b]pyrazin-5-yl]-butyl ester. LCMS: m/z 440.4 (M+H)⁺

### Example 8

### Synthesis of [6-isopropyl-3-(5-isopropyl-3,7-dimethyl-5H-pyrrolo[2,3-b]pyrazin-2-yl)-pyridin-2-yl]-methyl-amine and 2-(2-ethyl-6-isopropyl-pyridin-3-yl)-5-isopropyl-3,7-dimethyl-5H-pyrrolo[2,3-b]pyrazine

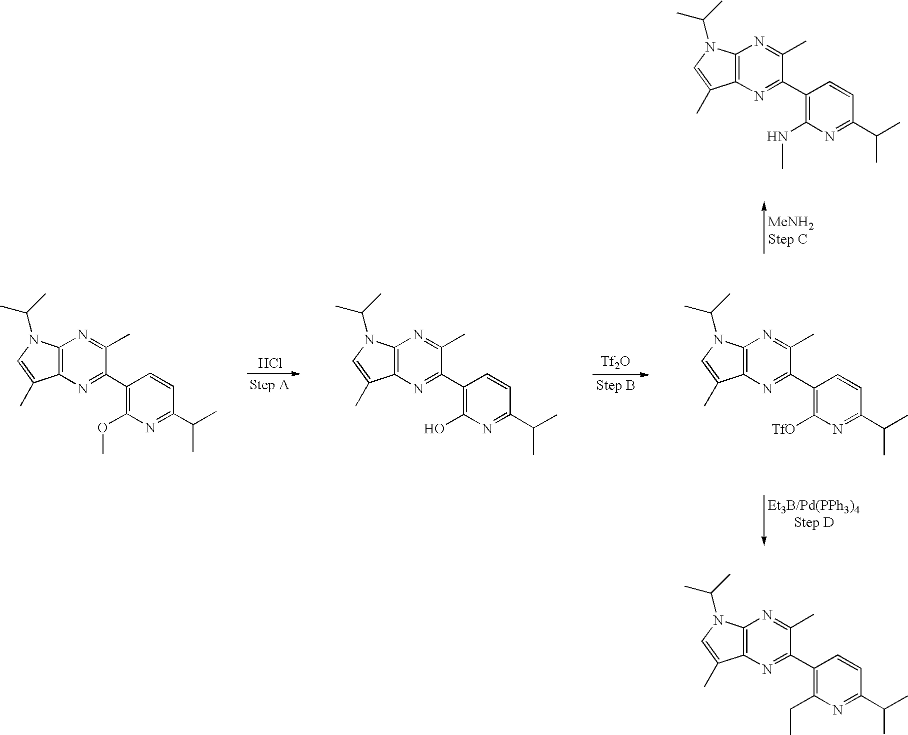

Step A

5-Isopropyl-2-(6-isopropyl-2-methoxy-pyridin-3-yl)-3,7-dimethyl-5H-pyrrolo[2,3-b]pyrazine (900 mg) is heated in HCl (4N, 6 mL) at 75° C. for 8 h. The mixture is then neutralized and extracted with CHCl₃ (2×25 mL). Evaporation affords 5-isopropyl-2-(6-isopropyl-2-hydroxy-pyridin-3-yl)-3,7-dimethyl-5H-pyrrolo[2,3-b]pyrazine LCMS: m/z 325.4 (M+H)⁺

Step B

5-Isopropyl-2-(6-isopropyl-2-hydroxy-pyridin-3-yl)-3,7-dimethyl-5H-pyrrolo[2,3-b]pyrazine (400 mg) is dissolved in CH₂Cl₂ (8 mL). Tf₂O (0.26 mL) and Et₃N (0.26 mL) are added at r.t. and the mixture is stirred for 30 min. The mixture is evaporated and dissolved in EtOAc/hexane (1:1) and washed with sat. NaHCO₃, H₂O and dried. Evaporation affords the triflate. LCMS: m/z 457.4 (M+H)⁺

Step C

The above triflate (215 mg) and methylamine (5M in NMP, 2 mL) are heated at 90° C. for 4 h. Subsequently, the crude mixture is put into water (10 mL), extracted with EtOAc/hexane (1:1, 2×15 mL), and dried over sodium sulfate. Purification on silica gel affords [6-isopropyl-3-(5-isopropyl-3,7-dimethyl-5H-pyrrolo[2,3-b]pyrazin-2-yl)-pyridin-2-yl]-methyl-amine. LCMS: m/z 338.3 (M+H)⁺

Step D

The above triflate (270 mg) is dissolved in toluene (5 mL). After 10 min of degassing, tetrakis(triphenylphosphine)palladium(0) (35 mg) is added, followed by 1 min of degassing. Upon addition of triethylborane (1N in hexane, 1.8 mL), aqueous 1N sodium carbonate solution (1.2 mL) and LiCl (125 mg), the reaction mixture is heated at 110° C. for 6 h. Subsequently, the crude mixture is put into water (100 mL), extracted with EtOAc/hexane (1:4, 3×20 mL), and dried over sodium sulfate. Purification on silica gel affords 2-(2-ethyl-6-isopropyl-pyridin-3-yl)-5-isopropyl-3,7-dimethyl-5H-pyrrolo[2,3-b]pyrazine. LCMS: m/z 337.2 (M+H)⁺

### Example 9

### Synthesis of 1-(1-ethyl-propyl)-5-(2-methoxy-4-trifluoromethoxy-phenyl)-3,6-dimethyl-1H-pyrrolo[3,2-b]pyridine

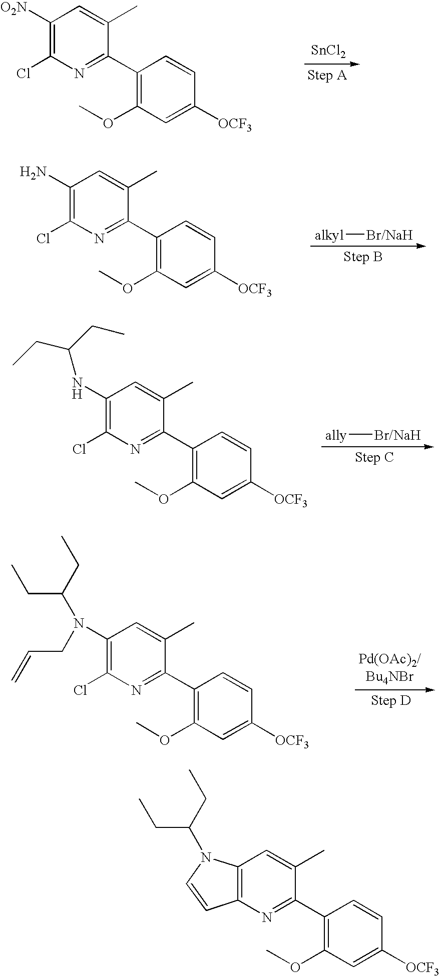

To a solution of the above nitro compound (2.63 g) in ether (30 mL) is added SnCl₂×2H₂O (6.54 g) in conc. HCl (20 mL)) dropwise at room temperature. After the addition is completed, the reaction mixture is stirred at room temperature for 1 h. The reaction mixture is basified with 10N NaOH (cooled with ice-bath) to pH 9-10. After extracting with ether (200 mL×3), the combined ether-layers are dried over Na₂SO₄ to give a crude mixture that is used in step B without any further purification. ¹H NMR (CDCl₃, δ ppm): 7.26 (1H, d, J=8.3 Hz), 6.93 (1H, s), 6.89 (1H, d, J=8.3 Hz), 6.79 (1H, s), 4.03 (2H, brs), 3.78 (3H, s), 2.05 (3H, s).

Step B

To a solution of the crude product (166 mg) from step A in DMSO (2 ml) is added NaH (60%, 60 mg). The reaction mixture is stirred at rt for 2 h, followed by addition of 3-bromopentane (226 mg). After being stirred at rt for 30 min, the yellowish mixture is quenched with water and extracted with EtOAc. The organic layer is washed with water twice, then brine to be finally dried over Na₂SO₄. The crude product is purified on silica gel. ¹H NMR (CDCl₃, δ ppm): 7.26 (1H, d, J=8.3 Hz), 6.87 (1H, d, J=8.3 Hz), 6.77 (1H, s), 6.76 (1H, s), 4.13 (1H, d, J=8.6 Hz), 3.74 (3H, s), 3.24˜3.29 (1 H, m), 2.07 (3H, s), 1.59-1.69 (2H, m), 1.49˜1.59 (2H, m), 0.97 (6H, t, J=7.3 Hz).

Step C

To a solution of the alkylation product of step B (403 mg) in NMP (2 mL) and tetrabutylammonium bromide (cat.) is added NaH (60%, 120 mg). The reaction mixture is stirred at rt for 2 h followed by addition of allyl bromide (2 eq.). After being stirred at 60° C. for 3 h, the mixture is quenched with water, extracted with EtOAc, and dried over Na₂SO₄. The crude product is purified on silica gel. ¹H NMR (CDCl₃, δ ppm): 7.29 (1H, d, J=8.2 Hz), 7.17 (1H, s), 6.86 (1H, d, J=8.2 Hz), 6.76 (1H, brs), 5.66˜5.75 (1H, m), 5.18 (1H, d, J=18 Hz), 5.05 (1H, d, J=10 Hz), 3.77˜3.81 (5H, CH₂, CH₃), 3.32 (1H, m), 2.07 (3H, s), 1.54-1.63 (4H, m), 0.95 (6H, t, J=7.5 Hz).

Step D

A mixture of the allylamine of step C (100 mg), Pd(OAc)₂ (5.1 mg), tetrabutylammonium bromide (72.9 mg), and K₂CO₃ (93 mg) in DMF (3 mL) is degassed and then heated to 80° C. overnight. The mixture is subsequently quenched with water, extracted with EtOAc, and dried over Na₂SO₄. Purification on silica gel yields the title compound. ¹H NMR (CDCl₃, δ ppm): 7.43 (1H, s), 7.35 (1H, d, J=8.3 Hz), 7.08 (1H, d, J=1.0 Hz), 6.91˜6.94 (1H, dm), 6.80 (1H, brs), 3.99 (1H, m), 3.77 (3H, s), 2.40 (3H, s), 2.21 (3H, s), 1.85-1.91 (4H, m), 0.80 (6H, brs).

### Example 10

### Synthesis of ethyl-{4-ethyl-5-[1-(1-ethyl-propyl)-3,6-dimethyl-1H-pyrrolo[3,2-b]pyridin-5-yl]-pyridin-2-yl}-methyl-amine

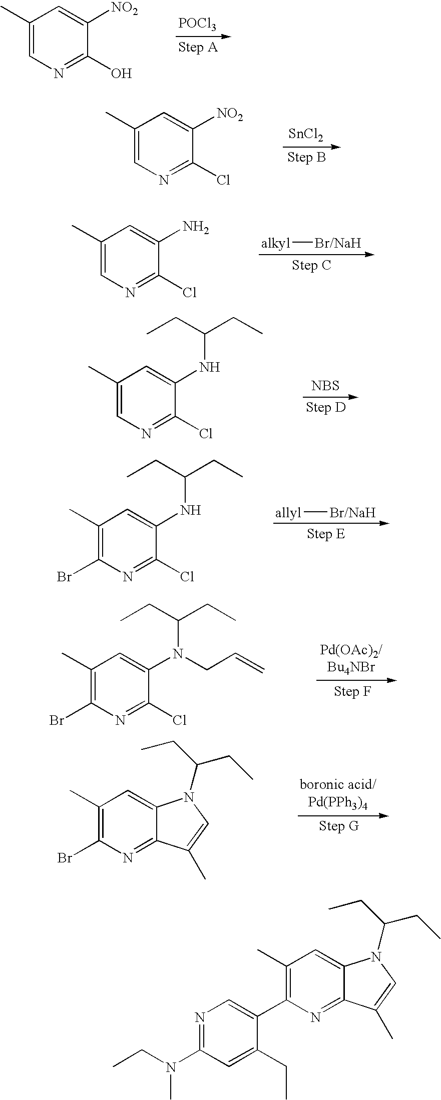

Step A

The shown nitropyridine (25 g) in POCl₃ (100 mL) is refluxed for 8 h. After completed reaction, the reaction mixture is concentrated at reduced pressure to dryness. Ice (100 g) is added to the residue, which is then neutralized with 2N NaOH. Extraction with EtOAc (200 mL×2) and drying over MgSO₄ yields a crude product which is used in step B without any further purification.

Step B

To a solution of the chloro compound from step A (20 g) in ethanol (300 mL) is added SnCl₂×2H₂O (132 g) portionwise. After the addition is completed, the mixture is stirred for an additional 2 h at 50° C. before the solvent is removed under reduced pressure. DCM (400 mL) is added and the suspension is neutralized with 10N NaOH and then filtered through celite. The filtrate is washed with water, brine, and finally dried over MgSO₄ to yield the amine. The crude mixture is used in step C without any further purification.

Step C

To a solution of the amine (13.5 g) from step B in NMP (80 mL) is added tetrabutylammonium bromide (0.3 g) and NaH (60%, 7.6 g) at 0° C. After being stirred at rt for 3 h, 3-bromopentane (1.5 eq.) is added. The reaction mixture is then stirred for an additional 2 h before being quenched with water and extracted with EtOAc. The organic layer is washed with water, brine, and dried over MgSO₄. Evaporation under reduced pressure yields a crude product which is used in step D without any further purification. ¹H NMR (CDCl₃, δ ppm): 7.48 (1H, s), 6.67 (1H, s ), 4.07 (1H, d, J=8.2 Hz), 3.2˜3.24 (1H, m), 2.23 (3H, s), 1.48-1.68 (4H, m), 0.93 (6H, t, J=7.3 Hz).

Step D

The crude material of step C (3.0 g) is dissolved in CHCl₃ (20 mL) and NBS (2.63 g) is added at room temperature. After being stirred at rt for 30 min, the reaction mixture is washed with water, brine, and dried over Na₂SO₄ before it is purified on silica gel to yield the bromide. ¹H NMR (CDCl₃, δ ppm): 6.74 (1H, s), 4.04 (1H, d, J=7.8 Hz), 3.17˜3.22 (1H, m), 2.29 (3H, s), 1.47-1.56 (2H, m), 1.56-1.66 (2H, m), 0.93 (6H, t, J=7.4 Hz).

Step E

To a solution of the bromide from step D (3.66 g) in NMP is added tetrabutylammonium bromide (0.1 g) and NaH (60%, 1.0 g) at rt. After being stirred at rt for 3 h, allyl bromide (3.0 g) is added and the reaction mixture is stirred for an additional 4 h. The reaction mixture is then quenched with water and extracted with EtOAc. The organic layer is washed with water, brine, and dried over MgSO₄ to yield a crude product, which is used in step F without any further purification. ¹H NMR (CDCl₃, δ ppm): 7.11 (1H, s), 5.56-5.66 (1H, m), 5.13 (1H, d, J=17.4 Hz), 5.13 (1H, d, J=10 Hz), 3.70-3.72 (2H, m), 3.22˜3.26 (1H, m), 2.29 (3H, s), 1.52-1.60 (4H, m), 0.91 (6H, t, J=7.4 Hz).

Step F

The crude material of step E (4.1 g), Pd(OAc)₂ (275 mg), tetrabutylammonium bromide (4.5 g), and K₂CO₃ (5.1 g) are dissolved in DMF (20 mL). After degassing, the mixture is heated to 80° C. overnight. The black solution is then diluted with EtOAc before being washed with H₂O, brine, and dried over MgSO₄. Purification on silica gel yields the bicyclic compound. ¹H NMR (CDCl₃, δ ppm): 7.43 (1H, s), 7.05 (1H, s), 3.89-3.92 (1H, m), 2.48 (3H, s), 2.36 (3H, s), 1.76-1.88 (4H, m), 0.72 (6H, t, J=7.3 Hz).

Step G

The bicyclic material of step F (118 mg), Pd(PPh₃)₄ (70 mg) and the previously described 4-ethyl-2-ethylmethylamino-3-pyridine boronic acid (104 mg) are dissolved in toluene (10 mL). Upon addition of 2N Na₂CO₃ (4 mL), the mixture is degassed and then heated overnight to 80° C. Subsequently, the mixture is diluted with EtOAc and washed with H₂O, brine, and finally dried over MgSO₄. Purification on silica gel yields the title compound. ¹H NMR (CDCl₃, δ ppm): 8.00 (1H, s), 7.43 (1H, s ), 7.07 (1H, brs), 6.45 (1H, s), 3.96-4.01 (1H, m), 3.62 (2H, q, J=7.0 Hz), 2.48 (3H, s), 3.06 (3H, s), 2.42 (2H, q, J=7.5 Hz), 2.39 (3H, s), 2.23 (3H, s), 1.81-1.90 (4H, m), 1.18 (3H, t, J=7.2 Hz), 1.03 (3H, t, J=7.5 Hz), 0.81 (6H, t, J=7.3 Hz).

### Example 11

### Synthesis of 5-(2-ethyl-6-isopropyl-pyridin-3-yl)-1-(1-ethyl-propyl)-3,6-dimethyl-1H-pyrrolo[3,2-b]pyridine

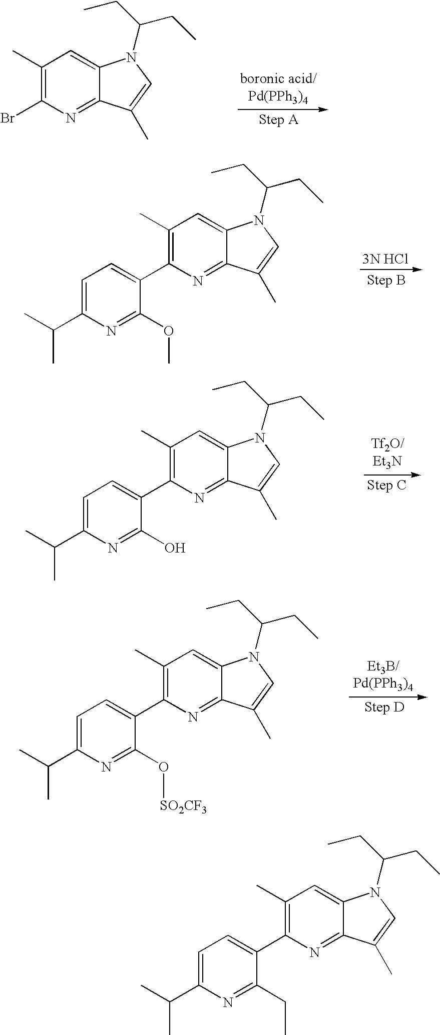

Step A

The previously described bicyclic bromide (590 mg), the also previously described 6-isopropyl-2-methoxy-3-pyridine boronic acid (507 mg), and Pd(PPh₃)₄ (115 mg) are dissolved in toluene (30 mL). Upon addition of 2N Na₂CO₃ (6 mL), the mixture is degassed and then heated overnight to 85° C. Subsequently, the mixture is diluted with EtOAc and washed with 2N NaOH, H₂O, brine, and finally dried over MgSO₄. Purification on silica gel yields the coupled product. ¹H NMR (CDCl₃, δ ppm): 7.55 (1H, d, J=7.3 Hz), 7.42 (1H, s), 7.06 (1H, d, J=1.1 Hz), 6.84 (1H, d, J=7.5 Hz), 3.96-4.00 (1H, m), 3.91 (3H, s), 2.98-3.01 (1H, m), 2.39 (3H, d, J=1.1 Hz), 2.25 (3H, s), 1.82-1.90 (4H, m), 1.31 (6H, d, J=7.0 Hz), 0.80 (6H, t, J=7.5 Hz).

Step B

The Suzuki-product of step A (718 mg) is dissolved in 3N HCl (50 mL) and heated to 70° C. overnight. The reaction mixture is cooled to ambient temperature, neutralized with 2N NaOH, and extracted with CHCl₃ (100 mL×2). Drying over MgSO₄ yields the pyridone, which is used in step C without any further purification.

Step C

The pyridone (700 mg) of step B is dissolved in CH₂Cl₂. Triethylamine (3 eq.) is added, followed by dropwise addition of Tf₂O (1.5 equivalents) at 0° C. After being stirred at rt for 2 h, the reaction mixture is washed with H₂O, brine, and dried over MgSO₄. The triflate is used in step D without any further purification.

Step D

The crude material of step C (48 mg), Pd(PPh₃)₄ (11.5 mg), and triethylborane (0.5 mL, 1N in hexane) are dissolved in toluene (2 mL). After addition of 2N Na₂CO₃ (0.5 mL), the mixture is degassed and then heated at 85° C. overnight. The solution is diluted with EtOAc and washed with 2N NaOH, H₂O, brine, and finally dried over MgSO₄. Purification on silica gel yields the title compound. ¹H NMR (CDCl₃, δ ppm): 7.46 (1H, d, J=7.9 Hz), 7.44 (1H, s ), 7.09 (1H, d, J=0.8 Hz), 7.06 (1H, d, J=7.7 Hz), 3.97-4.01 (1H, m), 3.10-3.13 (1H, m), 2.60 (2H, q, J=7.3 Hz), 2.39 (3H, d, J=0.8 Hz), 2.16 (3H, s), 1.81-1.90 (4H, m), 1.32 (6H, d, J=7.0 Hz), 1.15 (3H, t, J=7.3 Hz), 0.80 (6H, t, J=7.6 Hz).

### Example 12

### Synthesis of 5-(2-ethyl-6-isopropyl-pyridin-3-yl)-3,6-dimethyl-1-propyl-1H-pyrrolo[3,2-b]pyridine, [3-(3,6-dimethyl-1-propyl-1H-pyrrolo[3,2-b]pyridin-5-yl)-6-isopropyl-pyridin-2-yl]-methyl-amine and [3-(3,6-dimethyl-1-propyl-1H-pyrrolo[3,2-b]pyridin-5-yl)-6-isopropyl-pyridin-2-yl]-ethyl-amine

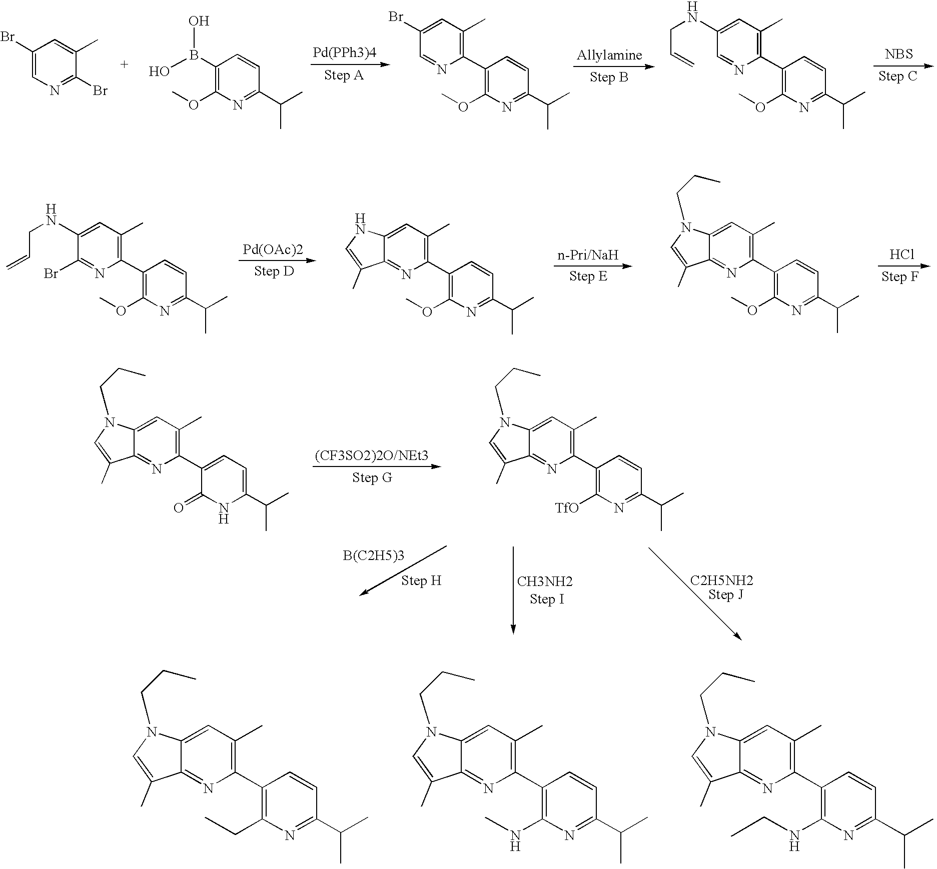

A mixture of 2,5-dibromo-3-methylpyridine (5.02 g, 0.02 mol)), 2-methoxy-6-isopropyl-3-pyridylboronic acid (4.10 g, 0.021 mol), Pd(PPh₃)₄ (924 mg), aqueous Na₂CO₃ solution (1.0M, 40 ml), and toluene (50 ml) is heated at 100° C. under the N2 atmosphere overnight. The reaction mixture is cooled to room temperature and separated. The aqueous layer is extracted with ethyl acetate. The combined organic layers are washed with brine and dried with Na₂SO₄. Purification by flash column with hexane/ethyl acetate gives product as clear oil. LCMS: m/z 323.3 (M+H)⁺

Step B

A mixture of bromide (9.63 g, 0.03 mol), allylamine (6.75 ml), BINAP (1.5 g), Pd₂(dba)₃ (10 g), NaO-t-Bu (5.77 g) in toluene (150 ml) is heated at 100° C. under N₂ atmosphere overnight. The reaction mixture is cooled to room temperature and quenched with water. The resulting mixture is separated and extracted with ethyl acetate. The combine organic layers are washed with brine and dried with Na₂SO₄. Purification by flash column with hexane/ethyl acetate gives product as clear oil. LCMS: m/z 298.3 (M+H)⁺

Step C

The starting material (6.67 g) is taken in anhydrous CHCl₃ (100 ml). 1.0 equivalent of NBS is added in one portion at 0° C. The reaction is complete in 0.5 hour. The reaction mixture is washed with brine and dried with Na₂SO₄. Purification by flash column with hexane/ethyl acetate gives product as clear oil. LCMS: m/z 376.4 (M+H)⁺

Step D

A mixture of bromide (6.6 g), tetrabutylammonium bromide (7.07 g), K₂CO₃ (7.28 g), Pd(OAc)₂ (150 mg) in DMF (70 ml) is heated at 80° C. under N2 atmosphere for 0.5 hour. The reaction mixture is cooled to room temperature and diluted with water. The resulting mixture is extracted with ethyl acetate. The combine organic layers are washed with brine and dried with Na₂SO₄. Purification by flash column with hexane/ethyl acetate gives product as off-white solid. LCMS: m/z 296.4 (M+H)⁺

Step E

NaH (100 mg, 60% in mineral oil) is added to a solution of starting material (58 mg) in anhydrous DMF (5 ml) and stirred for 10 minutes. 1-iodopropane (0.5 ml) is added and stirred for 0.05 hour. The reaction mixture is carefully quenched with 1 ml of methanol and diluted with water. The resulting mixture is extracted with ethyl acetate. The combine organic layers are washed with brine and dried with Na₂SO₄. Purification by flash column with hexane/ethyl acetate gives product as clear oil. LCMS: m/z 338.4 (M+H)⁺

Step F

Starting material (360 mg) is taken into 4N HCl (20 ml) and heated at 75° C. overnight. Reaction mixture is cooled to 0° C. and the pH value is adjusted to −12 by adding 10N aqueous NaOH solution. The resulting mixture is extracted with chloroform. The combine organic layers are washed with brine and dried with Na₂SO₄. Concentration gives crude product as an off-white solid. It is used for the next step reaction without further purification. LCMS: m/z 324.4 (M+H)⁺

Step G

Pyridone (330 mg) is taken in anhydrous methylene chloride (20 ml) and cooled to 0° C., Triflic anhydride (1.5 equiv.) is added followed by the addition of triethylamine (2 equiv.). The reaction is complete in 0.5 hour. The reaction mixture is washed with saturated NaHCO3 and dried with Na₂SO₄. The crude product is used for the next step reaction without further purification. LCMS: m/z 456.4 (M+H)⁺

Step H

A mixture of triflate (180 mg), LiCl (84 mg), Pd(PPh₃)₄ (23 mg), Na₂CO₃ (1.0M in water, 1 ml), B(C₂H₅)₃ (1.0M in hexane, 1.5 ml) in toluene (2 ml) is heated at 100° C. in sealed tube for 2 hours. The resulting mixture is cooled to room temperature and extracted with ethyl acetate. The combine organic layers are washed with brine and dried with Na₂SO₄. Purification by flash column with hexane/ethyl acetate gives 5-(2-ethyl-6-isopropyl-pyridin-3-yl)-3,6-dimethyl-1-propyl-1H-pyrrolo[3,2-b]pyridine as clear oil. LCMS: m/z 336.4 (M+H)⁺

Step I

Triflate (230 mg) is taken in anhydrous N-methylpyrrolidinone (2 ml), CH₃NH₂ is added as a solution of NMP (˜5.5M, 2 ml). The resulting mixture is heated at 85° C. in a sealed tube overnight. The reaction mixture is cooled to room temperature and diluted with water. The resulting mixture is extracted with ethyl acetate. The combine organic layers are washed with brine and dried with Na₂SO₄. Purification by flash column with hexane/ethyl acetate gives [3-(3,6-dimethyl-1-propyl-1H-pyrrolo[3,2-b]pyridin-5-yl)-6-isopropyl-pyridin-2-yl]-methyl-amine as clear oil. LCMS: m/z 337.4 (M+H)⁺

Step J

Triflate (420 mg) is taken in anhydrous N-methylpyrrolidinone (3 ml), C₂H₅NH₂ is added as a solution of THF (2.0M, 2 ml). The resulting mixture is heated at 85° C. in a sealed tube overnight. The reaction mixture is cooled to room temperature and diluted with water. The resulting mixture is extracted with ethyl acetate. The combine organic layers are washed with brine and dried with Na₂SO₄. Purification by flash column with hexane/ethyl acetate gives [3-(3,6-dimethyl-1-propyl-1H-pyrrolo[3,2-b]pyridin-5-yl)-6-isopropyl-pyridin-2-yl]-ethyl-amine as clear oil. LCMS: m/z 351.5 (M+H)⁺

### Example 13

### Synthesis of (R)-2-[5-(6-isopropyl-2-methoxy-pyridin-3-yl)-3,6-dimethyl-pyrrolo[3,2-b]pyridin-1-yl]-3-methoxy-propan-1-ol and 1-((S)-2-fluoro-1-methoxymethyl-ethyl)-5-(6-isopropyl-2-methoxy-pyridin-3-yl)-3,6-dimethyl-1H-pyrrolo[3,2-b]pyridine

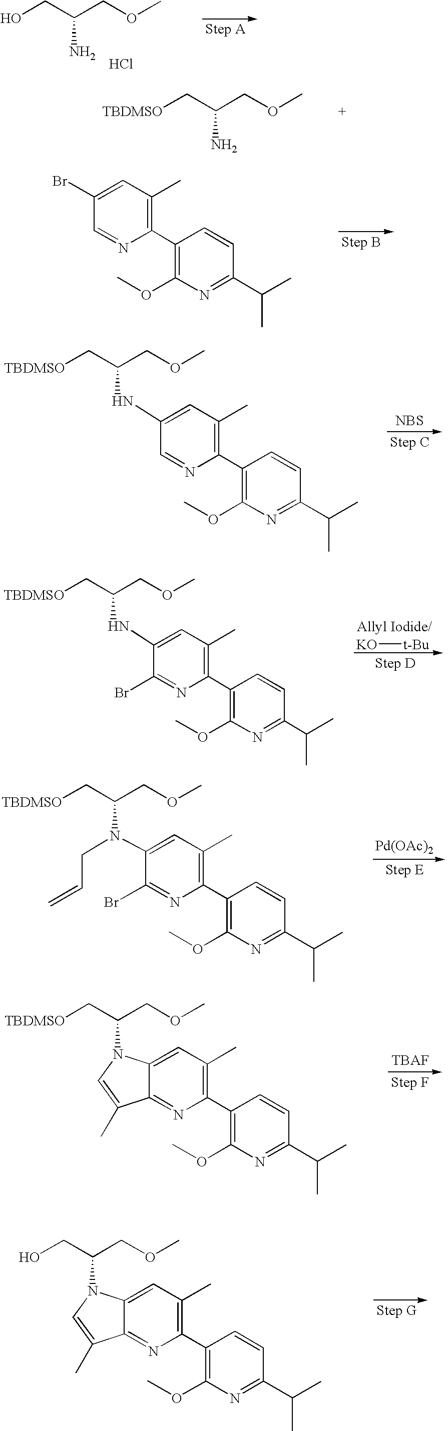

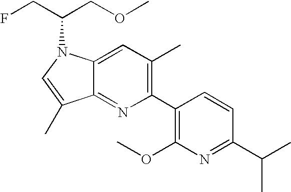

Step A

(R)-2-Amino-3-methoxy-propan-1-ol hydrochloride (CAS#: 148278-96-0) (6.74 g) and imidazole (13.2 g) are taken in anhydrous methylene chloride (300 ml). TBDMSCl (21.9 g) is added in one portion. The reaction is carried on overnight. The reaction mixture is washed with water (200 ml×3) and dried with Na₂SO₄. Concentration removes all volatiles. The crude product is used for next step reaction without further purification.

Step B

A mixture of bromide (6.42 g, 0.02 mol), amine (1.5 equiv.), BINAP (1.0 g), Pd₂(dba)₃ (0.6 g), NaO-t-Bu (4.0 g) in toluene (80 ml) is heated at 85° C. under N₂ atmosphere overnight. The reaction mixture is cooled to room temperature and quenched with water. The resulting mixture is separated and extracted with ethyl acetate. The combine organic layers are washed with brine and dried with Na₂SO₄. Purification by flash column with hexane/ethyl acetate gives product as clear oil. Rf: 0.3 (Hexane/ethyl acetate: 3/1)

Step C

The starting material (7.33 g) is taken in anhydrous CHCl₃ (100 ml). 0 equivalent of NBS is added in one portion at 0° C. The reaction is complete in 0.5 hour. The reaction mixture is washed with brine and dried with Na₂SO₄. Purification by flash column with hexane/ethyl acetate gives product as clear oil. Rf: 0.3 (Hexane/ethyl acetate: 15/1)

Step D

Starting material (5.94 g) is taken in anhydrous THF (100 ml). Allyl iodide (3.6 ml) is added followed by the addition of KO-t-Bu/THF solution (1.0M, 44 ml) at room temperature. The reaction is stirred at room temperature for 3 hours. The reaction mixture is quenched with water. The resulting mixture is separated and extracted with ethyl acetate. The combine organic layers are washed with brine and dried with Na₂SO₄. The crude product is used for the next step reaction without further purification. Rf: 0.3 (Hexane/ethyl acetate: 19/1)

Step E

The crude product (6.4 g) of previous reaction is taken in DMF (60 ml) followed by the addition of tetrabutylammonium bromide (4.45 g), K₂CO₃ (4.58 g), Pd(OAc)₂ (125 mg). The resulting mixture is heated at 85° C. under N₂ atmosphere for one hour. The reaction mixture is cooled to room temperature and diluted with water. The resulting mixture is extracted with ethyl acetate. The combine organic layers are washed with brine and dried with Na₂SO₄. Purification by flash column with hexane/ethyl acetate gives product as clear oil. 0.3 (Hexane/ethyl acetate: 5/1)

Step F

Starting material (5.43 g) is taken in THF (60 ml) followed by the addition of tetrabutylammonium fluoride (2 equiv.) at room temperature. The reaction is complete after 2 hours. The reaction mixture is washed with water, brine and dried with Na₂SO₄. Concentration gives (R)-2-[5-(6-isopropyl-2-methoxy-pyridin-3-yl)-3,6-dimethyl-pyrrolo[3,2-b]pyridin-1-yl]-3-methoxy-propan-1-ol as an off-white solid. LCMS: m/z 384.4 (M+H)⁺

Step G

Starting material (1.15 g, 3 mmol) is taken in anhydrous methylene chloride (50 ml) followed by the addition of [Bis(2-methoxyethyl)amino]sulfur trifluoride (2 equiv.) at room temperature. The reaction is stirred at room temperature overnight. The reaction mixture is carefully quenched with ice-water. The resulting mixture is separated and extracted with methylene chloride and dried with Na₂SO₄. Purification by flash column with hexane/ethyl acetate gives 1-((S)-2-fluoro-1-methoxymethyl-ethyl)-5-(6-isopropyl-2-methoxy-pyridin-3-yl)-3,6-dimethyl-1H-pyrrolo[3,2-b]pyridine as clear oil. 0.3 (Hexane/ethyl acetate: 5/1)

### Example 14

### Synthesis of 5-(6-Isopropyl-2-methoxy-pyridin-3-yl)-1-((S)-2-methoxy-1-methyl-ethyl)-3,6,7-trimethyl-1H-pyrrolo[3,2-b]pyridine

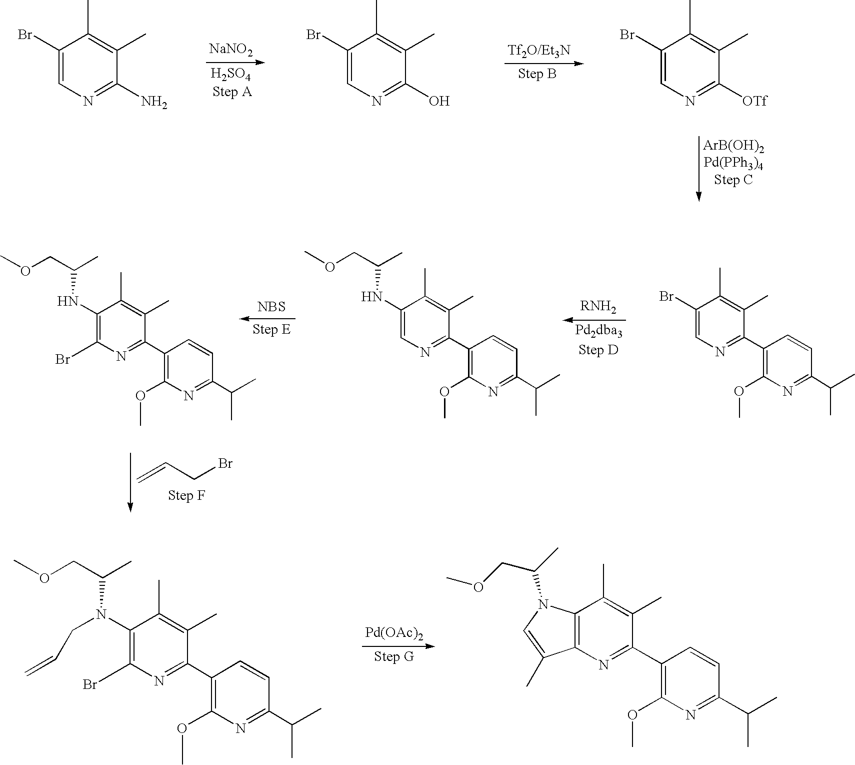

Step A

2-Amino-5-bromo-3,4-dimethylpyridine (201 mg) in H₂SO₄ (2.5N, 2.4 mL) is cooled to 0° C. and subsequently treated dropwise with sodium nitrite (104 mg) in H₂O (1 mL). The solid is collected and washed with H₂O and dried to afford 2-hydroxy-5-bromo-3,4-dimethylpyridine. LCMS: m/z 202.2 and 204.2 (M+H)⁺

Step B

2-Hydroxy-5-bromo-3,4-dimethylpyridine (165 mg) is dissolved in CHCl₃ (3 mL). Triflic anhydride (0.17 mL) and Et₃N (0.17 mL) are added at r.t. and the mixture is stirred for 30 min. The mixture is evaporated and dissolved in EtOAc/hexane (2:8) and washed with sat. NaHCO₃, H₂O and dried. Evaporation affords the triflate. LCMS: m/z 334.0 and 336.0 (M+H)⁺

Step C

The above triflate (272 mg) and 2-methoxy-6-isopropyl-3-pyridineboronic acid are dissolved in DME (3.5 mL). After 10 min of degassing, tetrakis(triphenylphosphine)palladium(0) (12 mg) is added, followed by 1 min of degassing. Upon addition of an aqueous 1N sodium carbonate solution (1.63 mL) and LiCl (140 mg), the reaction mixture is heated to 90° C. for 16 h. Subsequently, the crude mixture is put into water (100 mL), extracted with EtOAc/hexane (20:80, 3×20 mL), and dried over sodium sulfate. Purification on silica gel affords 3-(5-bromo-3,4-dimethyl-pyridin-2-yl)-2-methoxy-6-isopropylpyridine. LCMS: m/z 335.1 and 337.1 (M+H)⁺

Step D

3-(5-bromo-3,4-dimethyl-pyridin-2-yl)-2-methoxy-6-isopropylpyridine (85 mg), (S)-2-methoxy-1-methyl-ethylamine (34 mg), Pd₂dba₃ (12 mg), BINAP (16 mg) and t-BuONa (37 mg) are dissolved in toluene (3.5 mL). The reaction mixture is heated to 90° C. for 6 h. Subsequently, the crude mixture is put into water (10 mL), extracted with EtOAc/hexane (1:1, 2×10 mL), and dried over sodium sulfate. Purification on silica gel affords 3-{5-[(S)-2-methoxy-1-methyl-ethylamino]-3,4-dimethyl-pyridin-2-yl}-2-methoxy-6-isopropylpyridine. LCMS: m/z 344.4 (M+H)⁺

Step E

3-{5-[(S)-2-methoxy-1-methyl-ethylamino]-3,4-dimethyl-pyridin-2-yl}-2-methoxy-6-isopropylpyridine (42 mg) is dissolved in CHCl₃ (1 mL) Upon addition of NBS (24 mg), the reaction mixture is stirred at 25° C. for 30 min. Subsequently, the crude mixture is put into water (10 mL), extracted with EtOAc/hexane (1:4, 2×5 mL), and dried over sodium sulfate. Purification on silica gel affords 3-{6-bromo-5-[(S)-2-methoxy-1-methyl-ethylamino]-3,4-dimethyl-pyridin-2-yl}-2-methoxy-6-isopropylpyridine. LCMS: m/z 422.3 and 424.3 (M+H)⁺

Step F

3-{6-bromo-5-[(S)-2-methoxy-1-methyl-ethylamino]-3,4-dimethyl-pyridin-2-yl}-2-methoxy-6-isopropylpyridine (2.0 g) is dissolved in NMP (15 ml). Upon addition of NaH (60%, 380 mg), the reaction mixture is stirred at 25° C. for 30 min before allyl bromide (0.82 mL) is added. The reaction mixture is then heated to 50° C. overnight. Subsequently, the crude mixture is put into water (10 mL), extracted with EtOAc/hexane (1:4, 2×50 mL), and dried over sodium sulfate. Purification on silica gel affords 3-{6-bromo-5-[(S)-N-allyl-2-methoxy-1-methyl-ethylamino]-3,4-dimethyl-pyridin-2-yl}-2-methoxy-6-isopropylpyridine. LCMS: m/z 462.4 and 464.4 (M+H)⁺

Step G

3-{6-bromo-5-[(S)-N-allyl-2-methoxy-1-methyl-ethylamino]-3,4-dimethyl-pyridin-2-yl}-2-methoxy-6-isopropylpyridine (0.75 g) is dissolved in DMF (6 mL). After 10 min of degassing, Pd(OAc)₂ (36 mg) is added, followed by 1 min of degassing. Upon addition of potassium carbonate (670 mg) and Bn₄NBr (650 mg), the reaction mixture is heated to 90° C. for 2 h. Subsequently, the crude mixture is put into water (100 mL), extracted with EtOAc/hexane (1:2, 3×50 mL), and dried over sodium sulfate. Purification on silica gel affords 5-(6-Isopropyl-2-methoxy-pyridin-3-yl)-1-((S)-2-methoxy-1-methyl-ethyl)-3,6,7-trimethyl-1H-pyrrolo[3,2-b]pyridine. LCMS: m/z 382.3 (M+H)⁺

### Example 15

### Synthesis of 5-(2-ethyl-6-methoxy-pyridin-3-yl)-1-((S)-2-methoxy-1-methyl-ethyl)-3,6-dimethyl-1H-pyrrolo[3,2-b]pyridine

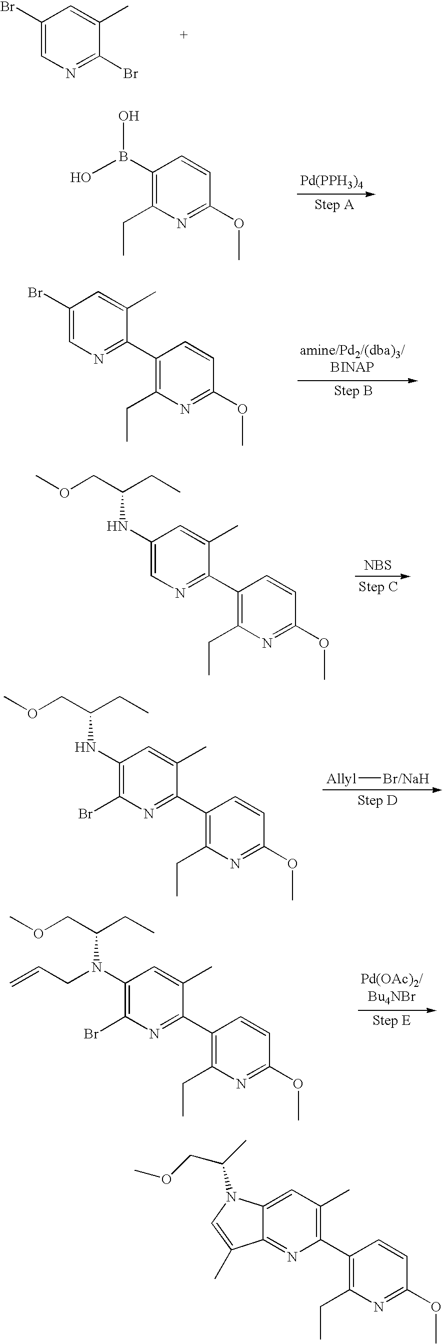

Step A

2,5-dibromo-3-methylpyridine (18.90 g) and the previously described 2-ethyl-6-methoxy-3-pyridine boronic acid (13.70 g) are dissolved in DME (200 mL). After degassing, tetrakis(triphenylphosphine)palladium(0) (3.60 g) is added. A second degassing is followed by addition of a 5N sodium carbonate solution (30 mL) whereupon the reaction is heated to 80° C. for 16 h. The yellowish mixture is then put into water (500 mL), extracted with DCM (2×300 mL), and dried over sodium sulfate. Purification on silica gel affords the coupled product. LCMS: m/z 306.94 (M+H)⁺

Step B

The purified compound (6.40 g) of step A and (S)-1-methoxy-2-aminopropane (2.04 g) are dissolved in toluene (80 mL) and briefly degassed. Subsequently, Pd₂(dba)₃ (1.03 g), rac-2,2′-bis(diphenylphosphino)-1,1′-binaphthyl (0.76 g), and sodium tert-butoxide (2.81 g) are added before the mixture is heated to 70° C. for 16 h. The black solution is then put into water (400 mL) and sat. sodium bicarbonate (100 mL), extracted with DCM (3×300 mL), and dried over magnesium sulfate. Flushing the crude material through a plug of silica gel affords the 5-aminopyridine as a semi-crude that is used in step C. LCMS: m/z 316.35 (M+H)⁺

Step C

The amino compound of step B is dissolved in chloroform (200 mL) and NBS (0.9-1.0 eq) is added in portions until TCL control verifies full conversion of the starting material. Subsequently, the yellowish mixture is put into water (200 mL), extracted with DCM (3×100 mL), and dried over magnesium sulfate. Purification on silica gel affords the bromide. LCMS: m/z 394.21 (M+H)⁺

Step D

The purified bromide (7.59 g) of step C and allyl bromide (2.04 mL) are dissolved in DMF (100 mL). Sodium hydride (1.16 g) is added in 3 portions and the reaction is stirred for 90 min at rt. After TLC control confirms some starting material is still remaining, 0.25 equivalents of both reagents are added to drive the reaction to completion. The mixture is then put into water (500 mL) and extracted with ethyl ether (2×300 mL). The combined organic layers are washed with water (100 mL), dried over magnesium sulfate, and purified on silica gel to afford the allylated amine. LCMS: m/z 434.23 (M+H)⁺

Step E

The allyl compound (7.89 g) of step D, tetrabutylammonium bromide (5.85 g), palladium acetate (0.41 g), and potassium carbonate (7.53 g) are dissolved in DMF (150 mL). After heating to 80° C. for 30 min, the mixture is worked-up according to step D. Final purification on silica gel affords the title compound. LCMS: m/z 354.39 (M+H)⁺

### Example 16

### Synthesis of 6-ethyl-2-methoxy-5-1-((S)-2-methoxy-1-methyl-ethyl)-3,6-dimethyl-1H-pyrrolo[3,2-b]pyridin-5-yl]-N-methyl-nicotinamide

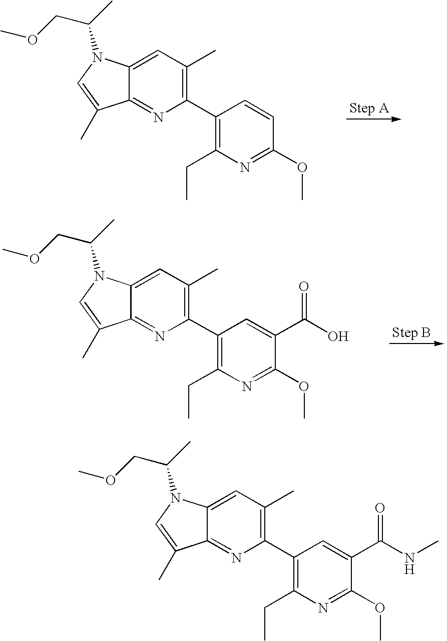

Step A

5-(2-Ethyl-6-methoxy-pyridin-3-yl)-1-((S)-2-methoxy-1-methyl-ethyl)-3,6-dimethyl-1H-pyrrolo[3,2-b]pyridine (135 mg) is dissolved in THF (10 mL) and then cooled to −40° C. Upon addition of t-BuLi (0.45 mL, 1.7N in pentane) the temperature is elevated to 0° C. and kept there for 30 min. Prior to injecting gaseous carbon dioxide, the temperature is brought to −78° C. After injection, the solution is kept at this temperature for another 10 min and is then put into 1N NaOH (100 mL). After washing the solution with ethyl ether (2×100 mL), the aqueous layer is neutralized and extracted with DCM (3×100 mL). The combined DCM-phases are dried over magnesium sulfate. The crude has sufficient purity and is used in step B without any further purification. LCMS: m/z 398.41 (M+H)⁺

Step B

The crude mixture (50 mg) of step A, BOP (84 mg), and Huenig base (67 μL) are dissolved in THF (5 mL). The mixture is stirred for 5 min before methylamine (250 μL, 2N in THF) is added. After stirring for 16 h, the yellowish solution is put into water (100 mL), extracted with DCM (3×100 mL), and dried over magnesium sulfate. Final purification on silica gel affords the title compound. LCMS: m/z 411.41 (M+H)⁺

### Example 17

### Synthesis of 5-(6-cyclopropylmethoxy-2-ethyl-pyridin-3-yl)-1-((S)-2-methoxy-1-methyl-ethyl)-3,6-dimethyl-1H-pyrrolo[3,2-b]pyridine

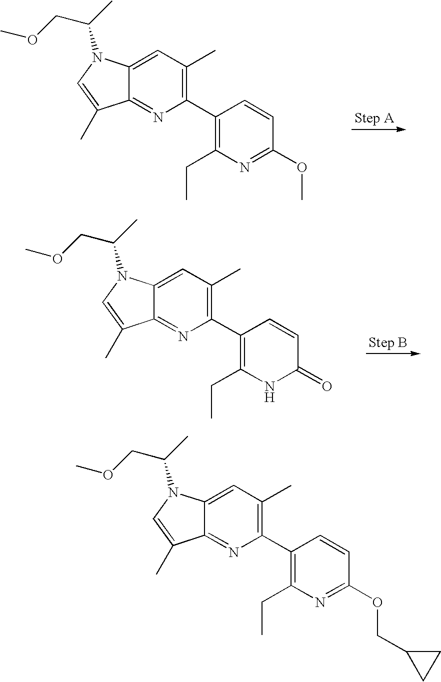

5-(2-Ethyl-6-methoxy-pyridin-3-yl)-1-((S)-2-methoxy-1-methyl-ethyl)-3,6-dimethyl-1H-pyrrolo[3,2-b]pyridine (5.00 g) is dissolved in 4N HCl (150 mL) and heated to 75° C. for 7 days. Once TLC control verifies mostly hydrolyzed material, 10N NaOH (60.0 mL) and sat. sodium bicarbonate (200 mL) are added. Extraction with DCM (3×200 mL), drying over magnesium sulfate, and purification on silica gel affords the pyridone. LCMS: m/z 340.06 (M+H)⁺

Step B

The pyridone (50 mg) of step C, bromomethylcyclopropane (500 mg), and potassium carbonate (500 mg) are dissolved in DMF (3.0 mL). After being stirred over night at rt, the mixture is put into water (100 mL), extracted with DCM (3×100 mL), and dried over magnesium sulfate. Purification on silica gel affords the title compound. LCMS: m/z 394.16 (M+H)⁺

### Example 18

### Synthesis of 5-(6-cyclopropyl-2-ethyl-pyridin-3-yl)-1-((S)-2-methoxy-1-methyl-ethyl)-3,6-dimethyl-1H-pyrrolo[3,2-b]pyridine and {6-ethyl-5-[1-((S)-2-methoxy-1-methyl-ethyl)-3,6-dimethyl-1H-pyrrolo[3,2-b]pyridin-5-yl]-pyridin-2-yl}-dimethyl-amine

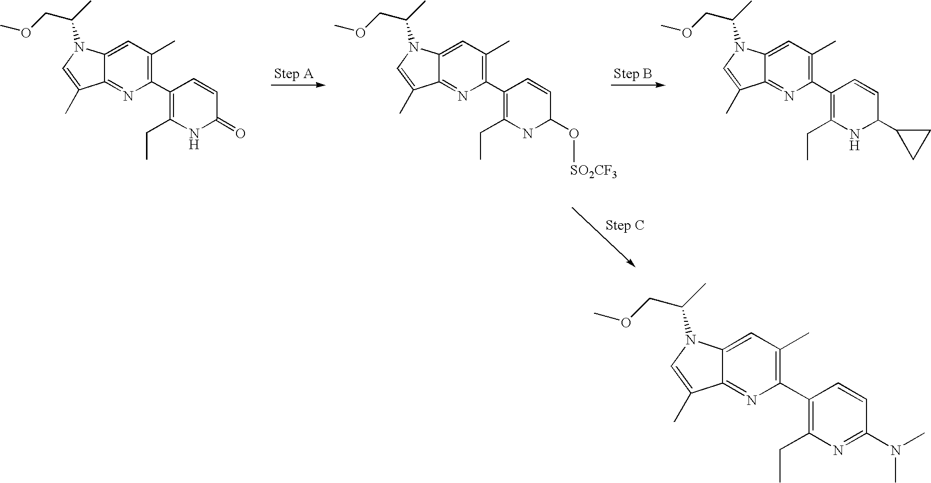

Step A

6-Ethyl-5-[1-((S)-2-methoxy-1-methyl-ethyl)-3,6-dimethyl-1H-pyrrolo[3,2-b]pyridin-5-yl]-1H-pyridin-2-one (2.00 g) and triethylamine (2.05 mL) are dissolved in DCM (100 mL). After being cooled to 0° C., trifluoromethanesulfonic anhydride is added and the reaction is stirred for 30 min at that temperature. Subsequently, the yellowish mixture is put into water (200 mL), extracted with DCM (3×200 mL), and dried over magnesium sulfate. Purification on silica gel affords the triflate. LCMS: m/z 472.26 (M+H)⁺

Step B

The triflate (50 mg) of step A and cyclopropyl boronic acid (91 mg) are dissolved in toluene (5 mL). After being degassed for 5 min, tetrakis(triphenylphosphine)palladium(0) (12 mg) is added and the mixture is degassed again. Adding a potassium carbonate solution (0.50 mL, 2N) is followed by heating to 110° C. for 16 h. Subsequently, the mixture is put into water (100 mL), extracted with DCM (3×100 mL), and dried over magnesium sulfate. Purification on silica gel affords the title compound. LCMS: m/z 364.45 (M+H)⁺

Step C

The triflate (100 mg) of step A is dissolved in a 5N NMP-solution of dimethylamine (1.50 mL) and subsequently heated to 80° C. for 8 h. The reaction mixture is then put into water (100 mL), extracted with DCM (3×100 mL), and dried over magnesium sulfate. Purification on silica gel affords the title compound. LCMS: m/z 367.43 (M+H)⁺

### Example 19

### Synthesis of (3S,4R)-3-(2-fluoro-ethoxy)-4-[5-(6-isopropyl-2-methoxy-pyridin-3-yl)-3,6-dimethyl-pyrrolo[3,2-b]pyridin-1-yl]-pyrrolidine-1-carboxylic acid benzyl ester

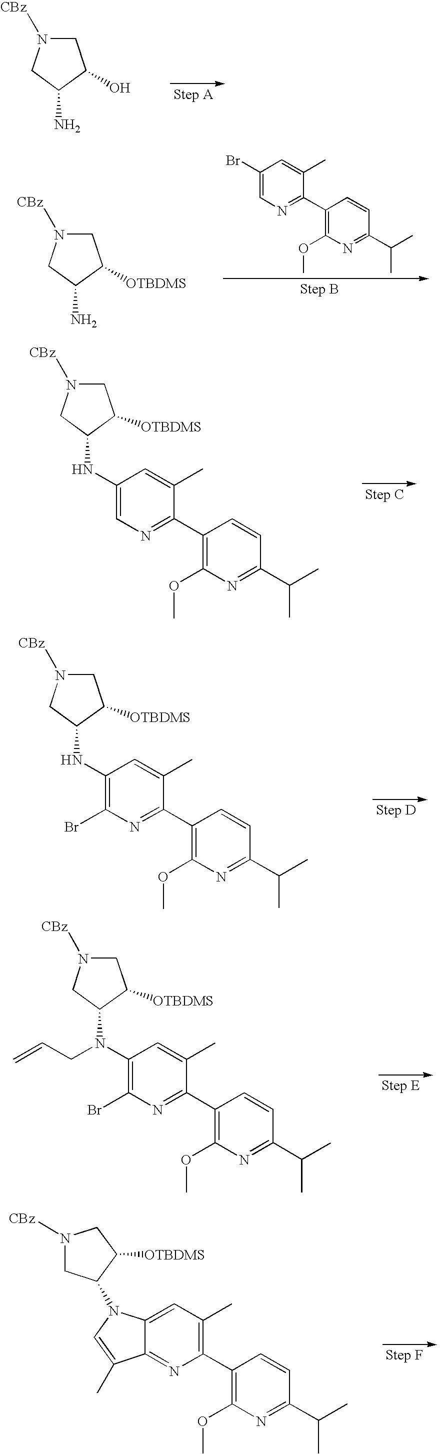

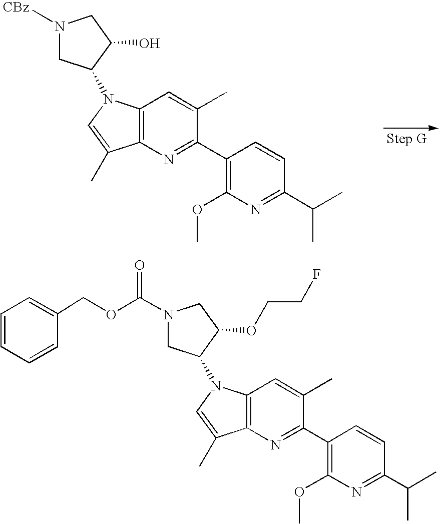

Step A

To a solution of (3R,4S)-3-amino-4-hydroxy-pyrrolidine-1-carboxylic acid benzyl ester (3 g) in DCM (15 mL) is added imidazole (1.3 g) at 0° C. tert-Butyidimethylsilyl chloride (1.9 g) is added to the above solution at 0° C.. After stirring at 0° C. for 30 min, the ice-bath is removed. The mixture is stirred at RT for 2 h and is poured into EtOAc (200 mL). The mixture is washed with water and brine, and is dried over MgSO₄. After removal of the solvent, the residue is purified by flash column chromatography to give (3R,4S)-3-amino-4-(tert-butyl-dimethyl-silanyloxy)-pyrrolidine-1-carboxylic acid benzyl ester as colorless oil. LCMS: Rt 1.40 min, m/z 351.07 (M+H)⁺

Step B

To a solution of (3R,4S)-3-amino-4-(tert-butyl-dimethyl-silanyloxy)-pyrrolidine-1-carboxylic acid benzyl ester (3 g) and 5-bromo-6′-isopropyl-2′-methoxy-3-methyl-[2,3′]bipyridinyl (3.02 g) in toluene (20 mL) is added Pd₂(dba)₃ (0.313 g) BINAP (0.43 g) and NaOtBu (1.15 g) at RT. The mixture is stirred at 80° C. for 22 h and is poured into water (150 mL). The mixture is extracted with EtOAc and the combined extracts are washed with brine. After drying over MgSO₄, the solvent is removed under reduced pressure. The residue is purified by flash column chromatography on silica gel to afford (3S,4R)-3-(tert-butyl-dimethyl-silanyloxy)-4-(6′-isopropyl-2′-methoxy-3-methyl-[2,3′]bipyridinyl-5-ylamino)-pyrrolidine-1-carboxylic acid benzyl ester as amorphous. LCMS Rt 1.62 min, m/z 591.15 (M+H)⁺

Step C

To a stirred solution of (3S,4R)-3-(tert-butyl-dimethyl-silanyloxy)-4-(6′-isopropyl-2′-methoxy-3-methyl-[2,3′]bipyridinyl-5-ylamino)-pyrrolidine-1-carboxylic acid benzyl ester (4.07 g) in chloroform (25 mL) is added NBS (1.23 g) at RT. After stirring at RT for 15 min, the solvent is evaporated under reduced pressure and the residue is purified by flash column chromatography on silica gel to give (3R,4S)-3-(6-bromo-6′-isopropyl-2′-methoxy-3-methyl-[2,3′]bipyridinyl-5-ylamino)-4-(tert-butyl-dimethyl-silanyloxy)-pyrrolidine-1-carboxylic acid benzyl ester as colorless amorphous. Rf (hexane:EtOAc=2:1)=0.55.

Step D

To a stirred solution of (3R,4S)-3-(6-bromo-6′-isopropyl-2′-methoxy-3-methyl-[2,3′]bipyridinyl-5-ylamino)-4-(tert-butyl-dimethyl-silanyloxy)-pyrrolidine-1-carboxylic acid benzyl ester (4.03 g) in THF (25 mL) is added a solution of KOtBu in THF (2.41 mL, 1 M) at RT. Allyl bromide (2.04 mL) is added to the above solution over 10 min at RT. The mixture is stirred at RT for 16 h and is poured in to water. The mixture is extracted with EtOAc. The combined extracts are washed with brine and are dried over MgSO₄. After removal of the solvent under reduced pressure, the residue is purified by column chromatography on silica gel to give (3R,4S)-3-[allyl-(6-bromo-6′-isopropyl-2′-methoxy-3-methyl-[2,3′]bipyridinyl-5-yl)-amino]-4-(tert-butyl-dimethyl-silanyloxy)-pyrrolidine-1-carboxylic acid benzyl ester as amorphous. LCMS Rt 1.93 min, m/z 709/711 (M+H)⁺

Step E

To a solution of (3R,4S)-3-[allyl-(6-bromo-6′-isopropyl-2′-methoxy-3-methyl-[2,3′]bipyridinyl-5-yl)-amino]-4-(tert-butyl-dimethyl-silanyloxy)-pyrrolidine-1-carboxylic acid benzyl ester (3.2 g) in DMF (20 mL) is added Pd(OAc)₂ (81 mg), K₂CO₃ (1.87 g) and tetrabutylammonium bromide (1.6 g) at RT. The mixture is stirred at 80° C. for 2 h and is poured into water. The mixture is extracted with EtOAc. The combined extracts are washed with brine and are dried over MgSO₄. After evaporation of the solvent under reduced pressure, the residue is purified by flash column chromatography on silica gel to give (3S,4R)-3-(tert-butyl-dimethyl-silanyloxy)-4-[5-(6-isopropyl-2-methoxy-pyridin-3-yl)-3,6-dimethyl-pyrrolo[3,2-b]pyridin-1-yl]-pyrrolidine-1-carboxylic acid benzyl ester as amorphous. LCMS Rt 1.62 min, m/z 629.18 (M+H)⁺

Step F

To a stirred solution of (3S,4R)-3-(tert-butyl-dimethyl-silanyloxy)-4-[5-(6-isopropyl-2-methoxy-pyridin-3-yl)-3,6-dimethyl-pyrrolo[3,2-b]pyridin-1-yl]-pyrrolidine-1-carboxylic acid benzyl ester (2.3 g) in THF (14 mL) is added a solution of tetrabutylammonium fluoride in THF (4.8 mL, 1M) at RT. The mixture is stirred at RT for 10 min and is poured into ice-water (80 mL). The mixture is extracted with EtOAc. The combined extracts are washed with brine and are dried over Mg SO₄. After removal of the solvent under reduced pressure, the residue is purified by flash column chromatography on silica gel to give (3S,4R)-3-hydroxy-4-[5-(6-isopropyl-2-methoxy-pyridin-3-yl)-3,6-dimethyl-pyrrolo[3,2-b]pyridin-1-yl]-pyrrolidine-1-carboxylic acid benzyl ester as colorless amorphous. LCMS Rt 1.45 min, m/z 515.10 (M+H)⁺

Step G

To a solution of (3S,4R)-3-hydroxy-4-[5-(6-isopropyl-2-methoxy-pyridin-3-yl)-3,6-dimethyl-pyrrolo[3,2-b]pyridin-1-yl]-pyrrolidine-1-carboxylic acid benzyl ester (1.88 g) in DMF (15 mL) is added sodium hydride (0.44 g) and bromofluoroethane (0.82 mL) at RT. After stirring at RT for 2.5 h, the mixture is poured into ice-water and is extracted with EtOAc. The combined extracts are washed with brine and are dried over MgSO₄. After evaporation of the solvent under the reduced pressure, the residue is purified by flash column chromatography on silica gel to afford (3S,4R)-3-(2-fluoro-ethoxy)-4-[5-(6-isopropyl-2-methoxy-pyridin-3-yl)-3,6-dimethyl-pyrrolo[3,2-b]pyridin-1-yl]-pyrrolidine-1-carboxylic acid benzyl ester as colorless amorphous. LCMS Rt 1.49 min, m/z 561.11 (M+H)⁺

### Example 20

### Synthesis of (3S,4R)-3-(2-fluoro-ethoxy)-4-[5-(6-isopropyl-2-methoxy-pyridin-3-yl)-3,6-dimethyl-pyrrolo[3,2-b]pyridin-1-yl]-pyrrolidine-1-carboxylic acid methyl ester

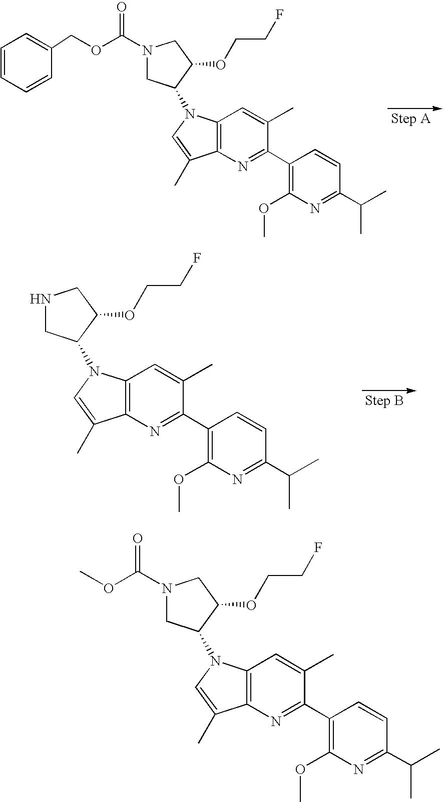

Step A

To a solution of (3S,4R)-3-(2-fluoro-ethoxy)-4-[5-(6-isopropyl-2-methoxy-pyridin-3-yl)-3,6-dimethyl-pyrrolo[3,2-b]pyridin-1-yl]-pyrrolidine-1-carboxylic acid benzyl ester (1.9 g) in EtOH (10 mL) is added 10% Pd/C (0.3 g) at RT. The suspension is stirred at RT under hydrogen for 14 h. The catalyst is removed by filtration and the filtrate is concentrated under reduced pressure. The residue is purified by flash column chromatography on silica gel to give 1-[(3R,4S)-4-(2-fluoro-ethoxy)-pyrrolidin-3-yl]-5-(6-isopropyl-2-methoxy-pyridin-3-yl)-3,6-dimethyl-1H-pyrrolo[3,2-b]pyridine as colorless amorphous. LCMS Rt 1.29 min, m/z 427.11 (M+H)⁺

Step B

To a stirred solution of 1-[(3R,4S)-4-(2-fluoro-ethoxy)-pyrrolidin-3-yl]-5-(6-isopropyl-2-methoxy-pyridin-3-yl)-3,6-dimethyl-1H-pyrrolo[3,2-b]pyridine (0.1 g) in DCM (1 mL) is added methyl chloroformate (0.03 mL) at RT. After stirring at RT for 15 min, the reaction is quenched with aqueous saturated Na₂CO₃ (3 mL). The mixture is extracted with EtOAc. The combined extracts are dried over MgSO₄ and are concentrated under reduced pressure. The residue is purified by preparative TLC to give (3S,4R)-3-(2-fluoro-ethoxy)-4-[5-(6-isopropyl-2-methoxy-pyridin-3-yl)-3,6-dimethyl-pyrrolo[3,2-b]pyridin-1-yl]-pyrrolidine-1-carboxylic acid methyl ester as colorless amorphous. LCMS Rt 1.39 min, m/z 485.13 (M+H)⁺

### Example 21

### Synthesis of 1-[(3R,4S)-4-(2-fluoro-ethoxy)-1-methanesulfonyl-pyrrolidin-3-yl]-5-(6-isopropyl-2-methoxy-pyridin-3-yl)-3,6-dimethyl-1H-pyrrolo[3,2-b]pyridine

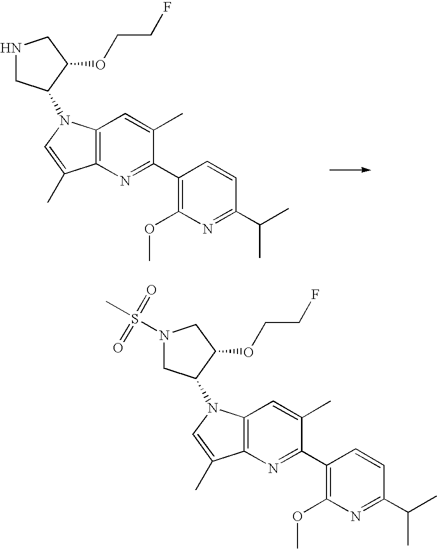

To a stirred solution of 1-[(3R,4S)-4-(2-fluoro-ethoxy)-pyrrolidin-3-yl]-5-(6-isopropyl-2-methoxy-pyridin-3-yl)-3,6-dimethyl-1H-pyrrolo[3,2-b]pyridine (0.1 g) in DCM (1 mL) is added methanesulfonyl chloride (0.03 mL) at RT. After stirring at RT for 15 min, the reaction is quenched with aqueous saturated Na₂CO₃ (3 mL). The mixture is extracted with EtOAc. The combined extracts are dried over MgSO₄ and are concentrated under reduced pressure. The residue is purified by preparative TLC to give 1-[(3R,4S)-4-(2-fluoro-ethoxy)-1-methanesulfonyl-pyrrolidin-3-yl]-5-(6-isopropyl-2-methoxy-pyridin-3-yl)-3,6-dimethyl-1H-pyrrolo[3,2-b]pyridine as colorless amorphous. LCMS Rt 1.35 min, m/z 505.10 (M+H)⁺

### Example 22

### Synthesis of 1-[(3R,4S)-4-(2-fluoro-ethoxy)-1-methyl-pyrrolidin-3-yl]-5-(6-isopropyl-2-methoxy-pyridin-3-yl)-3,6-dimethyl-1H-pyrrolo[3,2-b]pyridine

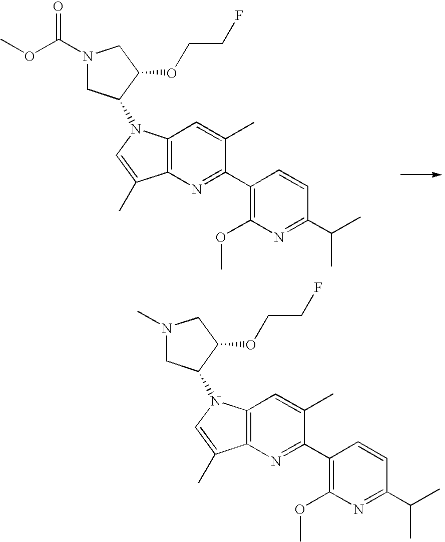

To a solution of (3S,4R)-3-(2-fluoro-ethoxy)-4-[5-(6-isopropyl-2-methoxy-pyridin-3-yl)-3,6-dimethyl-pyrrolo[3,2-b]pyridin-1-yl]-pyrrolidine-1-carboxylic acid methyl ester (77 mg) in THF (1 mL) is added a solution of LiAlH₄ in THF (1.5 mL, 1 M) at RT. After stirring at RT for 2 h, the reaction is quenched with water. The inorganic salts are removed by Celite filtration. The filtrates are concentrated under reduced pressure and the residue is purified by flash chromatography on silica gel to afford 1-[(3R,4S)-4-(2-fluoro-ethoxy)-1-methyl-pyrrolidin-3-yl]-5-(6-isopropyl-2-methoxy-pyridin-3-yl)-3,6-dimethyl-1H-pyrrolo[3,2-b]pyridine as colorless amorphous. LCMS Rt 1.24 min, m/z 441.14 (M+H)⁺

### Example 23

### Synthesis of (3S,4R)-3-(2-fluoro-ethoxy)-4-[5-(6-isopropyl-2-methoxy-pyridin-3-yl)-3,6-dimethyl-pyrrolo[3,2-b]pyridin-1-yl]-pyrrolidine-1-carboxylic acid 2-morpholin-4-yl-ethyl ester

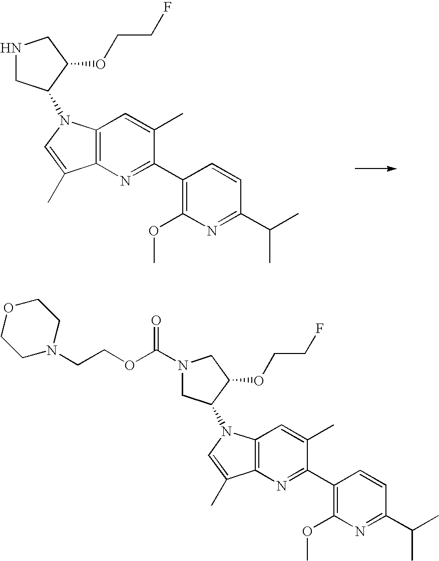

To a stirred solution of 4-(2-hyroxyethyl)morpholine (0.063 mL) in DCM (1 mL) is 1,1′-carbonyldiimidazole (84 mg) at RT. After stirring at RT for 30 min, 1-[(3R,4S)-4-(2-fluoro-ethoxy)-pyrrolidin-3-yl]-5-(6-isopropyl-2-methoxy-pyridin-3-yl)-3,6-dimethyl-1H-pyrrolo[3,2-b]pyridine (0.2 g) is added to the mixture. After stirring at RT for 1 day, the mixture is purified by preparative HPLC to give (3S,4R)-3-(2-fluoro-ethoxy)-4-[5-(6-isopropyl-2-methoxy-pyridin-3-yl)-3,6-dimethyl-pyrrolo[3,2-b]pyridin-1-y1]-pyrrolidine-1-carboxylic acid 2-morpholin-4-yl-ethyl ester as amorphous. LCMS Rt 1.38 min, m/z 584 (M+H)⁺

### Example 24

### Synthesis of 1-((S)-2-methoxy-1-methyl-ethyl)-5-(2-methoxy-4-trifluoromethoxy-phenyl)-6-methyl-1H-pyrrolo[3,2-b]pyridine and 3-bromo-1-((S)-2-methoxy-1-methyl-ethyl)-5-(2-methoxy-4-trifluoromethoxy-phenyl)-6-methyl-1H-pyrrolo[3,2-b]pyridine

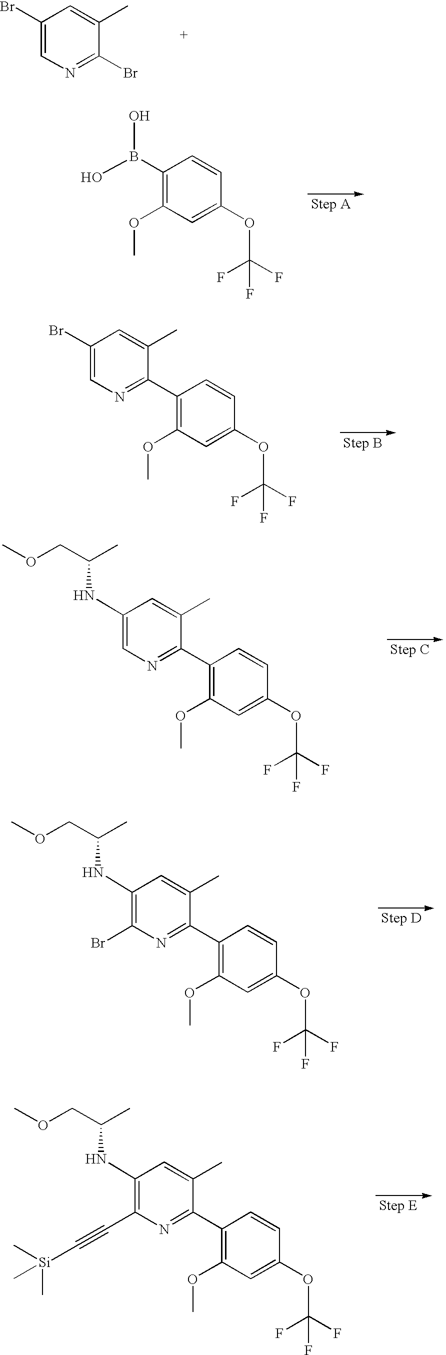

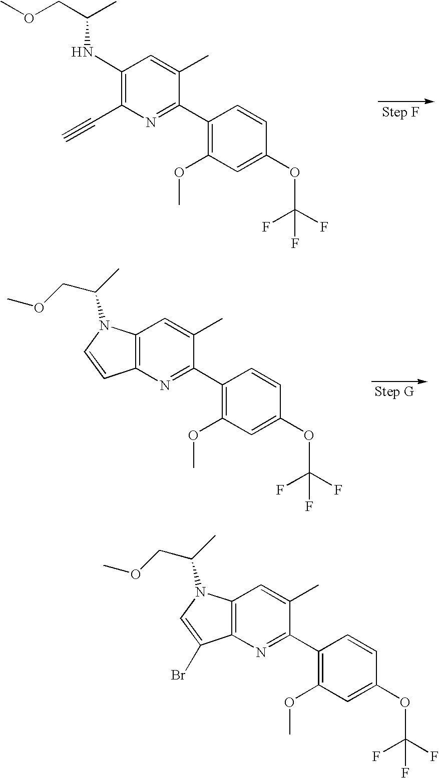

Step A

To a solution of 2,5-dibromo-3-methyl-pyridine (40 g) and 2-methoxy-4-trifluoromethoxy-phenyl boronic acid (39.5 g) in toluene (200 mL) is added Pd(Ph₃P)₄ (5.5 g) and 2M aqueous K₂CO₃ solution (160 mL) at RT. The mixture is stirred at 85° C. for 16 h. The mixture is poured into water and is extracted with EtOAc. The combined extracts are washed with brine and are dried over MgSO₄. After evaporation of the solvent the residue is purified by flash column chromatography on silica gel to give 5-bromo-2-(2-methoxy-4-trifluoromethoxy-phenyl)-3-methyl-pyridine as white solid. MS 362/364 (M+H)⁺

Step B

To a solution of give 5-bromo-2-(2-methoxy-4-trifluoromethoxy-phenyl)-3-methyl-pyridine (1.04 g) in toluene (20 mL) is added (S)-2-methoxy-1-methyl-ethylamine (0.28 g), Pd₂(dba)₃ (0.11 g), BINAP (0.14 g) and NaOtBu (0.39 g). The mixture is stirred at 80° C. for 15 h. The mixture is poured into water and is extracted with EtOAc. The combined extracts are washed with brine and dried over MgSO₄. After removal of the solvent under reduced pressure the residue is purified by flash column chromatography on silica gel to give ((S)-2-methoxy-1-methyl-ethyl)-[6-(2-methoxy-4-trifluoromethoxy-phenyl)-5-methyl-pyridin-3-yl]-amine as amorphous. Rf (hexane:EtOAc=2:1)=0.3

Step C

To a solution of ((S)-2-methoxy-1-methyl-ethyl)-[6-(2-methoxy-4-trifluoromethoxy-phenyl)-5-methyl-pyridin-3-yl]-amine (1 g) in chloroform (5 mL) is added NBS (0.48 g) at RT. After stirring at RT for 5 min, the mixture is directly purified by flash column chromatography on silica gel to give [2-bromo-6-(2-methoxy-4-trifluoromethoxy-phenyl)-5-methyl-pyridin-3-yl]-((S)-2-methoxy-1-methyl-ethyl)-amine as white solid. Rf (hexane:EtOAc=4:1)=0.3

Step D

To a solution of [2-bromo-6-(2-methoxy-4-trifluoromethoxy-phenyl)-5-methyl-pyridin-3-yl]-((S)-2-methoxy-1-methyl-ethyl)-amine (0.2 g) in DMF (1 mL) is added ethynyl-trimethyl-silane (0.08 mL), Et₃N (0.09 mL), PdCl₂(Ph₃P)₂ (6 mg) and CuI (1 mg) at RT. The mixture is stirred at RT for 14 h. The mixture is poured into water and is extracted with EtOAc. The combined extracts are washed with brine and are dried over MgSO₄. After evaporation of the solvent, the residue is purified by flash column chromatography on silica gel to give ((S)-2-bethoxy-1-methyl-ethyl)-[6-(2-methoxy-4-trifluoromethoxy-phenyl)-5-methyl-2-trimethylsilanylethynyl-pyridin-3-yl]-amine as colorless oil. LCMS Rt 1.74 min, m/z 467.15 (M+H)⁺

Step E

To a solution of ((S)-2-bethoxy-1-methyl-ethyl)-[6-(2-methoxy-4-trifluoromethoxy-phenyl)-5-methyl-2-trimethylsilanylethynyl-pyridin-3-yl]-amine (0.18 g) in THF (2 mL) is added a solution of nBu₄NF in THF (0.48 mL, 1 M) at RT. After stirring at RT for 15 min, EtOAc is added to the mixture. The solution is washed with water and brine, and is dried over MgSO₄. After removal of the solvent under reduced pressure, the residue is purified by flash column chromatography on silica gel to give [2-ethynyl-6-(2-methoxy-4-trifluoromethoxy-phenyl)-5-methyl-pyridin-3-yl]-((S)-2-methoxy-1-methyl-ethyl)-amine as colorless oil. LCMS Rt 1.58 min, m/z 395.09 (M+H)⁺

Step F

To a solution of give [2-ethynyl-6-(2-methoxy-4-trifluoromethoxy-phenyl)-5-methyl-pyridin-3-yl]-((S)-2-methoxy-1-methyl-ethyl)-amine (0.1 g) in NMP (3 mL) is added tBuOK (28 mg) at RT. The mixture is stirred at 80° C. for 1 h. The mixture is diluted with EtOAc and is washed with water and brine. After drying over MgSO₄, the solvent is evaporated. The residue is purified by flash column chromatography on silica gel to give 1-((S)-2-methoxy-1-methyl-ethyl)-5-(2-methoxy-4-trifluoromethoxy-phenyl)-6-methyl-1H-pyrrolo[3,2-b]pyridine as amorphous. LCMS 1.30 min, m/z 395.05 (M+H)⁺

Step G

To a solution of 1-((S)-2-methoxy-1-methyl-ethyl)-5-(2-methoxy-4-trifluoromethoxy-phenyl)-6-methyl-1H-pyrrolo[3,2-b]pyridine (65 mg) in chloroform (2 mL) is added NBS (32 mg). The mixture is stirred at RT for 30 min and is diluted with EtOAc. The mixture is washed with water and brine and dried over MgSO₄. After removal of the solvent under reduced pressure, the residue is purified by preparative TLC to give 3-bromo-1-((S)-2-methoxy-1-methyl-ethyl)-5-(2-methoxy-4-trifluoromethoxy-phenyl)-6-methyl-1H-pyrrolo[3,2-b]pyridine as white solid. LCMS Rt 1.54 min, m/z 472.96/474.96 (M+H)⁺

By using steps A-F of Example 24 the following compounds are prepared analogously:
5-(6-Isopropyl-2-methoxy-pyridin-3-yl)-1-((S)-2-methoxy-1-methyl-ethyl)-6-methyl-1H-pyrrolo[3,2-b]pyridine (LCMS Rt 1.40 min, m/z 354.15 (M+H)⁺)
(R)-2-[5-(6-Isopropyl-2-methoxy-pyridin-3-yl)-6-methyl-pyrrolo[3,2-b]pyridin-1-yl]-butan-1-ol (LCMS Rt 1.39 min, m/z 354.12 (M+H)⁺)

### Example 25

### Synthesis of 3-chloro-1-((S)-2-methoxy-1-methyl-ethyl)-5-(2-methoxy-4-trifluoromethoxy-phenyl)-6-methyl-1H-pyrrolo[3,2-b]pyridine and 1-[1-((S)-2-methoxy-1-methyl-ethyl)-5-(2-methoxy-4-trifluoromethoxy-phenyl)-6-methyl-1H-pyrrolo[3,2-b]pyridin-7-yl]-pyrrolidine-2,5-dione

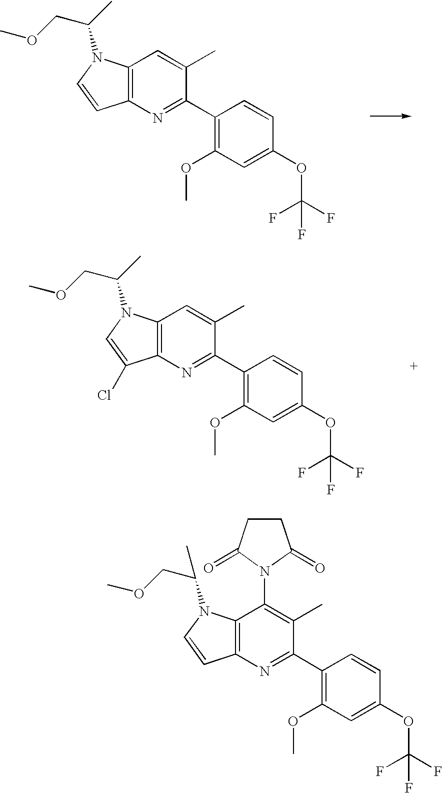

To a solution of 1-((S)-2-methoxy-1-methyl-ethyl)-5-(2-methoxy-4-trifluoromethoxy-phenyl)-6-methyl-1H-pyrrolo[3,2-b]pyridine (40 mg) in chloroform (1 mL) is added NCS (15 mg) at RT. After stirring at RT for 15 h, the mixture is directly purified by preparative TLC to give 3-chloro-1-((S)-2-methoxy-1-methyl-ethyl)-5-(2-methoxy-4-trifluoromethoxy-phenyl)-6-methyl-1H-pyrrolo[3,2-b]pyridine (white solid, LCMS 1.53 min, m/z 429.02/431.02 (M+H)⁺) and 1-[1-((S)-2-methoxy-1-methyl-ethyl)-5-(2-methoxy-4-trifluoromethoxy-phenyl)-6-methyl-1H-pyrrolo[3,2-b]pyridin-7-yl]-pyrrolidine-2,5-dione (amorphous, LCMS Rt 1.38 min, m/z 492.09 (M+H)⁺).

### Example 26

### Synthesis of 3-fluoro-1-((S)-2-methoxy-1-methyl-ethyl)-5-(2-methoxy-4-trifluoromethoxy-phenyl)-6-methyl-1H-pyrrolo[3,2-b]pyridine

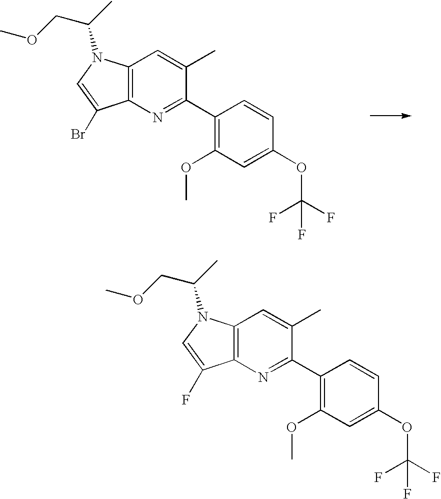

To a stirred solution of 3-bromo-1-((S)-2-methoxy-1-methyl-ethyl)-5-(2-methoxy-4-trifluoromethoxy-phenyl)-6-methyl-1H-pyrrolo[3,2-b]pyridine (17 mg) in THF (1 mL) is added a solution of t-BuLi in pentane (0.09 mL, 1.7 M) at −78° C. After stirring at the same temperature for 1 h, a solution of N-fluorobenzene-sulfonimide (46 mg) in THF (1 mL) is added. The mixture is stirred at −78° C. for 30 min and at 0° C. for 30 min. The mixture is poured into water and is extracted with EtOAc. The combined extracts are dried over MgSO₄ and are concentrated under reduced pressure. The residue is purified by preparative TLC to give 3-fluoro-1-((S)-2-methoxy-1-methyl-ethyl)-5-(2-methoxy-4-trifluoromethoxy-phenyl)-6-methyl-1H-pyrrolo[3,2-b]pyridine as amorphous. LCMS Rt 1.49 min, m/z 413.02 (M+H)⁺

### Example 27

### Synthesis of 3-bromo-5-(6-isopropyl-2-methoxy-pyridin-3-yl)-1-((S)-2-methoxy-1-methyl-ethyl)-6-methyl-1H-pyrrolo[3,2-b]pyridine and 5-(6-isopropyl-2-methoxy-pyridin-3-yl)-1-((S)-2-methoxy-1-methyl-ethyl)-6-methyl-1H-pyrrolo[3,2-b]pyridine-3-carbonitrile

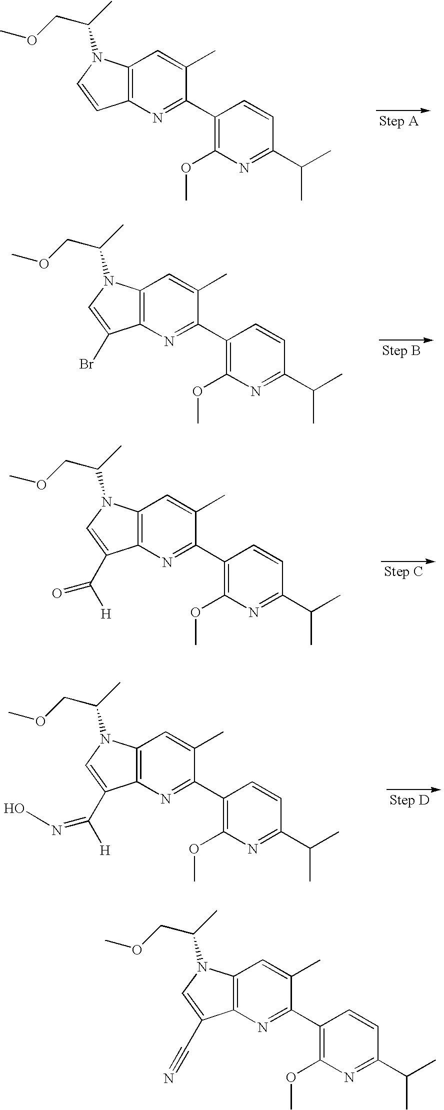

Step A

To a solution of 5-(6-isopropyl-2-methoxy-pyridin-3-yl)-1-((S)-2-methoxy-1-methyl-ethyl)-6-methyl-1H-pyrrolo[3,2-b]pyridine (1.25 g) in chloroform (10 mL) is added NBS (0.66 g) at 0° C. The mixture is stirred at RT for 2 h and is diluted with DCM. The mixture is washed with water and brine. After drying over MgSO₄, the solvent is removed under reduced pressure. The residue is purified by flash column chromatography on silica gel to give 3-bromo-5-(6-isopropyl-2-methoxy-pyridin-3-yl)-1-((S)-2-methoxy-1-methyl-ethyl)-6-methyl-1H-pyrrolo[3,2-b]pyridine as white crystal. LCMS Rt 1.59 min, m/z 432/434 (M+H)⁺

Step B

To a solution of 3-bromo-5-(6-isopropyl-2-methoxy-pyridin-3-yl)-1-((S)-2-methoxy-1-methyl-ethyl)-6-methyl-1H-pyrrolo[3,2-b]pyridine (0.4 g) in THF (4 mL) is treated with n-BuLi in hexane (0.44 mL, 1.6 M) at −70° C. After stirring at −70° C. for 40 min, DMF (0.11 mL) is added to the mixture. The mixture is stirred at −70° C. for 90 min. The reaction is quenched with water and the mixture is extracted with EtOAc. The extract is dried over MgSO₄ and is concentrated under reduced pressure. The residue is purified by preparative TLC to give 5-(6-isopropyl-2-methoxy-pyridin-3-yl)-1-((S)-2-methoxy-1-methyl-ethyl)-6-methyl-1H-pyrrolo[3,2-b]pyridine-3-carbaldehyde as colorless amorphous. LCMS Rt 1.50 min, m/z 382.20 (M+H)⁺

Step C

To a stirred solution of 5-(6-isopropyl-2-methoxy-pyridin-3-yl)-1-((S)-2-methoxy-1-methyl-ethyl)-6-methyl-1H-pyrrolo[3,2-b]pyridine-3-carbaldehyde (0.13 g) in DCM (3 mL) is added hydroxylamine hydrochloride (36 mg) and Et₃N (0.07 mL) at RT. The mixture is stirred at RT for 2 h and is diluted with EtOAc. The mixture is washed with water and dried over MgSO₄. The solvent is removed under reduced pressure to give 5-(6-isopropyl-2-methoxy-pyridin-3-yl)-1-((S)-2-methoxy-1-methyl-ethyl)-6-methyl-1H-pyrrolo[3,2-b]pyridine-3-carbaldehyde oxime as a mixture of syn- and anti-isomers. LCMS Rt 1.38 min, m/z 397.21 (M+H)⁺ and Rt 1.44 min, m/z 397.21 (M+H)⁺

Step D

To a solution of 5-(6-isopropyl-2-methoxy-pyridin-3-yl)-1-((S)-2-methoxy-1-methyl-ethyl)-6-methyl-1H-pyrrolo[3,2-b]pyridine-3-carbaldehyde oxime (0.136 g) in DCM (3 mL) is added Et₃N (0.47 mL) and methanesulfonyl chloride (0.13 mL) at RT. After stirring at RT for 15 h, the mixture is poured into water (30 mL) and is extracted with EtOAc. The extract is washed with water and brine and is dried over MgSO₄. After evaporation of the solvent, the residue is purified by flash column chromatography on silica gel to give 5-(6-isopropyl-2-methoxy-pyridin-3-y1)-1-((S)-2-methoxy-1-methyl-ethyl)-6-methyl-1H-pyrrolo[3,2-b]pyridine-3-carbonitrile as amorphous. LCMS Rt 1.59 min, m/z 379.19 (M+H)⁺

### Example 28

### Synthesis of (S)-2-[5-(2-methoxy-4-trifluoromethoxy-phenyl)-3,6-dimethyl-pyrrolo[3,2-b]pyridin-1-yl]-butan-1-ol and 1-((S)-1-methoxymethyl-propyl)-5-(2-methoxy-4-trifluoromethoxy-phenyl)-3,6-dimethyl-1H-pyrrolo[3,2-b]pyridine

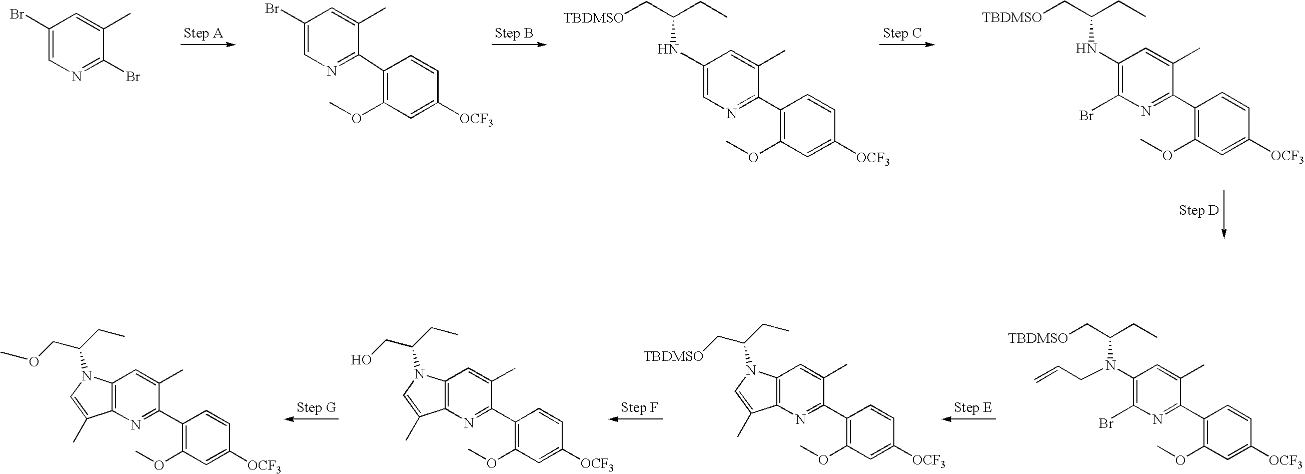

Step A

A mixture of 2,5-dibromo-3-methyl pyridine (40 g), 2-methoxy-4-trifluoromethoxy-1-phenylboronic acid (39.5 g) and 2M K₂CO₃ (159 ml) in toluene (300 ml) is degassed with N₂ for 2 min, followed by addition of Pd(PPh₃)₄ (5.5 g). The resulting mixture is stirred at 85° C. under N₂ for overnight. After reaction is complete, the mixture is poured into water (300 ml) and extracted with ethyl acetate (3×150 ml). The combined organic layers are washed with brine, dried over Na₂SO₄ and evaporated. The product 5-bromo-2-(2-methoxy-4-trifluoromethoxy-phenyl)-3-methyl-pyridine is obtained after flash chromatography (Hexane/EtOAc=20/1). TLC Rf 0.35 (Hexane/EtOAc=4/1).

Step B

A mixture of 5-bromo-2-(2-methoxy-4-trifluoromethoxy-phenyl)-3-methyl-pyridine (1.31 g), (S)-1-(tert-Butyl-dimethyl-silanyloxymethyl)-propylamine (885 mg), (+/−)BINAP (181 mg) and NaOBu^{t} (488 mg) in toluene (10 ml) is degassed with N₂ for 2 min, followed by addition of Pd₂(dba)₃ (133 mg). The resulting mixture is stirred at 70° C. under N₂ for 20 h. The mixture is poured into water and extracted with EtOAc (3×30 ml). The combined organic layers is washed with brine, dried over Na₂SO₄ and evaporated. The crude product is purified by flash chromatography (Hexane/EtOAc=3/1). m/z 485.5 (M+H)⁺.

Step C

257 mg of [(S)-1-(tert-Butyl-dimethyl-silanyloxymethyl)-propyl]-[6-(2-methoxy-4-trifluoromethoxy-phenyl)-5-methyl-pyridin-3-yl]-amine is dissolved in CHCl₃ (6 ml) and NBS (95 mg) is added at room temperature. After stirring at room temperature for 10 min, the mixture is diluted with CHCl₃ and washed with H₂O, brine and dried over Na₂SO₄. The pure product 2-bromo-6-(2-methoxy-4-trifluoromethoxy-phenyl)-5-methyl-pyridin-3-yl]-[(S)-1-(tert-butyl-dimethyl-silanyloxymethyl)-propyl]-amine is obtained after column chromatography (Hexane/EtOAc=8/1). MS m/z 563.3/565.3 (M+H)⁺.

Step D

2-Bromo-6-(2-methoxy-4-trifluoromethoxy-phenyl)-5-methyl-pyridin-3-yl]-[(S)-1-(tert-butyl-dimethyl-silanyloxymethyl)-propyl]-amine (230 mg) in anhydrous THF (6 ml) is added 1M KOBu^{t} (1.03 ml) followed by allyl bromide (71 μl) at room temperature and the resulting mixture is allowed to stir at ambient temperature for 20 h. The reaction is quenched by adding 10 ml H₂O and the mixture is extracted with EtOAc (3×15 ml). The combined organic layers are washed with brine, dried over Na₂SO₄ and evaporated. Flash column chromatography (Hexane/EtOAc=1 5/1) gives desired product. TLC Rf 0.55 (Hexane/EtOAc=10/1).

Step E

A mixture of allyl-[2-bromo-6-(2-methoxy-4-trifluoromethoxy-phenyl)-5-methyl-pyridin-3-yl]-[(S)-1-(tert-butyl-dimethyl-silanyloxymethyl)-propyl]-amine (1.0 g), tetrabutylammonium bromide (589 mg), K₂CO₃ (687 mg) in DMF (40 ml) is degassed with N₂ for 3 min, followed by addition of Pd(OAc)₂ (37 mg). The resulting mixture is stirred at 80° C. for 18 h. The reaction mixture is poured into water and extracted with EtOAc (3×25 ml). The combined organic layers are washed with brine, dried over Na₂SO₄ and evaporated. The crude product is purified by flash chromatography (Hexane/EtOAc=10/1). MS m/z 523.5 (M+H)⁺.

Step F

1-[(S)-1-(tert-Butyl-dimethyl-silanyloxymethyl)-propyl]-5-(2-methoxy-4-trifluoromethoxy-phenyl)-3,6-dimethyl-1H-pyrrolo[3,2-b]pyridine (1.19 g) in THF (30 ml) is added tetrabutylammonium fluoride (715 mg) and the mixture is allowed to stir at room temperature for 30 min. After the reaction is complete, the solvent is removed and the crude mixture is purified by flash chromatography (CH₂Cl₂/MeOH=5/1) to give desired product. LC MS m/z 409.01 (M+H)⁺.

Step G

(S)-2-[5-(2-methoxy-4-trifluoromethoxy-phenyl)-3,6-dimethyl-pyrrolo[3,2-b]pyridin-1-yl]-butan-1-ol (60 mg) in anhydrous THF (5 ml) is added 60% NaH (29 mg) and the mixture is allowed to stir at room temperature for 10 min before MeI (46 μl) is added. After stirring at room temperature for 1 h, the reaction is quenched by adding water (15 ml). The mixture is extracted with EtOAc (3×25 ml), dried over Na₂SO₄ and evaporated. The pure product 1-((S)-1-methoxymethyl-propyl)-5-(2-methoxy-4-trifluoromethoxy-phenyl)-3,6-dimethyl-1H-pyrrolo[3,2-b]pyridine is obtained by flash column chromatography (Hexane/EtOAc=3/1). LC MS m/z 423.03 (M+H)⁺.

### Example 29

### Synthesis of 1-((S)-1-chloromethyl-propyl)-5-(2-methoxy-4-trifluoromethoxy-phenyl)-3,6-dimethyl-1H-pyrrolo[3,2-b]pyridine and 5-(2-methoxy-4-trifluoromethoxy-phenyl)-3,6-dimethyl-1-((S)-1-pyrrolidin-1-ylmethyl-propyl)-1H-pyrrolo[3,2-b]pyridine

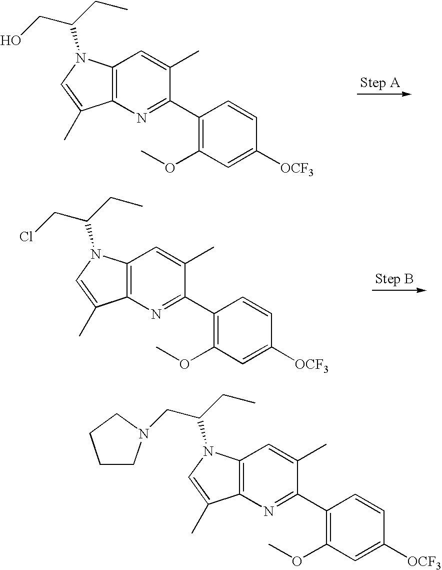

Step A

(S)-2-[5-(2-Methoxy-4-trifluoromethoxy-phenyl)-3,6-dimethyl-pyrrolo[3,2-b]pyridin-1-y]-butan-1-ol (330 mg) in ClCH₂CH₂Cl (20 ml) is cooled to 0° C. and SOCl₂ (1.77 ml) is added dropwise. The reaction is allowed to stir at room temperature for 12 h. After removal of the solvent, the crude product is purified by flash column (Hexane/EtOAc=3/1). MS m/z 427.4 (M+H)⁺.

Step B

A mixture of 1-((S)-1-chloromethyl-propyl)-5-(2-methoxy-4-trifluoromethoxy-phenyl)-3,6-dimethyl-1H-pyrrolo[3,2-b]pyridine (55 mg), KI (15 mg) and 0.8 ml pyrrolidine in DMSO (4 ml) is heated to 120° C. for 19 h. After starting material disappears, the mixture is poured into water and extracted with CH₂Cl₂ (3×20 ml). The combined organic layers are washed with brine, dried over Na₂SO₄ and evaporated. The pure product 5-(2-methoxy-4-trifluoromethoxy-phenyl)-3,6-dimethyl-1-((S)-1-pyrrolidin-1-ylmethyl-propyl)-1H-pyrrolo[3,2-b]pyridine is obtained by preparative TLC purification (CH₂Cl₂/MeOH=15/1). LC MS m/z 462.10 (M+H)⁺.

### Example 30

### Synthesis of methanesulfonic acid (S)-2-[5-(2-methoxy-4-trifluoromethoxy-phenyl)-3,6-dimethyl-pyrrolo[3,2-b]pyridin-1-yl]-butyl ester, 1-((S)-1-methanesulfonylmethyl-propyl)-5-(2-methoxy-4-trifluoromethoxy-phenyl)-3,6-dimethyl-1H-pyrrolo[3,2-b]pyridine, piperidine-1-carboxylic acid (S)-2-[5-(2-methoxy-4-trifluoromethoxy-phenyl)-3,6-dimethyl-pyrrolo[3,2-b]pyridin-1-yl]-butyl ester and cyclopentyl-carbamic acid (S)-2-[5-(2-methoxy-4-trifluoromethoxy-phenyl)-3,6-dimethyl-pyrrolo[3,2-b]pyridin-1-yl]-butyl ester

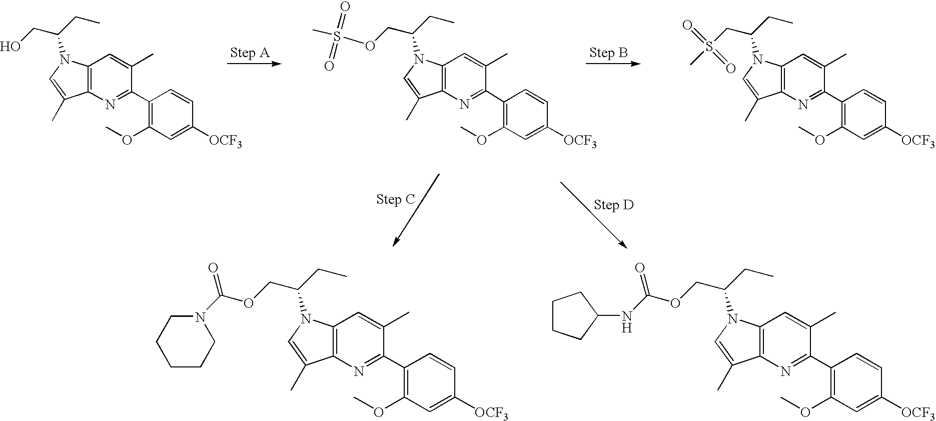

Step A

(S)-2-[5-(2-Methoxy-4-trifluoromethoxy-phenyl)-3,6-dimethyl-pyrrolo[3,2-b]pyridin-1-yl]-butan-1-ol (120 mg) in CH₂Cl₂ (6 ml) is cooled to 0° C. and the mixture is added triethylamine (82 μl) followed by methanesulfonyl chloride (45 μl). The mixture is allowed to stir at 0° C. to room temperature for 16 h. After removal of the solvent, the crude mixture is purified by column chromatography (CH₂Cl₂/MeOH=12/1). LC MS m/z 486.99 (M+H)⁺.

Step B

A mixture of methanesulfonic acid (S)-2-[5-(2-methoxy-4-trifluoromethoxy-phenyl)-3,6-dimethyl-pyrrolo[3,2-b]pyridin-1-yl]-butyl ester (39 mg), KI (5 mg) and CH₃SO₂Na (100 mg) in DMSO (2 ml) were heated to 80° C. for 17 h. The mixture is poured into water and extracted with EtOAc (3×15 ml). The combined organic layers are washed with brine, dried over Na₂SO₄ and evaporated. The pure product 1-((S)-1-methanesulfonylmethyl-propyl)-5-(2-methoxy-4-trifluoromethoxy-phenyl)-3,6-dimethyl-1H-pyrrolo[3,2-b]pyridine is obtained by preparative TLC purification (Hexane/EtOAc=1/1). LC MS m/z 471.03 (M+H)⁺.

Step C

2 ml of methanesulfonic acid (S)-2-[5-(2-methoxy-4-trifluoromethoxy-phenyl)-3,6-dimethyl-pyrrolo[3,2-b]pyridin-1-yl]-butyl ester (0.02 M in DMSO) is added 0.2 ml of piperidine (0.2 M in toluene), followed by NaHCO₃ (50 mg) and KI (10 mg). The resulting mixture is shaken at 80° C. for 18 h. The mixture is diluted with water, extracted with EtOAc (2×10 ml). The combined organic layers are washed with brine, dried over Na₂SO₄ and evaporated. The pure product piperidine-1-carboxylic acid (S)-2-[5-(2-methoxy-4-trifluoromethoxy-phenyl)-3,6-dimethyl-pyrrolo[3,2-b]pyridin-1-yl]-butyl ester is obtained by preparative TLC purification (Hexane/EtOAc=1/1). LC MS m/z 520.11 (M+H)⁺.

Step D

2 ml of methanesulfonic acid (S)-2-[5-(2-methoxy-4-trifluoromethoxy-phenyl)-3,6-dimethyl-pyrrolo[3,2-b]pyridin-1-yl]-butyl ester (0.02 M in DMSO) is added 0.2 ml of cyclopentylamine (0.2 M in toluene), followed by NaHCO₃ (50 mg) and KI (10 mg). The resulting mixture is shaken at 80° C. for 18 h. The mixture is diluted with water, extracted with EtOAc (2×10 ml). The combined organic layers are washed with brine, dried over Na₂SO₄ and evaporated. The pure product cyclopentyl-carbamic acid (S)-2-[5-(2-methoxy-4-trifluoromethoxy-phenyl)-3,6-dimethyl-pyrrolo[3,2-b]pyridin-1-yl]-butyl ester is obtained by preparative TLC purification (Hexane/EtOAc=1/1). LC MS m/z 520.12 (M+H)⁺.

### Example 31

### Synthesis of (R)-2-[6-ethyl-5-(6-isopropyl-2-methoxy-pyridin-3-yl)-3-methyl-pyrrolo[3,2-b]pyridin-1-yl]-propan-1-ol and 6-ethyl-5-(6-isopropyl-2-methoxy-pyridin-3-yl)-1-((R)-2-methoxy-1-methyl-ethyl)-3-methyl-1H-pyrrolo[3,2-b]pyridine

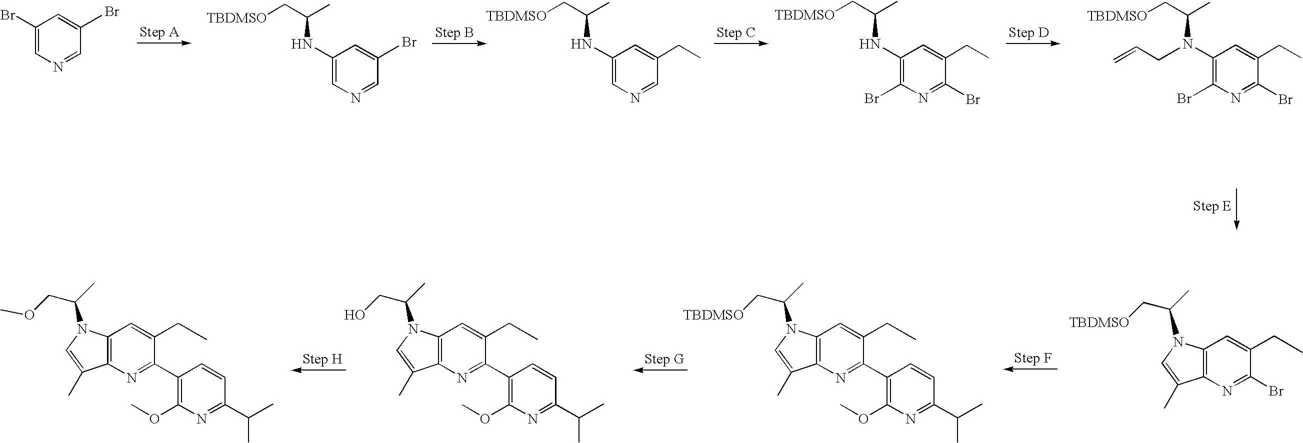

Step A

A mixture of 3,5-dibromopyridine (30.3 g), (R)-2-(tert-butyl-dimethyl-silanyloxy)-1-methyl-ethylamine (25.4 g), (+/−)BINAP (6.37 g) and NaOBu^{t} (17.18 g) in toluene (300 ml) is degassed for 5 min, followed by addition of Pd₂(dba)₃ (4.68 g). The resulting mixture is stirred at 70° C. for 4 h. The reaction mixture is poured into water (200 ml), extracted with EtOAc (3×150 ml). The combined organic layers are washed with brine, dried over Na₂SO₄ and evaporated. The desired product is obtained after flash column chromatography (Hexane/EtOAc=3/1). LC MS m/z 347.24 (M+H)⁺.

Step B

A mixture of (5-bromo-pyridin-3-yl)-[(R)-2-(tert-butyl-dimethyl-silanyloxy)-1-methyl-ethyl]-amine (22.74 g), 2M K₂CO₃ (99 ml) and 165 ml Et₃B (1M in hexane) in toluene (200 ml) is degassed with N₂ for 5 min, followed by addition of Pd(PPh₃)₄ (3.8 g). The resulting mixture is allowed to stir at 110° C. for 16 h. The mixture is poured into water (200 ml), extracted with EtOAc (3×200 ml), dried over Na₂SO₄ and evaporated. The crude product [(R)-2-(tert-butyl-dimethyl-silanyloxy)-1-methyl-ethyl]-(5-ethyl-pyridin-3-yl)-amine is used for next step without further purification. LC MS m/z 295.14 (M+H)⁺.

Step C

Crude product from last step is dissolved in CHCl₃ (250 ml) and NBS (2 eq.) is added in one portion at room temperature. After stirring at room temperature for 15 min, the solution is washed with water (2×100 ml). The organic phase is dried over Na₂SO₄ and evaporated. The crude product is purified by flash chromatography (Hexane/EtOAc=8/1). LC MS m/z 451.12/453.11 (M+H)⁺.

Step D

(R)-2-(tert-Butyl-dimethyl-silanyloxy)-1-methyl-ethyl]-(2,6-dibromo-5-ethyl-pyridin-3-yl)-amine (11.5 g) in anhydrous THF (180 ml) is added 1M KOBu^{t} (50.9 ml), followed by allyl iodide (3.48 ml). The resulting mixture is allowed to stir at room temperature for 22 h before it is quenched with water (100 ml). The mixture is extracted with EtOAc (3×150 ml). The combined organic layers are washed with brine, dried over Na₂SO₄ and evaporated. The pure product allyl-[(R)-2-(tert-butyl-dimethyl-silanyloxy)-1-methyl-ethyl]-(2,6-dibromo-5-ethyl-pyridin-3-yl)-amine is obtained after column chromatography (Hexane/EtOAc=10/1). TLC Rf 0.6 (Hexane/EtOAc=10/1).

Step E

A mixture of allyl-[(R)-2-(tert-butyl-dimethyl-silanyloxy)-1-methyl-ethyl]-(2,6-dibromo-5-ethyl-pyridin-3-yl)-amine (8.3 g), tetrabutylammonium bromide (6.0 g), K₂CO₃ (6.99 g) in DMF (100 ml) is degassed for 3 min, followed by addition of Pd(OAc)₂. The resulting mixture is stirred at 80° C. for 18 h. After the mixture is complete, the mixture is poured into H₂O (200 ml), extracted with EtOAc (3×100 ml). The combined organic layers are washed with brine, dried over Na₂SO₄ and evaporated. The crude product is purified by flash column chromatography (Hexane/EtOAc=10/1). TLC Rf 0.5 (Hexane/EtOAc=4/1).

Step F

A mixture of 5-bromo-1-[(R)-2-(tert-butyl-dimethyl-silanyloxy)-1-methyl-ethyl]-6-ethyl-3-methyl-1H-pyrrolo[3,2-b]pyridine (2.21 g), 2M K₂CO₃ (5.4 ml), and 2-methoxy-6-isopropyl-3-pyridylboronic acid (1.20 g) in DME (25 ml) is degassed with N₂ for 2 min, followed by addition of Pd(PPh₃)₄. The resulting mixture is stirred at 85° C. for 16 h before it is poured into water (80 ml), and extracted with EtOAc (3×30 ml). The combined organic layers are washed with brine, dried over Na₂SO₄ and evaporated. Flash column chromatography (Hexane/EtOAc=6/1) gives the pure product 1-[(R)-2-(tert-butyl-dimethyl-silanyloxy)-1-methyl-ethyl]-6-ethyl-5-(6-isopropyl-2-methoxy-pyridin-3-yl)-3-methyl-1H-pyrrolo[3,2-b]pyridine. LC MS m/z 482.18 (M+H)⁺.

Step G

1-[(R)-2-(tert-Butyl-dimethyl-silanyloxy)-1-methyl-ethyl]-6-ethyl-5-(6-isopropyl-2-methoxy-pyridin-3-yl)-3-methyl-1H-pyrrolo[3,2-b]pyridine (1.87 g) in THF (60 ml) is added tetrabutylammonium fluoride (1.53 g) and the mixture is allowed to stir at room temperature for 15 min before the solvent is evaporated. The crude product is purified by flash chromatography (Hexane/EtOAc=1/1). MS m/z 368.4 (M+H)⁺.

Step H

(R)-2-[6-Ethyl-5-(6-isopropyl-2-methoxy-pyridin-3-yl)-3-methyl-pyrrolo[3,2-b]pyridin-1-yl]-propan-1-ol (1.15 g) in anhydrous THF (40 ml) is added NaH (627 mg) and the mixture is stirred at room temperature for 5 min before MeI (978 μl) is added. The reaction mixture is stirred at room temperature for 3 h and then quenched with H₂O (50 ml) and extracted with EtOAc (3×40 ml). The combined organic layers are washed with brine, dried over Na₂SO₄ and evaporated. The pure product 6-ethyl-5-(6-isopropyl-2-methoxy-pyridin-3-yl)-1-((R)-2-methoxy-1-methyl-ethyl)-3-methyl-1H-pyrrolo[3,2-b]pyridine is obtained by flash chromatography (Hexane/EtOAc=4/1). LC MS m/z 382.44 (M+H)⁺.

### Example 32

### Synthesis of (S)-2-[6-bromo-5-(6-isopropyl-2-methoxy-pyridin-3-yl)-3-methyl-pyrrolo[3,2-b]pyridin-1-yl]-butan-1-ol, 6-bromo-5-(6-isopropyl-2-methoxy-pyridin-3-yl)-1-((S)-1-methoxymethyl-propyl)-3-methyl-1H-pyrrolo[3,2-b]pyridine and 5-(6-isopropyl-2-methoxy-pyridin-3-yl)-1-((S)-2-methoxy-1-methyl-propyl)-3-methyl-1H-pyrrolo[3,2-b]pyridine

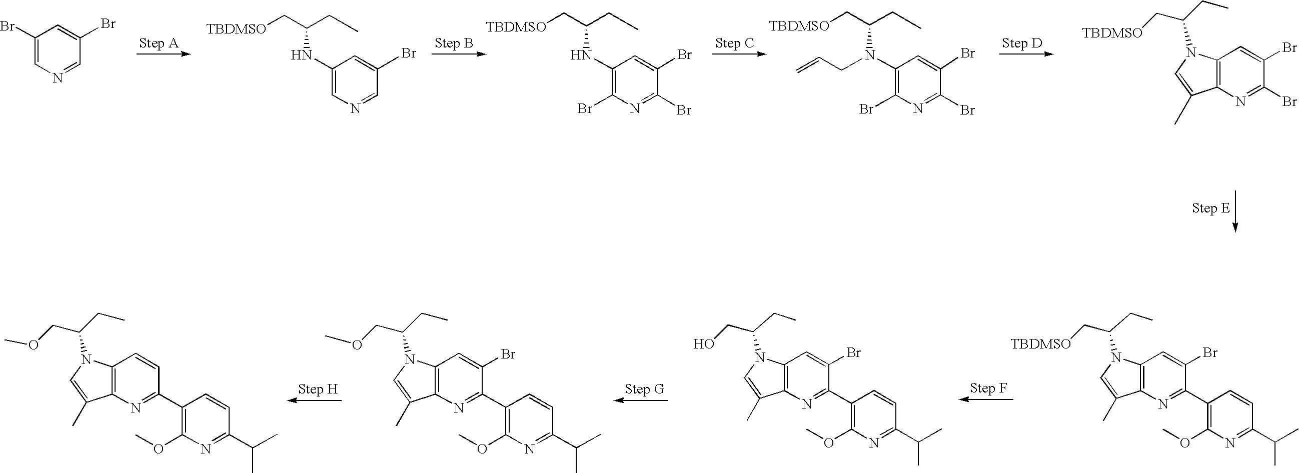

Step A

A mixture of 3,5-dibromopyridine (50 g), (S)-1-(tert-butyl-dimethyl-silanyloxymethyl)-propylamine (43.68 g), (+/−)BINAP (10.51 g) and NaOBu^{t} (28.35 g) in toluene (400 ml) is degassed with N₂ for 5 min, followed by addition of Pd₂(dba)₃ (7.73 g). The resulting mixture is stirred at 70° C. for 23 h. The reaction mixture is poured into water (200 ml), extracted with EtOAc (3×200 ml). The combined organic layers are washed with brine, dried over Na₂SO₄ and evaporated. The desired product is obtained after flash column chromatography (Hexane/EtOAc=5/1). TLC Rf 0.4 (Hexane/EtOAc=4/1).

Step B

(5-Bromo-pyridin-3-yl)-[(S)-1-(tert-butyl-dimethyl-silanyloxymethyl)-propyl]-amine (7.58 g) in CHCl₃ (150 ml) is added NBS (7.51 g) and the mixture is stirred at room temperature for 15 min before it is washed with H₂O (2×50 ml). The organic layer is dried over Na₂SO₄ and evaporated. The crude product is purified by column chromatography (Hexane/EtOAc=10/1). TLC Rf 0.7 (Hexane/EtOAc=4/1).

Step C

(S)-1-(tert-Butyl-dimethyl-silanyloxymethyl)-propyl]-(2,5,6-tribromo-pyridin-3-yl)-amine (6.33 g) in THF (60 ml) is added 24.5 ml KOBu^{t} (1 M in THF) followed by allyl iodide (1.68 ml). The resulting mixture is stirred at room temperature for 24 h before it is quenched with water (60 ml). The mixture is extracted with EtOAc (3×30 ml) and the combined organic layers are washed with brine, dried over Na₂SO₄ and evaporated. The pure product allyl-[(S)-1-(tert-butyl-dimethyl-silanyloxymethyl)-propyl]-(2,5,6-tribromo-pyridin-3-yl)-amine is obtained by flash column chromatography (Hexane/EtOAc=15/1). TLC Rf 0.6 (Hexane/EtOAc=10/1).

Step D

A mixture of allyl-[(S)-1-(tert-butyl-dimethyl-silanyloxymethyl)-propyl]-(2,5,6-tribromo-pyridin-3-yl)-amine (5.81 g), tetrabutylammonium bromide (3.7 g), K₂CO₃ (4.32 g) in DMF (25 ml) is degassed with N₂ for 2 min, followed by addition of Pd(OAc)₂ (214 mg). The resulting mixture is stirred at 80° C. for 1.5 h before it is poured into water (50 ml), and extracted with EtOAc (3×30 ml). The combined organic layers are washed with brine, dried over Na₂SO₄ and evaporated. The crude product is purified by flash column chromatography (Hexane/EtOAc=10/1). TLC Rf 0.3 (Hexane/EtOAc=10/1).

Step E

A mixture of 5,6-dibromo-1-[(S)-1-(tert-butyl-dimethyl-silanyloxymethyl)-propyl]-3-methyl-1H-pyrrolo[3,2-b]pyridine (3.66 g), 2M K₂CO₃ (22 ml), 2-methoxy-6-isopropyl-3-pyridylboronic acid (1.64 g) in DME is degassed with N₂ for 5 min, followed by addition of Pd(PPh₃)₄ (444 mg). The resulting mixture is allowed to stir at 85° C. for 3.5 h before it is poured into H₂O (50 ml), extracted with EtOAc (3×40 ml). The combined organic layers are washed with brine, dried over Na₂SO₄ and evaporated. The pure product 6-bromo-1-[(S)-1-(tert-butyl-dimethyl-silanyloxymethyl)-propyl]-5-(6-isopropyl-2-methoxy-pyridin-3-yl)-3-methyl-1H-pyrrolo[3,2-b]pyridine is obtained by flash column chromatography (Hexane/EtOAc=8/1). LC MS m/z 547.3 (M+H)⁺.

Step F

6-Bromo-1-[(S)-1-(tert-butyl-dimethyl-silanyloxymethyl)-propyl]-5-(6-isopropyl-2-methoxy-pyridin-3-yl)-3-methyl-1H-pyrrolo[3,2-b]pyridine (2.74 g) in THF (50 ml) is added tetrabutylammonium fluoride (1.97 g) and the resulting mixture is stirred at room temperature for 2 h. After removal of the solvent, the crude product is purified by column chromatography (Hexane/EtOAc=1/1). LC MS m/z 433.35 (M+H)⁺.

Step G

A mixture of (S)-2-[6-bromo-5-(6-isopropyl-2-methoxy-pyridin-3-yl)-3-methyl-pyrrolo[3,2-b]pyridin-1-yl]-butan-1-ol (2.0 g) in THF (40 ml) is added 60% NaH (463 mg) and the mixture is stirred at 0° C. for 10 min before MeI (578 μl) is added. After stirring at room temperature for 3.5 h, the mixture is poured into water (50 ml) and extracted with EtOAc (3×30 ml). The combined organic layers are washed with brine, dried over Na₂SO₄ and evaporated. Column chromatography (Hexane/EtOAc=6/1) gives the pure product 6-bromo-5-(6-isopropyl-2-methoxy-pyridin-3-yl)-1-((S)-1-methoxymethyl-propyl)-3-methyl-1H-pyrrolo[3,2-b]pyridine. LC MS m/z 447.37 (M+H)⁺.

Step H

6-Bromo-5-(6-isopropyl-2-methoxy-pyridin-3-yl)-1-((S)-1-methoxymethyl-propyl)-3-methyl-1H-pyrrolo[3,2-b]pyridine (450 mg) in EtOH (30 ml) is added 10% Pd/C (200 mg) under N₂ and the mixture is shaken under 40 psi H₂ pressure for 48 h. the catalyst is removed by filtering through celite. After removal of solvent, the desired product 5-(6-isopropyl-2-methoxy-pyridin-3-yl)-1-((S)-2-methoxy-1-methyl-propyl)-3-methyl-1H-pyrrolo[3,2-b]pyridine is obtained. MS m/z 368.4 (M+H)⁺.

### Example 33

### Synthesis of {3-[6-ethyl-1-((R)-2-methoxy-1-methyl-ethyl)-3-methyl-1H-pyrrolo[3,2-b]pyridin-5-yl]-6-isopropyl-pyridin-2-yl}-methyl-amine, 5-chloro-3-[6-ethyl-1-((R)-2-methoxy-1-methyl-ethyl)-3-methyl-1H-pyrrolo[3,2-b]pyridin-5-yl]-6-isopropyl-pyridin-2-yl}-methyl-amine, {5-bromo-3-[6-ethyl-1-((R)-2-methoxy-1-methyl-ethyl)-3-methyl-1H-pyrrolo[3,2-b]pyridin-5-yl]-6-isopropyl-pyridin-2-yl}-methyl-amine and {5-cyclopropyl-3-[6-ethyl-1-((R)-2-methoxy-1-methyl-ethyl)-3-methyl-1H-pyrrolo[3,2-b]pyridin-5-yl]-6-isopropyl-pyridin-2-yl}methyl-amine

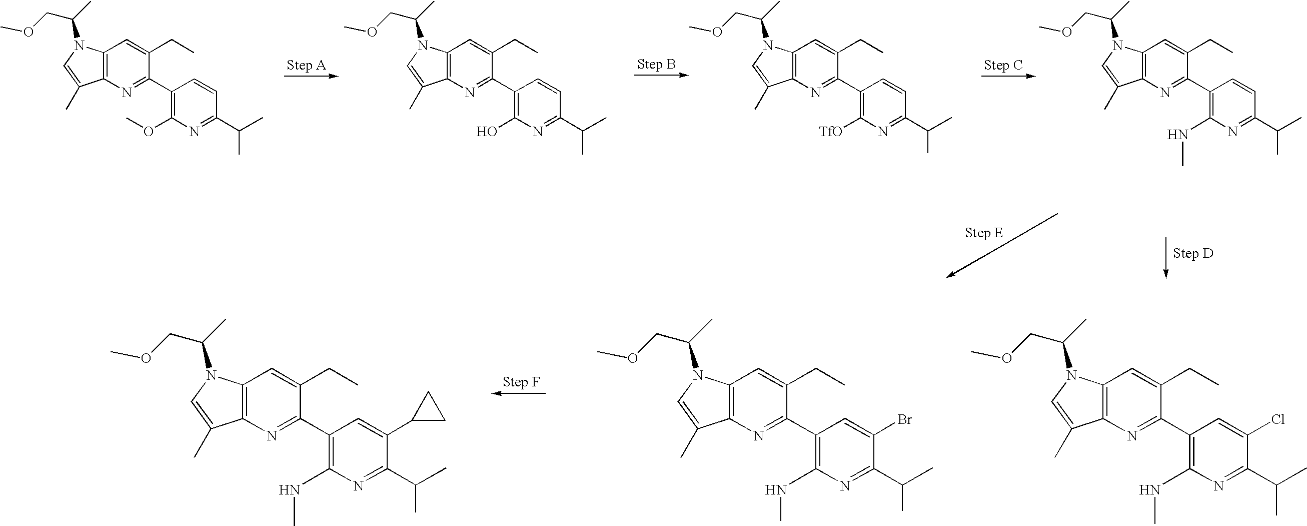

Step A

A mixture of 6-ethyl-5-(6-isopropyl-2-methoxy-pyridin-3-yl)-1-((R)-2-methoxy-1-methyl-ethyl)-3-methyl-1H-pyrrolo[3,2-b]pyridine (1.02 g) and 6N HCl (20 ml) is heated to 75° C. for 20 h. The mixture is cooled down to 0° C. and neutralized with 10 N NaOH to PH>10. The basic solution is extracted with CH₂Cl₂ (3×40 ml). The combined organic layers are washed with brine, dried over Na₂SO₄ and evaporated. The crude product 3-[6-ethyl-1-((R)-2-methoxy-1-methyl-ethyl)-3-methyl-1H-pyrrolo[3,2-b]pyridin-5-yl]-6-isopropyl-pyridin-2-ol is used for next step without further purification. TLC Rf 0.2 (CH₂Cl₂/MeOH=12/1).

Step B Crude product from previous step is dissolved in CH₂Cl₂ (30 ml) and the mixture is cooled to 0° C., followed by addition of triethylamine (1.11 ml) and trifluoromethanesulfonic anhydride (898 μl). After stirring at room temperature for 3 h, the mixture is poured into H₂O (30 ml) and extracted with EtOAc (3×30 ml). The combined organic layers are washed with brine, dried over Na₂SO₄ and evaporated. The pure desired product trifluoro-methanesulfonic acid 3-[6-ethyl-1-((R)-2-methoxy-1-methyl-ethyl)-3-methyl-1H-pyrrolo[3,2-b]pyridin-5-yl]-6-isopropyl-pyridin-2-yl ester is obtained by flash column chromatography (CH₂Cl₂/MeOH=6/1). TLC Rf 0.4 (CH₂Cl₂/MeOH=12/1).

Step C

A mixture of trifluoro-methanesulfonic acid 3-[6-ethyl-1-((R)-2-methoxy-1-methyl-ethyl)-3-methyl-1H-pyrrolo[3,2-b]pyridin-5-yl]-6-isopropyl-pyridin-2-yl ester (200 mg) and NMP (6 ml) is added 1 ml MeNH₂ (4M in NMP) and the mixture is heated to 80° C. for 20 h. The mixture is poured into water (20 ml), extracted with EtOAc (3×15 ml). The combined organic layers are washed with brine, dried over Na₂SO₄ and evaporated. The pure product is obtained after preparative TLC purification (Hexane/EtOAc=2/1). LC MS m/z 381.45 (M+H)⁺.

Step D

{3-[6-Ethyl-1-((R)-2-methoxy-1-methyl-ethyl)-3-methyl-1H-pyrrolo[3,2-b]pyridin-5-yl]-6-isopropyl-pyridin-2-yl}-methyl-amine (60 mg) in CHCl₃ (5 ml) is added N-chlorosuccinimide (23 mg) and the mixture is heated at 60° C. for 5 h. After the reaction is complete, the solvent is removed and the crude product is purified by preparative TLC (Hexane/EtOAc=4/1) to give 5-chloro-3-[6-ethyl-1-((R)-2-methoxy-1-methyl-ethyl)-3-methyl-1H-pyrrolo[3,2-b]pyridin-5-yl]-6-isopropyl-pyridin-2-yl}methyl-amine. MS m/z 415.4 (M+H)⁺.

Step E

A mixture of {3-[6-Ethyl-1-((R)-2-methoxy-1-methyl-ethyl)-3-methyl-1H-pyrrolo[3,2-b]pyridin-5-yl]-6-isopropyl-pyridin-2-yl}-methyl-amine (130 mg) in CH₃CN (5 ml) is cooled to 0° C. followed by addition of NBS (61 mg). The resulting mixture is stirred at 0° C. for 30 min and then it is diluted with H₂O (20 ml), extracted with EtOAc (3×25 ml). The combined organic layers are washed with brine, dried over Na₂SO₄ and evaporated. Flash column chromatography (Hexane/EtOAc=8/1) gives pure product {5-bromo-3-[6-ethyl-1-((R)-2-methoxy-1-methyl-ethyl)-3-methyl-1H-pyrrolo[3,2-b]pyridin-5-yl]-6-isopropyl-pyridin-2-yl}-methyl-amine. LC MS m/z 461.35 (M+H)⁺.

Step F

A mixture of {5-bromo-3-[6-ethyl-1-((R)-2-methoxy-1-methyl-ethyl)-3-methyl-1H-pyrrolo[3,2-b]pyridin-5-yl]-6-isopropyl-pyridin-2-yl}-methyl-amine (60 mg), 2M K₂CO₃ (1 ml), cyclopropyl boronic acid (56 mg) in toluene (5 ml) is degassed with N₂ for 2 min, followed by addition of Pd(PPh₃)₄ (15 mg). The resulting mixture is stirred at 110° C. for 16 h before it is poured into water and extracted with EtOAc (3×15 ml). The combined organic layers are washed with brine, dried over Na₂SO₄ and evaporated. The pure product {5-cyclopropyl-3-[6-ethyl-1-((R)-2-methoxy-1-methyl-ethyl)-3-methyl-1H-pyrrolo[3,2-b]pyridin-5-yl]-6-isopropyl-pyridin-2-yl}methyl-amine is obtained after preparative TLC purification (CH₂Cl₂/MeOH=20/1). MS m/z 421.5 (M+H)⁺.

### Example 34

### Synthesis of ethyl-{6-isopropyl-3-[6-methoxy-1-((S)-2-methoxy-1-methyl-ethyl)-3-methyl-1H-pyrrolo[3,2-b]pyridin-5-yl]-pyridin-2-yl}amine and 5-(2-ethyl-6-isopropyl-pyridin-3-yl)-6-methoxy-1-((S)-2-methoxy-1-methyl-ethyl)-3-methyl-1H-pyrrolo[3,2-b]pyridine

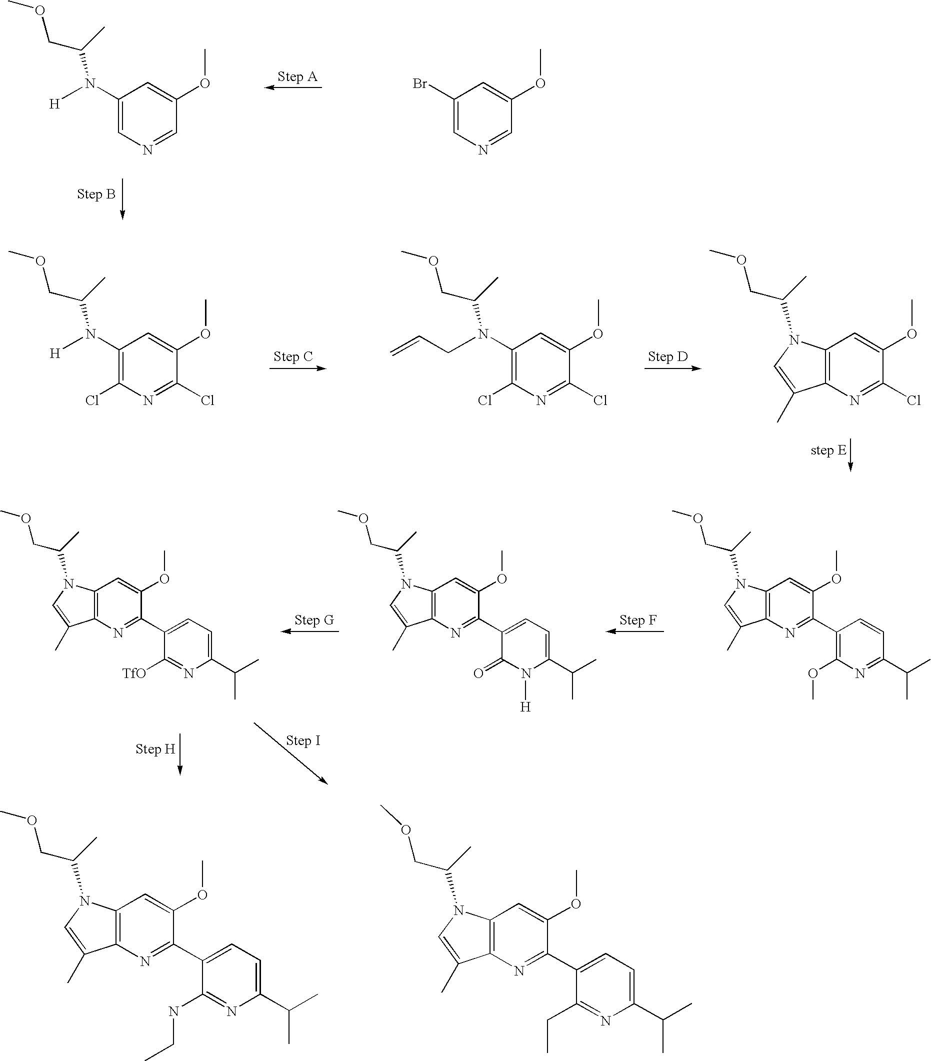

Step A

Analogous to the preparation of (5-bromo-pyridin-3-yl)-[(S)-1-(tert-butyl-dimethyl-silanyloxymethyl)-propyl]-amine the palladium mediated amination of 3-bromo-5-methoxypyridine (4.76 g) with (S)-1-methoxy-2-propylamine (4.4 mL) gives, after purification on silica gel ((S)-2-methoxy-1-methyl-ethyl)-(5-methoxy-pyridin-3-yl)-amine. LCMS: m/z 197.1 (M+H)⁺, Rt 2.47 mins.

Step B

The chlorination of ((S)-2-methoxy-1-methyl-ethyl)-(5-methoxy-pyridin-3-yl)-amine (5.30 g) with N-chlorosuccinimide (7.21 g) gives, after purification on silica gel (2,6-dichloro-5-methoxy-pyridin-3-yl)-((S)-2-methoxy-1-methyl-ethyl)-amine. LCMS: m/z 265.2/267.1/269.1 (M+H)⁺, Rt 3.03 mins.

Step C

Analogous to the synthesis of allyl-[(S)-1-(tert-butyl-dimethyl-silanyloxymethyl)-propyl]-(2,5,6-tribromo-pyridin-3-yl)-amine the allylation of (2,6-dichloro-5-methoxy-pyridin-3-yl)-((S)-2-methoxy-1-methyl-ethyl)-amine (5.38 g) with allyl iodide (4.0 mL) affords, after purification on silica gel allyl-(2,6-dichloro-5-methoxy-pyridin-3-yl)-((S)-2-methoxy-1-methyl-ethyl)-amine. LCMS: m/z 305.1/307.1/309.0 (M+H)⁺, Rt 3.49 mins.

Step D

Analogous to the synthesis of 5,6-dibromo-1-[(S)-1-(tert-butyl-dimethyl-silanyloxymethyl)-propyl]-3-methyl-1H-pyrrolo[3,2-b]pyridine the palladium mediated cyclization of allyl-(2,6-dichloro-5-methoxy-pyridin-3-yl)-((S)-2-methoxy-1-methyl-ethyl)-amine (5.13 g) affords, after purification on silica gel 5-chloro-6-methoxy-1-((S)-2-methoxy-1-methyl-ethyl)-3-methyl-1H-pyrrolo[3,2-b]pyridine. LCMS: m/z 269.1/271.1 (M+H)⁺, Rt 2.41 mins.

Step E

Analogous to the synthesis of 6-bromo-1-[(S)-1-(tert-butyl-dimethyl-silanyloxymethyl)-propyl]-5-(6-isopropyl-2-methoxy-pyridin-3-yl)-3-methyl-1H-pyrrolo[3,2-b]pyridine the palladium mediated coupling of 5-chloro-6-methoxy-1-((S)-2-methoxy-1-methyl-ethyl)-3-methyl-1H-pyrrolo[3,2-b]pyridine (1.9 g) with 6-isopropyl-2-methoxy-3-pyridineboronic acid (1.79 g) affords, after purification on silica gel 5-(6-isopropyl-2-methoxy-pyridin-3-yl)-6-methoxy-1-((S)-2-methoxy-1-methyl-ethyl)-3-methyl-1H-pyrrolo[3,2-b]pyridine. LCMS: m/z 384.2 (M+H)⁺, Rt 2.40 mins.

Step F

A solution of 5-(6-isopropyl-2-methoxy-pyridin-3-yl)-6-methoxy-1-((S)-2-methoxy-1-methyl-ethyl)-3-methyl-1H-pyrrolo[3,2-b]pyridine (890 mg) in concentrated hydrochloric acid (60 mL, 37%) is heated at 55° C. for 16 hours. The resulting solution is neutralized with sodium bicarbonate and diluted with a little water. The mixture is extracted with dichloromethane (4×50 mL) and dried over magnesium sulfate. Evaporation of the solvent followed by trituration with diethyl ether gives 6-isopropyl-3-[6-methoxy-1-((S)-2-methoxy-1-methyl-ethyl)-3-methyl-1H-pyrrolo[3,2-b]pyridin-5-yl]-pyridin-2-ol. LCMS: m/z 370.2 (M+H)⁺, Rt 2.01 mins.

Step G

Analogous to the synthesis of trifluoro-methanesulfonic acid 3-(3,6-dimethyl-1-propyl-1H-pyrrolo[3,2-b]pyridin-5-yl)-6-isopropyl-pyridin-2-yl ester the reaction of 6-isopropyl-3-[6-methoxy-1-((S)-2-methoxy-1-methyl-ethyl)-3-methyl-1H-pyrrolo[3,2-b]pyridin-5-yl]-pyridin-2-ol (200 mg) with triflic anhydride (0.11 mL) in the presence of triethyl amine (0.136 mL) gives, after purification on silica gel trifluoro-methanesulfonic acid 6-isopropyl-3-[6-methoxy-1-((S)-2-methoxy-1-methyl-ethyl)-3-methyl-1H-pyrrolo[3,2-b]pyridin-5-yl]-pyridin-2-yl ester. LCMS: m/z 502.1 (M+H)⁺, Rt=3.29 mins.

Step H

Analogous to the preparation of [3-(3,6-dimethyl-1-propyl-1H-pyrrolo[3,2-b]pyridin-5-yl)-6-isopropyl-pyridin-2-yl]-ethyl-amine the reaction of trifluoro-methanesulfonic acid 6-isopropyl-3-[6-methoxy-1-((S)-2-methoxy-1-methyl-ethyl)-3-methyl-1H-pyrrolo[3,2-b]pyridin-5-yl]-pyridin-2-yl ester (70 mg) with ethyl amine solution in THF (0.7 mL, 2M) affords, after purification on silica gel gives ethyl-{6-isopropyl-3-[6-methoxy-1-((S)-2-methoxy-1-methyl-ethyl)-3-methyl-1H-pyrrolo[3,2-b]pyridin-5-yl]-pyridin-2-yl}-amine. LCMS: m/z 397.3 (M+H)⁺, Rt=2.14 mins.

Step I

Analogous to the preparation of 5-(2-ethyl-6-isopropyl-pyridin-3-yl)-3,6-dimethyl-1-propyl-1H-pyrrolo[3,2-b]pyridine the reaction of trifluoro-methanesulfonic acid 6-isopropyl-3-[6-methoxy-1-((S)-2-methoxy-1-methyl-ethyl)-3-methyl-1H-pyrrolo[3,2-b]pyridin-5-yl]-pyridin-2-yl ester (180 mg) with triethylborane solution in hexanes (1.44 mL, 1.0M) affords, after purification on silica gel 5-(2-ethyl-6-isopropyl-pyridin-3-yl)-6-methoxy-1-((S)-2-methoxy-1-methyl-ethyl)-3-methyl-1H-pyrrolo[3,2-b]pyridine. LCMS: m/z 382.3 (M+H)⁺, Rt=1.94 mins.

Replacing the amine used in step H of Example 34 with various other amine reagents, the following compounds are synthesized:
{6-Isopropyl-3-[6-methoxy-1-((S)-2-methoxy-1-methyl-ethyl)-3-methyl-1H-pyrrolo[3,2-b]pyridin-5-yl]-pyridin-2-yl}-dimethyl-amine. Rt 1.97 min m/z 397.2 (M+H)⁺
{6-Isopropyl-3-[6-methoxy-1-((S)-2-methoxy-1-methyl-ethyl)-3-methyl-1H-pyrrolo[3,2-b]pyridin-5-yl]-pyridin-2-yl}-methyl-amine. Rt 1.93 min m/z 383.3 (M+H)⁺

### Example 35

### Synthesis of 6-chloro-5-(2-ethyl-6-isopropyl-pyridin-3-yl)-1-((S)-2-methoxy-1-methyl-ethyl)-3-methyl-1H-pyrrolo[3,2-b]pyridine, 6-Chloro-5-(6-isopropyl-pyridin-3-yl)-1-((S)-2-methoxy-1-methyl-ethyl)-3-methyl-1H-pyrrolo[3,2-b]pyridine and {3-[6-chloro-1-((S)-2-methoxy-1-methyl-ethyl)-3-methyl-1H-pyrrolo[3,2-b]pyridin-5-yl]-6-isopropyl-pyridin-2-yl}-methyl-amine

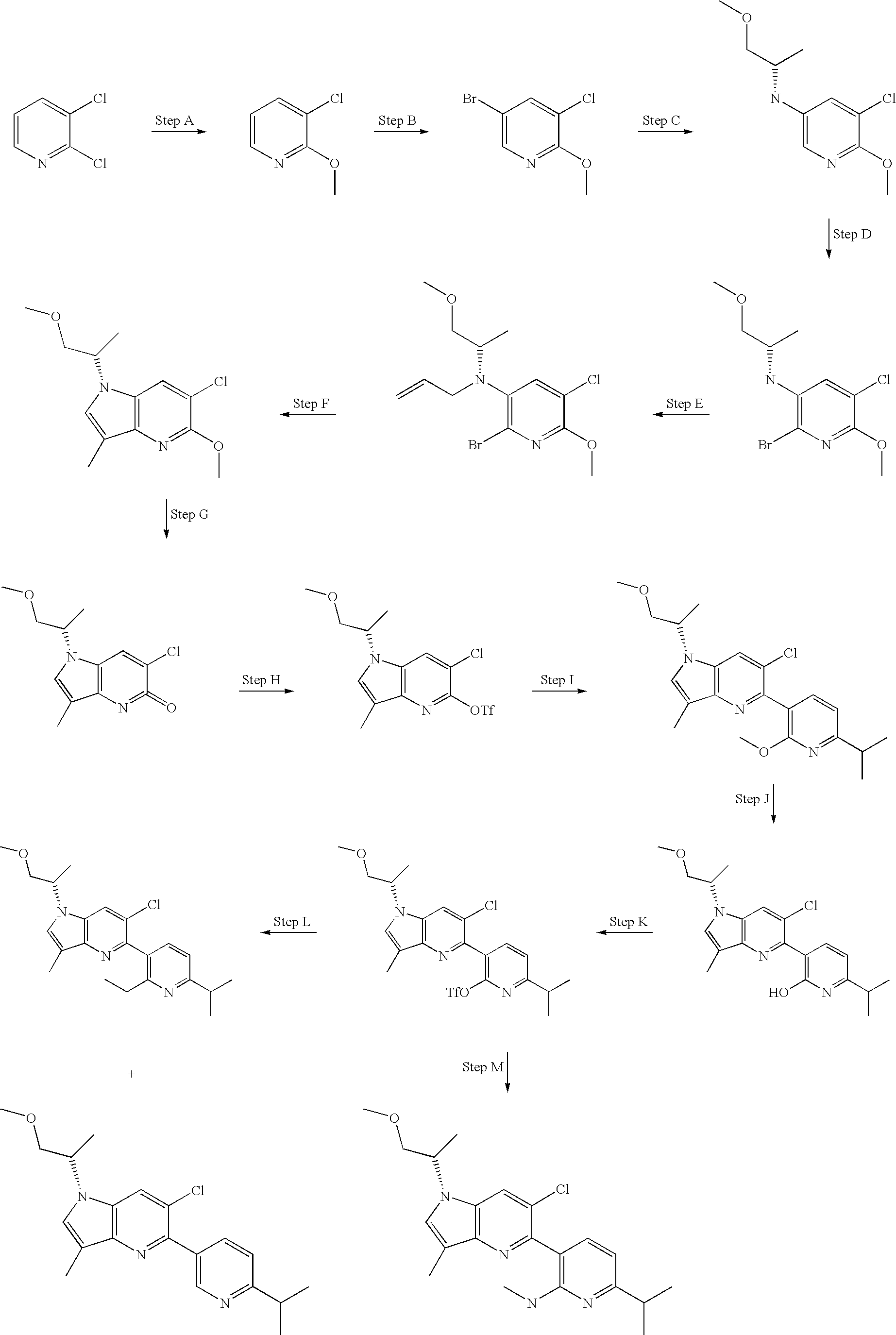

Step A

Similar to a procedure by Testaferre it al. (Tetrahedron 41, No7, 1373-1384, 1985) a suspension of 2,3-dichloropyridine (10 g) in sodium methoxide solution in methanol (62 mL, 25%) is heated to 55° C. for 15 hours. The suspension is filtered and the filtrate is evaporated to low volume. The mixture is diluted with saturated brine, extracted with diethyl ether (3×50 mL) and dried over magnesium sulfate. Evaporation directly gives 3-chloro-2-methoxy-pyridine. LCMS: m/z 144.0/146.0 (M+H)⁺, Rt 2.29 mins.

Step B

Similar to a procedure by Bargar et al. (J. Het Chem 22, 1583, 1985) a stirred suspension of 3-chloro-2-methoxy-pyridine (9.3 g) and sodium acetate (5.4 g) in glacial acetic acid (30 mL) is treated with bromine (6.7 mL) dropwise over 15 mins. After the exotherm has subsided, the mixture is heated at 80° C. for one hour. The reaction mixture is cooled to room temperature and diluted with ether (200 mL) and washed with sodium hydroxide solution (1M) and sodium thiosulphate solution (100 mL, 2M). The ether layer is dried over magnesium sulfate and evaporated to give 5-bromo-3-chloro-2-methoxy-pyridine. This compound is used without further purification in the next reaction.

Step C

Analogous to the preparation of ((S)-2-methoxy-1-methyl-ethyl)-(5-methoxy-pyridin-3-yl)-amine, the palladium mediated amination of 5-bromo-3-chloro-2-methoxy-pyridine (4.0 g) with (S)-1-methoxy-2-propylamine (2.1 mL) affords, after purification on silica gel (5-chloro-6-methoxy-pyridin-3-yl)-((S)-2-methoxy-1-methyl-ethyl)-amine. LCMS: m/z 231.1/233.1 (M+H)⁺, Rt 2.19 mins.

Step D

Analogous to the synthesis of (R)-2-(tert-butyl-dimethyl-silanyloxy)-1-methyl-ethyl]-(2,6-dibromo-5-ethyl-pyridin-3-yl)-amine, the bromination of (5-chloro-6-methoxy-pyridin-3-yl)-((S)-2-methoxy-1-methyl-ethyl)-amine (1.350 g) with N-bromosuccinimide (782 mg) gives, after purification on silica gel (2-bromo-5-chloro-6-methoxy-pyridin-3-yl)-((S)-2-methoxy-1-methyl-ethyl)-amine. LCMS: m/z 309.0/311.0/312.0 (M+H)⁺, Rt 3.00 mins.

Step E

Analogous to the synthesis of allyl-[(S)-1-(tert-butyl-dimethyl-silanyloxymethyl)-propyl]-(2,5,6-tribromo-pyridin-3-yl)-amine, the allylation of (2-bromo-5-chloro-6-methoxy-pyridin-3-yl)-((S)-2-methoxy-1-methyl-ethyl)-amine (1.05 g) with allyl iodide (0.68 mL) gives, after purification on silica gel allyl-(2-bromo-5-chloro-6-methoxy-pyridin-3-yl)-((S)-2-methoxy-1-methyl-ethyl)-amine. LCMS: m/z 349.0/351.0/353.0 (M+H)⁺, Rt 3.32 mins.

Step F

Analogous to the synthesis of 5-chloro-6-methoxy-1-((S)-2-methoxy-1-methyl-ethyl)-3-methyl-1H-pyrrolo[3,2-b]pyridine, the palladium mediated cyclization of allyl-(2-bromo-5-chloro-6-methoxy-pyridin-3-yl)-((S)-2-methoxy-1-methyl-ethyl)-amine (11 2 g) affords, after purification on silica gel affords 6-chloro-5-methoxy-1-((S)-2-methoxy-1-methyl-ethyl)-3-methyl-1H-pyrrolo[3,2-b]pyridine. LCMS: m/z 269.1/271.1 (M+H)⁺, Rt 2.92 mins.

Step G

6-Chloro-5-methoxy-1-((S)-2-methoxy-1-methyl-ethyl)-3-methyl-1H-pyrrolo[3,2-b]pyridine (153 mg) is reacted with sodium thiomethoxide (800 mg). Evaporation of the solvent extracts and trituration of the crude residue with diethyl ether gives 6-chloro-1-((S)-2-methoxy-1-methyl-ethyl)-3-methyl-1,4-dihydro-pyrrolo[3,2-b]pyridin-5-one. LCMS: m/z 255.1/257.1 (M+H)⁺, Rt 2.03 mins.

Step H

Analogous to the synthesis of trifluoro-methanesulfonic acid 6-isopropyl-3-[6-methoxy-1-((S)-2-methoxy-1-methyl-ethyl)-3-methyl-1H-pyrrolo[3,2-b]pyridin-5-yl]-pyridin-2-yl ester, reaction of 6-chloro-1-((S)-2-methoxy-1-methyl-ethyl)-3-methyl-1,4-dihydro-pyrrolo[3,2-b]pyridin-5-one (340 mg) with triflic anhydride (0.27 mL) in the presence of triethyl amine (0.34 mL) affords, after purification on silica gel trifluoro-methanesulfonic acid 6-chloro-1-((S)-2-methoxy-1-methyl-ethyl)-3-methyl-1H-pyrrolo[3,2-b]pyridin-5-yl ester. LCMS: m/z 387.0/389.0 (M+H)⁺, Rt=3.22 mins.

Step I

Analogous to the synthesis of 5-(6-isopropyl-2-methoxy-pyridin-3-yl)-6-methoxy-1-((S)-2-methoxy-1-methyl-ethyl)-3-methyl-1H-pyrrolo[3,2-b]pyridine, the palladium mediated coupling of trifluoro-methanesulfonic acid 6-chloro-1-((S)-2-methoxy-1-methyl-ethyl)-3-methyl-1H-pyrrolo[3,2-b]pyridin-5-yl ester (500 mg) with 6-isopropyl-2-methoxy-3-pyridineboronic acid (430 mg) affords, after purification on silica gel 6-chloro-5-(6-isopropyl-2-methoxy-pyridin-3-yl)-1-((S)-2-methoxy-1-methyl-ethyl)-3-methyl-1H-pyrrolo[3,2-b]pyridine. LCMS: m/z 388.2/390.2 (M+H)⁺, Rt 3.15 mins.

Step J

Analogous to the preparation of 6-chloro-1-((S)-2-methoxy-1-methyl-ethyl)-3-methyl-1,4-dihydro-pyrrolo[3,2-b]pyridin-5-one, 6-chloro-5-(6-isopropyl-2-methoxy-pyridin-3-yl)-1-((S)-2-methoxy-1-methyl-ethyl)-3-methyl-1H-pyrrolo[3,2-b]pyridine (300 mg) is reacted with sodium thiomethoxide (500 mg). Evaporation of the solvent extracts and trituration of the crude residue with diethyl ether gives 3-[6-chloro-1-((S)-2-methoxy-1-methyl-ethyl)-3-methyl-1H-pyrrolo[3,2-b]pyridin-5-yl]-6-isopropyl-1H-pyridin-2-one. LCMS: m/z 374.2/376.2 (M+H)⁺, Rt 2.23 mins.

Step K

Analogous to the preparation of trifluoro-methanesulfonic acid 6-isopropyl-3-[6-methoxy-1-((S)-2-methoxy-1-methyl-ethyl)-3-methyl-1H-pyrrolo[3,2-b]pyridin-5-yl]-pyridin-2-yl ester, reaction of 3-[6-chloro-1-((S)-2-methoxy-1-methyl-ethyl)-3-methyl-1H-pyrrolo[3,2-b]pyridin-5-yl]-6-isopropyl-1H-pyridin-2-one (250 mg) with triflic anhydride (0.14 mL) in the presence of triethyl amine (0.17 mL) gives, after purification on silica gel trifluoro-methanesulfonic acid 3-[6-chloro-1-((S)-2-methoxy-1-methyl-ethyl)-3-methyl-1H-pyrrolo[3,2-b]pyridin-5-yl]-6-isopropyl-pyridin-2-yl ester. LCMS: m/z 506.1/508.1 (M+H)⁺, Rt=4.02 mins.

Step L

Analogous to the preparation of 5-(2-ethyl-6-isopropyl-pyridin-3-yl)-6-methoxy-1-((S)-2-methoxy-1-methyl-ethyl)-3-methyl-1H-pyrrolo[3,2-b]pyridine, reaction of trifluoro-methanesulfonic acid 3-[6-chloro-1-((S)-2-methoxy-1-methyl-ethyl)-3-methyl-1H-pyrrolo[3,2-b]pyridin-5-yl]-6-isopropyl-pyridin-2-yl ester (145 mg) with triethylborane solution in hexanes (1.2 mL, 1.0M) affords, after purification on silica gel 6-chloro-5-(2-ethyl-6-isopropyl-pyridin-3-yl)-1-((S)-2-methoxy-1-methyl-ethyl)-3-methyl-1H-pyrrolo[3,2-b]pyridine. LCMS: m/z 386.2/388.2 (M+H)⁺, Rt=2.02 mins. Isolated as a byproduct of this reaction is 6-chloro-5-(6-isopropyl-pyridin-3-yl)-1-((S)-2-methoxy-1-methyl-ethyl)-3-methyl-1H-pyrrolo[3,2-b]pyridine. LCMS: m/z 358.2/360.2 (M+H)⁺, Rt=2.15 mins.

Step M

Analogous to the preparation of ethyl-{6-isopropyl-3-[6-methoxy-1-((S)-2-methoxy-1-methyl-ethyl)-3-methyl-1H-pyrrolo[3,2-b]pyridin-5-yl]-pyridin-2-yl}-amine, the reaction of trifluoro-methanesulfonic acid 3-[6-chloro-1-((S)-2-methoxy-1-methyl-ethyl)-3-methyl-1H-pyrrolo[3,2-b]pyridin-5-yl]-6-isopropyl-pyridin-2-yl ester (85 mg) with methyl amine solution in THF (0.9 mL, 2M) gives, after purification on silica gel {3-[6-chloro-1-((S)-2-methoxy-1-methyl-ethyl)-3-methyl-1H-pyrrolo[3,2-b]pyridin-5-yl]-6-isopropyl-pyridin-2-yl}-methyl-amine. LCMS: m/z 387.2/389.2 (M+H)⁺, Rt=2.09 mins.

Replacing the amine in step M of Example 35 with various other amine reagents, the following compounds are synthesized:
{3-[6-Chloro-1-(2-methoxy-1-methyl-ethyl)-3-methyl-1H-pyrrolo[3,2-b]pyridin-5-yl]-6-isopropyl-pyridin-2-yl}-dimethyl-amine Rt 2.12 min m/z 401.2 (M+H)⁺
{3-[6-Chloro-1-(2-methoxy-1-methyl-ethyl)-3-methyl-1H-pyrrolo[3,2-b]pyridin-5-yl]-6-isopropyl-pyridin-2-yl}-ethyl-amine Rt 2.22 min m/z 401.2 (M+H)⁺

### Example 36

### Synthesis of {3-[6-chloro-1-((R)-1-fluoromethyl-2-methoxy-ethyl)-3-methyl-1H-pyrrolo[3,2-b]pyridin-5-yl]-6-isopropyl-pyridin-2-yl}-methyl-amine and 6-chloro-5-(2-ethyl-6-isopropyl-pyridin-3-yl)-1-((R)-1-fluoromethyl-2-methoxy-ethyl)-3-methyl-1H-pyrrolo[3,2-b]pyridine

Substituting (R)-1-(tert-Butyl-dimethyl-silanyloxymethyl)-2-methoxy-ethylamine for (S)-1-methoxy-2-propylamine in the scheme of Example 35 and introducing the fluoro group after step F as described in steps F and G of Example 13 gives {3-[6-chloro-1-((R)-1-fluoromethyl-2-methoxy-ethyl)-3-methyl-1H-pyrrolo[3,2-b]pyridin-5-yl]-6-isopropyl-pyridin-2-yl}-methyl-amine, Rt 2.09 min m/z 405.2 (M+H)⁺ and 6-chloro-5-(2-ethyl-6-isopropyl-pyridin-3-yl)-1-((R)-1-fluoromethyl-2-methoxy-ethyl)-3-methyl-1H-pyrrolo[3,2-b]pyridine, Rt 2.34 min m/z 448.12 (M+H)⁺.

### Example 37

### Synthesis of {5-Chloro-3-[1-((R)-1-fluoromethyl-2-methoxy-ethyl)-3,6-dimethyl-1H-pyrrolo[3,2-b]pyridin-5-yl]-6-isopropyl-pyridin-2-yl}-methyl-amine

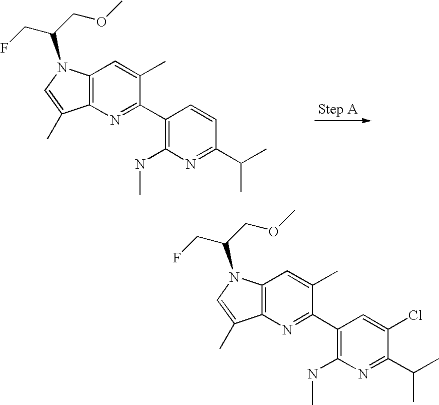

Step A

N-Chlorosuccinimide (33 mg) is added to a solution of {3-[1-(1-fluoromethyl-2-methoxy-ethyl)-3,6-dimethyl-1H-pyrrolo[3,2-b]pyridin-5-yl]-6-isopropyl-pyridin-2-yl}-methyl-amine (94 mg) in chloroform (3 mL). After 18 hr additional N-chlorosuccinimide (10 mg) is added and then after a further 5 min water (10 mL) and dichloromethane (10 mL) are added to the reaction mixture. The organic layer is separated, dried, and evaporated to give, after chromatography over silica gel, {5-Chloro-3-[1-((R)-1-fluoromethyl-2-methoxy-ethyl)-3,6-dimethyl-1H-pyrrolo[3,2-b]pyridin-5-yl]-6-isopropyl-pyridin-2-yl}-methyl-amine. Rt 2.67 min m/z 419.2 (M+H)⁺.

### Example 38

### Synthesis of 1-((R)-1-fluoromethyl-2-methoxy-ethyl)-5-(6-isopropyl-2-methyl-pyridin-3-yl)-3,6-dimethyl-1H-pyrrolo[3,2-b]pyridine

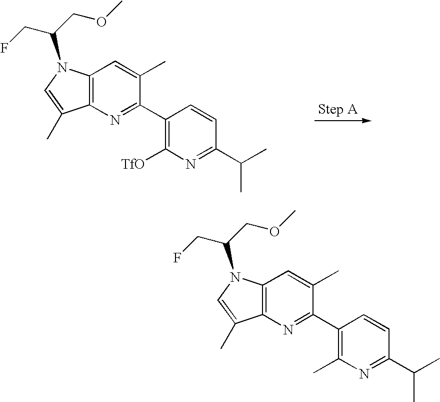

Step A

Trifluoro-methanesulfonic acid 3-[1-(1-fluoromethyl-2-methoxy-ethyl)-3,6-dimethyl-1H-pyrrolo[3,2-b]pyridin-5-yl]-6-isopropyl-pyridin-2-yl ester (97 mg), bis(triphenylphosphine)palladium(II) chloride (4 mg) and lithium chloride (25 mg) are introduced in a glass tube that was then filled with nitrogen. DMF (2 mL) and tetramethyltin (30 uL) are added, the tube is closed with a cap and the reaction mixture is heated at 100° C. overnight. Water (2 mL) and EtOAc (2 mL) are added and then the organic layer is separated. The aqueous layer is extracted three more times with EtOAc and then the combined organic phase is dried and evaporated to give, after silica gel purification, 1-((R)-1-Fluoromethyl-2-methoxy-ethyl)-5-(6-isopropyl-2-methyl-pyridin-3-yl)-3,6-dimethyl-1H-pyrrolo[3,2-b]pyridine. Rt 1.83 min m/z 370.2 (M+H)⁺.

### Example 39

In a manner analogous to the synthesis of 1-((R)-1-fluoromethyl-2-methoxy-ethyl)-5-(6-isopropyl-2-methyl-pyridin-3-yl)-3,6-dimethyl-1H-pyrrolo[3,2-b]pyridine, trifluoro-methanesulfonic acid 3-[6-chloro-1-((S)-2-methoxy-1-methyl-ethyl)-3-methyl-1H-pyrrolo[3,2-b]pyridin-5-yl]-6-isopropyl-pyridin-2-yl ester affords 6-chloro-1-((R)-1-fluoro-methyl-2-methoxy-ethyl)-5-(6-isopropyl-2-methyl-pyridin-3-yl)-3-methyl-1H-pyrrolo[3,2-b]pyridine. Rt 2.17 min m/z 390.11 (M+H)⁺.

### Example 40

### Synthesis of Ethyl-{3-[1-((R)-1-fluoromethyl-2-methoxy-ethyl)-3,6-dimethyl-1H-pyrrolo[3,2-b]pyridin-5-yl]-6-isopropyl-pyridin-2-yl}-amine

In a manner analogous to the synthesis of {3-[1-((R)-1-Fluoromethyl-2-methoxy-ethyl)-3,6-dimethyl-1H-pyrrolo[3,2-b]pyridin-5-yl]-6-isopropyl-pyridin-2-yl}-methyl-amine, trifluoro-methanesulfonic acid 3-[1-(1-fluoromethyl-2-methoxy-ethyl)-3,6-dimethyl-1H-pyrrolo[3,2-b]pyridin-5-yl]-6-isopropyl-pyridin-2-yl ester and ethylamine afford ethyl-{3-[1-((R)-1-fluoromethyl-2-methoxy-ethyl)-3,6-dimethyl-1H-pyrrolo[3,2-b]pyridin-5-yl]-6-isopropyl-pyridin-2-yl}-amine. Rt 2.03 min m/z 399.2 (M+H)⁺.

### Example 41

### Synthesis of 2-bromo-7-(1-ethyl-propyl)-3-(2-methoxy-4-trifluoromethoxy-phenyl)-5-methyl-5H-pyrrolo[2,3-b]pyrazine, 7-(1-ethyl-propyl)-3-(2-methoxy-4-trifluoromethoxy-phenyl)-5-methyl-5H-pyrrolo[2,3-b]pyrazine and 2-ethyl-7-(1-ethyl-propyl)-3-(2-methoxy-4-trifluoromethoxy-phenyl)-5-methyl-5H-pyrrolo[2,3-b]pyrazine

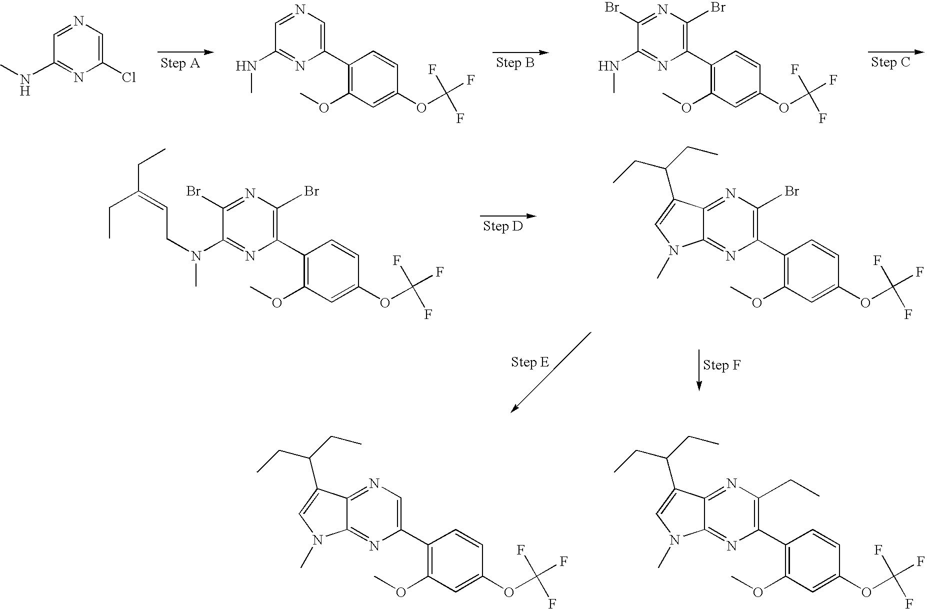

Step A

A mixture of (6-chloro-pyrazin-2-yl)-methyl-amine (431 mg, 3 mmol), 2-methoxy-4-trifluoromethoxyphenyl boronic acid (780 mg, 3.3 mmol) in 2M Na₂CO₃ (3 mL, 6 mmol) and toluene (3 mL) is treated with Pd(PPh₃)₄ (50 mg) under nitrogen at 80° C. for 16 h. After cooling to room temperature, the product is extracted with ethyl acetate (3×10 mL), dried, concentrated and purified by silica gel column chromatography to give [6-(2-methoxy-4-trifluoromethoxy-phenyl)-pyrazin-2-yl]-methyl-amine. LC-MS (M+1): 300.

Step B

To a solution of [6-(2-methoxy-4-trifluoromethoxy-phenyl)-pyrazin-2-yl]-methyl-amine (1.2 g, 4 mmol) in chloroform (20 mL) is added NBS (1.78 g, 10 mmol) in one portion at 0° C. The mixture is then stirred at room temperature for 15 min followed by concentration and purification by silica gel column chromatography to provide [3,5-dibromo-6-(2-methoxy-4-trifluoromethoxy-phenyl)-pyrazin-2-yl]-methyl-amine. LC-MS (M+1): 458.

Step C

To a solution of NaH (95%, 65 mg, 4.5 mmol) and Bu₄NBr (144 mg, 0.45 mmol) in anhydrous NMP (2 mL) is added dropwise a solution of [3,5-dibromo-6-(2-methoxy-4-trifluoromethoxy-phenyl)-pyrazin-2-yl]-methyl-amine (1.37 g, 3 mmol) in NMP (10 mL) under nitrogen at room temperature in 5 min. The mixture is continued stirring at room temperature for 1 h, followed by addition of 1-chloro-3-ethyl-pent-2-ene (594 mg, 4.5 mmol). The mixture is then heated at 65° C. for 16 h. After cooling to room temperature, the product is extracted with ethyl acetate (3×20 mL), washed with water (2×8 mL) and brine (10 mL), dried, concentrated and purified by silica gel column chromatography to give [3,5-dibromo-6-(2-methoxy-4-trifluoromethoxy-phenyl)-pyrazin-2-yl]-(3-ethyl-pent-2-enyl)-methyl-amine.

Step D

A mixture of [3,5-dibromo-6-(2-methoxy-4-trifluoromethoxy-phenyl)-pyrazin-2-yl]-(3-ethyl-pent-2-enyl)-methyl-amine (1.77 g, 3.2 mmol), Bu₄NBr (1.02 g, 3.2 mmol), K₂CO₃ (1.33 g, 9.6 mmol) and Pd(OAc)₂ in anhydrous DMF (20 mL) under nitrogen is heated at 90° C. for 1 h. After cooling to room temperature, the reaction is quenched by addition of water (10 mL). The product is extracted with ethyl acetate (3×20 mL), washed with water (2×8 mL) and brine (10 mL), dried, concentrated and purified by silica gel column chromatography to give 2-bromo-7-(1-ethyl-propyl)-3-(2-methoxy-4-trifluoromethoxy-phenyl)-5-methyl-5H-pyrrolo[2,3-b]pyrazine. LC-MS (M+1): 472

Step E

A solution of 2-bromo-7-(1-ethyl-propyl)-3-(2-methoxy-4-trifluoromethoxy-phenyl)-5-methyl-5H-pyrrolo[2,3-b]pyrazine (50 mg, 0.106 mmol) in ethyl acetate (5 mL) is hydrogenated with 5% Pd—C (10 mg) under atmosphere at room temperature overnight. After filtration and concentration, the product is purified by silica gel column chromatography to give 7-(1-ethyl-propyl)-3-(2-methoxy-4-trifluoromethoxy-phenyl)-5-methyl-5H-pyrrolo[2,3-b]pyrazine. ¹H NMR (CDCl₃, δ): 0.85 (t, J=7.2 Hz, 6H), 1.82 (m, 4H), 2.87 (m, 1H), 3.87 (s, 1H), 3.88 (s, 3H), 6.86 (s, 1H), 6.98 (d, J=8.4 Hz), 7.19 (s, 1H), 7.87 (, d, J=8.4 Hz, 1H), 8.90 (s, 1H); LC-MS (M+1): 394.

Step F

A mixture of 2-bromo-7-(1-ethyl-propyl)-3-(2-methoxy-4-trifluoromethoxy-phenyl)-5-methyl-5H-pyrrolo[2,3-b]pyrazine (47 mg, 0.1 mmol), Et₃B (1M solution in hexane, 0.2 mL, 0.2 mmol) in 2M Na₂CO₃ (0.5 mL, 1 mmol) and toluene (1 mL) is treated with Pd(PPh₃)₄ (10 mg) under nitrogen at 90° C. for 16 h. After cooling to room temperature, the product is extracted with ethyl acetate (3×10 mL), dried, concentrated and purified by silica gel column chromatography to give 2-ethyl-7-(1-ethyl-propyl)-3-(2-methoxy-4-trifluoromethoxy-phenyl)-5-methyl-5H-pyrrolo[2,3-b]pyrazine. ¹H NMR (CDCl₃, δ): 0.87 (t, J=7.6 Hz, 6H), 1.17 (t, J=7.6 Hz, 3H), 1.82 (m, 4H), 2.69 (m, 2H), 2.92 (m, 1H), 3.77 (s, 1H), 3.81 (s, 3H), 6.83 (s, 1H), 6.94 (d, J=8.0 Hz), 7.12 (s, 1H), 7.31 (d, J=8.0 Hz, 1H); LC-MS (M+1): 408.

### Example 42

### Synthesis of 2-methyl-7-(1-ethyl-propyl)-3-(2-methoxy-4-trifluoromethoxy-phenyl)-5-methyl-5H-pyrrolo[2,3-b]pyrazine

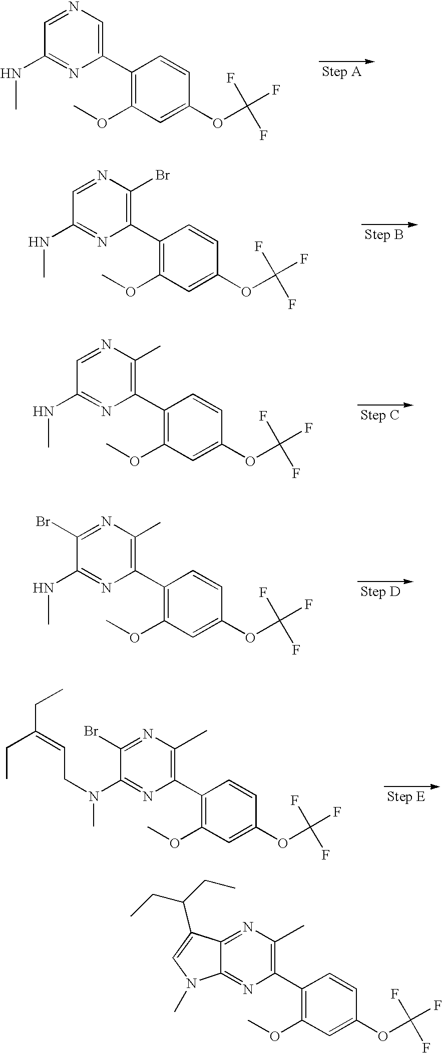

Step A

To a solution of [6-(2-methoxy-4-trifluoromethoxy-phenyl)-pyrazin-2-yl]-methyl-amine (3 g, 10 mmol) in chloroform (25 mL) is added NBS (2.13 g, 12 mmol) in one portion at 0° C. The mixture is then stirred at room temperature for 30 min followed by concentration and purification by silica gel column chromatography to provide [5-bromo-6-(2-methoxy-4-trifluoromethoxy-phenyl)-pyrazin-2-yl]-methyl-amine. LC-MS (M+1): 378.

Step B

A mixture of [5-bromo-6-(2-methoxy-4-trifluoromethoxy-phenyl)-pyrazin-2-yl]-methyl-amine (120 mg, 0.32 mmol), MeB(OH)₂ (192 mm, 3.2 mmol) in 2M Na₂CO₃ (2 mL, 4 mmol) and toluene (2 mL) is treated with Pd(PPh₃)₄ (20 mg) under nitrogen at 85° C. for 16 h. After cooling to room temperature, the product is extracted with ethyl acetate (3×10 mL), dried, concentrated and purified by silica gel column chromatography to give [6-(2-methoxy-4-trifluoromethoxy-phenyl)-5-methyl-pyrazin-2-yl]-methyl-amine.

Step C

[3-bromo-6-(2-methoxy-4-trifluoromethoxy-phenyl)-5-methyl-pyrazin-2-yl]-methyl-amine is prepared by the same procedure as described in step A. LC-MS: 392.

Step D

[3-bromo-6-(2-methoxy-4-trifluoromethoxy-phenyl)-5-methyl-pyrazin-2-yl]-(3-ethyl-pent-2-enyl)-methyl-amine is prepared by the same procedure for [3,5-dibromo-6-(2-methoxy-4-trifluoromethoxy-phenyl)-pyrazin-2-yl]-(3-ethyl-pent-2-enyl)-methyl-amine.

Step E

7-(1-Ethyl-propyl)-3-(2-methoxy-4-trifluoromethoxy-phenyl)-2,5-dimethyl-5H-pyrrolo[2,3-b]pyrazine is prepared by the same procedure for 2-bromo-7-(1-ethyl-propyl)-3-(2-methoxy-4-trifluoromethoxy-phenyl)-5-methyl-5H-pyrrolo[2,3-b]pyrazine. ¹H NMR (CDCl₃, δ): 0.86 (t, J=7.6 Hz, 6H), 1.83 (m, 4H), 2.42 (s, 3H), 2.93 (m, 1H), 3.78 (s, 1H), 3.81 (s, 3H), 6.83 (s, 1H), 6.94 (d, J=8.0 Hz), 7.12 (s, 1H), 7.31 (d, J=8.0 Hz, 1H); LC-MS (M+1): 422.

### Example 43

### Synthesis of N,N-diethyl-{4-ethyl-5-[2-ethyl-7-(1-ethyl-propyl)-5-methyl-5H-pyrrolo[2,3-b]pyrazin-3-yl]-pyridin-2-yl}-amine

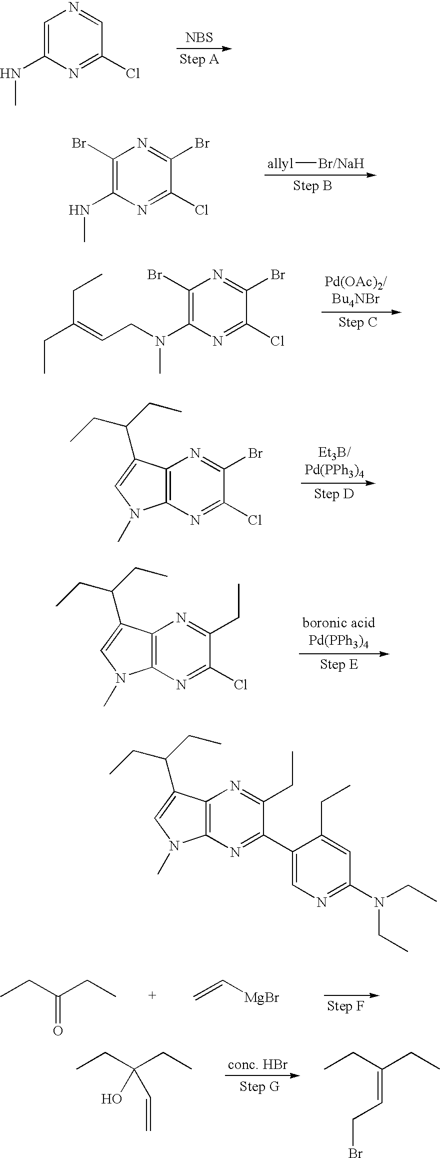

Step A

The previously described 2-chloro-4-methylaminopyrazine (40.0 g) is dissolved in chloroform (500 mL) and NBS (104.0 g) is added. After being stirred for 16 h, the yellowish mixture is put into water (500 mL) and sat. sodium bicarbonate (100 mL), extracted with ethyl acetate/hexane (⅓, 2×400 mL), and dried over magnesium sulfate. The crude is then flushed through a plug of silica gel (ethyl acetate/hexane=1/3) and used without any other purification. TLC: Rf=0.63 (ethyl acetate/hex=1/3)

Step B

The crude dibromide (73.69 g) of step A and the later described 3,3-diethylallyl bromide (84.40 g, step F+G) are dissolved in DMF (400 mL). Sodium hydride (15.50 g) is added in portions and the reaction is stirred for 30 min at rt. The mixture is then put into water (2000 mL) and extracted with ethyl acetate/hexane (1/6, 4×700 mL). The combined organic layers are washed with water (200 mL), dried over magnesium sulfate, and filtered directly through a plug of silica gel (200 g). The crude material is used directly in step C. TLC: Rf=0.90 (EtOAc/hex=1/6)

Step C

The crude allyl compound (116.0 g) of step B, tetrabutylammonium bromide (75.3 g), palladium acetate (5.2 g), and potassium carbonate (97.0 g) are dissolved in DMF (1200 mL). After being heated to 80° C. for 6 h, the mixture is worked-up according to step B. Final purification on silica gel affords the bicyclic compound. TLC: Rf=0.59 (EtOAc/hex=1/6)

Step D

The bicyclic compound (1.83 g) of step C is dissolved in toluene (50 mL). After degassing, tetrakis(triphenylphosphine)palladium(0) (0.67 g) is added. A second degassing is followed by addition of triethylborane (28.9 mL, 1N in hexane) and of a 2N potassium carbonate solution (6.0 mL) whereupon the reaction is heated to 80° C. for 36 h. The yellowish mixture is then put into water (200 mL), extracted with DCM (3×150 mL), and dried over magnesium sulfate. Purification on silica gel affords the ethyl derivative. LCMS: m/z 266.14 (M+H)⁺

Step E

The ethyl derivative (500 mg) of step D and the previously described 2-diethylamino-4-ethyl-5-pyridine boronic acid (526 mg) are dissolved in DME (15 mL). After degassing, tetrakis(triphenylphosphine)palladium(0) (183 mg) is added. A second degassing is followed by addition of a 1N sodium carbonate solution (3.2 mL) whereupon the reaction is heated to 80° C. for 40 h. The yellowish mixture is then put into water (100 mL), extracted with DCM (3×100 mL), and dried over magnesium sulfate. Purification on silica gel affords the title compound. LCMS: m/z 408.37 (M+H)⁺

Step F

3-Pentanone (73.9 mL) in THF (300 mL) is slowly added to vinyl magnesium bromide (800 mL, 1N in THF) at rt. After being stirred for 24 h, the mixture is put into water (2500 mL) and sat. sodium bicarbonate (500 mL), extracted with DCM (1×1500 mL, 2×500 mL), and dried over magnesium sulfate. The crude mixture is used without any further purification in step G.

Step G

The crude mixture (82.0 g) of step F is dissolved in conc. HBr (250 mL). After 20 min or once NMR control shows completed conversion, the dark mixture is put into water (500 mL), extracted with DCM (3×250 mL), and dried over magnesium sulfate. The crude mixture is used without any further purification in step B.

### Example 44

### Synthesis of 2-[3-(6-isopropyl-2-methoxy-pyridin-3-yl)-2,5-dimethyl-5H-pyrrolo[2,3-b]pyrazin-7-yl]-propan-1-ol

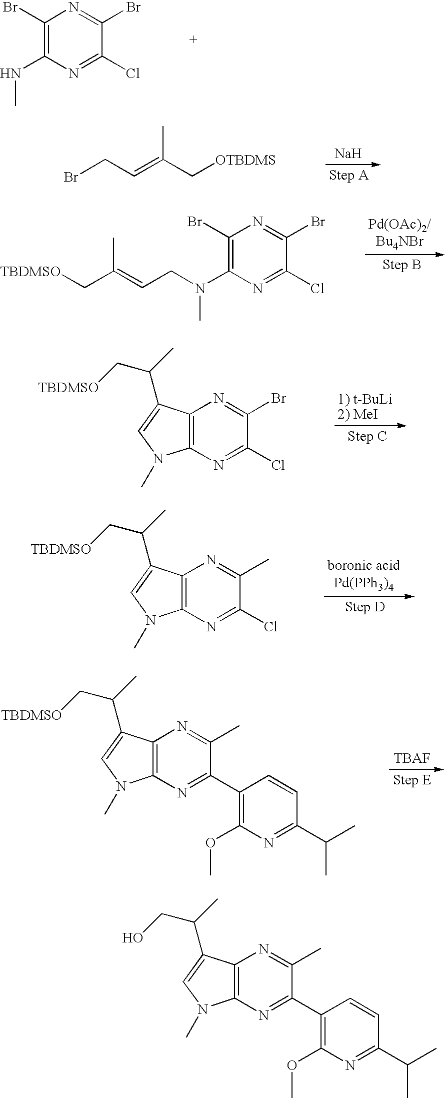

Step A

The previously described 2,6-dibromo-3-chloro-5-methylaminopyrazine (550 mg) and the shown allylic bromide (560 mg, synthesized identically to the Me-regioisomere described by 15 Enders et al., Synlett 2002, 2280) are dissolved in DMF (10 mL). After addition of sodium hydride (91 mg), the dark red reaction mixture is stirred for 15 min. Subsequently, the mixture is put into water (200 mL) and sat. sodium bicarbonate (100 ml), extracted with ethyl ether (2×100 mL), and dried over magnesium sulfate. Purification on silica gel affords the allylic compound. TLC: Rf=0.69 (EtOAc/hex=1/6)

Step B

The allylic compound (892 mg) of step A, tetrabutylammonium bromide (575 mg), palladium acetate (40 mg), and potassium carbonate (737 mg) are dissolved in DMF (10 mL). After heating to 80° C. for 30 min, the mixture is worked-up according to step A. Purification on silica gel affords the Heck-product. LCMS: m/z 417.93 (M+H)⁺

Step C

The Heck product (356 mg) of step B is dissolved in THF (2.5 mL) and added to a solution of t-BuLi (1.05 mL, 1.7N in pentane) in THF (8.5 mL) at −78° C. After being stirred for 10 min, methyl iodide (0.21 mL) is added and the reaction mixture is stirred for another 1 h at −78° C. Subsequently, the mixture is put into water (100 mL) and sat. sodium bicarbonate (50 ml), extracted with DCM (3×100 mL), and dried over magnesium sulfate. Purification on silica gel affords the methyl derivative. LCMS: m/z 354.12 (M+H)⁺

Step D

The methyl product of step C (238 mg) and the previously described 2-isopropyl-6-methoxy-5-pyridine boronic acid (158 mg) are dissolved in DME (5.0 mL). After degassing, tetrakis(triphenylphosphine)palladium(0) (77 mg) is added. A second degassing is followed by addition of a 1N sodium carbonate solution (1.35 mL) whereupon the reaction is heated to 80° C. for 3 h. The yellowish mixture is then put into water (100 mL), extracted with DCM (3×100 mL), and dried over magnesium sulfate. Purification on silica gel affords the coupled product. LCMS: m/z 469.15 (M+H)⁺

Step E

The Suzuki product of step D is dissolved in THF (5.0 mL). After addition of TBAF monohydrate (650 mg), the reaction mixture is stirred for 30 min. Subsequently, the yellow solution is put into water (100 mL), extracted with DCM (3×100 mL), and dried over magnesium sulfate. Purification on silica gel affords the title compound. LCMS: m/z 355.16 (M+H )⁺

### Example 45

### Synthesis of 3-(6-isopropyl-2-methoxy-pyridin-3-yl)-7-(2-methoxy-1-methyl-ethyl)-2,5-dimethyl-5H-pyrrolo[2,3-b]pyrazine

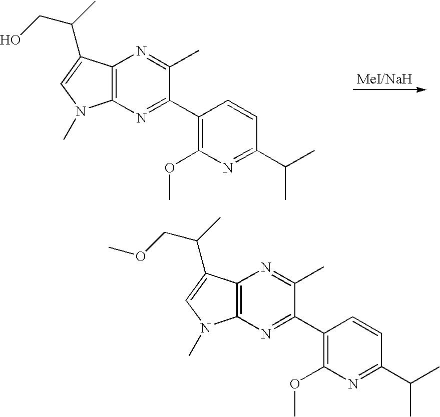

2-[3-(6-Isopropyl-2-methoxy-pyridin-3-yl)-2,5-dimethyl-5H-pyrrolo[2,3-b]pyrazin-7-yl]-propan-1-ol (33 mg) is dissolved in THF (5.0 mL). After addition of sodium hydride (74 mg), the cloudy mixture is stirred for 5 min before methyl iodide (0.23 mL) is added. The reaction is stirred for 16 h, put into water (100 mL) and sat. sodium bicarbonate (10 ml), extracted with DCM (3×100 mL), and dried over magnesium sulfate. Purification on silica gel affords the title compound. LCMS: m/z 369.15 (M+H)⁺

### Example 46

### Synthesis of 3-(6-isopropyl-2-methoxy-pyridin-3-yl)-2,5-dimethyl-7-(1-methyl-2-morpholin-4-yl-ethyl)-5H-pyrrolo[2,3-b]pyrazine

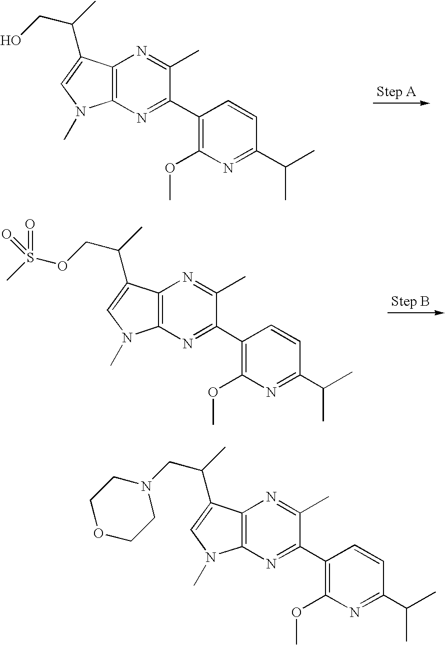

Step A

2-[3-(6-Isopropyl-2-methoxy-pyridin-3-yl)-2,5-dimethyl-5H-pyrrolo[2,3-b]pyrazin-7-yl]-propan-1-ol (142 mg) is dissolved in DCM (5.0 mL) and cooled to 0° C. Mesyl chloride (34 μL) and triethylamine (78 μL) are added before the reaction is stirred for 30 min at 0° C. Subsequently, the yellow solution is put into water (100 mL), extracted with DCM (3×100 mL), and dried over magnesium sulfate. The crude mixture is carried on to step B without any further purification. LCMS: m/z 433.07 (M+H )⁺

Step B

The mesylate (54 mg) of step A is dissolved in acetonitrile (1.0 mL). After addition of morpholine (200 mg), the reaction is heated to 80° C. for 3 h. Subsequently, the clear solution is put into water (100 mL), extracted with DCM (3×100 mL), and dried over magnesium sulfate. Purification on silica gel affords the title compound. LCMS: m/z 424.13 (M+H)⁺

### Example 47

### Synthesis of 3-(1-ethyl-propyl)-6-(2-methoxy-4-trifluoromethoxy-phenyl)-1,5-dimethyl-1H-pyrrolo[2,3-b]pyridine

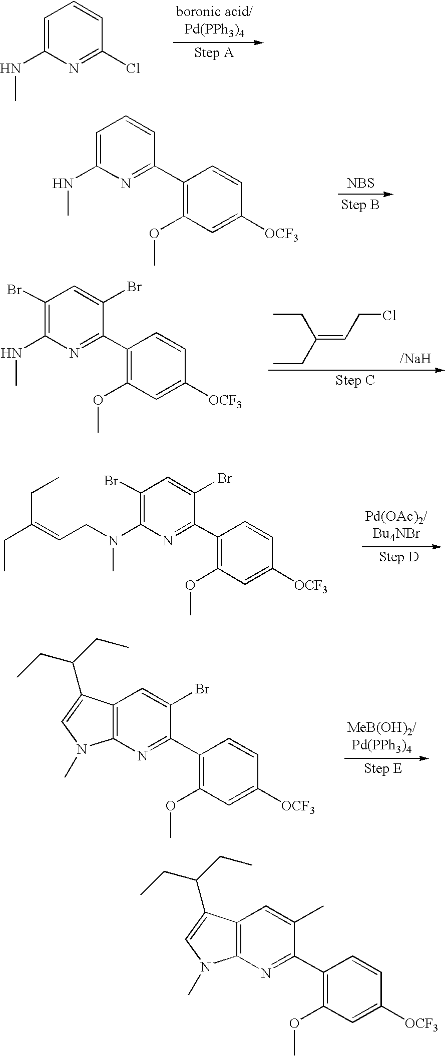

Step A

The previously described 2-chloro-6-methylaminopyridine (670 mg), the also previously described 2-methoxy-4-trifluoromethoxyphenyl boronic acid (1.37 g), and Pd(PPh₃)₄ (115 mg) are dissolved in toluene (30 mL). After addition of 2N Na₂CO₃ (6 mL), the mixture is degassed and then heated at 85° C. overnight. The solution is diluted with EtOAc and washed with 2N NaOH, H₂O, brine, and dried over MgSO₄. Purification on silica gel yields the Suzuki product. ¹H NMR (CDCl₃, δ ppm): 7.78 (1H, d, J=8.4 Hz), 7.49 (1H, t, J=7.6 Hz), 7.08 (1H, d, J=7.6 Hz), 6.90 (1H, dd, J=7.4, 2.0 Hz), 6.80 (1H, d, J=2.0 Hz), 6.34 (1H, J=8.4 Hz), 4.66 (1H, brs), 3.84 (3H, s), 2.94 (3H, d, J=5.0 Hz).

Step B

To a cooled solution of the Suzuki product from Step A (1.0 g) in CHCl₃ is added a solution of NBS (1.22 g) in CHCl₃ at 0° C. over 30 min. After being stirred at rt for 1 hr, the reaction mixture is evaporated. The residue is then purified on silica gel to yield the bromide. ¹H NMR (CDCl₃, δ ppm): 7.80 (1H, s), 7.28 (1H, d, J=8.4 Hz), 6.90 (1H, dd, J=8.4, 1.1 Hz), 6.79 (1H, s), 5.03 (1H, brs), 3.82 (3H, s), 2.98 (3H, d, J=5.0 Hz).

Step C

To a solution of the crude bromide (1.1 g) from step B in NMP (10 mL) is added NaH (60%, 0.195 g). After being stirred for 2 h, freshly distilled 3,3-diethylallyl chloride (0.414 g, prepared analogously to the previously described 3,3-diethylallyl bromide) is added to the reaction mixture. Stirring for an additional 1 h is followed by quenching with H₂O and extraction with EtOAc. The organic layer is washed with H₂O, brine, and dried over Na₂SO₄. Purification on silica gel yields the allyl compound. ¹H NMR (CDCl₃, δ ppm): 7.95 (1H, s), 7.28 (1H, d, J=8.3 Hz), 6.90 (1H, d, J=8.3 Hz), 6.79 (1H, s), 5.30 (1H, m), 3.92 (2H, d, J=6.6 Hz), 3.81 (3H, s), 2.87 (3H, s), 2.06 (4H, m), 1.01 (3H, t, J=7.5 Hz), 0.94 (3H, t, J=7.5 Hz).

Step D

The allyl compound of step C (330 mg), Pd(OAc)₂ (40 mg), tetrabutylammonium bromide (219 mg), and K₂CO₃ (250 mg) are dissolved in DMF (3 mL), degassed, and heated to 80° C. overnight. The mixture is then diluted with EtOAc and washed with H₂O, brine, and dried over MgSO₄. Purification on silica gel yields the Heck product. ¹H NMR (CDCl₃, δ ppm): 8.11 (1H, s), 7.31 (1H, d, J=8.3 Hz), 6.94 (2H, m), 6.82 (1H, d, J=1.7 Hz), 3.81 (3H, s),3.80 (3H, s), 2.58 (1H, m), 1.62-1.79 (4H, m), 0.85 (6H, t, J=7.3 Hz).

Step E

The Heck product of step D (80 mg), methylboronic acid (60 mg), and Pd(PPh₃)₄ (10 mg) are dissolved in toluene (5 mL). After addition of 2N Na₂CO₃ (3 mL), the reaction mixture is degassed and then heated to 85° C. overnight. Subsequently, the solution is diluted with EtOAc and washed with 2N NaOH, H₂O, and brine before being dried over MgSO₄. Purification on silica gel yields the title compound. ¹H NMR (CDCl₃, δ ppm): 7.73 (1H, s), 7.32 (1H, d, J=8.2 Hz), 6.93 (1H, d, J=8.2 Hz), 6.88 (1H, s), 6.82 (1H, s), 3.81 (3H, s), 3.78 (3H, s), 2.18 (3H, s), 1.68-1.79 (4H, m), 0.86 (6H, t, J=7.3 Hz).

### Example 48

### Synthesis of 5-chloro-3-(1-ethyl-propyl)-6-(2-methoxy-4-trifluoromethoxy-phenyl)-1-methyl-1H-pyrrolo[2,3-b]pyridine

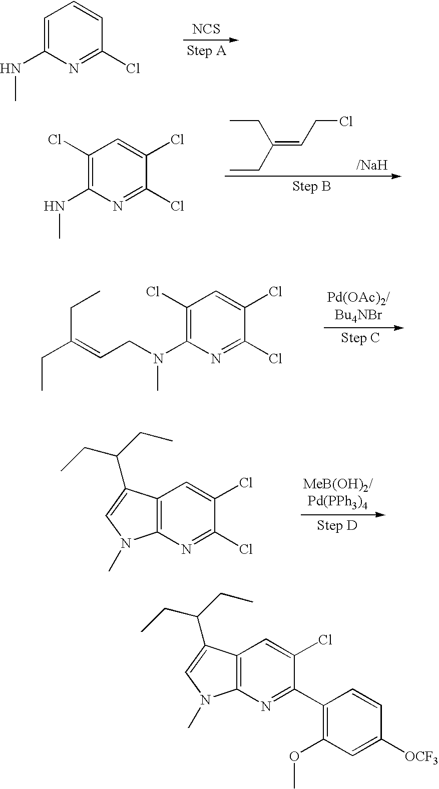

Step A

NCS (2.95 g) is added to a solution of the previously described 2-chloro-6-methylaminopyridine (1.43 g) in acetonitrile (40 mL) whereupon the reaction mixture is heated to 70° C. for 48 h. Subsequently, the yellow solution is diluted with EtOAc, washed with H₂O, brine, and dried over Na₂SO₄. Purification on silica gel yields the trichloride. ¹H NMR (CDCl₃, δ ppm): 7.50 (1H, s), 5.07 (1H, brs), 3.04 (3H, d, J=5.0 Hz).

Step B

To a solution of the trichloride from step A (1.03 g) in NMP (20 mL) is added tetrabutylammonium bromide (0.2 g) and NaH (60%, 0.38 g). After being stirred at rt for 3 h, 3,3-diethylallyl chloride (0.97 g, prepared analogously to the previously described 3,3-diethylallyl bromide) is added and the reaction mixture is stirred for an additional 36 h. The yellow solution is then quenched with water and extracted with EtOAc. The organic layer is washed with water, brine, and dried over MgSO₄ to yield the crude allylamine which was used in step C without any further purification. ¹H NMR (CDCl₃, δ ppm): 7.58 (1H, s), 5.23 (1H, t, J=6.7 Hz), 3.96 (2H, d, J=6.7 Hz), 2.92 (3H, s), 2.05-2.09 (4H, m), 0.94-1.00 (6H, m).

Step C

The allyl compound of step B (100 mg), Pd(OAc)₂ (10 mg), TBAB (116 mg), and K₂CO₃ (132 mg) are dissolved in DMF (2 mL), degassed, and heated to 80° C. overnight. The mixture is then diluted with EtOAc and washed with H₂O, brine, and dried over MgSO₄. Purification on silica gel yields the Heck product.

Step D

The Heck product of step C, the previously described 2-methoxy-4-trifluoromethoxyphenyl boronic acid, and Pd(PPh₃)₄ are dissolved in toluene. After addition of a 2N Na₂CO₃, the reaction mixture is degassed and then heated to 85° C. overnight. Subsequently, the solution is diluted with EtOAc and washed with 2N NaOH, H₂O, and brine before being dried over MgSO₄. Purification on silica gel yields the title compound. ¹H NMR (CDCl₃, δ ppm): 7.73 (1H, s), 7.39 (1H, d, J=8.2 Hz), 6.95 (1H, s), 6.93 (1H, d, J=8.2 Hz), 6.82 (1H, s), 3.81 (6H, brs), 2.59 (1H, m), 1.68-1.79 (4H, m), 0.84 (6H, t, J=7.3 Hz).

### Example 49

### Synthesis of 6-(6-Isopropyl-2-methoxy-pyridin-3-yl)-3-(1-methoxymethyl-propyl)-1,5-dimethyl-1H-pyrrolo[2,3-b]pyridine

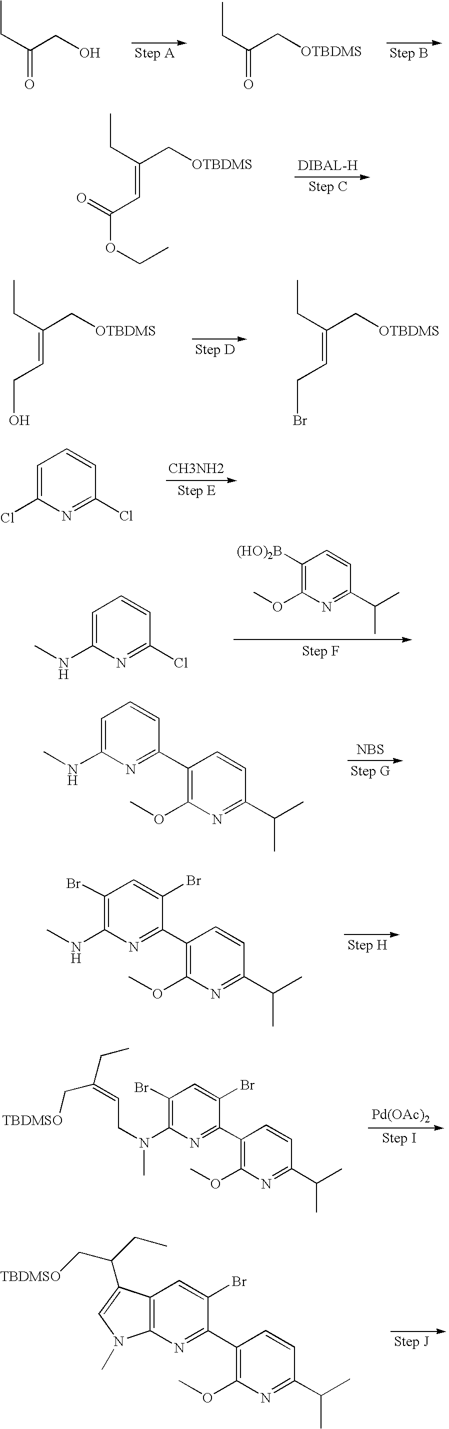

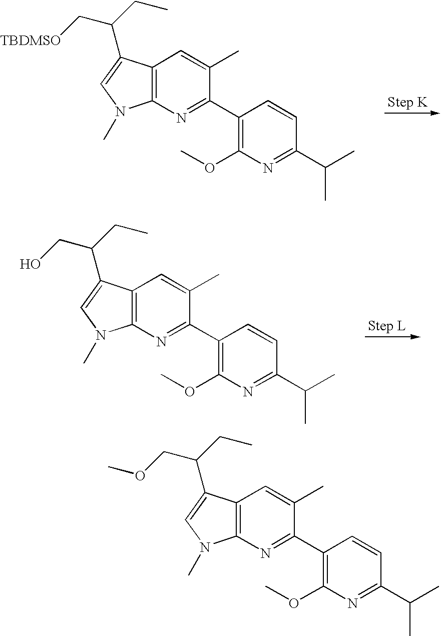

Step A

TBDMSCl (20 g) is added to a cold (0° C.) solution of 4-hydroxy-2-butanone (17.6 g), DMAP (200 mg), imidazole (10.8 g) in DMF (160 ml). The reaction mixture is warmed naturally to room temperature and stirred for 24 hours. The reaction mixture is added with water and extracted with ethyl acetate and dried with Na₂SO₄. Purification by column with hexane/ethyl acetate gives product. Rf: 0.4 (hexane/ethyl acetate: 8:1)

Step B

Triethyl phosphonoacetate (17.3 ml) is added as a solution of THF (30 ml) to a cold (0° C.) suspension of NaH (0.131 mol) in anhydrous THF (80 ml). The resulting mixture is stirred at 0° C. for 1 hour before ketone (17.67 g) is added as a solution of THF (10 ml). The reaction is continued at room temperature for another 2 hours. Saturated aqueous NH₄Cl is carefully added and separated. Aqueous layer is extracted with ether. The combined organic layers are washed with water, brine. Purification by column with hexane/ethyl acetate gives product. Rf: 0.4 (hexane/ethyl acetate: 15:1)

Step C

Starting material (21.3 g) is treated with DIBAL-H (1.0M in toluene, 196 ml) at 0° C. for 6 hours. Water is carefully added to quench the excess DIBAL. The reaction mixture is filtered and washed with ethyl acetate. The filtrate is concentrated to afford the crude product. Rf: 0.4 (hexane/ethyl acetate: 3:1).

Step D

Starting material (8.75 g) is taken in anhydrous methylene chloride (110 ml), triethylamine is added. The resulting mixture is cooled to −40° C. MsCl is added dropwise and the reaction is continued for 1 hour at −40° C. before LiBr (13.2 g) is added as a solution of THF (120 ml). The resulting reaction mixture is warmed naturally to room temperature and continued for another 1 hour. The reaction is quenched with water and separated. Aqueous layer is extracted with ether. The combined organic layers are washed with brine and dried with Na₂SO₄. The crude product can be used for the next step reaction without further purification. Rf: 0.4 (hexane/ethyl acetate: 20:1).

Step E

2,6-dichloropyridine (17 g) and CH₃NH₂ aqueous solution (40%, 26.8 g) are taken in THF (100 ml) in a sealed tube and is heated at 80° C. for 24 hours. The reaction is cooled to room temperature and diluted with water. The resulting mixture is separated and extracted with ethyl acetate. The combined organic layers are washed with brine and dried with Na₂SO₄. The crude product is used for the next step reaction without further purification. LCMS: 143.3 (M+H)⁺

Step F

A mixture of 2-chloro-6-methylamino-pyridine (3.56, 0.025 mol)), 2-methoxy-6-isopropyl-3-pyridylboronic acid (6.33 g), Pd(PPh₃)₄ (577 mg), aqueous Na₂CO₃ solution (1.0M, 50 ml), and toluene (50 ml) is heated overnight at 100° C. under a dinitrogen atmosphere. The reaction mixture is cooled to room temperature and separated. The aqueous layer is extracted with ethyl acetate. The combined organic layers are washed with brine and dried with Na₂SO₄. The crude product is used for the next step reaction without further purification. Rf: 0.4 (hexane/ethyl acetate: 4:1).

Step G

The crude starting material is taken in anhydrous CHCl₃ (100 ml). 4.0 equivalent of NBS is added in one portion at 0° C.. The reaction is complete in 0.5 hour. The reaction mixture is washed with water and dried with Na₂SO₄. Purification by flash column with hexane/ethyl acetate gives product as clear oil. LCMS: m/z 496.1 (M+H)⁺

Step H

NaH (795 mg, 60% in mineral oil) is added to a solution of starting material (6.34 g) in anhydrous DMF (100 ml) and stirred at room temperature for 10 minutes. Bromide (4.93 g) prepared in Step D is added dropwise and the resulting mixture is stirred for 3 hours. The reaction mixture is carefully quenched with water. The resulting mixture is extracted with ethyl acetate. The combine organic layers are washed with brine and dried with Na₂SO₄. Purification by flash column with hexane/ethyl acetate gives product as clear oil. Rf: 0.4 (hexane/ethyl acetate: 12:1).

Step I

A mixture of bromide (9.26 g), tetrabutylammonium bromide (5.95 g), K₂CO₃ (6.12 g), Pd(OAc)₂ (1.0 g) in DMF (80 ml) is heated at 80° C. under N2 atmosphere for 20 minutes. The reaction mixture is cooled to room temperature and diluted with water. The resulting mixture is extracted with ethyl acetate. The combine organic layers are washed with brine and dried with Na₂SO₄. Purification by flash column with hexane/ethyl acetate gives product as clear oil. LCMS: m/z 548.4 (M+H)⁺

Step J

To a solution of t-BuLi (1.7M/pentane, 7 ml) in THF (30 ml) at −78° C. is added a solution of bromide (3.07 g) in THF (5 ml). The resulting mixture is stirred at −78° C. for 10 minutes before iodomethane (1.4 ml) is added. The reaction is continued for 30 minutes. The reaction is carefully quenched with EtOH. The resulting mixture is washed with water and brine, dried with Na₂SO₄. Purification by flash column with hexane/ethyl acetate gives product as clear oil. Rf: 0.4 (hexane/ethyl acetate: 10:1).

Step K

Starting material (1.26 g) is taken in THF (50 ml) followed by the addition of tetrabutylammonium fluoride (1.5 equiv.) at room temperature. The reaction is complete after 4 hours. The reaction mixture is washed with water, brine and dried with Na₂SO₄. Purification by flash column with hexane/ethyl acetate gives product. LCMS: m/z 368.3 (M+H)⁺

Step L

Starting material (100 mg) is taken in anhydrous DMF (4 ml), NaH (52 mg, 60%) is added followed by the addition of CH₃I (5 equiv.). The reaction is continued overnight. The reaction is quenched with water and extracted with ethyl acetate. The combine organic layers are washed with brine and dried with Na₂SO₄. Purification by flash column with hexane/ethyl acetate gives product as clear oil. LCMS: m/z 382.3 (M+H)⁺

### Example 50

The compounds shown in the table are analogously prepared according to the procedures given in the above schemes and further illustrated in the above examples.

| Cmpd | | | | |
|---|---|---|---|---|
| # | STRUCTURE | COMPOUND NAME | MS, m/z | Rt(min) |
| 101 | 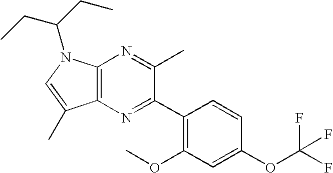 | 5-(1-Ethyl-propyl)-2- (2-methoxy-4- trifluoromethoxy- phenyl)-3,7-dimethyl- 5H-pyrrolo[2,3-]pyrazine | 408.21 | 1.77 |
| 102 | 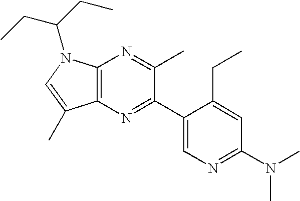 | {4-Ethyl-5-[5-(1-ethyl- propyl)-3,7-dimethyl- 5H-pyrrolo[2,3- ]pyrazin-2-yl]-pyridin- 2-yl}-dimethyl-amine | 366.22 | 1.41 |
| 103 | 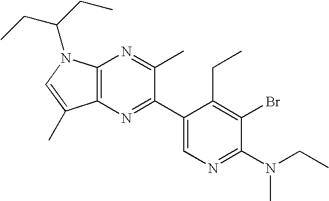 | {3-Bromo-4-ethyl-5-[5- (1-ethyl-propyl)-3,7- dimethyl-5H- pyrrolo[2,3-b]pyrazin- 2-yl]-pyridin-2-yl}- ethyl-methyl-amine | 458.12 | 1.94 |
| 104 | 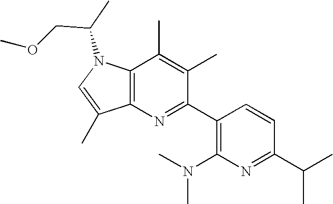 | Ethyl-{4-ethyl-5-[5-(1- ethyl-propyl)-3,7- dimethyl-5H- pyrrolo[2,3-b]pyrazin- 2-yl]-pyridin-2-yl}- methyl-amine | 380.23 | 1.66 |
| 105 | 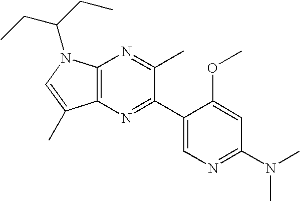 | {5-[5-(1-Ethyl-propyl)- 3,7-dimethyl-5H- pyrrolo[2,3-b]pyrazin- 2-yl]-4-methoxy- pyridin-2-yl}-dimethyl-amine | 368.19 | 1.39 |
| 106 | 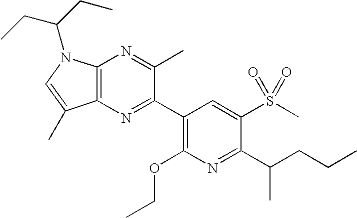 | 2-[2-Ethoxy-5- methanesulfonyl-6-(1- methyl-but-3-enyl)- pyridin-3-yl]-5-(1- ethyl-propyl)-3,7- dimethyl-5H- pyrrolo[2,3-b]pyrazine | 485.17 | 1.82 |
| 107 | 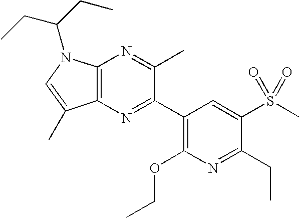 | 2-(2-Ethoxy-6-ethyl-5- methanesulfonyl- pyridin-3-yl)-5-(1- ethyl-propyl)-3,7- dimethyl-5H- pyrrolo[2,3-b]pyrazine | 445.16 | 1.74 |
| 108 | 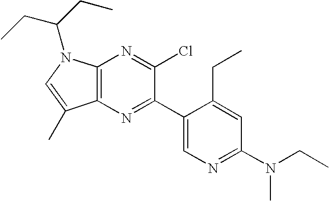 | {5-[3-Chloro-5-(1- ethyl-propyl)-7-methyl- 5H-pyrrolo[2,3- b]pyrazin-2-yl]-4- ethyl-pyridin-2-yl}- ethyl-methyl-amine | 399.96 | 1.50 |
| 109 | 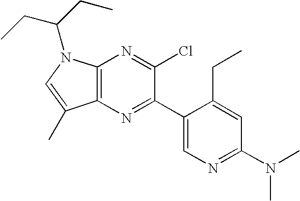 | {5-[3-Chloro-5-(1- ethyl-propyl)-7-methyl- 5H-pyrrolo[2,3- b]pyrazin-2-yl]-4- ethyl-pyridin-2-yl}- dimethyl-amine | 386.20 | 1.48 |
| 110 | 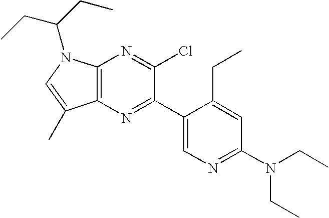 | {5-[3-Chloro-5-(1- ethyl-propyl)-7-methyl- 5H-pyrrolo[2,3- b]pyrazin-2-yl]-4-ethyl- pyridin-2-yl}-diethyl-amine | 414.23 | 1.51 |
| 111 | 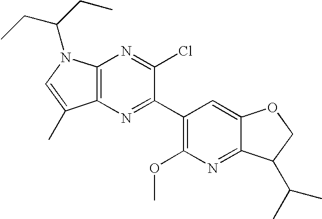 | 3-Chloro-5-(1-ethyl- propyl)-2-(3-isopropyl- 5-methoxy-2,3- dihydro-furo[3,2-b]pyridin-6-yl)-7- methyl-5H-pyrrolo[2,3- b]pyrazine | 429.08 | 1.53 |
| 112 | 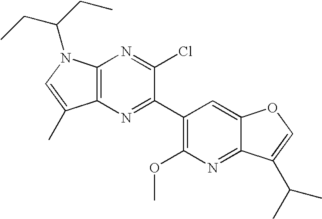 | 3-Chloro-5-(1-ethyl- propyl)-2-(3-isopropyl- 5-methoxy-furo[3,2- b]pyridin-6-yl)-7- methyl-5H-pyrrolo[2,3- b]pyrazine | 427.12 | 1.52 |
| 113 | 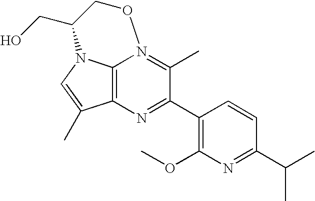 | (R)-2-[2-(6-Isopropyl- 2-methoxy-pyridin-3- yl)-3,7-dimethyl- pyrrolo[2,3-b]pyrazin- 5-yl]-3-methoxy- propan-1-ol | 385.5 | |
| 114 | 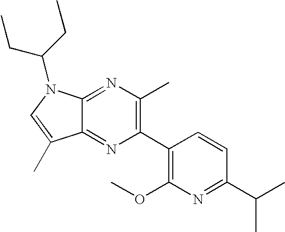 | 5-(1-Ethyl-propyl)-2- (6-isopropyl-2- methoxy-pyridin-3-yl)- 3,7-dimethyl-5H- pyrrolo[2,3-b]pyrazine | 367.5 | |
| 115 | 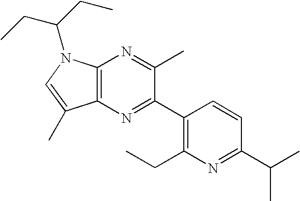 | 2-(2-Ethyl-6-isopropyl- pyridin-3-yl)-5-(1- ethyl-propyl)-3,7- dimethyl-5H- pyrrolo[2,3-b]pyrazine | 365.5 | |
| 116 | 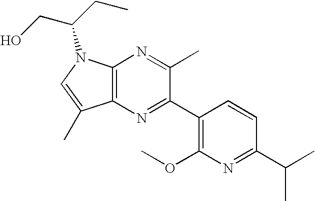 | 2-[(S)-2-(6-Isopropyl- 2-methoxy-pyridin-3- yl)-3,7-dimethyl- pyrrolo[2,3-b]pyrazin- 5-yl]-butan-1-ol | 369.4 | |
| 117 | 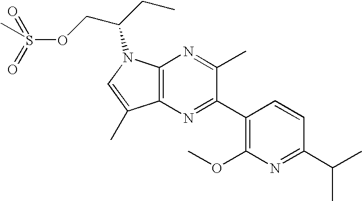 | Methanesulfonic acid 2-[(S)-2-(6-isopropyl-2- methoxy-pyridin-3-yl)- 3,7-dimethyl- pyrrolo[2,3-b]pyrazin- 5-yl]-butyl ester | 447.4 | |
| 118 | 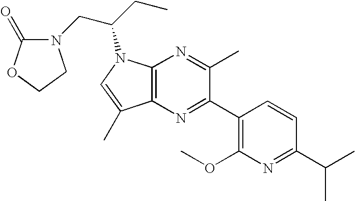 | 3-{2-[(S)-2-(6- Isopropyl-2-methoxy- pyridin-3-yl)-3,7- dimethyl-pyrrolo[2,3- b]pyrazin-5-yl]-butyl}- oxazolidin-2-one | 438.4 | |
| 119 | 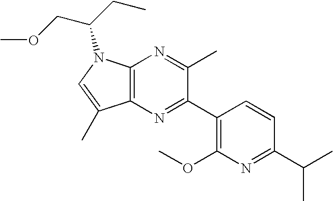 | (S)-2-(6-Isopropyl-2- methoxy-pyridin-3-yl)- 5-(1-methoxymethyl- propyl)-3,7-dimethyl-5H- pyrrolo[2,3-b]pyrazine | 383.2 | |
| 120 | 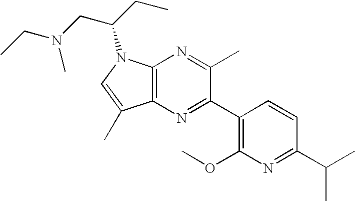 | Ethyl-{2-[(S)-2-(6- isopropyl-2-methoxy- pyridin-3-yl)-3,7- dimethyl-pyrrolo[2,3- b]pyrazin-5-yl]-butyl}- methyl-amine | 410.4 | |
| 121 | 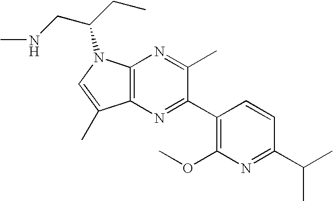 | {2-[(S)-2-(6-Isopropyl- 2-methoxy-pyridin-3- yl)-3,7-dimethyl- pyrrolo[2,3-b]pyrazin-5-yl]- butyl}-methyl-amine | 382.3 | |
| 122 | 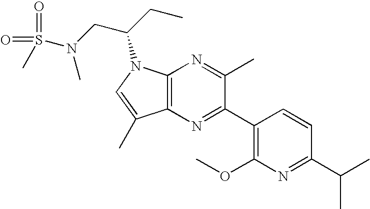 | N-{2-[(S)-2-(6- Isopropyl-2-methoxy- pyridin-3-yl)-3,7- dimethyl-pyrrolo[2,3- b]pyrazin-5-yl]-butyl}- N-methyl- methanesulfonamide | 460.3 | |
| 123 | 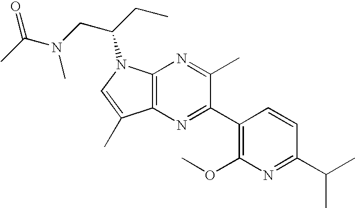 | N-{2-[(S)-2-(6- Isopropyl-2-methoxy- pyridin-3-yl)-3,7- dimethyl-pyrrolo[2,3- b]pyrazin-5-yl]-butyl}- N-methyl-acetamide | 424.5 | |
| 124 | 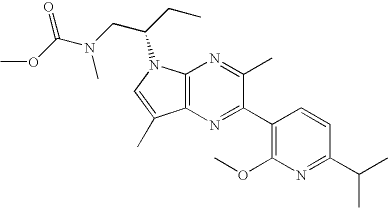 | {2-[(S)-2-(6-Isopropyl- 2-methoxy-pyridin-3- yl)-3,7-dimethyl- pyrrolo[2,3-b]pyrazin- 5-yl]-butyl}-methyl- carbamic acid methyl ester | 440.4 | |
| 125 | 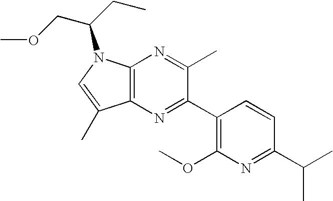 | (R)-2-(6-Isopropyl-2- methoxy-pyridin-3-yl)- 5-(1-methoxymethyl- propyl)-3,7-dimethyl- 5H-pyrrolo[2,3-b]pyrazine | 383.3 | |
| 126 | 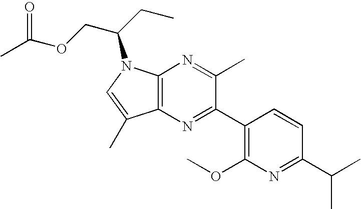 | Acetic acid 2-[(R)-2-(6- isopropyl-2-methoxy- pyridin-3-yl)-3,7- dimethyl-pyrrolo[2,3- b]pyrazin-5-yl]-butyl ester | 411.4 | |
| 127 | 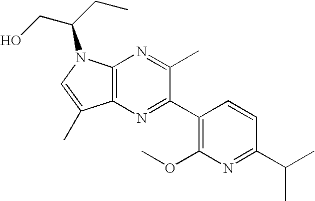 | 2-[(R)-2-(6-Isopropyl- 2-methoxy-pyridin-3- yl)-3,7-dimethyl- pyrrolo[2,3-b]pyrazin- 5-yl]-butan-1-ol | 369.4 | |
| 128 | 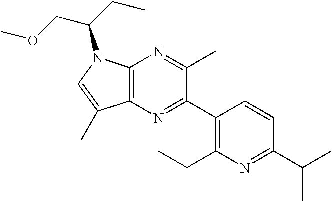 | (R)-2-(2-Ethyl-6- isopropyl-pyridin-3-yl)- 5-(1-methoxymethyl- propyl)-3,7-dimethyl-5H- pyrrolo[2,3-b]pyrazine | 381.4 | |
| 129 | 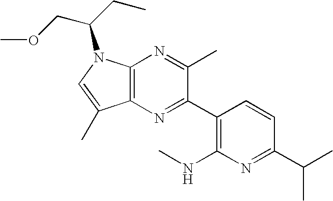 | {6-Isopropyl-3-[(R)-5- (1-methoxymethyl- propyl)-3,7-dimethyl- 5H-pyrrolo[2,3- b]pyrazin-2-yl]-pyridin- 2-yl}-methyl-amine | 382.3 | |
| 130 | 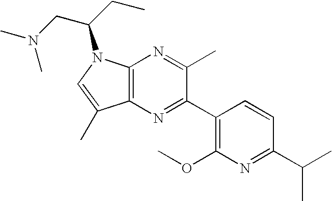 | {2-[(R)-2-(6-Isopropyl- 2-methoxy-pyridin-3- yl)-3,7-dimethyl- pyrrolo[2,3-b]pyrazin- 5-yl]-butyl}-dimethyl-amine | 396.4 | |
| 131 | 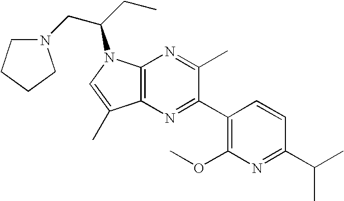 | (R)-2-(6-Isopropyl-2- methoxy-pyridin-3-yl)- 3,7-dimethyl-5-(1- pyrrolidin-1-ylmethyl- propyl)-5H- pyrrolo[2,3-b]pyrazine | 422.2 | |
| 132 | 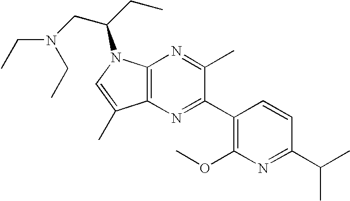 | Diethyl-{2-[(R)-2-(6- isopropyl-2-methoxy- pyridin-3-yl)-3,7- dimethyl-pyrrolo[2,3- b]pyrazin-5-yl]-butyl}- amine | 424.1 | |
| 133 | 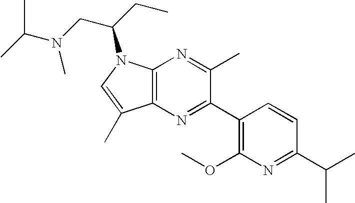 | Isopropyl-{2-[(R)-2-(6- isopropyl-2-methoxy- pyridin-3-yl)-3,7- dimethyl- pyrrolo[2,3-b]pyrazin- 5-yl]-butyl}-methyl- amine | 424.3 | |
| 134 | 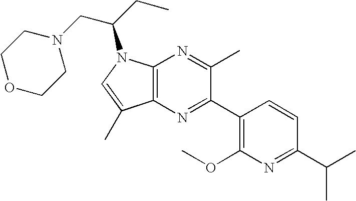 | (R)-2-(6-Isopropyl-2- methoxy-pyridin-3-yl)- 3,7-dimethyl-5-(1- morpholin-4-ylmethyl- propyl)-5H- pyrrolo[2,3-b]pyrazine | 438.5 | |
| 135 | 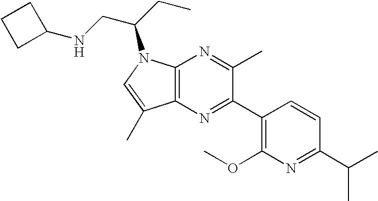 | Cyclobutyl-{2-[(R)-2- (6-isopropyl-2- methoxy-pyridin-3-yl)- 3,7-dimethyl- pyrrolo[2,3-b]pyrazin- 5-yl]-butyl}-amine | 422.5 | |
| 136 | 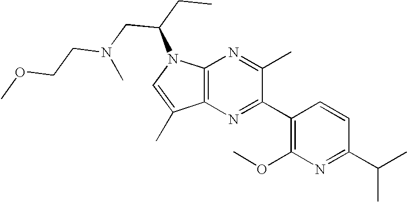 | {2-[(R)-2-(6-Isopropyl- 2-methoxy-pyridin-3- yl)-3,7-dimethyl- pyrrolo[2,3-b]pyrazin- 5-yl]-butyl}-(2- methoxy-ethyl)-methyl- amine | 440.4 | |
| 137 | 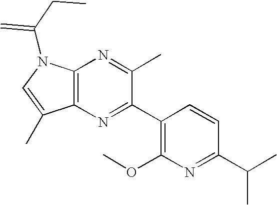 | 2-(6-Isopropyl-2- methoxy-pyridin-3-yl)- 3,7-dimethyl-5-(1- methylene-propyl)-5H- pyrrolo[2,3-b]pyrazine | 351.4 | |
| 138 | 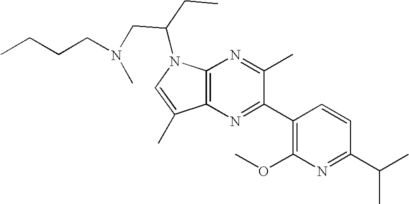 | Butyl-ethyl-{2-[(R)-2- (6-isopropyl-2- methoxy-pyridin-3-yl)- 3,7-dimethyl-pyrrolo[2,3- b]pyrazin- 5-yl]-butyl}-amine | 452.5 | |
| 139 | 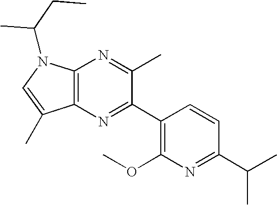 | 5-sec-Butyl-2-(6- isopropyl-2-methoxy- pyridin-3-yl)-3,7- dimethyl-5H- pyrrolo[2,3-b]pyrazine | 353.4 | |
| 140 | 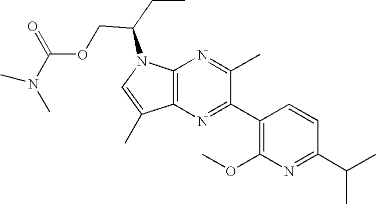 | Dimethyl-carbamic acid 2-[(R)-2-(6- isopropyl-2-methoxy- pyridin-3-yl)-3,7- dimethyl-pyrrolo[2,3- b]pyrazin-5-yl]-butyl ester | 440.4 | |
| 141 | 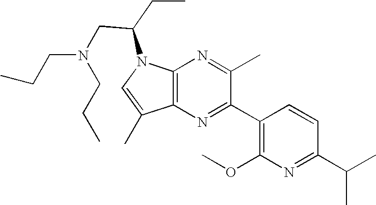 | {2-[(R)-2-(6-Isopropyl- 2-methoxy-pyridin-3- yl)-3,7-dimethyl- pyrrolo[2,3-b]pyrazin- 5-yl]-butyl}-dipropyl- amine | 452.5 | |
| 142 | 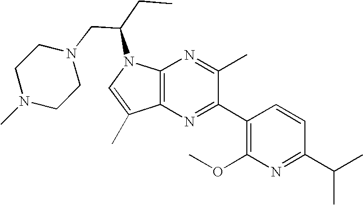 | 2-(6-Isopropyl-2- methoxy-pyridin-3-yl)- 3,7-dimethyl-5-[(R)-1- (4-methyl-piperazin-1- ylmethyl)-propyl]-5H- pyrrolo[2,3-b]pyrazine | 451.5 | |
| 143 | 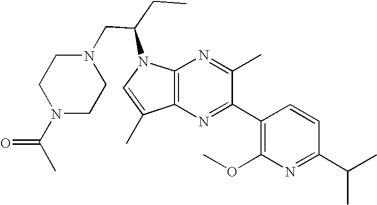 | 1-(4-{(R)-2-[2-(6- Isopropyl-2-methoxy- pyridin-3-yl)-3,7- dimethyl-pyrrolo[2,3- b]pyrazin-5-yl]-butyl}- piperazin-1-yl)-ethanone | 479.5 | |
| 144 | 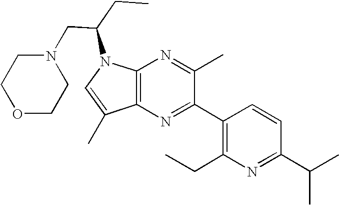 | 2-(2-Ethyl-6-isopropyl- pyridin-3-yl)-3,7- dimethyl-5-((R)-1- morpholin-4-ylmethyl- propyl)-5H- pyrrolo[2,3-b]pyrazine | 436.4 | |
| 145 | 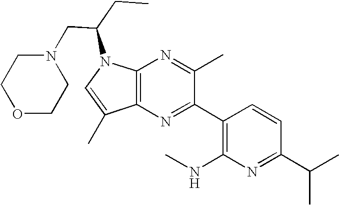 | {3-[3,7-Dimethyl-5- ((R)-1-morpholin-4- ylmethyl-propyl)-5H- pyrrolo[2,3-b]pyrazin-2-yl]-6- isopropyl-pyridin-2- yl}-methyl-amine | 437.3 | |
| 146 | 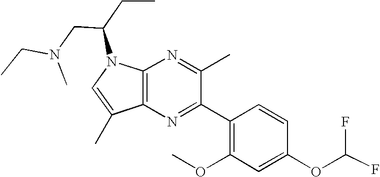 | {(R)-2-[2-(4- Difluoromethoxy-2- methoxy-phenyl)-3,7- dimethyl-pyrrolo[2,3- b]pyrazin-5-yl]-butyl}- ethyl-methyl-amine | 433.5 | |
| 147 | 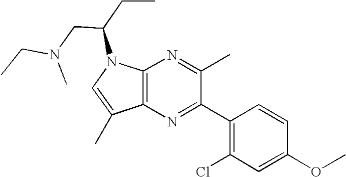 | {(R)-2-[2-(2-Chloro-4- methoxy-phenyl)-3,7- dimethyl-pyrrolo[2,3- b]pyrazin-5-yl]-butyl}- ethyl-methyl-amine | 401.5, 403.5 | |
| 148 | 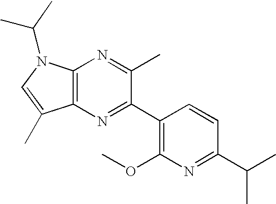 | 5-Isopropyl-2-(6- isopropyl-2-methoxy- pyridin-3-yl)-3,7- dimethyl-5H- pyrrolo[2,3-b]pyrazine | 339.1 | |
| 149 | 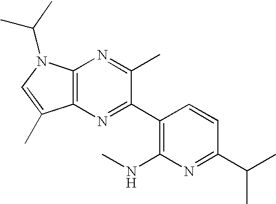 | [6-Isopropyl-3-(5- isopropyl-3,7-dimethyl- 5H-pyrrolo[2,3- b]pyrazin-2-yl)-pyridin- 2-yl]-methyl-amine | 338.3 | |
| 150 | 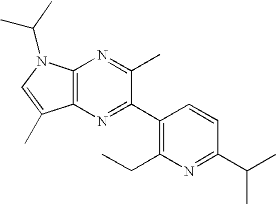 | 2-(2-Ethyl-6-isopropyl- pyridin-3-yl)-5- isopropyl-3,7-dimethyl- 5H-pyrrolo[2,3- b]pyrazine | 337.2 | |
| 151 | 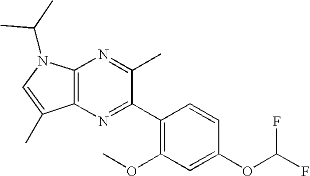 | 2-(4-Difluoromethoxy- 2-methoxy-phenyl)-5- isopropyl-3,7-dimethyl- 5H-pyrrolo[2,3- b]pyrazine | 362.1 | |
| 152 | 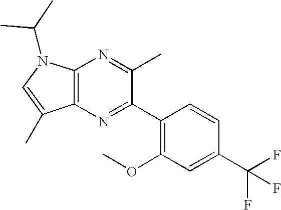 | 5-Isopropyl-2-(2- methoxy-4- trifluoromethyl- phenyl)-3,7-dimethyl- 5H-pyrrolo[2,3-b]pyrazine | 364.1 | |
| 153 | 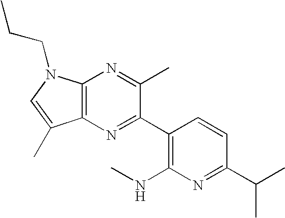 | [3-(3,7-Dimethyl-5- propyl-5H-pyrrolo[2,3- b]pyrazin-2-yl)-6- isopropyl-pyridin-2-yl]- methyl-amine | 338.1 | |
| 154 | 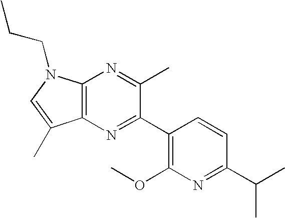 | 2-(6-Isopropyl-2- methoxy-pyridin-3-yl)- 3,7-dimethyl-5-propyl- 5H-pyrrolo[2,3-b]pyrazine | 339.1 | |
| 155 | 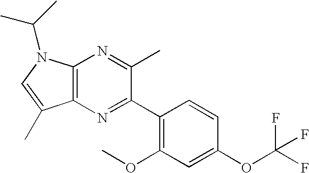 | 5-Isopropyl-2-(2- methoxy-4- trifluoromethoxy- phenyl)-3,7-dimethyl- 5H-pyrrolo[2,3-b]pyrazine | 380.0 | |
| 156 | 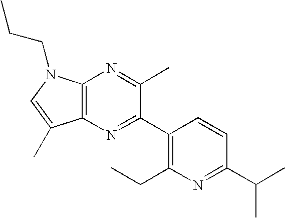 | 2-(2-Ethyl-6-isopropyl- pyridin-3-yl)-3,7- dimethyl-5-propyl-5H- pyrrolo[2,3-b]pyrazine | 337.2 | |
| 157 | 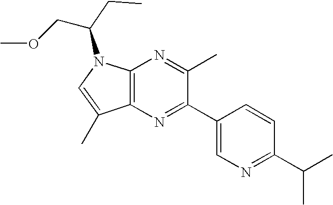 | (R)-2-(6-Isopropyl- pyridin-3-yl)-5-(1- methoxymethyl- propyl)-3,7-dimethyl- 5H-pyrrolo[2,3-b]pyrazine | 353.3 | |
| 158 | 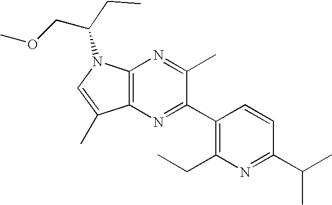 | (S)-2-(2-Ethyl-6- isopropyl-pyridin-3-yl)- 5-(1-methoxymethyl- propyl)-3,7-dimethyl-5H- pyrrolo[2,3-b]pyrazine | 381.1 | |
| 159 | 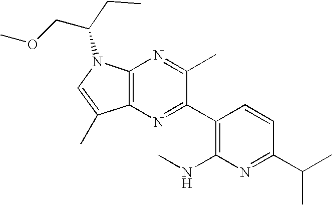 | {6-Isopropyl-3-[(S)-5- (1-methoxymethyl- propyl)-3,7-dimethyl- 5H-pyrrolo[2,3-b]pyrazin- 2-yl]-pyridin-2- yl}-methyl-amine | 382.3 | |
| 160 | 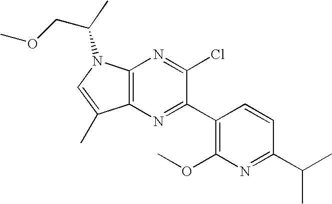 | (S)-3-Chloro-2-(6- isopropyl-2-methoxy- pyridin-3-yl)-5-(2- methoxy-1-methyl- ethyl)-7-methyl-5H- pyrrolo[2,3-b]pyrazine | 389.4, 391.4 | |
| 161 | 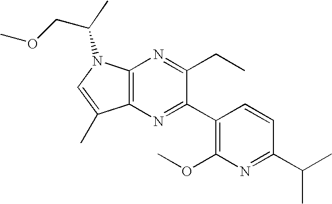 | (S)-3-Ethyl-2-(6- isopropyl-2-methoxy- pyridin-3-yl)-5-(2- methoxy-1-methyl- ethyl)-7-methyl-5H- pyrrolo[2,3-b]pyrazine | 383.4 | |
| 162 | 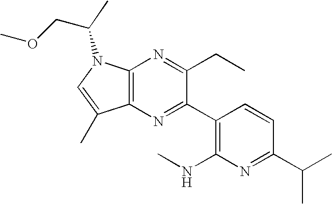 | {3-[3-Ethyl-5-((S)-2- methoxy-1-methyl- ethyl)-7-methyl-5H- pyrrolo[2,3-b]pyrazin- 2-yl]-6-isopropyl- pyridin-2-yl}-methyl-amine | 382.4 | |
| 163 | 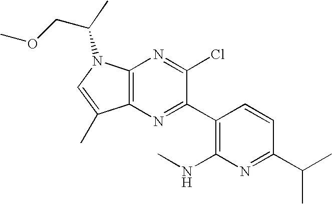 | {3-[3-Chloro-5-((S)-2- methoxy-1-methyl- ethyl)-7-methyl-5H- pyrrolo[2,3-b]pyrazin- 2-yl]-6-isopropyl- pyridin-2-yl}-methyl-amine | 388.4, 390.4 | |
| 164 | 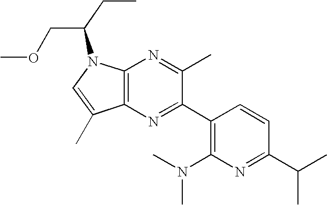 | {6-Isopropyl-3-[5-((R)- 1-methoxymethyl- propyl)-3,7-dimethyl- 5H-pyrrolo [2,3-b]pyrazin-2-yl]- pyridin-2-yl}-dimethyl- amine | 396.5 | |
| 165 | 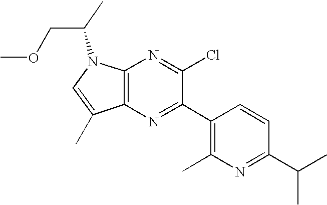 | 3-Chloro-2-(6- isopropyl-2-methyl- pyridin-3-yl)-5-((S)-2- methoxy-1-methyl- ethyl)-7-methyl-5H- pyrrolo[2,3-b]pyrazine | 373.3, 375.3 | |
| 166 | 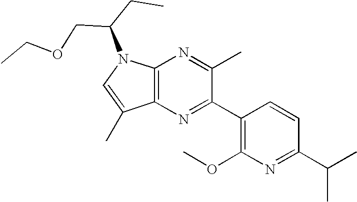 | 5-((R)-1-Ethoxymethyl- propyl)-2-(6-isopropyl- 2-methoxy-pyridin-3- yl)-3,7-dimethyl-5H- pyrrolo[2,3-b]pyrazine | 397.5 | |
| 167 | 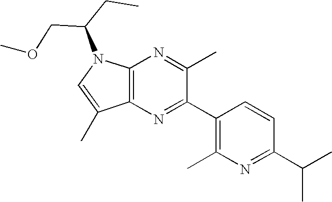 | 2-(6-Isopropyl-2- methyl-pyridin-3-yl)-5- ((R)-1-methoxymethyl- propyl)-3,7-dimethyl-5H- pyrrolo[2,3-b]pyrazine | 367.5 | |
| 168 | 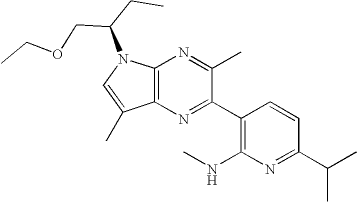 | {3-[5-((R)-1- Ethoxymethyl-propyl)- 3,7-dimethyl-5H- pyrrolo[2,3-b]pyrazin- 2-yl]-6-isopropyl- pyridin-2-yl}-methyl- amine | 396.5 | |
| 169 | 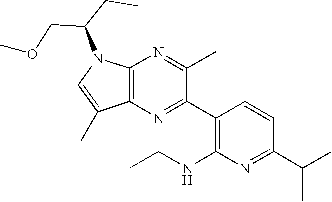 | Ethyl-{6-isopropyl-3- [5-((R)-1- methoxymethyl- propyl)-3,7-dimethyl- 5H-pyrrolo[2,3-b]pyrazin-2- yl]-pyridin-2-yl}-amine | 396.5 | |
| 170 | 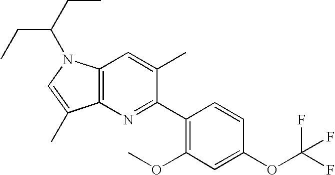 | 1-(1-Ethyl-propyl)-5- (2-methoxy-4- trifluoromethoxy- phenyl)-3,6-dimethyl- 1H-pyrrolo[3,2-b]pyridine | | |
| 171 | 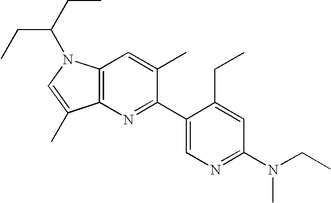 | Ethyl-{4-ethyl-5-[1-(1- ethyl-propyl)-3,6- dimethyl-1H- pyrrolo[3,2-b]pyridin- 5-yl]-pyridin-2-yl}- methyl-amine | 379.5 | |
| 172 | 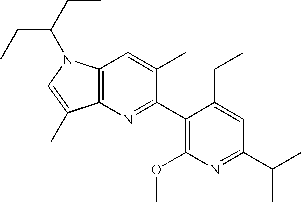 | 1-(1-Ethyl-propyl)-5- (6-isopropyl-2- methoxy-pyridin-3-yl)- 3,6-dimethyl-1H- pyrrolo[3,2-b]pyridine | 366.4 | |
| 173 | 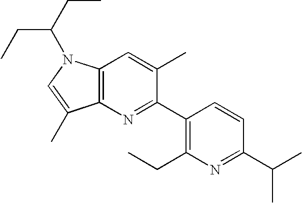 | 5-(2-Ethyl-6-isopropyl- pyridin-3-yl)-1-(1- ethyl-propyl)-3,6- dimethyl-1H- pyrrolo[3,2-b]pyridine | 364.5 | |
| 174 | 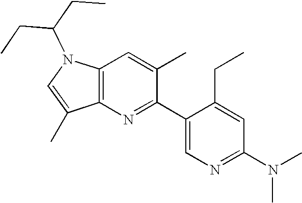 | {4-Ethyl-5-[1-(1-ethyl- propyl)-3,6-dimethyl- 1H-pyrrolo[3,2- b]pyridin-5-yl]-pyridin- 2-yl}-dimethyl-amine | 365.5 | |
| 175 | 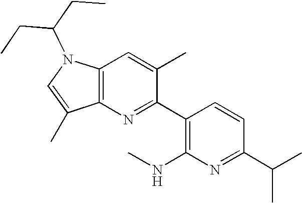 | {3-[1-(1-Ethyl-propyl)- 3,6-dimethyl-1H- pyrrolo[3,2-b]pyridin- 5-yl]-6-isopropyl- pyridin-2-yl}-methyl- amine | 365.5 | |
| 176 | 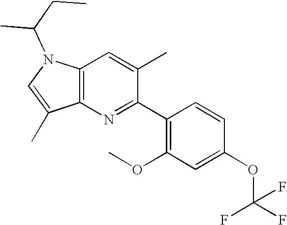 | 1-sec-Butyl-5-(2- methoxy-4- trifluoromethoxy- phenyl)-3,6-dimethyl- 1H-pyrrolo[3,2-b]pyridine | 393.3 | |
| 177 | 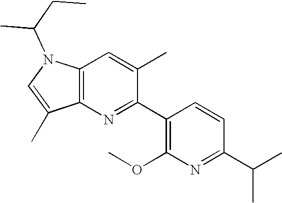 | 1-sec-Butyl-5-(6- isopropyl-2-methoxy- pyridin-3-yl)-3,6- dimethyl-1H- pyrrolo[3,2-b]pyridine | 352.4 | |
| 178 | 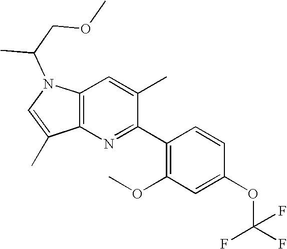 | 1-(2-Methoxy-1- methyl-ethyl)-5-(2- methoxy-4- trifluoromethoxy- phenyl)-3,6-dimethyl-1H- pyrrolo[3,2-b]pyridine | 409.3 | |
| 179 | 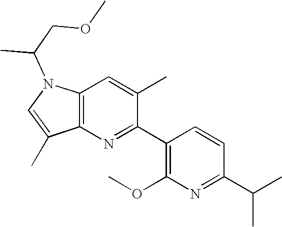 | 5-(6-Isopropyl-2- methoxy-pyridin-3-yl)- 1-(2-methoxy-1- methyl-ethyl)-3,6- dimethyl-1H- pyrrolo[3,2-b]pyridine | 368.1 | |
| 180 | 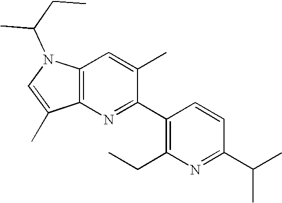 | 1-sec-Butyl-5-(2-ethyl- 6-isopropyl-pyridin-3- yl)-3,6-dimethyl-1H- pyrrolo[3,2-b]pyridine | 350.5 | |
| 181 | 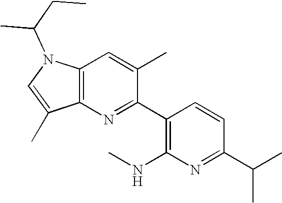 | [3-(1-sec-Butyl-3,6- dimethyl-1H- pyrrolo[3,2-b]pyridin- 5-yl)-6-isopropyl- pyridin-2-yl]-methyl- amine | 351.4 | |
| 182 | 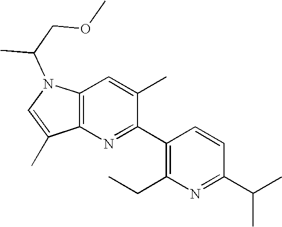 | 5-(2-Ethyl-6-isopropyl- pyridin-3-yl)-1-(2- methoxy-1-methyl- ethyl)-3,6-dimethyl- 1H-pyrrolo[3,2-b]pyridine | 366.3 | |
| 183 | 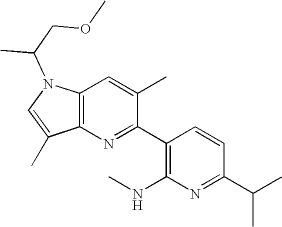 | {6-Isopropyl-3-[1-(2- methoxy-1-methyl- ethyl)-3,6-dimethyl- 1H-pyrrolo[3,2-b]pyridin- 5-yl]-pyridin-2- yl}-methyl-amine | 367.5 | |
| 184 | 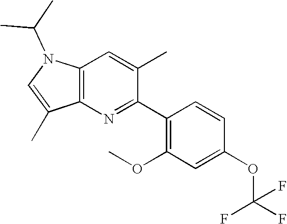 | 1-Isopropyl-5-(2- methoxy-4- trifluoromethoxy- phenyl)-3,6-dimethyl- 1H-pyrrolo[3,2- b]pyridine | 379.4 | |
| 185 | 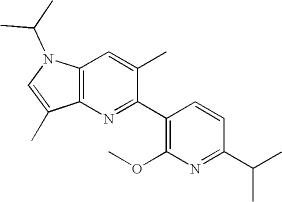 | 1-Isopropyl-5-(6- isopropyl-2-methoxy- pyridin-3-yl)-3,6- dimethyl-1H- pyrrolo[3,2-b]pyridine | 338.3 | |
| 186 | 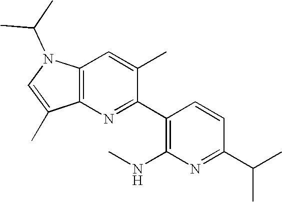 | [6-Isopropyl-3-(1- isopropyl-3,6-dimethyl- 1H-pyrrolo[3,2- b]pyridin-5-yl)-pyridin- 2-yl]-methyl-amine | 337.4 | |
| 187 | 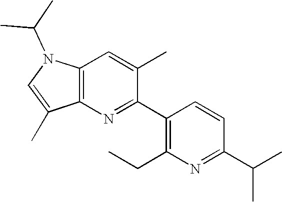 | 5-(2-Ethyl-6-isopropyl- pyridin-3-yl)-1- isopropyl-3,6-dimethyl- 1H-pyrrolo[3,2-b]pyridine | 336.4 | |
| 188 | 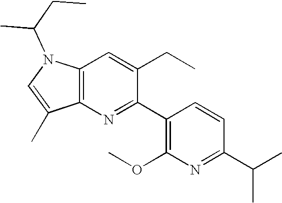 | 1-sec-Butyl-6-ethyl-5- (6-isopropyl-2- methoxy-pyridin-3-yl)- 3-methyl-1H- pyrrolo[3,2-b]pyridine | 366.4 | |
| 189 | 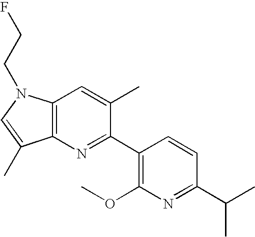 | 1-(2-Fluoro-ethyl)-5-(6- isopropyl-2-methoxy- pyridin-3-yl)-3,6- dimethyl-1H- pyrrolo[3,2-b]pyridine | 342.4 | |
| 190 | 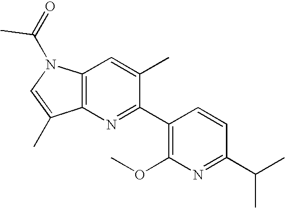 | 1-[5-(6-Isopropyl-2- ethoxy-pyridin-3-yl)- 3,6-dimethyl- pyrrolo[3,2-b]pyridin- 1-yl]-ethanone | 338.3 | |
| 191 | 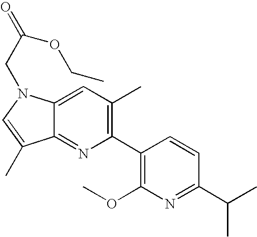 | [5-(6-Isopropyl-2- methoxy-pyridin-3-yl)- 3,6-dimethyl- pyrrolo[3,2-b]pyridin- 1-yl]-acetic acid ethyl ester | 382.3 | |
| 192 | 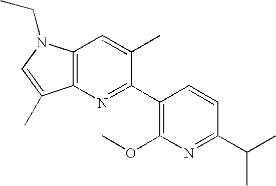 | 1-Ethyl-5-(6-isopropyl- 2-methoxy-pyridin-3- yl)-3,6-dimethyl-1H- pyrrolo[3,2-b]pyridine | 324.4 | |
| 193 | 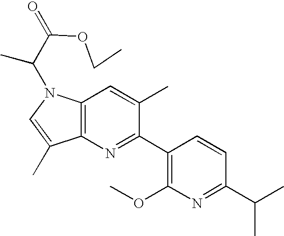 | 2-[5-(6-Isopropyl-2- methoxy-pyridin-3-yl)- 3,6-dimethyl- pyrrolo[3,2-b]pyridin- 1-yl]-propionic acid ethyl ester | 396.4 | |
| 194 | 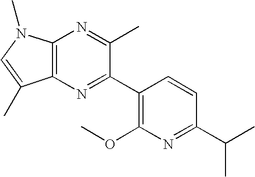 | 5-(6-Isopropyl-2- methoxy-pyridin-3-yl)- 1,3,6-trimethyl-1H- pyrrolo[3,2-b]pyridine | 310.4 | |
| 195 | 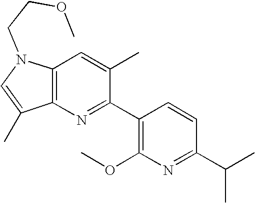 | 5-(6-Isopropyl-2- methoxy-pyridin-3-yl)- 1-(2-methoxy-ethyl)- 3,6-dimethyl-1H- pyrrolo[3,2-b]pyridine | 354.1 | |
| 196 | 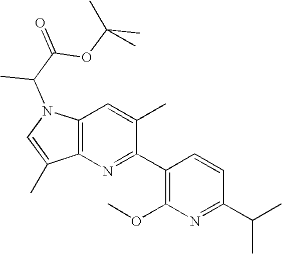 | 2-[5-(6-Isopropyl-2- methoxy-pyridin-3-yl)- 3,6-dimethyl- pyrrolo[3,2-b]pyridin- 1-yl]-propionic acid tert-butyl ester | 424.3 | |
| 197 | 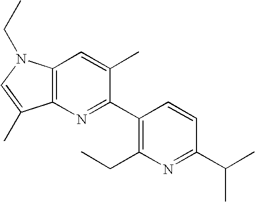 | 1-Ethyl-5-(2-ethyl-6- isopropyl-pyridin-3-yl)- 3,6-dimethyl-1H- pyrrolo[3,2-b]pyridine | 322.5 | |
| 198 | 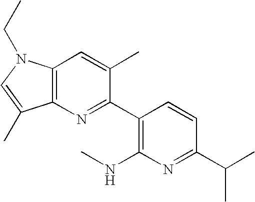 | [3-(1-Ethyl-3,6- dimethyl-1H- pyrrolo[3,2-b]pyridin- 5-yl)-6-isopropyl- pyridin-2-yl]-methyl- amine | 323.5 | |
| 199 | 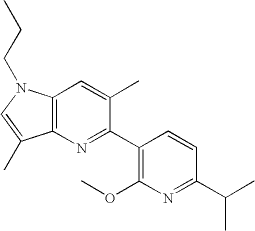 | 5-(6-Isopropyl-2- methoxy-pyridin-3-yl)- 3,6-dimethyl-1-propyl- 1H-pyrrolo[3,2- b]pyridine | 338.4 | |
| 200 | 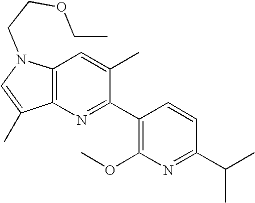 | 1-(2-Ethoxy-ethyl)-5- (6-isopropyl-2- methoxy-pyridin-3-yl)- 3,6-dimethyl-1H- pyrrolo[3,2-b]pyridine | 368.3 | |
| 201 | 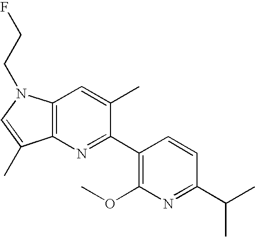 | 5-(2-Ethyl-6-isopropyl- pyridin-3-yl)-1-(2- fluoro-ethyl)-3,6- dimethyl-1H- pyrrolo[3,2-b]pyridine | 340.2 | |
| 202 | 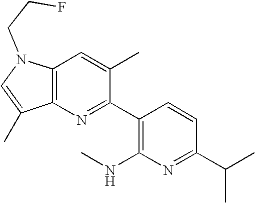 | {3-[1-(2-Fluoro-ethyl)- 3,6-dimethyl-1H- pyrrolo[3,2-b]pyridin- 5-yl]-6-isopropyl- pyridin-2-yl}-methyl- amine | 341.4 | |
| 203 | 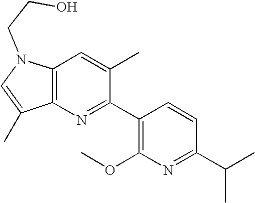 | 2-[5-(6-Isopropyl-2- methoxy-pyridin-3-yl)- 3,6-dimethyl- pyrrolo[3,2-b]pyridin- 1-yl]-ethanol | 340.17 | 2.07 |
| 204 | 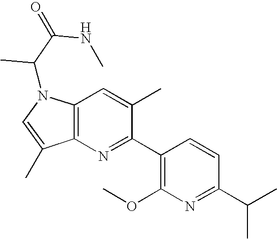 | 2-[5-(6-Isopropyl-2- methoxy-pyridin-3-yl)- 3,6-dimethyl- pyrrolo[3,2-b]pyridin- 1-yl]-N-methyl propionamide | 381.4 | |
| 205 | 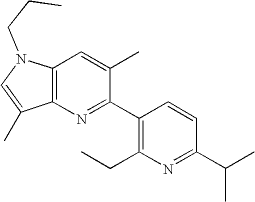 | 5-(2-Ethyl-6-isopropyl- pyridin-3-yl)-3,6- dimethyl-1-propyl-1H- pyrrolo[3,2-b]pyridine | 336.4 | |
| 206 | 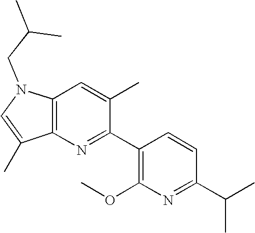 | 1-Isobutyl-5-(6- isopropyl-2-methoxy- pyridin-3-yl)-3,6- dimethyl-1H- pyrrolo[3,2-b]pyridine | 352.4 | |
| 207 | 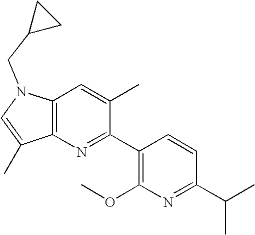 | 1-Cyclopropylmethyl- 5-(6-isopropyl-2- methoxy-pyridin-3-yl)- 3,6-dimethyl-1H- pyrrolo[3,2-b]pyridine | 350.4 | |
| 208 | 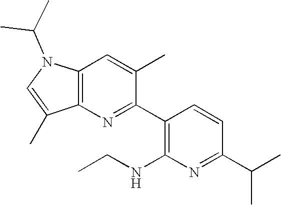 | Ethyl-[6-isopropyl-3- (1-isopropyl-3,6- dimethyl-1H- pyrrolo[3,2-b]pyridin- 5-yl)-pyridin-2-yl]- amine | 351.4 | |
| 209 | 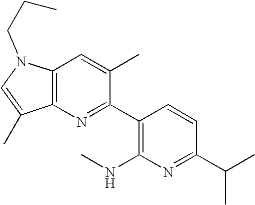 | [3-(3,6-Dimethyl-1- propyl-1H-pyrrolo[3,2- b]pyridin-5-yl)-6- isopropyl-pyridin-2-yl]- methyl-amine | 337.4 | |
| 210 | 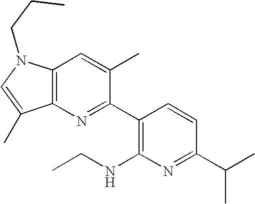 | [3-(3,6-Dimethyl-1- propyl-1H-pyrrolo[3,2- b]pyridin-5-yl)-6- isopropyl-pyridin-2-yl]- ethyl-amine | 351.5 | |
| 211 | 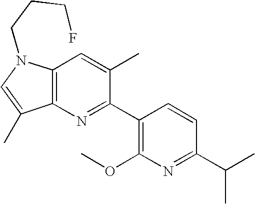 | 1-(3-Fluoro-propyl)-5- (6-isopropyl-2- methoxy-pyridin-3-yl)- 3,6-dimethyl-1H- pyrrolo[3,2-b]pyridine | 356.4 | |
| 212 | 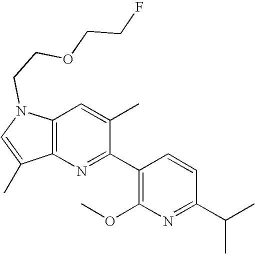 | 1-[2-(2-Fluoro-ethoxy)- ethyl]-5-(6-isopropyl-2- methoxy-pyridin-3-yl)- 3,6-dimethyl-1H- pyrrolo[3,2-b]pyridine | 386.4 | |
| 213 | 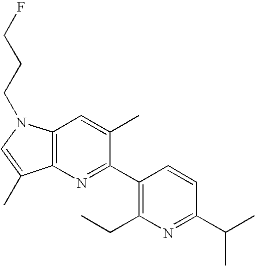 | 5-(2-Ethyl-6-isopropyl- pyridin-3-yl)-1-(3- fluoro-propyl)-3,6- dimethyl- 1H-pyrrolo[3,2-b]pyridine | 354.20 | 1.96 |
| 214 | 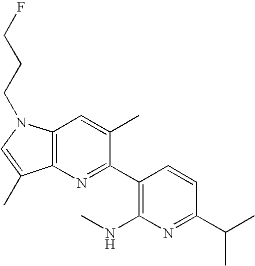 | {3-[1-(3-Fluoro- propyl)-3,6-dimethyl- 1H-pyrrolo[3,2- b]pyridin-5-yl]-6- isopropyl-pyridin-2- yl}-methyl-amine | 355.19 | 1.79 |
| 215 | 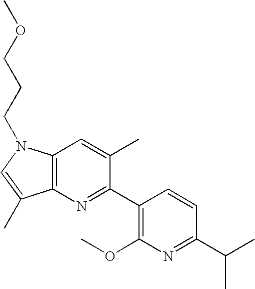 | 5-(6-Isopropyl-2- methoxy-pyridin-3-yl)- 1-(3-methoxy-propyl)- 3,6-dimethyl-1H- pyrrolo[3,2-b]pyridine | 368.4 | |
| 216 | 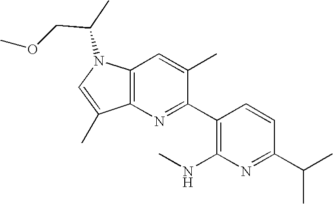 | {6-Isopropyl-3-[1-((S)- 2-methoxy-1-methyl- ethyl)-3,6-dimethyl- 1H-pyrrolo[3,2- b]pyridin-5-yl]-pyridin- 2-yl}-methyl-amine | 367.2 | |
| 217 | 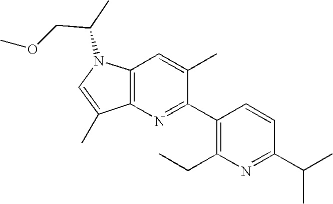 | 5-(2-Ethyl-6-isopropyl- pyridin-3-yl)-1-((S)-2- methoxy-1-methyl- ethyl)-3,6-dimethyl-1H- pyrrolo[3,2-b]pyridine | 366.4 | |
| 218 | 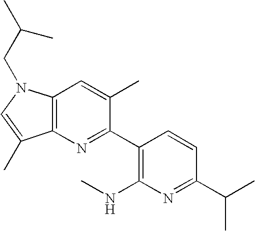 | [3-(1-Isobutyl-3,6- dimethyl-1H- pyrrolo[3,2-b]pyridin- 5-yl)-6-isopropyl- pyridin-2-yl]-methyl-amine | 351.5 | |
| 219 | 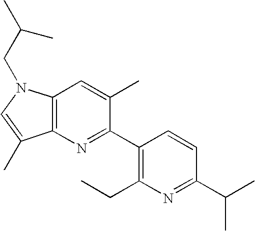 | 5-(2-Ethyl-6-isopropyl- pyridin-3-yl)-1- isobutyl-3,6-dimethyl- 1H-pyrrolo[3,2- b]pyridine | 350.5 | |
| 220 | 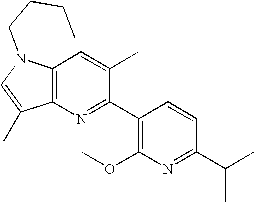 | 1-Butyl-5-(6-isopropyl- 2-methoxy-pyridin-3- yl)-3,6-dimethyl-1H- pyrrolo[3,2-b]pyridine | 352.4 | |
| 221 | 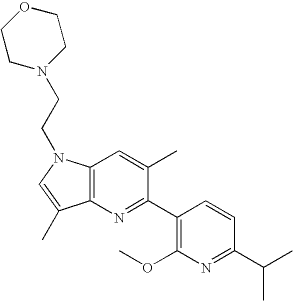 | 5-(6-Isopropyl-2- methoxy-pyridin-3-yl)- 3,6-dimethyl-1-(2- morpholin-4-yl-ethyl)- 1H-pyrrolo[3,2-b]pyridine | 409.5 | |
| 222 | 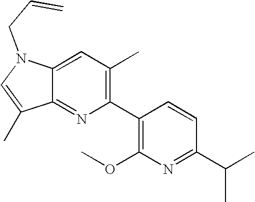 | 1-Allyl-5-(6-isopropyl- 2-methoxy-pyridin-3- yl)-3,6-dimethyl-1H- pyrrolo[3,2-b]pyridine | Rf: 033 (Hexane- Ethyl- acetate: 4:1) | |
| 223 | 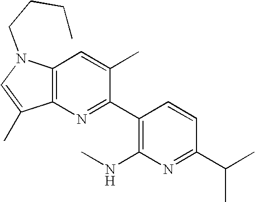 | [3-(1-Butyl-3,6- dimethyl-1H- pyrrolo[3,2-b]pyridin- 5-yl)-6-isopropyl- pyridin-2-yl]-methyl- amine | 351.5 | |
| 224 | 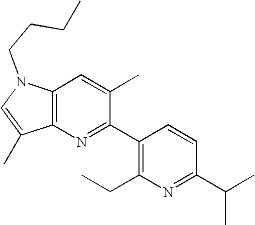 | 1-Butyl-5-(2-ethyl-6- isopropyl-pyridin-3-yl)- 3,6-dimethyl-1H- pyrrolo[3,2-b]pyridine | 350.5 | |
| 225 | 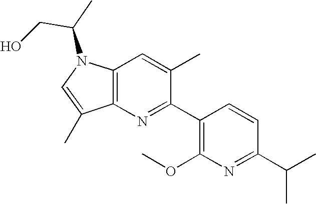 | (R)-2-[5-(6-Isopropyl- 2-methoxy-pyridin-3- yl)-3,6-dimethyl- pyrrolo[3,2-b]pyridin- 1-yl]-propan-1-ol | 354.4 | |
| 226 | 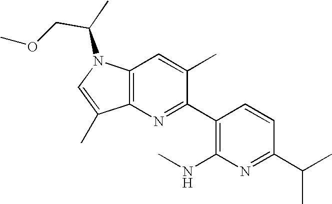 | {6-Isopropyl-3-[1-((R)- 2-methoxy-1-methyl- ethyl)-3,6-dimethyl- 1H-pyrrolo[3,2- b]pyridin-5-yl]-pyridin- 2-yl}-methyl-amine | 367.5 | |
| 227 | 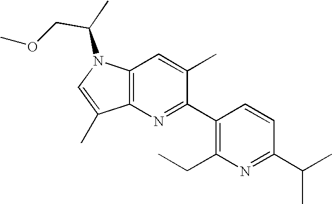 | 5-(2-Ethyl-6-isopropyl- pyridin-3-yl)-1-((R)-2- methoxy-1-methyl- ethyl)- 3,6-dimethyl-1H- pyrrolo[3,2-b]pyridine | 366.4 | |
| 228 | 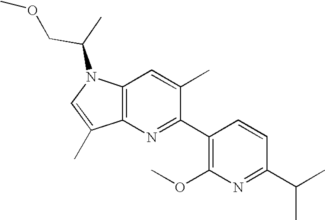 | 5-(6-Isopropyl-2- methoxy-pyridin-3-yl)- 1-((R)-2-methoxy-1- methyl-ethyl)-3,6- dimethyl-1H- pyrrolo[3,2-b]pyridine | Rf: 0.4 (Hexane- Ethyl- acetate: 2:1) | |
| 229 | 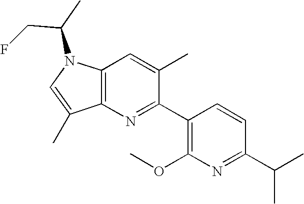 | 1-((R)-2-Fluoro-1- methyl-ethyl)-5-(6- isopropyl-2-methoxy- pyridin-3-yl)-3,6- dimethyl-1H- pyrrolo[3,2-b]pyridine | 356.4 | |
| 230 | 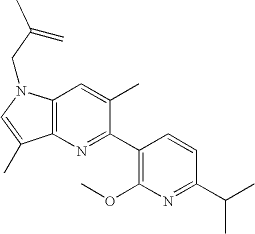 | 5-(6-Isopropyl-2- methoxy-pyridin-3-yl)- 3,6-dimethyl-1-(2- methyl-allyl)-1H- pyrrolo[3,2-b]pyridine | 350.5 | |
| 231 | 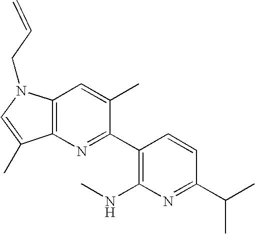 | [3-(1-Allyl-3,6- dimethyl-1H- pyrrolo[3,2-b]pyridin- 5-yl)-6-isopropyl- pyridin-2-yl]-methyl- amine | 335.3 | |
| 232 | 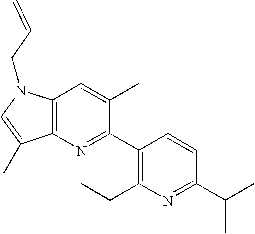 | 1-Allyl-5-(2-ethyl-6- isopropyl-pyridin-3-yl)- 3,6-dimethyl-1H- pyrrolo[3,2-b]pyridine | 334.4 | |
| 233 | 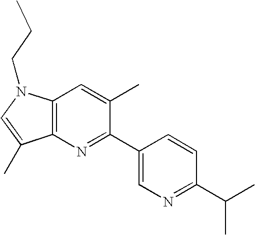 | 5-(6-Isopropyl-pyridin- 3-yl)-3,6-dimethyl-1- propyl-1H-pyrrolo[3,2- b]pyridine | 308.3 | |
| 234 | 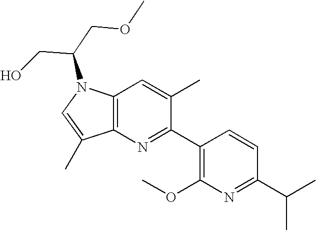 | (S)-2-[5-(6-Isopropyl- 2-methoxy-pyridin-3- yl)-3,6-dimethyl- pyrrolo[3,2-b]pyridin- 1-yl]-3-methoxy- propan-1-ol | Rf: 0.3 (Hexane/ ethyl acetate: 1:1) | |
| 235 | 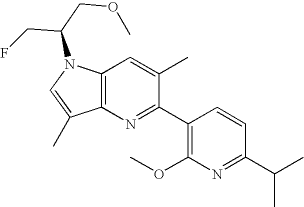 | 1-((R)-1-Fluoromethyl- 2-methoxy-ethyl)-5-(6- isopropyl-2-methoxy- pyridin-3-yl)-3,6- dimethyl-1H- pyrrolo[3,2-b]pyridine | 386.6 | |
| 236 | 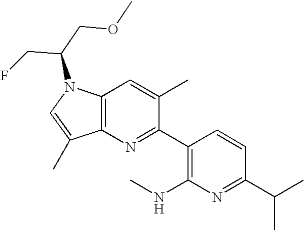 | {3-[1-((R)-1- Fluoromethyl-2- methoxy-ethyl)-3,6- dimethyl-1H- pyrrolo[3,2-b]pyridin- 5-yl]-6-isopropyl- pyridin-2-yl}-methyl- amine | 385.5 | |
| 237 | 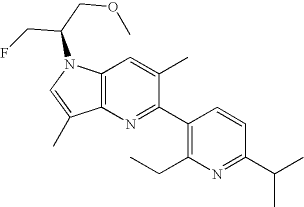 | 5-(2-Ethyl-6-isopropyl- pyridin-3-yl)-1-((R)-1- fluoromethyl-2- methoxy-ethyl)-3,6- dimethyl-1H- pyrrolo[3,2-b]pyridine | 384.4 | |
| 238 | 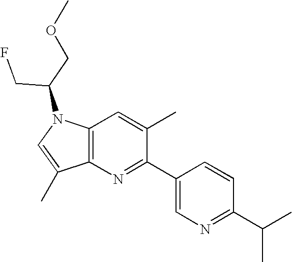 | 1-((R)-1-Fluoromethyl- 2-methoxy-ethyl)-5-(6- isopropyl-pyridin-3-yl)- 3,6-dimethyl-1H- pyrrolo[3,2-b]pyridine | 356.4 | |
| 239 | 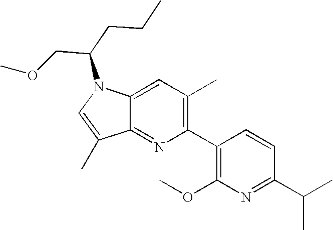 | 5-(6-Isopropyl-2- methoxy-pyridin-3-yl)- 1-(1-methoxymethyl- butyl)-3,6-dimethyl- 1H-pyrrolo[3,2- b]pyridine | 396.5 | |
| 240 | 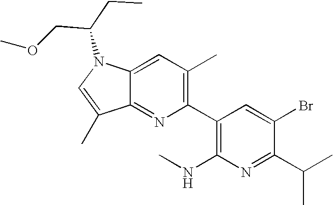 | {5-Bromo-6-isopropyl- 3-[1-((S)-2-methoxy-1- methyl-ethyl)-3,6- dimethyl-1H- pyrrolo[3,2-b]pyridin- 5-yl]-pyridin-2-yl}- methyl-amine | 445.3 | |
| 241 | 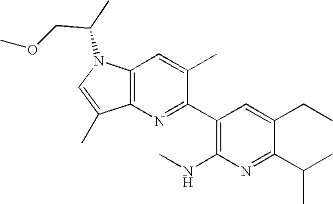 | {5-Ethyl-6-isopropyl-3- [1-((S)-2-methoxy-1- methyl-ethyl)-3,6- dimethyl-1H- pyrrolo[3,2-b]pyridin- 5-yl]-pyridin- 2-yl}-methyl-amine | 395.5 | |
| 242 | 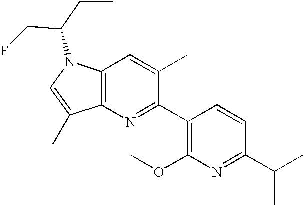 | 1-((S)-1-Fluoromethyl- propyl)-5-(6-isopropyl- 2-methoxy-pyridin-3- yl)-3,6-dimethyl-1H- pyrrolo[3,2-b]pyridine | 370.44 | 1.55 |
| 243 | 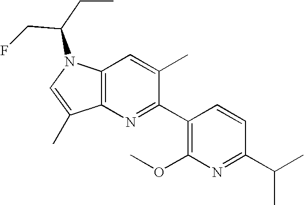 | 1-((R)-1-Fluoromethyl- propyl)-5-(6-isopropyl- 2-methoxy-pyridin-3- yl)-3,6-dimethyl-1H- pyrrolo[3,2-b]pyridine | 370.5 | |
| 244 | 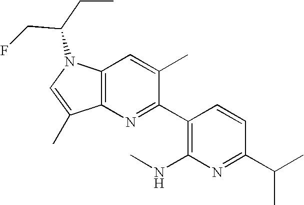 | {3-[1-((S)-1- Fluoromethyl-propyl)- 3,6-dimethyl-1H- pyrrolo[3,2-b]pyridin- 5-yl]-6-isopropyl- pyridin-2-yl}-methyl- amine | 369.5 | |
| 245 | 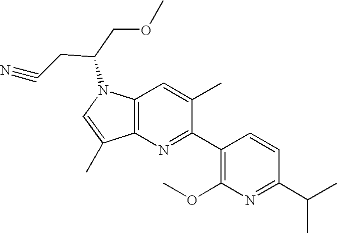 | (S)-3-[5-(6-Isopropyl- 2-methoxy-pyridin-3- yl)-3,6-dimethyl- pyrrolo[3,2-b]pyridin- 1-yl]-4-methoxy- butyronitrile | 393.4 | |
| 246 | 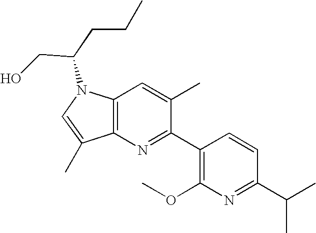 | (R)-2-{5-(6-Isopropyl- 2-methoxy-pyridin-3- yl)-3,6-dimethyl- pyrrolo[3,2-b]pyridin- 1-yl]-pentan-1-ol | 382.5 | |
| 247 | 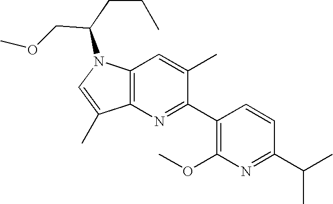 | 5-(6-Isopropyl-2- methoxy-pyridin-3-yl)- 1-((R)-1- methoxymethyl-butyl)- 3,6-dimethyl-1H- pyrrolo[3,2-b]pyridine | 396.5 | |
| 248 | 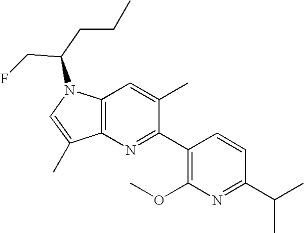 | 1-((R)-1-Fluoromethyl- butyl)-5-(6-isopropyl-2- methoxy-pyridin-3-yl)- 3,6-dimethyl-1H- pyrrolo[3,2-b]pyridine | 384.5 | |
| 249 | 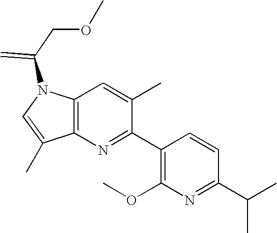 | 5-(6-Isopropyl-2- methoxy-pyridin-3-yl)- 1-(1-methoxymethyl- vinyl)-3,6-dimethyl- 1H-pyrrolo[3,2-b]pyridine | 366.4 | |
| 250 | 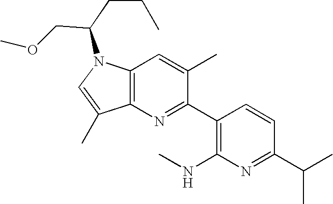 | {6-Isopropyl-3-[1-((R)- 1-methoxymethyl- butyl)-3,6-dimethyl- 1H-pyrrolo[3,2- b]pyridin-5-yl]-pyridin- 2-yl}-methyl-amine | 395.6 | |
| 251 | 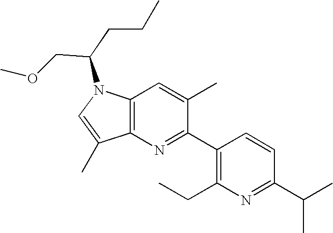 | 5-(2-Ethyl-6-isopropyl- pyridin-3-yl)-1-((R)-1- methoxymethyl-butyl)- 3,6-dimethyl-1H- pyrrolo[3,2-b]pyridine | 394.4 | |
| 252 | 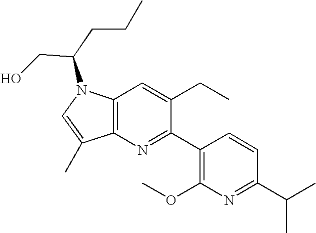 | (S)-2-[6-Ethyl-5-(6- isopropyl-2-methoxy- pyridin-3-yl)-3-methyl- pyrrolo[3,2-b]pyridin- 1-yl]-3-methoxy- propan-1-ol | Rf: 0.4 (Hexane/ Ethyl acetate: 1:1) | |
| 253 | 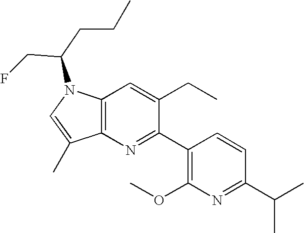 | 6-Ethyl-1-((R)-1- fluoromethyl-2- methoxy-ethyl)-5-(6- isopropyl-2-methoxy- pyridin-3-yl)-3-methyl- 1H-pyrrolo[3,2- b]pyridine | 400.5 | |
| 254 | 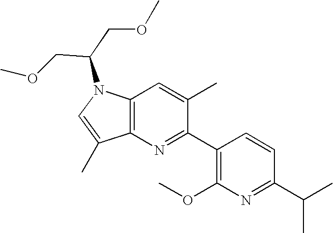 | 5-(6-Isopropyl-2- methoxy-pyridin-3-yl)- 1-(2-methoxy-1- methoxymethyl-ethyl)- 3,6-dimethyl-1H- pyrrolo[3,2-b]pyridine | 398.5 | |
| 255 | 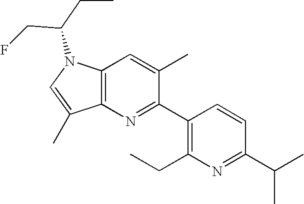 | 5-(2-Ethyl-6-isopropyl- pyridin-3-yl)-1-((S)-1- fluoromethyl-propyl)- 3,6-dimethyl-1H- pyrrolo[3,2-b]pyridine | 368.4 | |
| 256 | 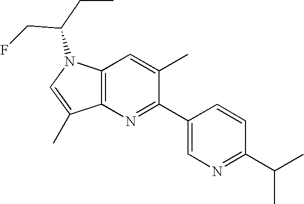 | 1-((S)-1-Fluoromethyl- propyl)-5-(6-isopropyl- pyridin-3-yl)-3,6- dimethyl-1H- pyrrolo[3,2-b]pyridine | 340.4 | |
| 257 | 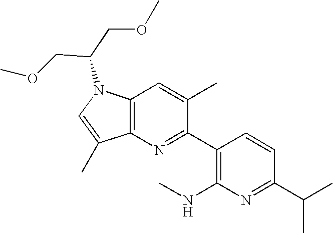 | {6-Isopropyl-3-[1-(2- methoxy-1- methoxymethyl-ethyl)- 3,6-dimethyl-1H- pyrrolo[3,2-b]pyridin- 5-yl]-pyridin-2-yl}- methyl-amine | 397.5 | |
| 258 | 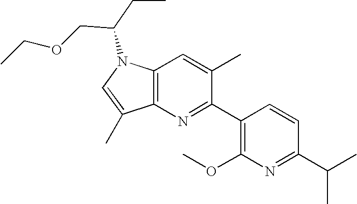 | 1-((S)-1-Ethoxymethyl- propyl)-5-(6-isopropyl- 2-methoxy-pyridin-3- yl)-3,6-dimethyl-1H- pyrrolo[3,2-b]pyridine | 396.5 | |
| 259 | 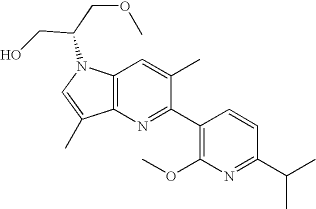 | (R)-2-[5-(6-Isopropyl- 2-methoxy-pyridin-3- yl)-3,6-dimethyl- pyrrolo[3,2-b]pyridin- 1-yl]-3-methoxy- propan-1-ol | 384.4 | |
| 260 | 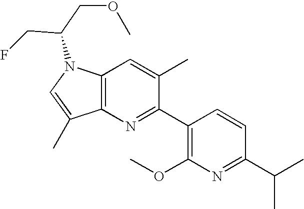 | 1-((S)-1-Fluoromethyl- 2-methoxy-ethyl)-5-(6- isopropyl-2-methoxy- pyridin-3-yl)-3,6- dimethyl-1H- pyrrolo[3,2-b]pyridine | Rf: 0.3 (Hexane/ Ethyl accetate: 3/1) | |
| 261 | 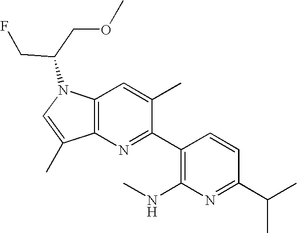 | {3-{1-((S)-1- Fluoromethyl-2- methoxy-ethyl)-3,6- dimethyl-1H- pyrrolo[3,2-b]pyridin-5- yl]-6-isopropyl- pyridin-2- yl}-methyl-amine | Rf: 0.3 (Hexane/ Ethyl acetate: 2.5/1) | |
| 262 | 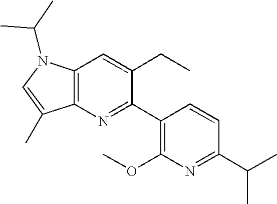 | 6-Ethyl-1-isopropyl-5- (6-isopropyl-2- methoxy-pyridin-3-yl)- 3-methyl-1H- pyrrolo[3,2-b]pyridine | 352.4 | |
| 263 | 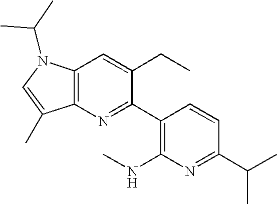 | [3-(6-Ethyl-1- isopropyl-3-methyl-1H- pyrrolo[3,2-b]pyridin- 5-yl)-6-isopropyl- pyridin-2-yl]-methyl- amine | 351.4 | |
| 264 | 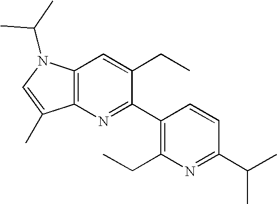 | 6-Ethyl-5-(2-ethyl-6- isopropyl-pyridin-3-yl)- 1-isopropyl-3-methyl- 1H-pyrrolo[3,2-b]pyridine | 350.4 | |
| 265 | 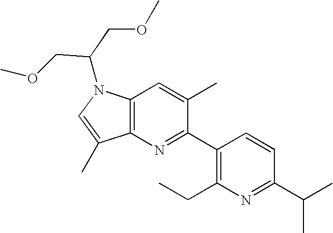 | 5-(2-Ethyl-6-isopropyl- pyridin-3-yl)-1-(2- methoxy-1- methoxymethyl-ethyl)- 3,6-dimethyl-1H- pyrrolo[3,2-b]pyridine | 396.5 | |
| 266 | 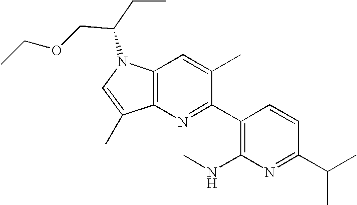 | {3-[1-((S)-1- Ethoxymethyl-propyl)- 3,6-dimethyl-1H- pyrrolo[3,2-b]pyridin- 5-yl]-6-isopropyl- pyridin-2-yl}-methyl- amine | 395.5 | |
| 267 | 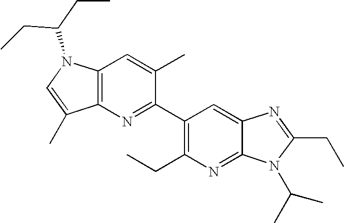 | 2,5-Diethyl-6-[1-(1- ethyl-propyl)-3,6- dimethyl-1H- pyrrolo[3,2-b]pyridin- 5-yl]-3-isopropyl-3H- imidazo[4,5-b]pyridine | 432.21 | 2.32 |
| 268 | 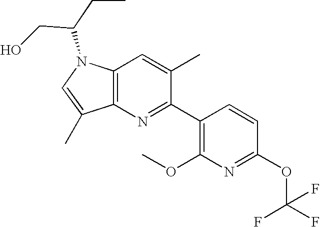 | (S)-2-[5-(2-Methoxy-4- trifluoromethoxy- phenyl)-3,6-dimethyl- pyrrolo[3,2-b]pyridin-1-yl]- butan-1-ol | 409.3 | |
| 269 | 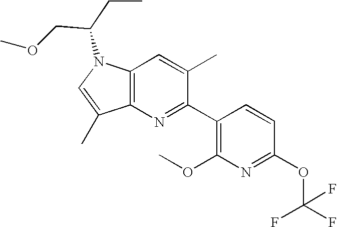 | 1-((S)-1-Methoxymethyl- propyl)-5-(2-methoxy- 4-trifluoromethoxy- phenyl)-3,6-dimethyl- 1H-pyrrolo[3,2- b]pyridine | 423.3 | |
| 270 | 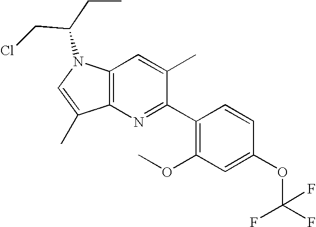 | 1-((S)-1-Chloromethyl- propyl)-5-(2-methoxy- 4-trifluoromethoxy- phenyl)- 3,6-dimethyl-1H- pyrrolo[3,2-b]pyridine | 427.4 | |
| 271 | 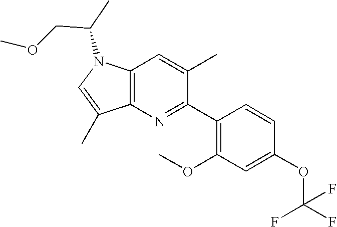 | 1-((S)-2-Methoxy-1- methyl-ethyl)-5-(2- methoxy-4- trifluoromethoxy- phenyl)-3,6-dimethyl- 1H-pyrrolo[3,2-b]pyridine | 409.4 | |
| 272 | 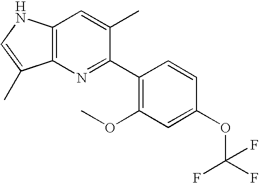 | 5-(2-Methoxy-4- trifluoromethoxy- phenyl)-3,6-dimethyl- 1H-pyrrolo[3,2-b]pyridine | 337.1 | |
| 273 | 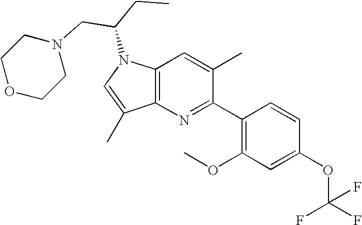 | 5-(2-Methoxy-4- trifluoromethoxy- phenyl)-3,6-dimethyl- 1-((S)-1-morpholin-4- ylmethyl-propyl)-1H- pyrrolo[3,2-b]pyridine | 478.06 | 1.91 |
| 274 | 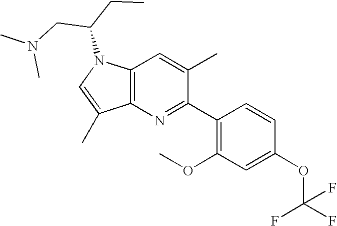 | {(S)-2-[5-(2-Methoxy- 4-trifluoromethoxy- phenyl)-3,6-dimethyl- pyrrolo[3,2-b]pyridin- 1-yl]-butyl}-dimethyl- amine | 436.04 | 1.84 |
| 275 | 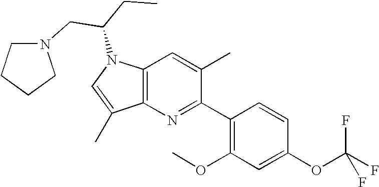 | 5-(2-Methoxy-4- trifluoromethoxy- phenyl)-3,6-dimethyl- 1-((S)-1-pyrrolidin-1- ylmethyl-propyl)-1H- pyrrolo[3,2-b]pyridine | 462.4 | |
| 276 | 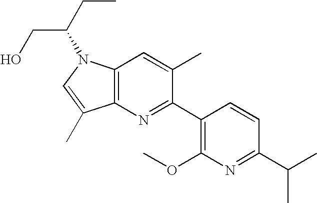 | (S)-2-[5-(6-Isopropyl- 2-methoxy-pyridin-3- yl)-3,6-dimethyl- pyrrolo[3,2-b]pyridin- 1-yl]-butan-1-ol | 368.3 | |
| 277 | 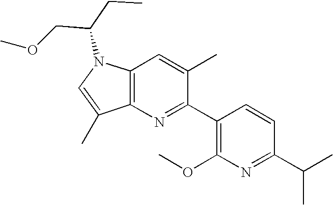 | 5-(6-Isopropyl-2- methoxy-pyridin-3-yl)- 1-((S)-1-methoxymethyl- propyl)-3,6-dimethyl-1H- pyrrolo[3,2-b]pyridine | 382.3 | |
| 278 | 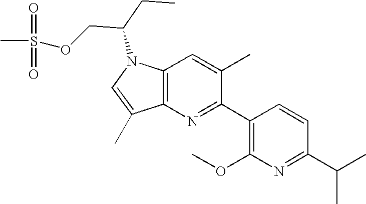 | Methanesulfonic acid (S)-2-[5-(6-isopropyl-2- methoxy-pyridin-3-yl)- 3,6-dimethyl- pyrrolo[3,2-b]pyridin- 1-yl]-butyl ester | 446.04 | 2.23 |
| 279 | 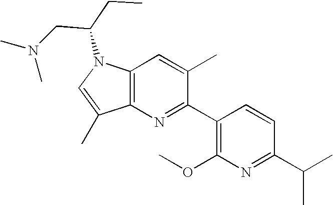 | {(S)-2-[5-(6-Isopropyl- 2-methoxy-pyridin-3- yl)-3,6-dimethyl- pyrrolo[3,2-b]pyridin- 1-yl]-butyl}-dimethyl- amine | 395.13 | 1.87 |
| 280 | 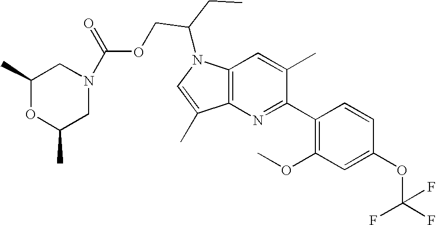 | (2R,6S)-2,6-Dimethyl- morpholine-4- carboxylic acid 2-[5-(2- methoxy-4- trifluoromethoxy- phenyl)-3,6-dimethyl- pyrrolo[3,2-b]pyridin- 1-yl]-butyl ester | 550.1 | 2.31 |
| 281 | 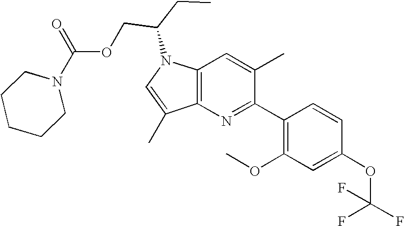 | Piperidine-1-carboxylic acid (S)-2-[5-(2- methoxy-4- trifluoromethoxy- phenyl)-3,6-dimethyl- pyrrolo[3,2-b]pyridin- 1-yl]-butyl ester | 520.11 | 2.36 |
| 282 | 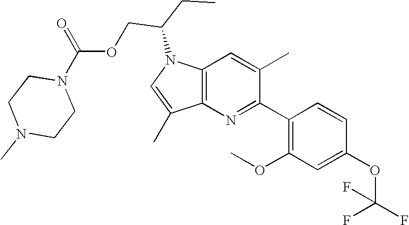 | 4-Methyl-piperazine-1- carboxylic acid (S)-2- [5-(2-methoxy-4- trifluoromethoxy- phenyl)-3,6-dimethyl- pyrrolo[3,2-b]pyridin- 1-yl]-butyl ester | 535.13 | 1.25 |
| 283 | 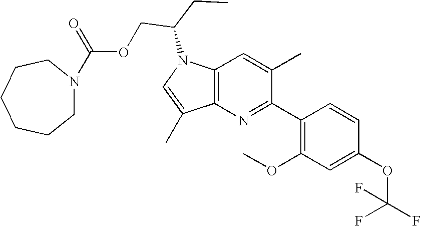 | Azepane-1-carboxylic acid (S)-2-[5-(2- methoxy-4- trifluoromethoxy- phenyl)-3,6-dimethyl- pyrrolo[3,2-b]pyridin- 1-yl]-butyl ester | 534.13 | 1.45 |
| 284 | 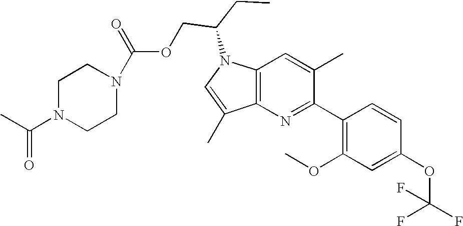 | 4-Acetyl-piperazine-1- carboxylic acid (S)-2- [5-(2-methoxy-4- trifluoromethoxy- phenyl)-3,6-dimethyl- pyrrolo[3,2-b]pyridin- 1-yl]-butyl ester | 563.12 | 1.35 |
| 285 | 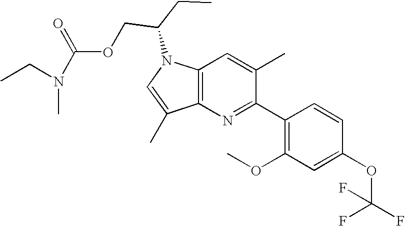 | Ethyl-methyl-carbamic acid (S)-2-[5-(2- methoxy-4- trifluoromethoxy- phenyl)-3,6-dimethyl- pyrrolo[3,2-b]pyridin- 1-yl]-butyl ester | 494.10 | 1.40 |
| 286 | 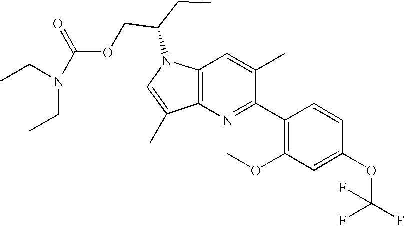 | Diethyl-carbamic acid (S)-2-[5-(2-methoxy-4- trifluoromethoxy- phenyl)-3,6-dimethyl- pyrrolo[3,2-b]pyridin- 1-yl]-butyl ester | 508.12 | 1.41 |
| 287 | 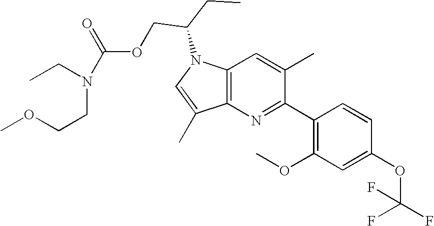 | Ethyl-(2-methoxy- ethyl)-carbamic acid (S)-2-[5-(2-methoxy-4- trifluoromethoxy- phenyl)-3,6-dimethyl- pyrrolo[3,2-b]pyridin- 1-yl]-butyl ester | 538.12 | 1.41 |
| 288 | 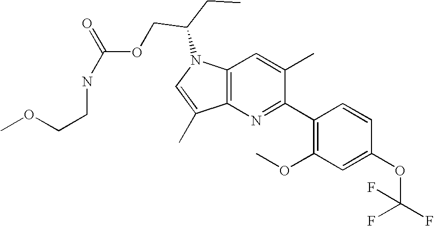 | (2-Methoxy-ethyl)- carbamic acid (S)-2-[5- (2-methoxy-4- trifluoromethoxy- phenyl)-3,6-dimethyl- pyrrolo[3,2-b]pyridin- 1-yl]-butyl ester | 510.09 | 2.18 |
| 289 | 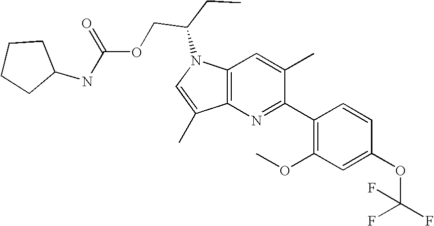 | Cyclopentyl-carbamic acid (S)-2-[5-(2- methoxy-4- trifluoromethoxy- phenyl)-3,6-dimethyl- pyrrolo[3,2-b]pyridin- 1-yl]-butyl ester | 520.12 | 1.43 |
| 290 | 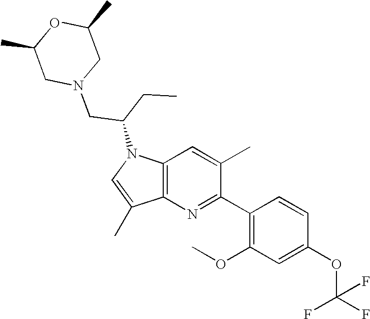 | 1-[(S)-1-((2S,6R)-2,6- Dimethyl-morpholin-4- ylmethyl)-propyl]-5-(2- methoxy-4- trifluoromethoxy- phenyl)-3,6-dimethyl- 1H-pyrrolo[3,2-b]pyridine | 506.13 | 1.30 |
| 291 | 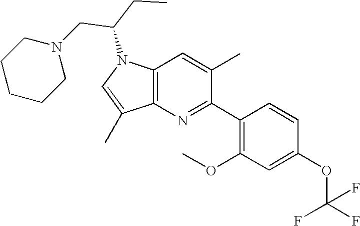 | 5-(2-Methoxy-4- trifluoromethoxy- phenyl)-3,6-dimethyl- 1-((S)-1-piperidin-1- ylmethyl-propyl)-1H- pyrrolo[3,2-b]pyridine | 476.13 | 1.85 |
| 292 | 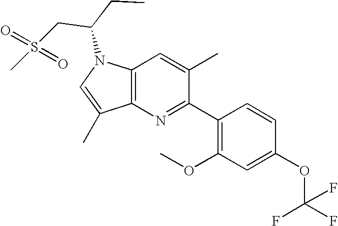 | 1-((S)-1- Methanesulfonylmethyl- propyl)-5-(2-methoxy- 4-trifluoromethoxy- phenyl)-3,6-dimethyl- 1H-pyrrolo[3,2-b]pyridine | 471.03 | 2.11 |
| 293 | 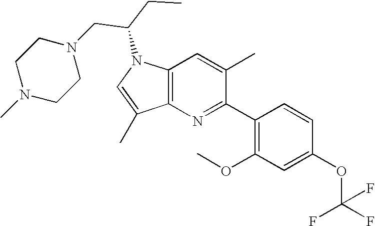 | 5-(2-Methoxy-4- trifluoromethoxy- phenyl)-3,6-dimethyl- 1-[(S)-1-(4-methyl- piperazin-1-ylmethyl)- propyl]-1H- pyrrolo[3,2-b]pyridine | 491.13 | 1.84 |
| 294 | 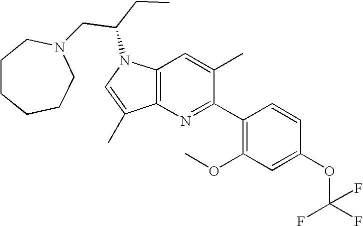 | 1-((S)-1-Azepan-1- ylmethyl-propyl)- 5-(2-methoxy-4- trifluoromethoxy- phenyl)-3,6-dimethyl- 1H-pyrrolo[3,2-b]pyridine | 490.14 | 1.89 |
| 295 | 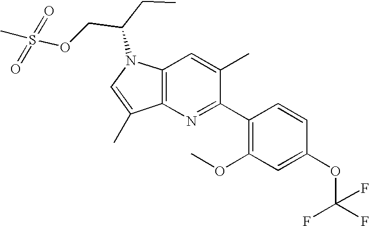 | Methanesulfonic acid (S)-2-[5-(2-methoxy-4- trifluoromethoxy- phenyl)-3,6-dimethyl- pyrrolo[3,2-b]pyridin- 1-yl]-butyl ester | 487.04 | 2.16 |
| 296 | 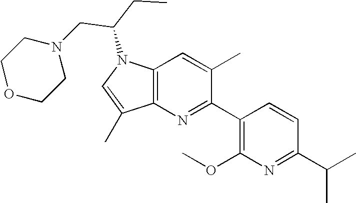 | 5-(6-Isopropyl-2- methoxy-pyridin-3-yl)- 3,6-dimethyl-1-((S)-1- morpholin-4-ylmethyl- propyl)-1H- pyrrolo[3,2-b]pyridine | 437.16 | 2.02 |
| 297 | 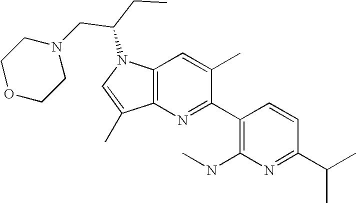 | {3-[3,6-Dimethyl-1- ((S)-1-morpholin-4- ylmethyl-propyl)-1H- pyrrolo[3,2-b]pyridin- 5-yl]-6-isopropyl- pyridin-2- yl}-methyl-amine | 436.19 | 1.57 |
| 298 | 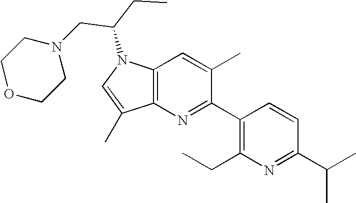 | 5-(2-Ethyl-6-isopropyl- pyridin-3-yl)-3,6- dimethyl-1-((S)-1- morpholin-4-ylmethyl- propyl)-1H- pyrrolo[3,2-b]pyridine | 435.20 | 1.57 |
| 299 | 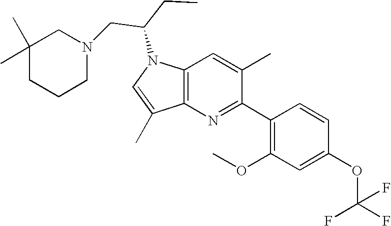 | 1-[(S)-1-(3,3-Dimethyl- piperidin-1-ylmethyl)- propyl]-5-(2-methoxy- 4-trifluoromethoxy- phenyl)-3,6-dimethyl- 1H-pyrrolo[3,2-b]pyridine | 504.5 | |
| 300 | 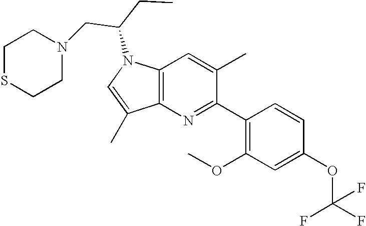 | 5-(2-Methoxy-4- trifluoromethoxy- phenyl)-3,6-dimethyl- 1-((S)-1-thiomorpholin- 4-ylmethyl-propyl)-1H- pyrrolo[3,2-b]pyridine | 494.07 | 1.97 |
| 301 | 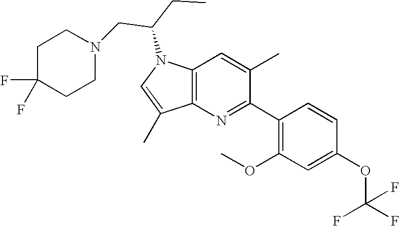 | 1-[(S)-1-(4,4-Difluoro- piperidin-1-ylmethyl)- propyl]-5-(2-methoxy- 4-trifluoromethoxy- phenyl)-3,6-dimethyl- 1H-pyrrolo[3,2-b]pyridine | 512.10 | 2.13 |
| 302 | 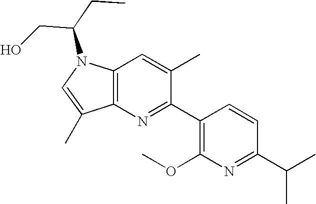 | (R)-2-[5-(6-Isopropyl- 2-methoxy-pyridin-3- yl)-3,6-dimethyl- pyrrolo[3,2-b]pyridin- 1-yl]-butan-1-ol | 368.16 | 2.21 |
| 303 | 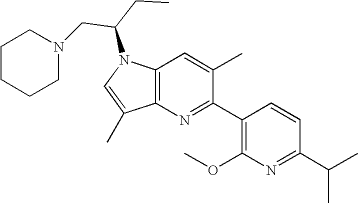 | 5-(6-Isopropyl-2- methoxy-pyridin-3-yl)- 3,6-dimethyl-1-((R)-1- morpholin-4-ylmethyl- propyl)-1H- pyrrolo[3,2-b]pyridine | 437.16 | 1.95 |
| 304 | 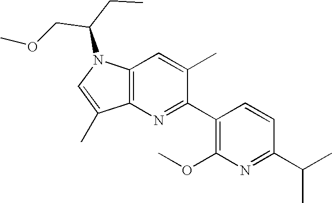 | 5-(6-Isopropyl-2- methoxy-pyridin-3-yl)- 1-((R)-1-methoxymethyl- propyl)- 3,6-dimethyl-1H- pyrrolo[3,2-b]pyridine | 382.16 | 2.28 |
| 305 | 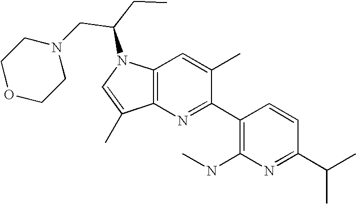 | {3-[3,6-Dimethyl-1- ((R)-1-morpholin-4- ylmethyl-propyl)-1H- pyrrolo[3,2- b]pyridin-4-yl]-6- isopropyl-pyridin-2- yl}-methyl-amine | 436.16 | 1.58 |
| 306 | 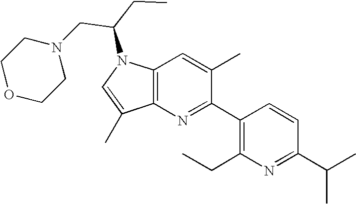 | 5-(2-Ethyl-6-isopropyl- pyridin-3-yl)-3,6- dimethyl-1-((R)-1- morpholin-4-ylmethyl- propyl)-1H- pyrrolo[3,2-b]pyridine | 435.15 | 1.59 |
| 307 | 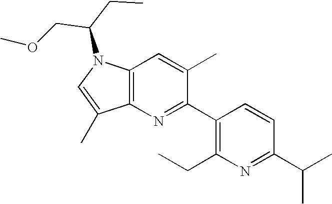 | 5-(2-Ethyl-6-isopropyl- pyridin-3-yl)-1-((R)-1- methoxymethyl- propyl)-3,6-dimethyl-1H- pyrrolo[3,2-b]pyridine | 380.5 | |
| 308 | 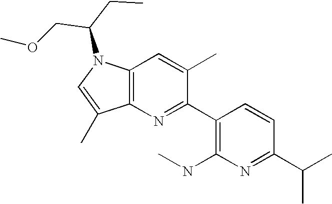 | {6-Isopropyl-3-[1-((R)- 1-methoxymethyl- propyl)-3,6-dimethyl- 1H-pyrrolo[3,2- b]pyridin-5-yl]-pyridin- 2-yl}-methyl-amine | 381.17 | 1.89 |
| 309 | 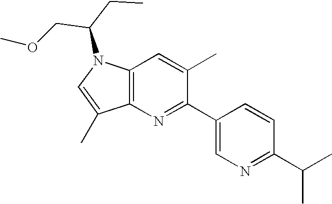 | 5-(6-Isopropyl-pyridin- 3-yl)-1-((R)-1- methoxymethyl- propyl)-3,6-dimethyl- 1H-pyrrolo[3,2-b]pyridine | 352.4 | |
| 310 | 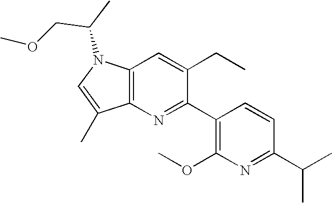 | 6-Ethyl-5-(6-isopropyl- 2-methoxy-pyridin-3- yl)-1-((S)-2-methoxy-1- methyl-ethyl)-3- methyl-1H- pyrrolo[3,2-b]pyridine | 382.19 | 2.31 |
| 311 | 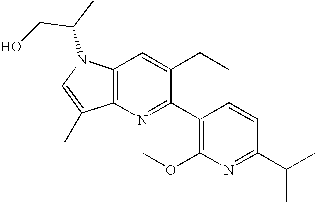 | (S)-2-[6-Ethyl-5-(6- isopropyl-2-methoxy- pyridin-3-yl)-3-methyl- pyrrolo[3,2-b]pyridin- 1-yl]-propan-1-ol | 368.3 | |
| 312 | 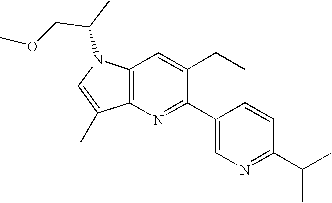 | 6-Ethyl-5-(6-isopropyl- pyridin-3-yl)-1-((S)-2- methoxy-1-methyl- ethyl)-3-methyl-1H- pyrrolo[3,2-b]pyridine | 352.18 | 2.03 |
| 313 | 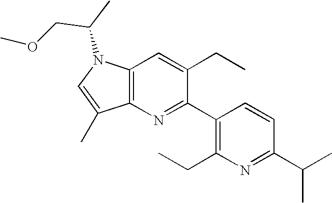 | 6-Ethyl-5-(2-ethyl-6- isopropyl-pyridin-3-yl)- 1-((S)-2-methoxy-1- methyl-ethyl)-3- methyl-1H-pyrrolo[3,2- b]pyridine | 380.20 | 2.08 |
| 314 | 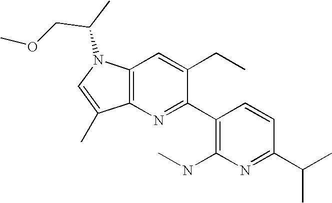 | {3-[6-Ethyl-1-((S)-2- methoxy-1-methyl- ethyl)-3-methyl-1H- pyrrolo[3,2-b]pyridin- 5-yl]-6-isopropyl- pyridin-2-yl}-methyl- amine | 381.19 | 1.90 |
| 315 | 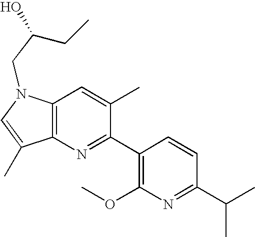 | (R)-1-[5-(6-Isopropyl- 2-methoxy-pyridin-3- yl)-3,6-dimethyl- pyrrolo[3,2-b]pyridin- 1-yl]-butan-2-ol | 368.36 | 2.16 |
| 316 | 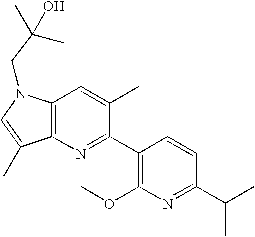 | 1-[5-(6-Isopropyl-2- methoxy-pyridin-3-yl)- 3,6-dimethyl- pyrrolo[3,2-b]pyridin- 1-yl]-2-methyl-propan-2-ol | 368.37 | 2.18 |
| 317 | 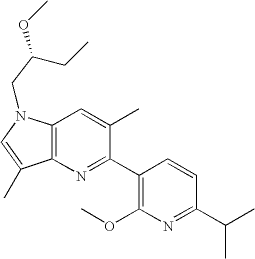 | 5-(6-Isopropyl-2- methoxy-pyridin-3-yl)- 1-((R)-2-methoxy- butyl)-3,6-dimethyl- 1H-pyrrolo[3,2-b]pyridine | 382.32 | 2.30 |
| 318 | 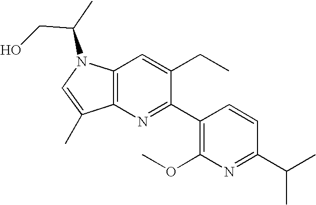 | (R)-2-[6-Ethyl-5-(6- isopropyl-2-methoxy- pyridin-3-yl)-3-methyl- pyrrolo[3,2-b]pyridin- 1-yl]-propan-1-ol | 368.4 | |
| 319 | 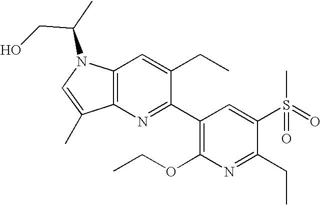 | (R)-2-[5-(2-Ethoxy-6- ethyl-5-methanesulfonyl- pyridin-3-yl)-6-ethyl-3- methyl-pyrrolo[3,2- b]pyridin-1-yl]-propan-1-ol | 446.35 | 2.04 |
| 320 | 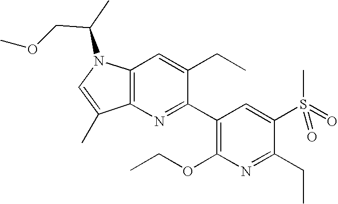 | 5-(2-Ethoxy-6-ethyl-5- methanesulfonyl- pyridin-3-yl)-6-ethyl-1- ((R)-2-methoxy-1- methyl-ethyl)-3- methyl-1H-pyrrolo[3,2- b]pyridine | 460.32 | 2.19 |
| 321 | 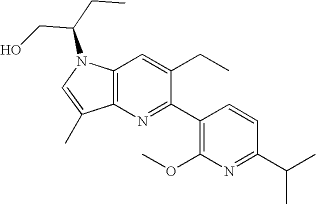 | (R)-2-[6-Ethyl-5-(6- isopropyl-2-methoxy- pyridin-3-yl)-3-methyl- pyrrolo[3,2-b]pyridin- 1-yl]-butan-1-ol | 382.43 | 2.26 |
| 323 | 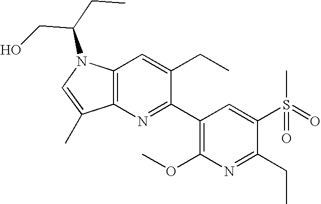 | (R)-2-[5-(2-Ethoxy-6- ethyl-5- methanesulfonyl- pyridin-3-yl)-6-ethyl-3- methyl-pyrrolo[3,2- b]pyridin-1-yl]-butan- 1-ol | 460.16 | 1.37 |
| 324 | 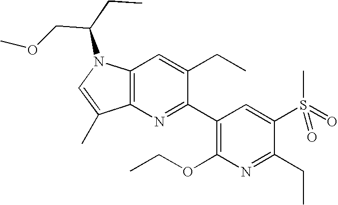 | 5-(2-Ethoxy-6-ethyl-5- methanesulfonyl- pyridin-3-yl)-6-ethyl-1- ((R)-1-methoxymethyl- propyl)-3-methyl-1H- pyrrolo[3,2-b]pyridine | 474.19 | 1.42 |
| 325 | 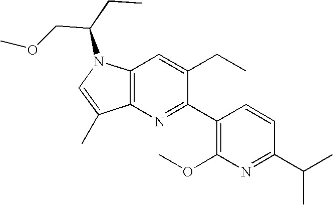 | 6-Ethyl-5-(6-isopropyl- 2-methoxy-pyridin- 3-yl)-1-((R)-1- methoxymethyl- propyl)-3-methyl-1H- pyrrolo[3,2-b]pyridine | 396.42 | 2.35 |
| 326 | 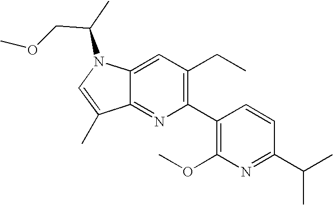 | 6-Ethyl-5-(6-isopropyl- 2-methoxy-pyridin-3- yl)-1-((R)-2-methoxy- 1-methyl-ethyl)-3- methyl-1H-pyrrolo[3,2- b]pyridine | 382.44 | 2.29 |
| 327 | 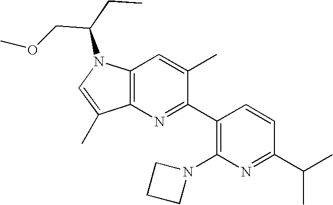 | 5-(2-Azetidin-1-yl-6- isopropyl-pyridin-3-yl)- 1-((R)-1-methoxymethyl- propyl)-3,6-dimethyl- 1H-pyrrolo[3,2-b]pyridine | 407.43 | 2.02 |
| 328 | 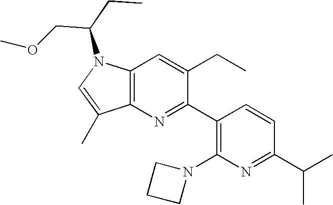 | 5-(2-Azetidin-1-yl-6- isopropyl-pyridin-3-yl)- 6-ethyl-1-((R)-1- methoxymethyl- propyl)-3-methyl-1H- pyrrolo[3,2-b]pyridine | 421.44 | 2.13 |
| 329 | 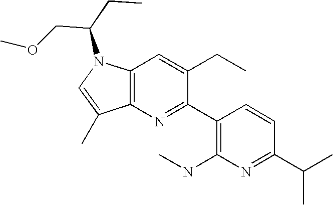 | 3-[6-Ethyl-1-((R)-1- methoxymethyl- propyl)-3-methyl-1H- pyrrolo[3,2-b]pyridin- 5-yl]-6-isopropyl- pyridin-2-yl}-methyl- amine | 395.45 | 1.99 |
| 330 | 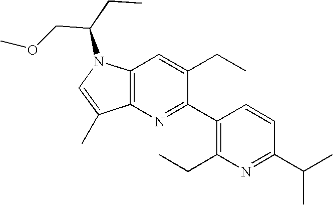 | 6-Ethyl-5-(2-ethyl-6- isopropyl-pyridin-3-yl)- 1-((R)-1- methoxymethyl- propyl)-3-methyl-1H- pyrrolo[3,2-b]pyridine | 394.45 | 2.14 |
| 331 | 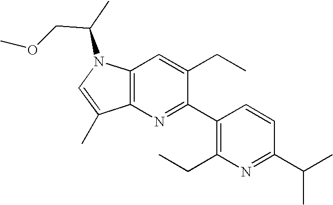 | 6-Ethyl-5-(2-ethyl-6- isopropyl-pyridin-3-yl)- 1-((R)-2-methoxy-1- methyl-ethyl)-3- methyl-1H-pyrrolo[3,2- b]pyridine | 380.45 | 2.07 |
| 332 | 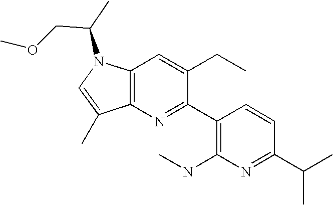 | {3-[6-Ethyl-1-((R)-2- methoxy-1-methyl- ethyl)-3-methyl-1H- pyrrolo[3,2-b]pyridin- 5-yl]-6-isopropyl- pyridin-2-yl}-methyl- amine | 381.45 | 1.88 |
| 333 | 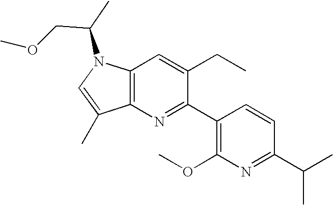 | 6-Ethyl-5-(4-isopropyl- 2-methoxy-phenyl)-1- ((R)-2-methoxy-1- methyl-ethyl)-3- methyl-1H-pyrrolo[3,2- b]pyridine | 381.47 | 2.30 |
| 334 | 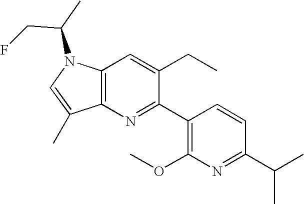 | 6-Ethyl-1-((R)-2- fluoro-1-methyl-ethyl)- 5-(4-isopropyl-2- methoxy-phenyl)-3- methyl-1H-pyrrolo[3,2- b]pyridine | 369.46 | 2.28 |
| 335 | 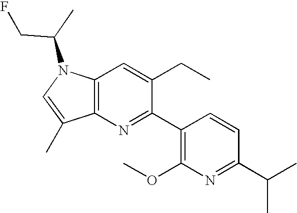 | 6-Ethyl-1-((R)-2- fluoro-1-methyl-ethyl)- 5-(6-isopropyl-2- methoxy-pyridin-3-yl)- 3-methyl-1H- pyrrolo[3,2-b]pyridine | 370.45 | 2.31 |
| 336 | 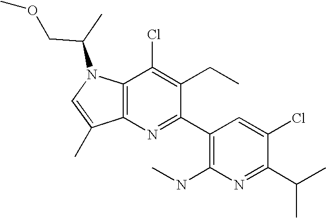 | {5-Chloro-3-[7-chloro- 6-ethyl-1-((R)-2- methoxy-1-methyl- ethyl)-3-methyl-1H- pyrrolo[3,2-b]pyridin- 5-yl]-6-isopropyl- pyridin-2-yl}-methyl- amine | 449.33/ 451.33 | 2.91 |
| 337 | 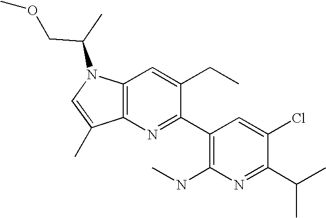 | {5-Chloro-3-[6-ethyl-1- ((R)-2-methoxy-1- methyl-ethyl)-3- methyl-1H-pyrrolo[3,2- b]pyridin-5-yl]-6- isopropyl-pyridin-2- yl}-methyl-amine | 415.40/ 417.39 | 2.53 |
| 338 | 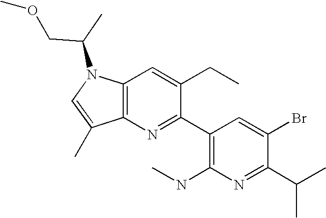 | {5-Bromo-3-[6-ethyl-1- ((R)-2-methoxy-1- methyl-ethyl)-3- methyl-1H-pyrrolo[3,2- b]pyridin-5-yl]-6- isopropyl-pyridin-2- yl}-methyl-amine | 459.35/ 461.35 | 2.55 |
| 339 | 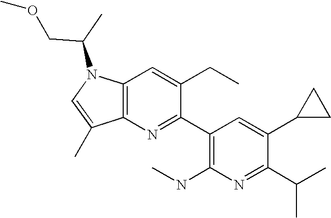 | {5-Cyclopropyl-3-[6- ethyl-1-((R)-2- methoxy-1-methyl- ethyl)-3-methyl-1H- pyrrolo[3,2-b]pyridin- 5-yl]-6-isopropyl- pyridin-2-yl}-methyl- amine | 421.47 | 2.38 |
| 340 | 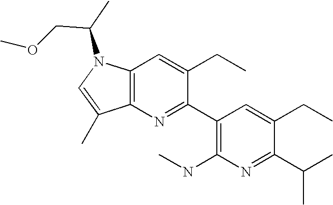 | {5-Ethyl-3-[6-ethyl-1- ((R)-2-methoxy-1- methyl-ethyl)-3- methyl-1H-pyrrolo[3,2- b]pyridin-5-yl]-6- isopropyl-pyridin-2- yl}-methyl-amine | 409.49 | 2.29 |
| 341 | 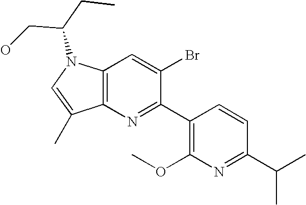 | (S)-2-[6-Bromo-5-(6- isopropyl-2-methoxy- pyridin-3-yl)-3-methyl- pyrrolo[3,2-b]pyridin- 1-yl]-butan-1-ol | 431.35/ 433.35 | 2.62 |
| 342 | 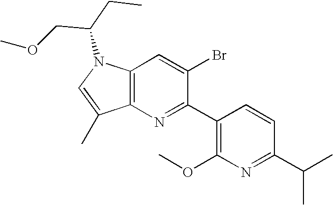 | 6-Bromo-5-(6- isopropyl-2-methoxy- pyridin-3-yl)-1-((S)-1- methoxymethyl- propyl)-3-methyl-1H- pyrrolo[3,2-b]pyridine | 445.38/ 447.37 | 2.80 |
| 343 | 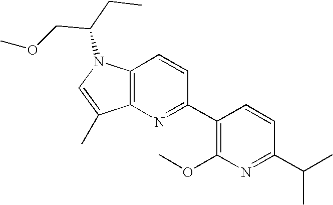 | 5-(6-Isopropyl-2- methoxy-pyridin-3-yl)- 1-((S)-1-methoxymethyl- propyl)-3-methyl-1H- pyrrolo[3,2-b]pyridine | 367.15 | 2.22 |
| 344 | 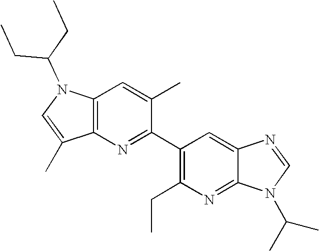 | 5-Ethyl-6-[1-(1-ethyl- propyl)-3,6-dimethyl- 1H-pyrrolo[3,2- b]pyridin-5- yl]-3-isopropyl-3H- imidazo[4,5-b]pyridine | 404.33 | 1.44 |
| 345 | 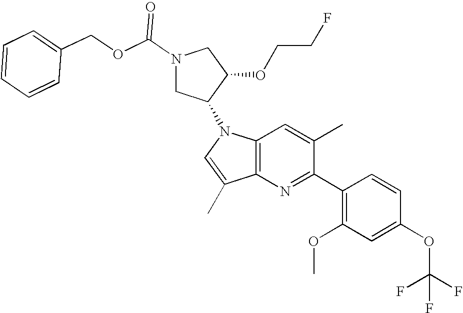 | (3S,4R)-3-(2-Fluoro- ethoxy)-4-[5-(2- methoxy-4- trifluoromethoxy- phenyl)-3,6-dimethyl- pyrrolo[3,2-b]pyridin- 1-yl]-pyrrolidine-1- carboxylic acid benzyl ester | 602.06 | 1.47 |
| 346 | 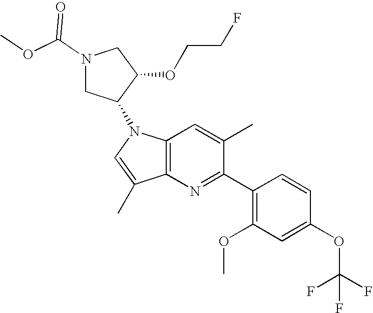 | (3S,4R)-3-(2-Fluoro- ethoxy)-4-[5-(2- methoxy-4- trifluoromethoxy- phenyl)-3,6-dimethyl- pyrrolo[3,2-b]pyridin- 1-yl]-pyrrolidine-1- carboxylic acid methyl ester | 526.02 | 1.35 |
| 347 | 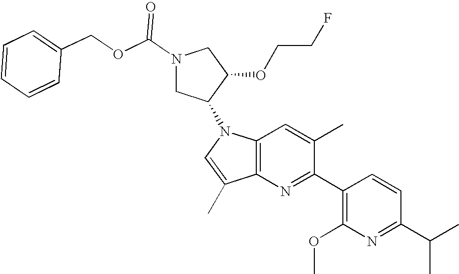 | (3S,4R)-3-(2-Fluoro- ethoxy)-4-[5-(6- isopropyl-2-methoxy- pyridin-3-yl)-3,6- dimethyl-pyrrolo[3,2-b]pyridin-1-yl]- pyrrolidine-1- carboxylic acid benzyl ester | 561.11 | 1.49 |
| 348 | 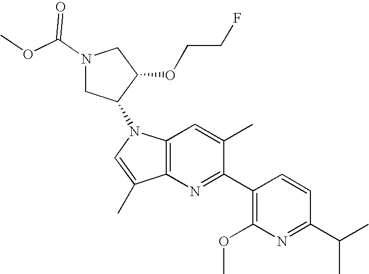 | (3S,4R)-3-(2-Fluoro- ethoxy)-4-[5-(6- isopropyl-2-methoxy- pyridin-3-yl)-3,6- dimethyl-pyrrolo[3,2-b]pyridin-1-yl]- pyrrolidine-1- carboxylic acid methyl ester | 485.13 | 1.39 |
| 349 | 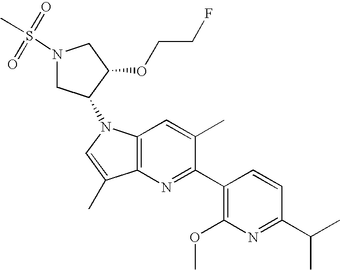 | 1-[(3R,4S)-4-(2-Fluoro- ethoxy)-1- methanesulfonyl- pyrrolidin-3-yl]-5-(6- isopropyl-2-methoxy- pyridin-3-yl)-3,6- dimethyl-1H- pyrrolo[3,2-b]pyridine | 505.10 | 1.35 |
| 350 | 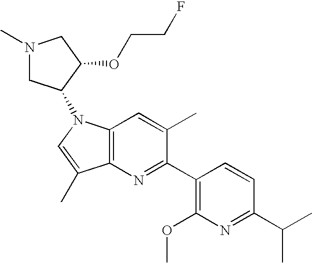 | 1-[(3R,4S)-4-(2-Fluoro- ethoxy)-1-methyl- pyrrolidin-3-yl]-5-(6- isopropyl-2-methoxy- pyridin-3-yl)-3,6- dimethyl-1H- pyrrolo[3,2-b]pyridine | 441.14 | 1.24 |
| 351 | 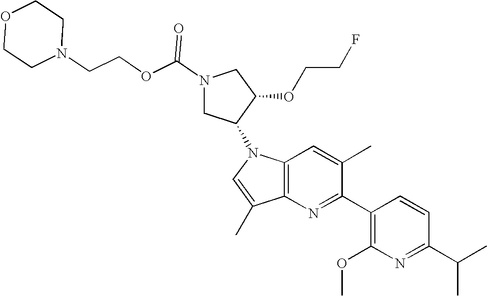 | (3S,4R)-3-(2-Fluoro- ethoxy)-4-[5-(6- isopropyl-2-methoxy- pyridin-3-yl)- 3,6-dimethyl- pyrrolo[3,2-b]pyridin- 1-yl]-pyrrolidine-1- carboxylic acid 2- morpholin-4-yl- ethyl ester | 584 | 1.38 |
| 352 | 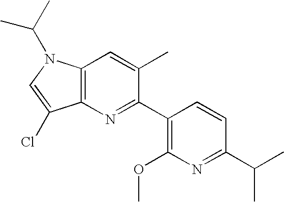 | 3-Chloro-1-isopropyl- 5-(6-isopropyl-2- methoxy-pyridin-3-yl)- 6-methyl-1H- pyrrolo[3,2-b]pyridine | 358.09 360.08 | 1.60 |
| 353 | 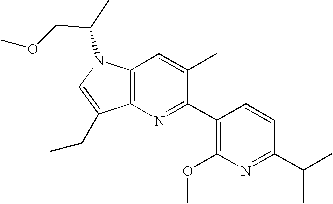 | 3-Ethyl-5-(6-isopropyl- 2-methoxy-pyridin-3- yl)-1-((S)-2-methoxy-1- methyl-ethyl)-6- methyl-1H-pyrrolo[3,2- b]pyridine | 382.19 | 1.44 |
| 354 | 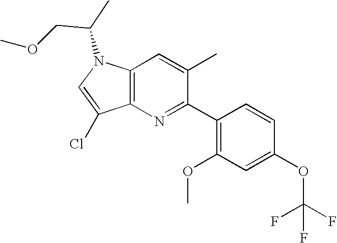 | 3-Chloro-1-((S)-2- methoxy-1-methyl- ethyl)-5-(2-methoxy-4- trifluoromethoxy- phenyl)-6-methyl-1H- pyrrolo[3,2-b]pyridine | 429.02 431.02 | 1.53 |
| 355 | 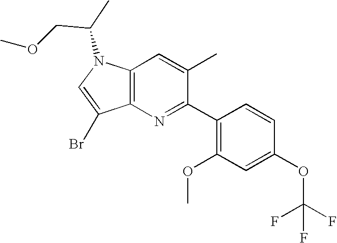 | 3-Bromo-1-((S)-2- methoxy-1-methyl- ethyl)-5-(2-methoxy-4- trifluoromethoxy- phenyl)-6-methyl-1H- pyrrolo[3,2-b]pyridine | 472.96 472.96 | 1.54 |
| 356 | 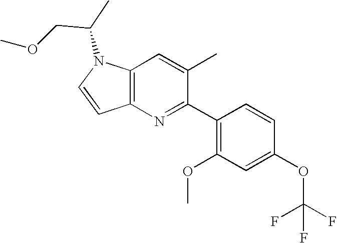 | 1-((S)-2-Methoxy-1- methyl-ethyl)-5-(2- methoxy-4- trifluoromethoxy- phenyl)-6-methyl-1H- pyrrolo[3,2-b]pyridine | 395.04 | 1.34 |
| 357 | 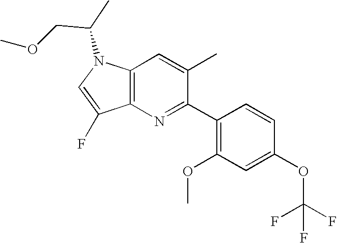 | 3-Fluoro-1-((S)-2- methoxy-1-methyl- ethyl)-5-(2-methoxy-4- trifluoromethoxy- phenyl)-6-methyl-1H- pyrrolo[3,2-b]pyridine | 413.02 | 1.49 |
| 358 | 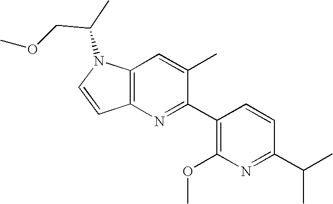 | 5-(6-Isopropyl-2- methoxy-pyridin-3-yl)- 1-((S)-2-methoxy-1- methyl-ethyl)-6- methyl-1H-pyrrolo[3,2- b]pyridine | 354.15 | 1.40 |
| 359 | 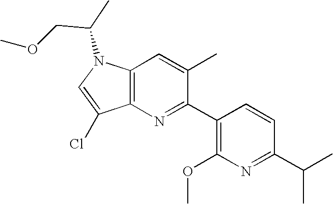 | 3-Chloro-5-(6- isopropyl-2-methoxy- pyridin-3-yl)-1-((S)-2- methoxy-1-methyl- ethyl)-6-methyl-1H- pyrrolo[3,2-b]pyridine | 388.10 390.10 | 1.56 |
| 360 | 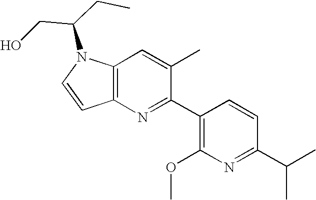 | (R)-2-[5-(6-Isopropyl- 2-methoxy-pyridin-3- yl)-6-methyl- pyrrolo[3,2-b]pyridin- 1-yl]-butan-1-ol | 354.12 | 1.39 |
| 361 | 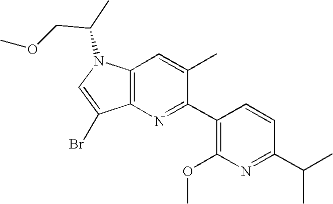 | 3-Bromo-5-(6- isopropyl-2-methoxy- pyridin-3-yl)-1-((S)-2- methoxy-1-methyl- ethyl)-6-methyl-1H- prrolo[3,2-b]pyridine | 432 434 | 1.59 |
| 362 | 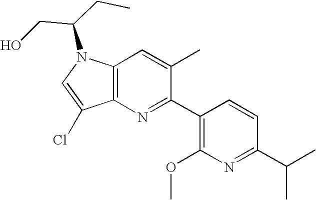 | (R)-2-[3-Chloro-5-(6- isopropyl-2-methoxy- pyridin-3-yl)-6-methyl- pyrrolo[3,2-b]pyridin- 1-yl]-butan-1-ol | 388.10 390.10 | 1.56 |
| 363 | 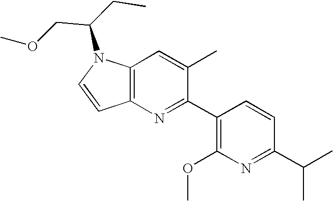 | 5-(6-Isopropyl-2- methoxy-pyridin-3-yl)- 1-((R)-1- methoxymethyl-propyl)- 6-methyl-1H- pyrrolo[3,2-b]pyridine | 368.26 | 1.44 |
| 364 | 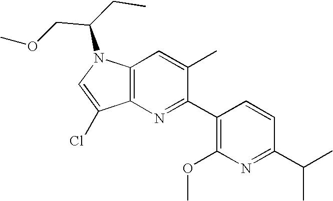 | 3-Chloro-5-(6- isopropyl-2-methoxy- pyridin-3-yl)-1-((R)-1- methoxymethyl- propyl)-6-methyl-1H- pyrrolo[3,2-b]pyridine | 402 404 | 1.62 |
| 365 | 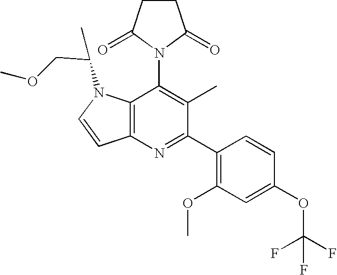 | 1-[1-((S)-2-Methoxy-1- methyl-ethyl)-5-(2- methoxy-4- trifluoromethoxy- phenyl)-6-methyl-1H- pyrrolo[3,2-b]pyridin- 7-yl]-pyrrolidine-2,5- dione | 492.09 | 1.38 |
| 366 | 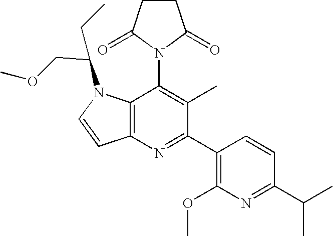 | 1-[5-(6-Isopropyl-2- methoxy-pyridin-3-yl)- 1-((R)-1- methoxymethyl- propyl)-6-methyl-1H- pyrrolo[3,2-b]pyridin- 7-yl]-pyrrolidine-2,5- dione | 465.21 | 1.48 |
| 367 | 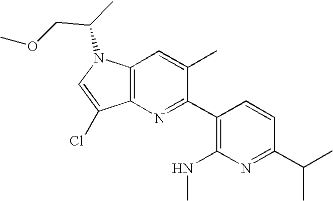 | {3-[3-Chloro-1-((S)-2- methoxy-1-methyl- ethyl)-6-methyl-1H- pyrrolo[3,2-b]pyridin- 5-yl]-6-isopropyl- pyridin-2- yl}-methyl-amine | 387 389 | 1.32 |
| 368 | 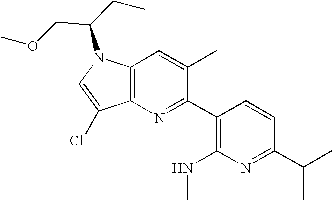 | {3-[3-Chloro-1-((R)-1- methoxymethyl- propyl)-6-methyl-1H- pyrrolo[3,2-b]pyridin-5-yl]-6- isopropyl-pyridin-2- yl}-methyl-amine | 401.21 403.20 | 1.35 |
| 369 | 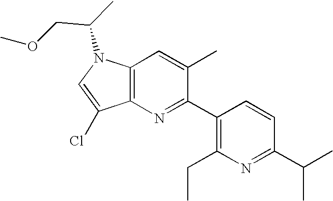 | 3-Chloro-5-(2-ethyl-6- isopropyl-pyridin-3-yl)- 1-((S)-2-methoxy-1- methyl-ethyl)-6- methyl-1H-pyrrolo[3,2- b]pyridine | 386.19 388.19 | 1.36 |
| 370 | 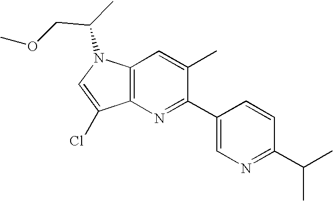 | 3-Chloro-5-(6- isopropyl-pyridin-3-yl)- 1-((S)-2-methoxy-1- methyl-ethyl)-6- methyl-1H-pyrrolo[3,2- b]pyridine | 358.14 360.12 | 1.45 |
| 371 | 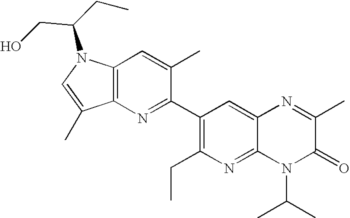 | 6-Ethyl-7-[1-((R)-1- hydroxymethyl- propyl)-3,6-dimethyl- 1H-pyrrolo[3,2- b]pyridin-5-yl]-4- isopropyl-2-methyl-4H- pyrido[2,3-b]pyrazin- 3-one | 448.23 | 1.40 |
| 372 | 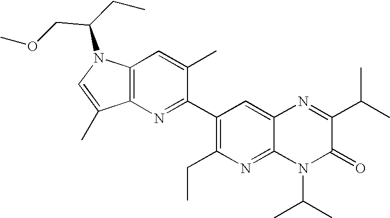 | 6-Ethyl-2,4- diisopropyl-7-[1-((R)- 1-methoxymethyl- propyl)-3,6-dimethyl- 1H-pyrrolo[3,2- b]pyridin-5-yl]-4H- pyrido[2,3-b]pyrazin- 3-one | 490.31 | 1.53 |
| 373 | 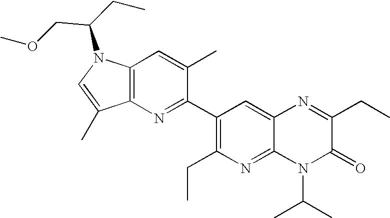 | 2,6-Diethyl-4- isopropyl-7-[1-((R)-1- methoxymethyl- propyl)-3,6-dimethyl- 1H-pyrrolo[3,2- b]pyridin-5-yl]-4H- pyrido[2,3-b]pyrazin- 3-one | 476.29 | 1.48 |
| 374 | 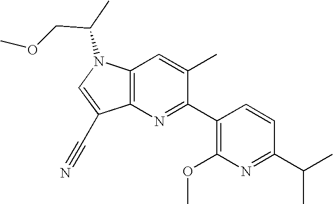 | 5-(6-Isopropyl-2- methoxy-pyridin-3-yl)- 1-((S)-2-methoxy-1- methyl-ethyl)-6- methyl-1H-pyrrolo[3,2- b]pyridine-3- carbonitrile | 379.19 | 1.59 |
| 375 | 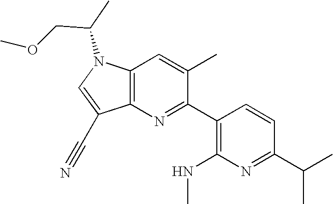 | 5-(6-Isopropyl-2- methylamino-pyridin-3- yl)-1-((S)-2-methoxy-1- methyl-ethyl)-6- methyl-1H-pyrrolo[3,2- b]pyridine-3- carbonitrile | 378.20 | 1.24 |
| 376 | 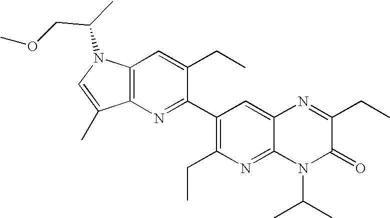 | 6-Ethyl-7-[6-ethyl-1- ((S)-2-methoxy-1- methyl-ethyl)-3- methyl-1H-pyrrolo[3,2- b]pyridin-5-yl]-4- isopropyl-2-methyl-4H- pyrido[2,3-b]pyrazin- 3-one | 462.27 | 1.46 |
| 377 | 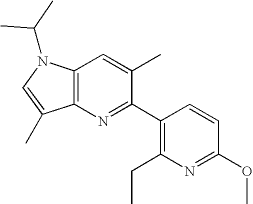 | 5-(2-Ethyl-6-methoxy- pyridin-3-yl)-1- isopropyl-3,6-dimethyl- 1H-pyrrolo[3,2-b]pyridine | 324.40 | 1.40 |
| 378 | 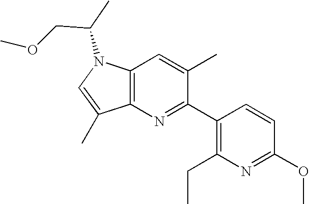 | 5-(2-Ethyl-6-methoxy- pyridin-3-yl)-1-((S)-2- methoxy-1-methyl- ethyl)-3,6-dimethyl- 1H-pyrrolo[3,2-b]pyridine | 354.36 | 1.39 |
| 379 | 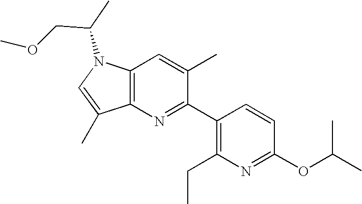 | 5-(2-Ethyl-6- isopropoxy-pyridin-3- yl)-1-((S)-2-methoxy-1- methyl-ethyl)-3,6- dimethyl-1H- pyrrolo[3,2-b]pyridine | 382.42 | 1.52 |
| 380 | 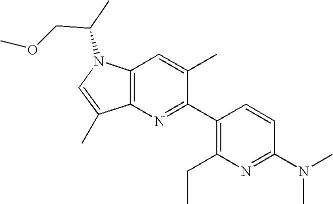 | {6-Ethyl-5-[1-((S)-2- methoxy-1-methyl- ethyl)-3,6-dimethyl- 1H-pyrrolo[3,2- b]pyridin-5-yl]-pyridin- 2-yl}-dimethyl-amine | 367.43 | 1.32 |
| 381 | 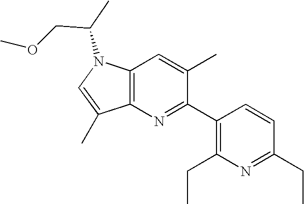 | 5-(2,6-Diethyl-pyridin- 3-yl)-1-((S)-2-methoxy- 1-methyl-ethyl)-3,6- dimethyl-1H- pyrrolo[3,2-b]pyridine | 352.45 | 1.32 |
| 382 | 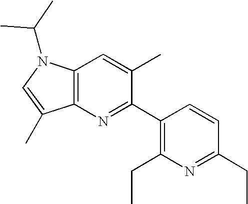 | 5-(2,6-Diethyl-pyridin- 3-yl)-1-isopropyl-3,6- dimethyl-1H- pyrrolo[3,2-b]pyridine | 322.45 | 1.36 |
| 383 | 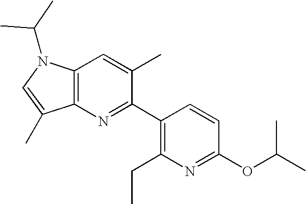 | 5-(2-Ethyl-6- isopropoxy-pyridin-3- yl)-1-isopropyl-3,6- dimethyl-1H- pyrrolo[3,2-b]pyridine | 352.45 | 1.54 |
| 384 | 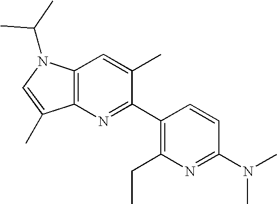 | [6-Ethyl-5-(1- isopropyl-3,6-dimethyl- 1H-pyrrolo[3,2- b]pyridin-5-yl)-pyridin- 2-yl]-dimethyl-amine | 337.44 | 1.31 |
| 385 | 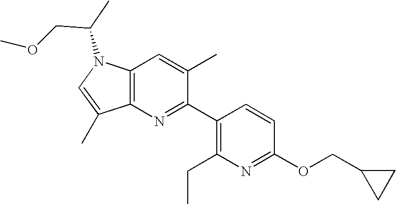 | 5-(6-Cyclopropylmethoxy-2- ethyl-pyridin-3-yl)-1- ((S)-2-methoxy-1- methyl-ethyl)-3,6- dimethyl-1H- pyrrolo[3,2-b]pyridine | 394.16 | 1.43 |
| 386 | 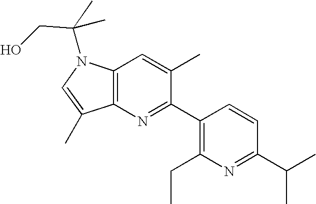 | 2-[5-(6-Isopropyl-2- methoxy-pyridin-3-yl)- 3,6-dimethyl- pyrrolo[3,2-b]pyridin- 1-yl]-2-methyl- propan-1-ol | 368.44 | 1.56 |
| 387 | 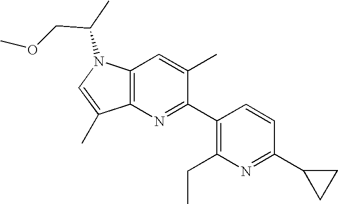 | 5-(6-Cyclopropyl-2- ethyl-pyridin-3-yl)-1- ((S)-2-methoxy-1- methyl-ethyl)-3,6- dimethyl-1H- pyrrolo[3,2-b]pyridine | 364.45 | 1.42 |
| 388 | 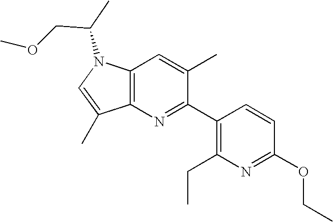 | 5-(6-Ethoxy-2-ethyl- pyridin-3-yl)-1-((S)-2- methoxy-1-methyl- ethyl)-3,6-dimethyl- 1H-pyrrolo[3,2-b]pyridine | 368.48 | 1.50 |
| 389 | 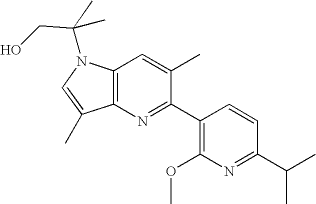 | 5-(6-Isopropyl-2- methoxy-pyridin-3-yl)- 1-(2-methoxy-1,1- dimethyl-ethyl)-3,6- dimethyl-1H- pyrrolo[3,2-b]pyridine | 382.53 | 1.60 |
| 390 | 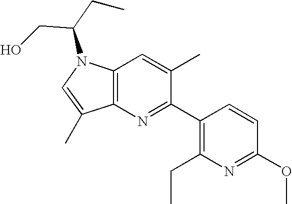 | (R)-2-[5-(2-Ethyl-6- methoxy-pyridin-3-yl) 3,6-dimethyl- pyrrolo[3,2-b]pyridin- 1-yl]-butan-1-ol | 354.46 | 1.49 |
| 391 | 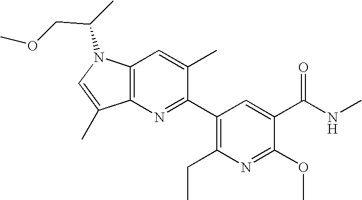 | 6-Ethyl-2-methoxy-5- [1-((S)-2-methoxy-1- methyl-ethyl)-3,6- dimethyl-1H- pyrrolo[3,2-b]pyridin- 5-yl]-N-methyl- nicotinamide | 411.41 | 1.44 |
| 392 | 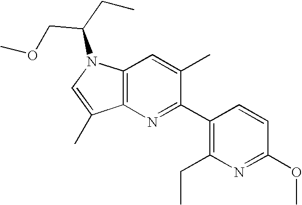 | 5-(2-Ethyl-6-methoxy- pyridin-3-yl)-1-((R)-1- methoxymethyl- propyl)-3,6-dimethyl- 1H-pyrrolo[3,2-b]pyridine | 368.47 | 1.50 |
| 393 | 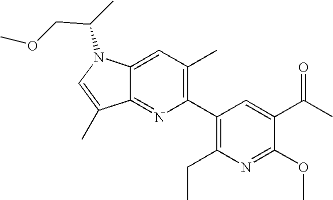 | 1-{6-Ethyl-2-methoxy- 5-[1-((S)-2-methoxy-1- methyl-ethyl)-3,6- dimethyl-1H- pyrrolo[3,2-b]pyridin- 5-yl]-pyridin-3-yl}- ethanone | 396.43 | 1.43 |
| 394 | 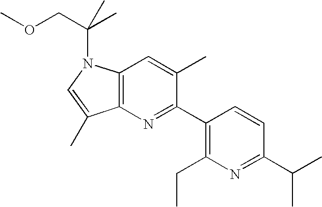 | 5-(2-Ethyl-6-isopropyl- pyridin-3-yl)-1-(2- methoxy-1,1-dimethyl- ethyl)-3,6-dimethyl- 1H-pyrrolo[3,2-b]pyridine | 380.49 | 1.41 |
| 395 | 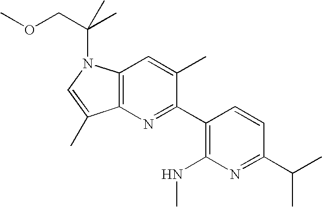 | {6-Isopropyl-3-[1-(2- methoxy-1,1-dimethyl- ethyl)-3,6-dimethyl- 1H-pyrrolo[3,2- b]pyridin-5-yl]-pyridin- 2-yl}-methyl-amine | 381.49 | 1.37 |
| 396 | 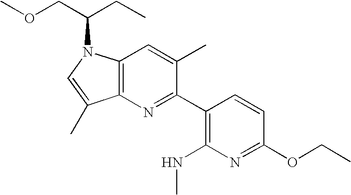 | 5-(6-Ethoxy-2-ethyl- pyridin-3-yl)-1-((R)-1- methoxymethyl- propyl)-3,6-dimethyl- 1H-pyrrolo[3,2-b]pyridine | 382.48 | 1.49 |
| 397 | 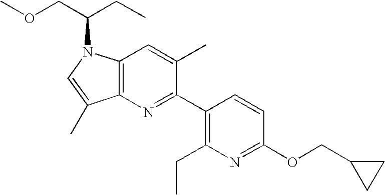 | 5-(6- Cyclopropylmethoxy-2- ethyl-pyridin-3-yl)-1- ((R)-1-methoxymethyl- propyl)-3,6-dimethyl- 1H-pyrrolo[3,2-b]pyridine | 408.46 | 1.56 |
| 398 | 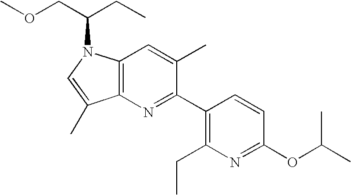 | 5-(2-Ethyl-6- isopropoxy-pyridin-3- yl)-1-((R)-1- methoxymethyl- propyl)-3,6-dimethyl- 1H-pyrrolo[3,2-b]pyridine | 396.51 | 1.54 |
| 399 | 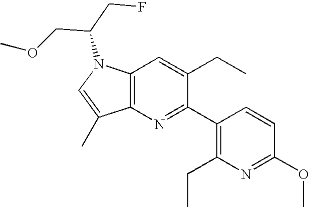 | 6-Ethyl-5-(2-ethyl-6- methoxy-pyridin-3-yl)- 1-((R)-2-fluoro-1- methoxymethyl-ethyl)- 3-methyl-1H- pyrrolo[3,2-b]pyridine | 386.44 | 1.44 |
| 400 | 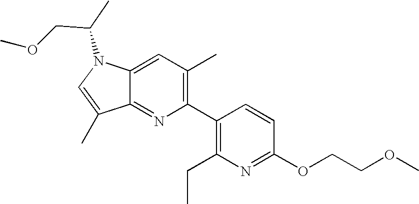 | 5-[2-Ethyl-6-(2- methoxy-ethoxy)- pyridin-3-yl]-1-((S)-2- ethyl)-3,6-dimethyl- methoxy-1-methyl- ethyl)-3,6-dimethyl- 1H-pyrrolo[3,2-b]pyridine | 398.34 | 1.41 |
| 401 | 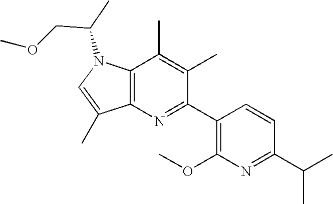 | 5-(6-Isopropyl-2- methoxy-pyridin-3-yl)- 1-((S)-2-methoxy-1- methyl-ethyl)-3,6,7- trimethyl-1H- pyrrolo[3,2-b]pyridine | 382.3 | |
| 402 | 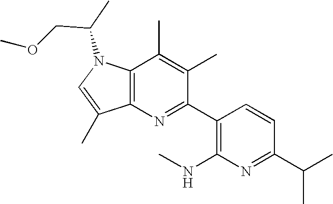 | {6-Isopropyl-3-[1-((S)- 2-methoxy-1-methyl- ethyl)-3,6,7-trimethyl- 1H-pyrrolo[3,2- b]pyridin-5-yl]-pyridin- 2-yl}-methyl-amine | 381.2 | |
| 403 | 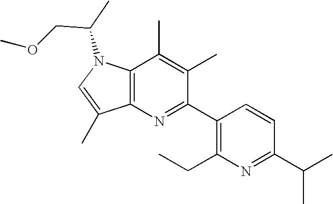 | 5-(2-Ethyl-6-isopropyl- pyridin-3-yl)-1-((S)-2- methoxy-1-methyl- ethyl)- 3,6,7-trimethyl-1H- pyrrolo[3,2-b]pyridine | 380.2 | |
| 404 | 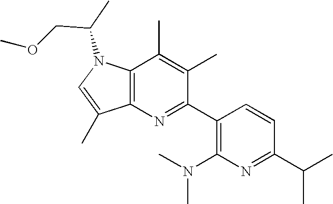 | {6-Isopropyl-3-[1-((S)- 2-methoxy-1-methyl- ethyl)-3,6,7-trimethyl- 1H-pyrrolo[3,2- b]pyridin-5-yl]-pyridin- 2-yl}-dimethyl-amine | 395.3 | |
| 405 | 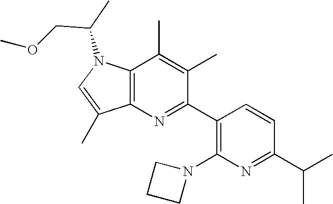 | 5-(2-Azetidin-1-yl-6- isopropyl-pyridin-3-yl)- 1-((S)-2-methoxy-1- methyl-ethyl)-3,6,7- trimethyl-1H- pyrrolo[3,2-b]pyridine | 407.6 | |
| 406 | 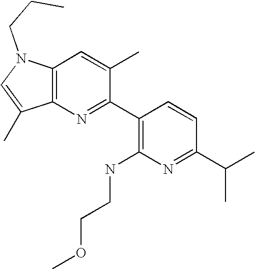 | [3-(3,6-Dimethyl-1- propyl-1H-pyrrolo[3,2- b]pyridin-5-yl)-6- isopropyl-pyridin-2-yl]- (2-methoxy-ethyl)-amine | 381.3 | 1.95 |
| 407 | 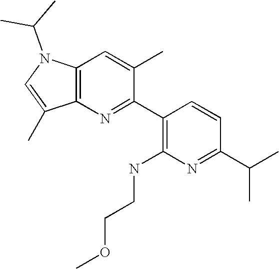 | 6-Isopropyl-3-(1- isopropyl-3,6-dimethyl- 1H-pyrrolo[3,2- b]pyridin-5-yl)-pyridin- 2-yl]-(2-methoxy- ethyl)-amine | 381.3 | 1.88 |
| 408 | 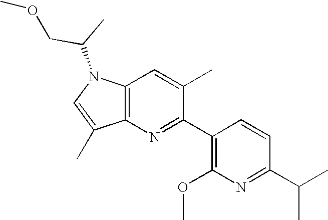 | 5-(6-Isopropyl-2- methoxy-pyridin-3-yl)- 1-((S)-2-methoxy-1- methyl-ethyl)-3,6- dimethyl-1H- pyrrolo[3,2-b]pyridine | 368.2 | 2.28 |
| 409 | 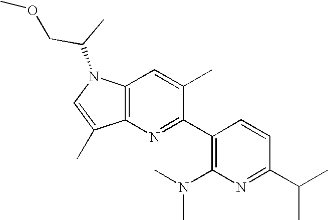 | {6-Isopropyl-3-[1-((S)- 2-methoxy-1-methyl- ethyl)-3,6-dimethyl- 1H-pyrrolo[3,2-b]pyridin-5-yl]- pyridin-2-yl}-dimethyl- amine | 381.3 | 2.00 |
| 410 | 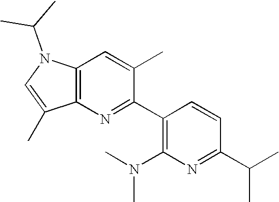 | [6-Isopropyl-3-(1- isopropyl-3,6-dimethyl- 1H-pyrrolo[3,2- b]pyridin-5-yl)-pyridin- 2-yl]-dimethyl-amine | 351.4 | 2.07 |
| 411 | 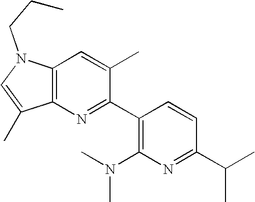 | [3-(3,6-Dimethyl-1- propyl-1H-pyrrolo[3,2- b]pyridin-5-yl)-6- isopropyl-pyridin-2-yl]- dimethyl-amine | 351.4 | 2.12 |
| 412 | 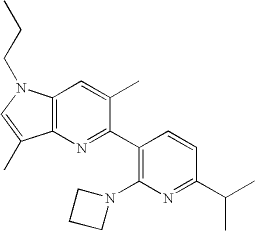 | 5-(2-Azetidin-1-yl-6- isopropyl-pyridin-3-yl)- 3,6-dimethyl-1-propyl- 1H-pyrrolo[3,2- b]pyridine | 363.4 | 1.87 |
| 413 | 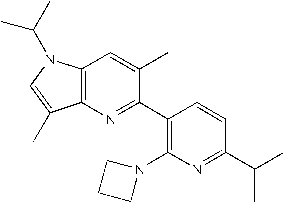 | 5-(2-Azetidin-1-yl-6- isopropyl-pyridin-3-yl)- 1-isopropyl-3,6- dimethyl-1H- pyrrolo[3,2-b]pyridine | 363.4 | 1.82 |
| 414 | 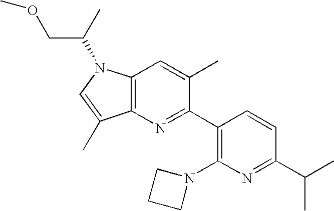 | 5-(2-Azetidin-1-yl-6- isopropyl-pyridin-3-yl)- 1-((S)-2-methoxy-1- methyl-ethyl)-3,6- dimethyl-1H- pyrrolo[3,2-b]pyridine | 393.4 | 1.75 |
| 415 | 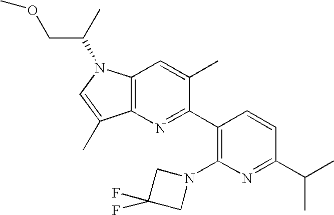 | 5-[2-(3,3-Difluoro- azetidin-1-yl)-6- isopropyl-pyridin-3-yl]- 1-((S)-2-methoxy-1- methyl-ethyl)-3,6- dimethyl-1H- pyrrolo[3,2-b]pyridine | 429.2 | 2.38 |
| 416 | 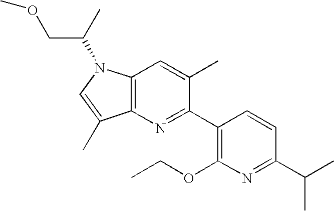 | 5-(2-Ethoxy-6- isopropyl-pyridin-3-yl)- 1-((S)-2-methoxy-1- methyl-ethyl)-3,6- dimethyl-1H- pyrrolo[3,2-b]pyridine | 382.1 | 2.52 |
| 417 | 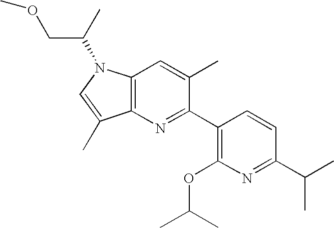 | 5-(2-Isopropoxy-6- isopropyl-pyridin-3-yl)- 1-((S)-2-methoxy-1- methyl-ethyl)-3,6- dimethyl-1H- pyrrolo[3,2-b]pyridine | 396.2 | 2.57 |
| 418 | 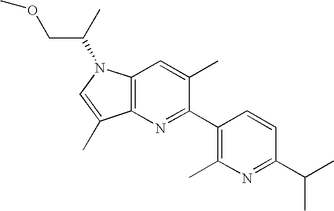 | 5-(6-Isopropyl-2- methyl-pyridin-3-yl)-1- ((S)-2-methoxy-1- methyl-ethyl)-3,6- dimethyl-1H- pyrrolo[3,2-b]pyridine | 352.1 | 1.60 |
| 419 | 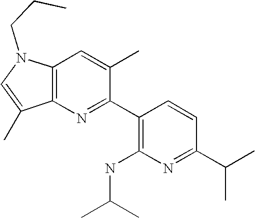 | [3-(3,6-Dimethyl-1- propyl-1H-pyrrolo[3,2- b]pyridin-5-yl)-6- isopropyl-pyridin-2-yl]- isopropyl-amine | 365.2 | 2.13 |
| 420 | 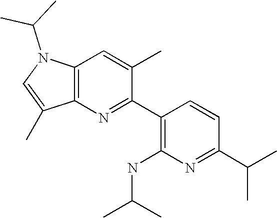 | Isopropyl-[6-isopropyl- 3-(1-isopropyl-3,6- dimethyl-1H- pyrrolo[3,2-b]pyridin- 5-yl)-pyridin-2-yl]- amine | 365.2 | 2.1 |
| 421 | 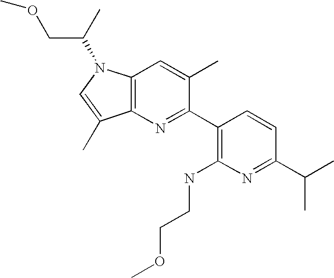 | {6-Isopropyl-3-[1-(2- methoxy-1-methyl- ethyl)-3,6-dimethyl- 1H-pyrrolo[3, 2-b]pyridin-5-yl]- pyridin-2-yl}-(2- ethoxy-ethyl)-amine | 411.2 | 1.83 |
| 422 | 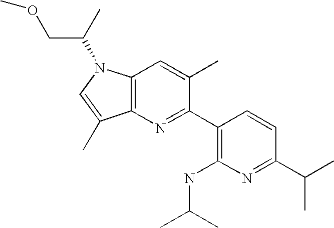 | Isopropyl-{6-isopropyl- 3-[1-((S)-2-methoxy-1- methyl-ethyl)-3,6- dimethyl-1H- pyrrolo[3,2-b]pyridin- 5-yl]-pyridin-2-yl}- amine | 395.2 | 2.02 |
| 423 | 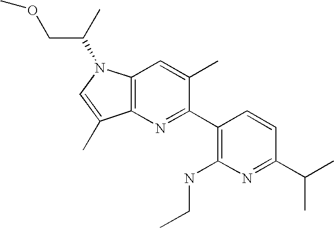 | Ethyl-{6-isopropyl-3- [1-((S)-2-methoxy-1- methyl-ethyl)-3,6- dimethyl-1H- pyrrolo[3,2-b]pyridin- 5-yl]-pyridin-2-yl}- amine | 381.2 | 1.77 |
| 424 | 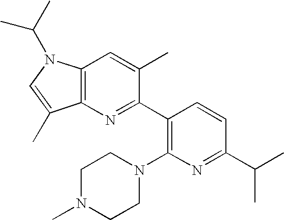 | 1-Isopropyl-5-[6- isopropyl-2-(4-methyl- piperazin-1-yl)-pyridin- 3-yl]-3,6-dimethyl-1H- pyrrolo[3,2-b]pyridine | 406.3 | 1.67 |
| 425 | 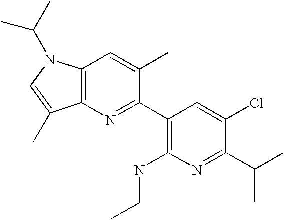 | [5-Chloro-6-isopropyl- 3-(1-isopropyl-3,6- dimethyl-1H- pyrrolo[3,2-b]pyridin- 5-yl)-pyridin-2-yl]- ethyl-amine | 385.2 | 2.78 |
| 426 | 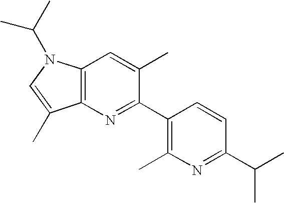 | 1-Isopropyl-5-(6- isopropyl-2-methyl- pyridin-3-yl)-3,6- dimethyl-1H- pyrrolo[3,2-b]pyridine | 322.2 | 1.68 |
| 427 | 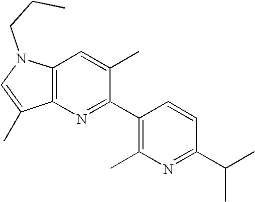 | 5-(6-Isopropyl-2- methyl-pyridin-3-yl)- 3,6-dimethyl-1-propyl- 1H-pyrrolo[3,2-b]pyridine | 322.2 | 1.72 |
| 428 | 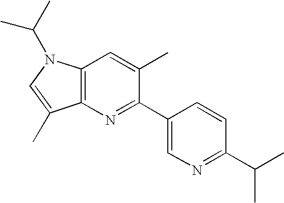 | 1-Isopropyl-5-(6- isopropyl-pyridin-3-yl)- 3,6-dimethyl-1H- pyrrolo[3,2-b]pyridine | 308.2 | 1.85 |
| 429 | 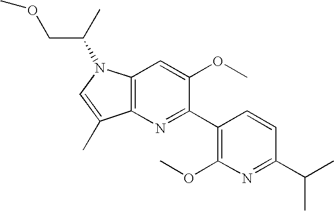 | 5-(6-Isopropyl-2- methoxy-pyridin-3-yl)- 6-methoxy-1-((S)-2- methoxy-1-methyl- ethyl)-3-methyl-1H- pyrrolo[3,2-b]pyridine | 384.2 | 2.40 |
| 430 | 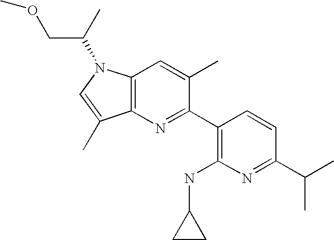 | Cyclopropyl-{6- isopropyl-3-[1-((S)-2- methoxy-1-methyl- ethyl)-3,6-dimethyl- 1H-pyrrolo[3,2- b]pyridin-5-yl]-pyridin- 2-yl}-amine | 393.3 | 1.7 |
| 431 | 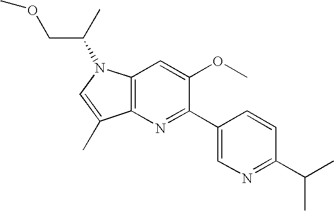 | 5-(6-Isopropyl-pyridin- 3-yl)-6-methoxy-1-((S)- 2-methoxy-1-methyl- ethyl)-3-methyl-1H- pyrrolo[3,2-b]pyridine | 354.2 | 2.02 |
| 432 | 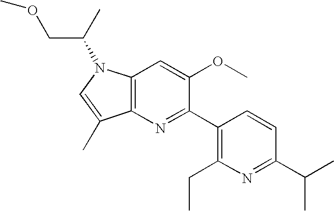 | 5-(2-Ethyl-6-isopropyl- pyridin-3-yl)-6- methoxy-1-((S)-2- methoxy-1-methyl- ethyl)-3-methyl-1H- pyrrolo[3,2-b]pyridine | 382.3 | 1.94 |
| 433 | 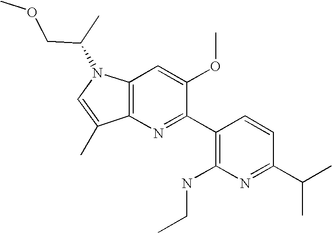 | Ethyl-{6-isopropyl-3- [6-methoxy-1-((S)-2- methoxy-1-methyl- ethyl)-3-methyl-1H- pyrrolo[3,2-b]pyridin- 5-yl]-pyridin-2-yl}- amine | 397.3 | 2.14 |
| 434 | 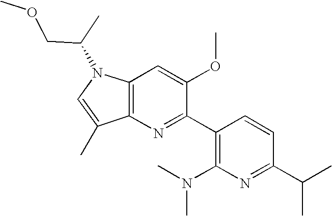 | {6-Isopropyl-3-[6- methoxy-1-((S)-2- methoxy-1-methyl- ethyl)-3-methyl-1H- pyrrolo[3,2-b]pyridin- 5-yl]-pyridin-2-yl}- dimethyl-amine | 397.2 | 1.97 |
| 435 | 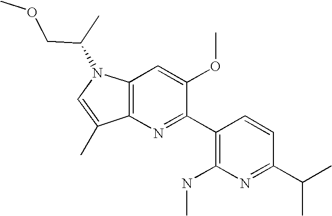 | {6-Isopropyl-3-[6- methoxy-1-((S)-2- methoxy-1-methyl- ethyl)-3-methyl-1H- pyrrolo[3,2-b]pyridin- 5-yl]-pyridin-2-yl}- methyl-amine | 383.3 | 1.93 |
| 436 | 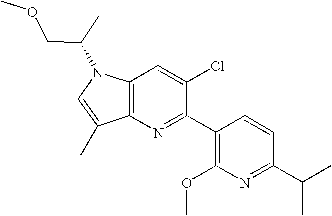 | 6-Chloro-5-(6- isopropyl-2-methoxy- pyridin-3-yl)-1-((S)-2- methoxy-1-methyl- ethyl)-3-methyl-1H- pyrrolo[3,2-b]pyridine | 388.2 | 3.15 |
| 437 | 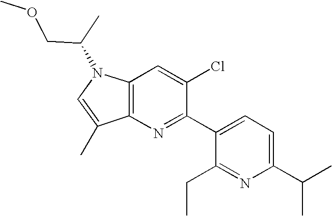 | 6-Chloro-5-(2-ethyl-6- isopropyl-pyridin-3-yl)- 1-((S)-2-methoxy-1- methyl-ethyl)-3- methyl-1H-pyrrolo[3,2- b]pyridine | 386.2 | 2.02 |
| 438 | 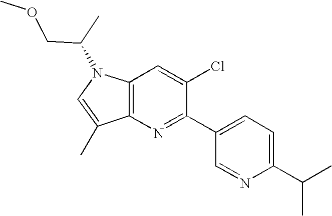 | 6-Chloro-5-(6- isopropyl-pyridin-3-yl)- 1-((S)-2-methoxy-1- methyl-ethyl)-3- methyl-1H-pyrrolo[3,2- b]pyridine | 358.2 | 2.15 |
| 439 | 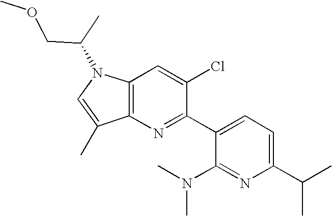 | {3-[6-Chloro-1-((S)-2- methoxy-1-methyl- ethyl)-3-methyl-1H- pyrrolo[3,2-b]pyridin- 5-yl]-6-isopropyl- pyridin-2-yl}-dimethyl- amine | 401.2 | 2.12 |
| 440 | 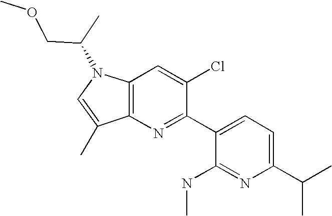 | {3-[6-Chloro-1-((S)-2- methoxy-1-methyl- ethyl)-3-methyl-1H- pyrrolo[3,2-b]pyridin- 5-yl]-6-isopropyl- pyridin-2-yl}-methyl- amine | 387.2 | 2.09 |
| 441 | 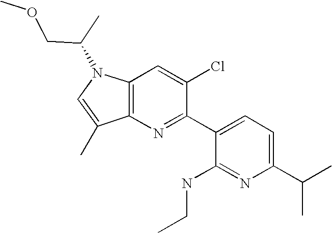 | {3-[6-Chloro-1-((S)-2- methoxy-1-methyl- ethyl)-3-methyl-1H- pyrrolo[3,2-b]pyridin- 5-yl]-6-isopropyl- pyridin-2-yl}-ethyl- amine | 401.2 | 2.22 |
| 442 | 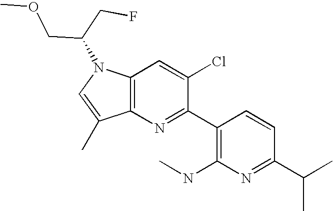 | {3-[6-Chloro-1-((R)-1- fluoromethyl-2- methoxy-ethyl)-3- methyl-1H-pyrrolo[3,2- b]pyridin-5-yl]-6- isopropyl-pyridin-2- yl}-methyl-amine | 405.2 | 2.09 |
| 443 | 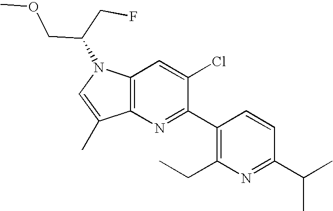 | 6-Chloro-5-(2-ethyl-6- isopropyl-pyridin-3-yl)- 1-((R)-1-fluoromethyl- 2-methoxy-ethyl)-3- methyl-1H-pyrrolo[3,2- b]pyridine | 448.12 | 2.34 |
| 444 | 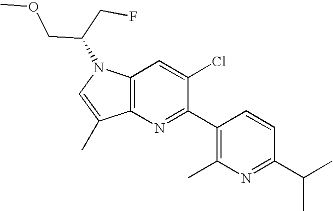 | 6-Chloro-1-((R)-1- fluoro-methyl-2- methoxy-ethyl)-5-(6- isopropyl-2-methyl- pyridin-3-yl)-3-methyl- 1H-pyrrolo[3,2-b]pyridine | 390.11 | 2.17 |
| 445 | 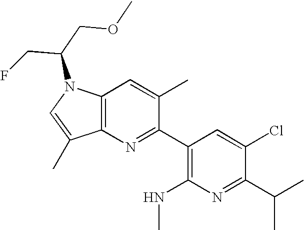 | {5-Chloro-3-[1-((R)-1- fluoromethyl-2- methoxy-ethyl)-3,6- dimethyl-1H- pyrrolo[3,2-b]pyridin- 5-yl]-6-isopropyl- pyridin-2-yl}-methyl- amine | 419.2 | 2.67 |
| 446 | 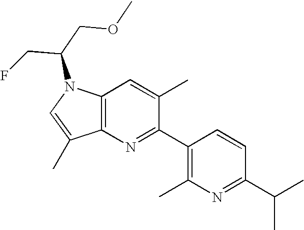 | 1-(R)-1-Fluoromethyl- 2-methoxy-ethyl)-5-(6- isopropyl-2-methyl- pyridin-3-yl)-3,6- dimethyl-1H- pyrrolo[3,2-b]pyridine | 370.20 | 1.83 |
| 447 | 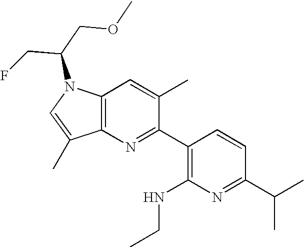 | Ethyl-{3-[1-((R)-1- fluoromethyl-2- methoxy-ethyl)-3,6- dimethyl-1H- pyrrolo[3,2-b]pyridin- 5-yl]-6-isopropyl- pyridin-2-yl}-amine | 399.20 | 2.03 |
| 448 | 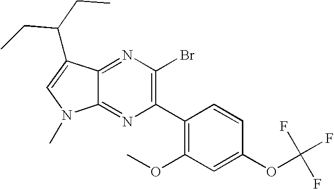 | 2-Bromo-7-(1-ethyl- propyl)-3-(2-methoxy- 4-trifluoromethoxy- phenyl)-5-methyl-5H- pyrrolo[2,3-b]pyrazine | 472.30 | |
| 449 | 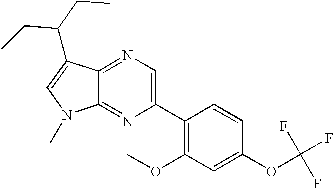 | 7-(1-Ethyl-propyl)-3- (2-methoxy-4- trifluoromethoxy- phenyl)-5-methyl-5H- pyrrolo[2,3-b]pyrazine | 394.3 | |
| 450 | 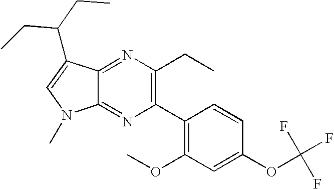 | 2-Ethyl-7-(1-ethyl- propyl)-3-(2-methoxy- 4-trifluoromethoxy- phenyl)-5-methyl-5H- pyrrolo[2,3-b]pyrazine | 422.5 | |
| 451 | 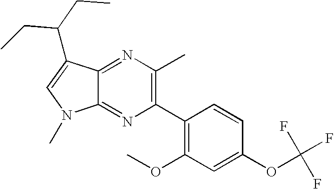 | 7-(1-Ethyl-propyl)-3- (2-methoxy-4- trifluoromethoxy- phenyl)-2,5-dimethyl- 5H-pyrrolo[2,3- b]pyrazine | 408.5 | |
| 452 | 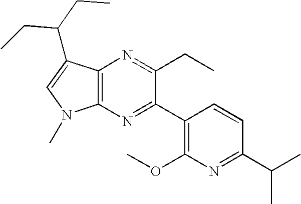 | 2-Ethyl-7-(1-ethyl- propyl)-3-(6-isopropyl- 2-methoxy-pyridin-3- yl)-5-methyl-5H- pyrrolo[2,3-b]pyrazine | 381.2 | |
| 453 | 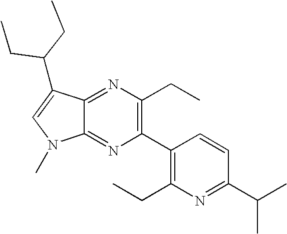 | 2-Ethyl-3-(2-ethyl-6- isopropyl-pyridin-3-yl)- 7-(1-ethyl-propyl)-5- methyl-5H-pyrrolo[2,3- b]pyrazine | 379.4 | |
| 454 | 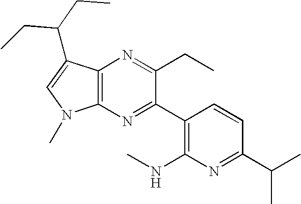 | {3-[2-Ethyl-7-(1-ethyl- propyl)-5-methyl-5H- pyrrolo[2,3-b]pyrazin- 3-yl]-6-isopropyl- pyridin-2-yl}-methyl- amine | 380.5 | |
| 455 | 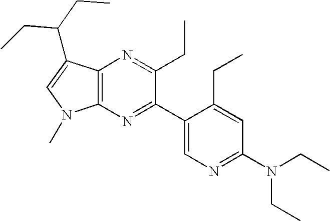 | Diethyl-{4-ethyl-5-[2- ethyl-7-(1-ethyl- propyl)-5-methyl-5H- pyrrolo[2,3-b]pyrazin- 3-yl]-pyridin-2-yl}- amine | 408.37 | 1.54 |
| 456 | 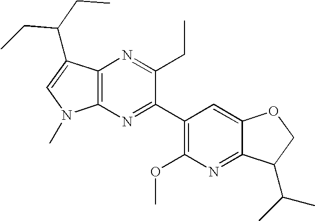 | 2-Ethyl-7-(1-ethyl- propyl)-3-(3-isopropyl- 5-methoxy-2,3- dihydro-furo[3,2- b]pyridin-6-yl)-5- methyl-5H-pyrrolo[2,3- b]pyrazine | 423.14 | 1.92 |
| 457 | 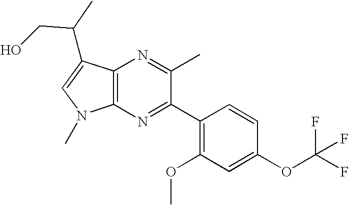 | 2-[3-(2-Methoxy-4- trifluoromethoxy- phenyl)-2,5-dimethyl- 5H-pyrrolo[2,3- b]pyrazin-7-yl]-propan- 1-ol | 396.05 | 1.70 |
| 458 | 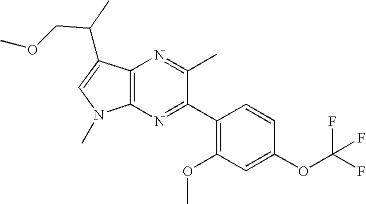 | 7-(2-Methoxy-1- methyl-ethyl)-3-(2- methoxy-4- trifluoromethoxy- phenyl)-2,5-dimethyl- 5H-pyrrolo[2,3- b]pyrazine | 410.03 | 1.77 |
| 459 | 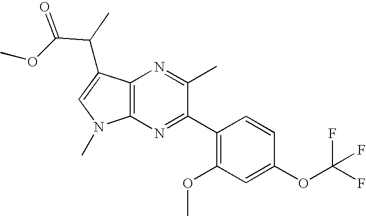 | 2-[3-(2-Methoxy-4- trifluoromethoxy- phenyl)-2,5-dimethyl- 5H-pyrrolo[2,3- b]pyrazin-7-yl]- propionic acid methyl ester | 424.00 | 1.74 |
| 460 | 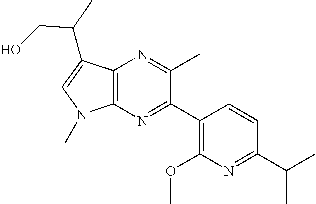 | 2-[3-(6-Isopropyl-2- methoxy-pyridin-3-yl)- 2,5-dimethyl-5H- pyrrolo[2,3- b-]pyrazin-7-yl]- propan-1-ol | 355.16 | 1.76 |
| 461 | 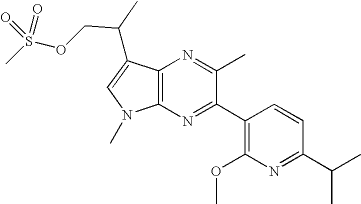 | Methanesulfonic acid 2-[3-(6-isopropyl-2- methoxy-pyridin-3-yl)- 2,5-dimethyl-5H- pyrrolo[2,3-b]pyrazin- 7-yl]-propyl ester | 433.07 | 1.77 |
| 462 | 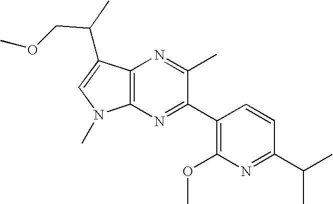 | 3-(6-Isopropyl-2- methoxy-pyridin-3-yl)- 7-(2-methoxy-1- methyl-ethyl)-2,5- dimethyl-5H- pyrrolo[2,3-b]pyrazine | 369.15 | 1.83 |
| 463 | 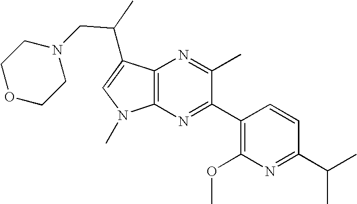 | 3-(6-Isopropyl-2- methoxy-pyridin-3-yl)- 2,5-dimethyl-7-(1- methyl-2-morpholin-4- yl-ethyl)-5H- pyrrolo[2,3-b]pyrazine | 424.13 | 1.53 |
| 464 | 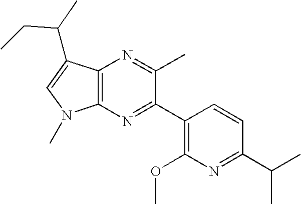 | 7-sec-Butyl-3-(6- isopropyl-2-methoxy- pyridin-3-yl)-2,5- dimethyl-5H- pyrrolo[2,3-b]pyrazine | 353.18 | 1.89 |
| 465 | 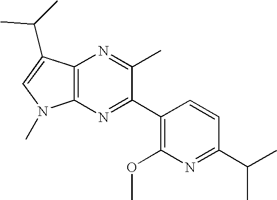 | 7-Isopropyl-3-(6- isopropyl-2-methoxy- pyridin-3-yl)-2,5- dimethyl-5H- pyrrolo[2,3-b]pyrazine | 339.19 | 1.87 |
| 466 | 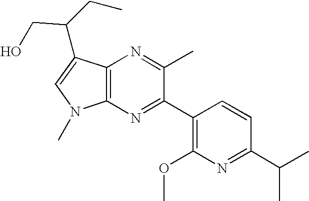 | 2-[3-(6-Isopropyl-2- methoxy-pyridin-3-yl)- 2,5-dimethyl-5H- pyrrolo[2,3-b]pyrazin- 7-yl]-butan-1-ol | 369.15 | 1.78 |
| 467 | 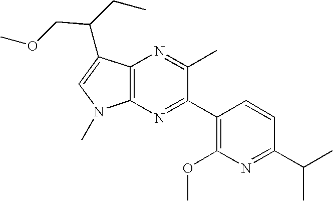 | 3-(6-Isopropyl-2- methoxy-pyridin-3-yl)- 7-(1-methoxymethyl- propyl)-2,5-dimethyl- 5H-pyrrolo[2,3- b]pyrazine | 383.16 | 1.85 |
| 468 | 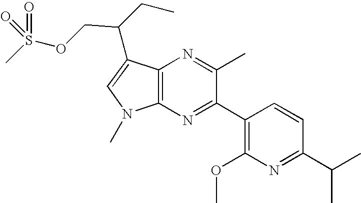 | Methanesulfonic acid 2-[3-(6-isopropyl-2- methoxy-pyridin-3-yl)- 2,5-dimethyl-5H- pyrrolo[2,3-b]pyrazin- 7-yl]-butyl ester | 447.06 | 1.78 |
| 469 | 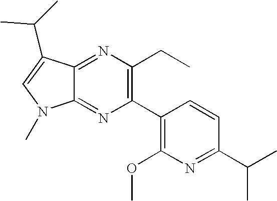 | 2-Ethyl-7-isopropyl-3- (6-isopropyl-2- methoxy-pyridin-3-yl)- 5-methyl-5H- pyrrolo[2,3-b]pyrazine | 353.18 | 1.92 |
| 470 | 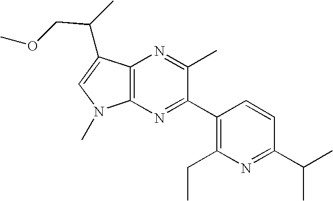 | 3-(2-Ethyl-6-isopropyl- pyridin-3-yl)-7-(2- methoxy-1-methyl- ethyl)-2,5-dimethyl- 5H-pyrrolo[2,3- b]pyrazine | 367.18 | 1.36 |
| 471 | 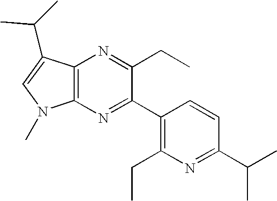 | 2-Ethyl-3-(2-ethyl-6- isopropyl-pyridin-3-yl)- 7-isopropyl-5-methyl- 5H-pyrrolo[2,3- b]pyrazine | 351.24 | 1.70 |
| 472 | 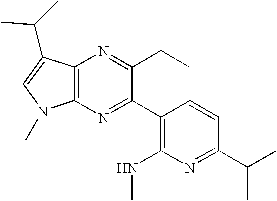 | [3-(2-Ethyl-7- isopropyl-5-methyl-5H- pyrrolo[2,3-b]pyrazin- 3-yl)-6-isopropyl- pyridin-2-yl]-methyl- amine | 352.21 | 1.50 |
| 473 | 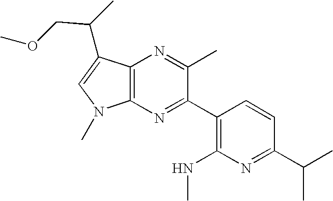 | {6-Isopropyl-3-[7-(2- methoxy-1-methyl- ethyl)-2,5-dimethyl- 5H-pyrrolo[2,3- b]pyrazin-3-yl]-pyridin- 2-yl}-methyl-amine | 368.19 | 1.37 |
| 474 | 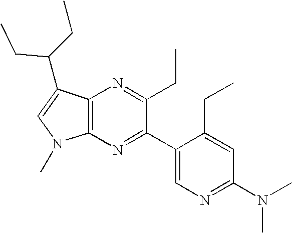 | {4-Ethyl-5-[2-ethyl-7- (1-ethyl-propyl)-5- methyl-5H-pyrrolo[2,3- b]pyrazin-3-yl]-pyridin- 2-yl}-dimethyl-amine | 380.5 | |
| 475 | 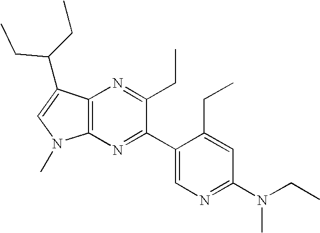 | Ethyl-{4-ethyl-5-[2- ethyl-7-(1-ethyl- propyl)-5-methyl-5H- pyrrolo[2,3-b]pyrazin- 3-yl]-pyridin-2-yl}- methyl-amine | 394.5 | |
| 476 | 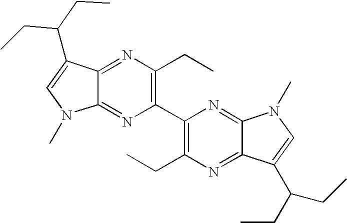 | 2,2′-Diethyl-7,7′-bis-(1- ethyl-propyl)-5,5′- dimethyl-5H,5′H- [3,3′]bi[pyrrolo[2,3- b]pyrazinyl] | 461.30 | 1.97 |
| 477 | 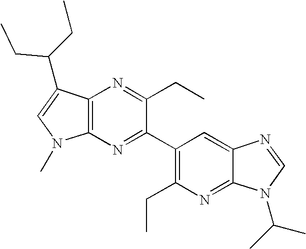 | 5-Ethyl-6-[2-ethyl-7-(1- ethyl-propyl)-5-methyl- 5H-pyrrolo[2,3- b]pyrazin-3-yl]-3- isopropyl-3H- imidazo[4,5-b]pyridine | 419.38 | 1.78 |
| 478 | 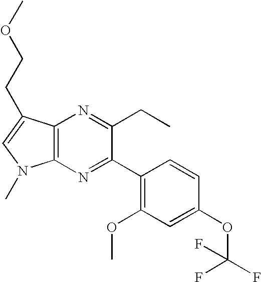 | 2-Ethyl-7-(2-methoxy- ethyl)-3-(2-methoxy-4- trifluoromethoxy- phenyl)-5-methyl-5H- pyrrolo[2,3-b]pyrazine | 410.06 | |
| 479 | 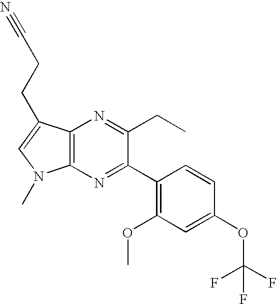 | 3-[2-Ethyl-3-(2- methoxy-4- trifluoromethoxy- phenyl)-5-methyl-5H- pyrrolo[2,3-b]pyrazin- 7-yl]-propionitrile | 405.07 | |
| 480 | 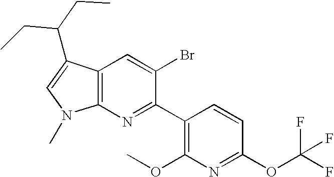 | 5-Bromo-3-(1-ethyl- propyl)-6-(2-methoxy- 4-trifluoromethoxy- phenyl)-1-methyl-1H- pyrrolo[2,3-b]pyridine | | |
| 481 | 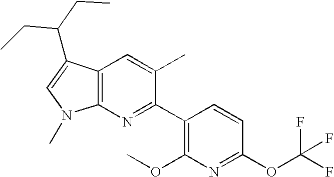 | 3-(1-Ethyl-propyl)-6- (2-methoxy-4- trifluoromethoxy- phenyl)-1,5-dimethyl- 1H-pyrrolo[2,3- b]pyridine | 407.6 | |
| 482 | 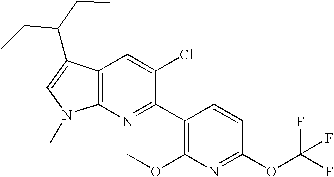 | 5-Chloro-3-(1-ethyl- propyl)-6-(2-methoxy- 4-trifluoromethoxy- phenyl)-1-methyl-1H- pyrrolo[2,3-b]pyridine | | |
| 483 | 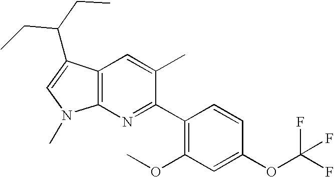 | 3-(1-Ethyl-propyl)-6- (2-methoxy-4- trifluoromethoxy- phenyl)-1,5-dimethyl- 1H-pyrrolo[2,3- b]pyridine | 407.6 | |
| 484 | 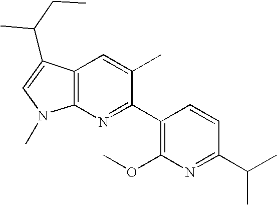 | 3-sec-Butyl-6-(6- isopropyl-2-methoxy- pyridin-3-yl)-1,5- dimethyl-1H- pyrrolo[2,3-b]pyridine | 352.5 | |
| 485 | 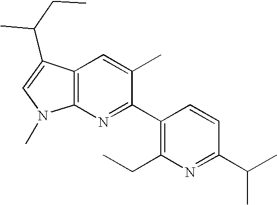 | 3-sec-Butyl-6-(2-ethyl- 6-isopropyl-pyridin-3- yl)-1,5-dimethyl-1H- pyrrolo[2,3-b]pyridine | 350.5 | |
| 486 | 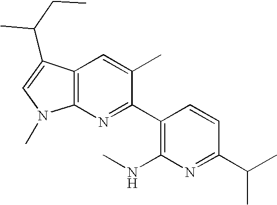 | [3-(3-sec-Butyl-1,5- dimethyl-1H- pyrrolo[2,3-b]pyridin- 6-yl)-6-isopropyl- pyridin-2-yl]-methyl- amine | 351.4 | |
| 487 | 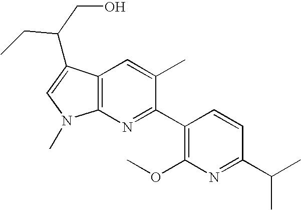 | 2-[6-(6-Isopropyl-2- methoxy-pyridin-3-yl)- 1,5-dimethyl-1H- pyrrolo[2,3-b]pyridin- 3-yl]-butan-1-ol | 368.3 | |
| 488 | 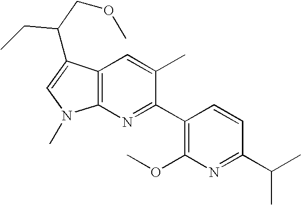 | 6-(6-Isopropyl-2- methoxy-pyridin-3-yl)- 3-(1-methoxymethyl- propyl)-1,5-dimethyl- 1H-pyrrolo[2,3- b]pyridine | 382.3 | |
| 489 | 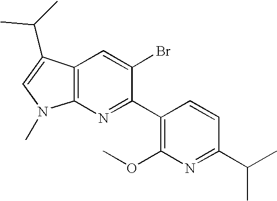 | 5-Bromo-3-isopropyl- 6-(6-isopropyl-2- methoxy-pyridin-3-yl)- 1-methyl-1H- pyrrolo[2,3-b]pyridine | 402.5 | |
| 490 | 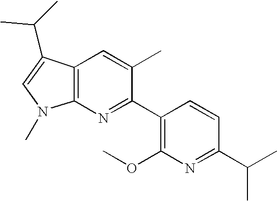 | 3-Isopropyl-6-(6- isopropyl-2-methoxy- pyridin-3-yl)-1,5- dimethyl-1H- pyrrolo[2,3-b]pyridine | 338.3 | |
| 491 | 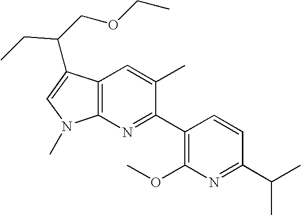 | 3-(1-Ethoxymethyl- propyl)-6-(6-isopropyl- 2-methoxy-pyridin-3- yl)-1,5-dimethyl-1H- pyrrolo[2,3-b]pyridine | 396.5 | |
| 492 | 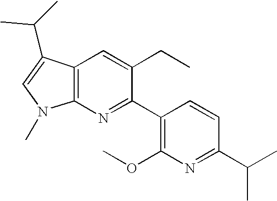 | 5-Ethyl-3-isopropyl-6- (6-isopropyl-2- methoxy-pyridin-3-yl)- 1-methyl-1H- pyrrolo[2,3-b]pyridine | 352.17 | 3.14 |

### Example 51

### Assay for CRF Receptor Binding Activity

As discussed above, the following assay is defined herein as a standard in vitro CRF receptor binding assay.

The pharmaceutical utility of compounds of this invention is indicated by the following assay for CRF1 receptor activity. The CRF receptor binding is performed using a modified version of the assay described by Grigoriadis and De Souza (Methods in Neurosciences, Vol. 5, 1991). IMR-32 human neuroblastoma cells, a cell-line that naturally expresses the CRF1 receptor, are grown in IMR-32 Medium, which consists of EMEM w/Earle's BSS (JRH Biosciences, Cat #51411) plus, as supplements, 2 mM L-Glutamine, 10% Fetal Bovine Serum, 25 mM HEPES (pH 7.2), 1 mM Sodium Pyruvate and Non-Essential Amino Acids (JRH Biosciences, Cat #58572). The cells are grown to confluence and split three times (all splits and harvest are carried out using NO-ZYME—JRH Biosciences, Cat #59226). The cells are first split 1:2, incubated for 3 days and split 1:3, and finally incubated for 4 days and split 1:5. The cells are then incubated for an additional 4 days before being differentiated by treatment with 5-bromo-2′deoxyuridine (BrdU, Sigma, Cat #B9285). The medium is replaced every 3-4 days with IMR-32 medium w/2.5 uM BrdU and the cells are harvested after 10 days of BrdU treatment and washed with calcium and magnesium-free PBS.

To prepare receptor containing membranes cells are homogenized in wash buffer (50 mM Tris HCl, 10 mM MgCl₂, 2 mM EGTA, pH 7.4) and centrifuged at 48,000×g for 10 minutes at 4° C. The pellet is re-suspended in wash buffer and the homogenization and centrifugation steps are performed two additional times.

Membrane pellets (containing CRF receptors) are re-suspended in 50 mM Tris buffer pH 7.7 containing 10 mM MgCl₂ and 2 mM EDTA and centrifuged for 10 minutes at 48,000 g. Membranes are washed again and brought to a final concentration of 1500 ug/ml in binding buffer (Tris buffer above with 0.1% BSA, 15 mM bacitracin and 0.01 mg/ml aprotinin.). For the binding assay, 100 ul of the membrane preparation are added to 96 well microtube plates containing 100 ul of ¹²⁵I-CRF (SA 2200 Ci/mmol, final concentration of 100 pM) and 50 ul of test compound. Binding is carried out at room temperature for 2 hours. Plates are then harvested on a BRANDEL 96 well cell harvester and filters are counted for gamma emissions on a Wallac 1205 BETAPLATE liquid scintillation counter. Non-specific binding is defined by 1 mM cold CRF. IC₅₀ values are calculated with the non-linear curve fitting program RS/1 (BBN Software Products Corp., Cambridge, Mass.). The binding affinity for the compounds of Formula I expressed as IC₅₀ value, generally ranges from about 0.5 nanomolar to about 10 micromolar. Preferred compounds of Formula I exhibit IC₅₀ values of less than or equal to 1.5 micromolar, more preferred compounds of Formula I exhibit IC₅₀ values of less than 500 nanomolar, still more preferred compounds of Formula I exhibit IC₅₀ values of less than 100 nanomolar, and most preferred compound of Formula I exhibit IC₅₀ values of less than 10 nanomolar. The compounds shown in Examples 1-33 have been tested in this assay and found to exhibit IC₅₀ values of less than or equal to 4 micromolar.

### Example 52

### Preparation of Radiolabeled Probe Compounds of the Invention

The compounds of the invention are prepared as radiolabeled probes by carrying out their synthesis using precursors comprising at least one atom that is a radioisotope. The radioisotope is preferably selected from of at least one of carbon (preferably ¹⁴C), hydrogen (preferably ³H), sulfur (preferably ³⁵S), or iodine (preferably ¹²⁵I). Such radiolabeled probes are conveniently synthesized by a radioisotope supplier specializing in custom synthesis of radiolabeled probe compounds. Such suppliers include Amersham Corporation, Arlington Heights, Ill.; Cambridge Isotope Laboratories, Inc. Andover, Mass.; SRI International, Menlo Park, Calif.; Wizard Laboratories, West Sacramento, Calif.; ChemSyn Laboratories, Lexena, Kans.; American Radiolabeled Chemicals, Inc., St. Louis, Mo.; and Moravek Biochemicals Inc., Brea, Calif.

Tritium labeled probe compounds are also conveniently prepared catalytically via platinum-catalyzed exchange in tritiated acetic acid, acid-catalyzed exchange in tritiated trifluoroacetic acid, or heterogeneous-catalyzed exchange with tritium gas. Such preparations are also conveniently carried out as a custom radiolabeling by any of the suppliers listed in the preceding paragraph using the compound of the invention as substrate. In addition, certain precursors may be subjected to tritium-halogen exchange with tritium gas, tritium gas reduction of unsaturated bonds, or reduction using sodium borotritide, as appropriate.

### Example 53

### Receptor Autoradiography

Receptor autoradiography (receptor mapping) is carried out in vitro as described by Kuhar in sections 8.1.1 to 8.1.9 of Current Protocols in Pharmacology (1998) John Wiley & Sons, New York, using radiolabeled compounds of the invention prepared as described in the preceding Examples.

### Example 54

### Additional Aspects of Preferred Compounds of the Invention

The most preferred compounds of the invention are suitable for pharmaceutical use in treating human patients. Accordingly, such preferred compounds are non-toxic. They do not exhibit single or multiple dose acute or long-term toxicity, mutagenicity (e.g., as determined in a bacterial reverse mutation assay such as an Ames test), teratogenicity, tumorogenicity, or the like, and rarely trigger adverse effects (side effects) when administered at therapeutically effective dosages.

Preferably, administration of such preferred compounds of the invention at certain doses (i.e., doses yielding therapeutically effective in vivo concentrations or preferably doses of 10, 50, 100, 150, or 200 mg/kg administered parenterally or preferably orally) does not result in prolongation of heart QT intervals (i.e., as determined by electrocardiography, e.g., in guinea pigs, minipigs or dogs). When administered daily for 5 or preferably ten days, such doses of such preferred compounds also do not cause liver enlargement resulting in an increase of liver to body weight ratio of more than 100%, preferably not more than 75% and more preferably not more than 50% over matched controls in laboratory rodents (e.g., mice or rats). In another aspect such doses of such preferred compounds also preferably do not cause liver enlargement resulting in an increase of liver to body weight ratio of more than 50%, preferably preferably not more than 25%, and more preferably not more than 10% over matched untreated controls in dogs or other non-rodent mammals.

In yet another aspect such doses of such preferred compounds also preferably do not promote the release of liver enzymes (e.g., ALT, LDH, or AST) from hepatocytes in vivo. Preferably such doses do not elevate serum levels of such enzymes by more than 100%, preferably not by more than 75% and more preferably not by more than 50% over matched untreated controls in laboratory rodents. Similarly, concentrations (in culture media or other such solutions that are contacted and incubated with cells in vitro) equivalent to two, fold, preferably five-fold, and most preferably ten-fold the minimum in vivo therapeutic concentration do not cause release of any of such liver enzymes from hepatocytes into culture medium in vitro above baseline levels seen in media from untreated cells.

Because side effects are often due to undesirable receptor activation or antagonism, preferred compounds of the invention exert their receptor-modulatory effects with high selectivity. This means that they do not bind to certain other receptors (other than CRF receptors) with high affinity, but rather only bind to, activate, or inhibit the activity of such other receptors with affinity constants of greater than 100 nanomolar, preferably greater than 1 micromolar, more preferably greater than 10 micromolar and most preferably greater than 100 micromolar. Such receptors preferably are selected from the group including ion channel receptors, including sodium ion channel receptors, neurotransmitter receptors such as alpha- and beta-adrenergic receptors, muscarinic receptors (particularly ml, m2, and m3 receptors), dopamine receptors, and metabotropic glutamate receptors; and also include histamine receptors and cytokine receptors, e.g., interleukin receptors, particularly IL-8 receptors. The group of other receptors to which preferred compounds do not bind with high affinity also includes GABA_{A} receptors, bioactive peptide receptors (including NPY and VIP receptors), neurokinin receptors, bradykinin receptors (e.g., BK1 receptors and BK2 receptors), and hormone receptors (including thyrotropin releasing hormone receptors and melanocyte-concentrating hormone receptors).

### Example 55

### Absence of Sodium Ion Channel Activity

Preferred compounds of the invention do not exhibit activity as sodium ion channel blockers. Sodium channel activity may be measured a standard in vitro sodium channel binding assays such as the assay given by Brown et al. (J. Neurosci. 1986, 265, 17995-18004). Preferred compounds of the invention exhibit less than 15 percent inhibition, and more preferably less than 10 percent inhibition, of sodium channel specific ligand binding when present at a concentration of 4 uM. The sodium ion channel specific ligand used may be labeled batrachotoxinin, tetrodotoxin, or saxitoxin. Such assays, including the assay of Brown referred to above, are performed as a commercial service by CEREP, Inc., Redmond, Wash.

Alternatively, sodium ion channel activity may be measured in vivo in an assay of anti-epileptic activity. Anti-epileptic activity of compounds may be measured by the ability of the compounds to inhibit hind limb extension in the supra maximal electro shock model. Male Han Wistar rats (150-200 mg) are dosed i.p. with a suspension of 1 to 20 mg of test compound in 0.25% methylcellulose 2 hr. prior to test. A visual observation is carried out just prior to testing for the presence of ataxia. Using auricular electrodes a current of 200 mA, duration 200 millisec, is applied and the presence or absence of hind limb extension is noted. Preferred compounds of the invention do not exhibit significant anti-epileptic activity at the p<0.1 level of significance or more preferably at the p<0.05 level of significance as measured using a standard parametric assay of statistical significance such as a student's T test.

### Example 56

### Microsomal In Vitro Half-Life

Compound half-life values (t_{1/2} values) may be determined via the following standard liver microsomal half-life assay. Pooled Human liver microsomes are obtained from XenoTech LLC, 3800 Cambridge St. Kansas's City, Kans., 66103 (catalog #H0610). Such liver microsomes may also be obtained from In Vitro Technologies, 1450 South Rolling Road, Baltamore, Md. 21227, or from Tissue Transformation Technologies, Edison Corporate Center, 175 May Street, Suite 600, Edison, N.J. 08837. Reactions are preformed as follows:

Reagents:

Phosphate buffer: 19 mL 0.1 M NaH₂PO₄, 81 mL 0.1 Na₂HPO₄, adjusted to pH 7.4 with H₃PO₄.

CoFactor Mixture: 16.2 mg NADP, 45.4 mg Glucose-6-phosphate in 4 mL 100 mM MgCl₂.

Glucose-6-phosphate dehydrogenase: 214.3 ul glucose-6-phosphate dehydrogenase suspension (Boehringer-Manheim catalog no. 0737224, distributed by Roche Molecular Biochemicals, 9115 Hague Road, P.O. Box 50414, Indianapolis, Ind. 46250) is diluted into 1285.7 ul distilled water.

Starting Reaction Mixture: 3 mL CoFactor Mixture, 1.2 mL Glucose-6-phosphate dehydrogenase.

Reaction:

6 test reactions are prepared, each containing 25 ul microsomes, 5 ul of a 100 uM solution of test compound, and 399 ul 0.1 M phosphate buffer. A seventh reaction is prepared as a positive control containing 25 ul microsomes, 399 ul 0.1 M phosphate buffer, and 5 ul of a 100 uM solution of a compound with known metabolic properties (e.g. DIAZEPAM or CLOZEPINE). Reactions are preincubated at 39° C. for 10 minutes. 71 ul Starting Reaction Mixture is added to 5 of the 6 test reactions and to the positive control, 71 ul 100 mM MgCl₂ is added to the sixth test reaction, which is used as a negative control. At each time point (0, 1, 3, 5, and 10 minutes) 75 ul of each reaction mix is pipetted into a well of a 96-well deep-well plate containing 75 ul ice-cold acetonitrile. Samples are vortexed and centrifuged 10 minutes at 3500 rpm (Sorval T 6000D centrifuge, H1000B rotor). 75 ul of supernatant from each reaction is transferred to a well of a 96-well plate containing 150 ul of a 0.5 uM solution of a compound with a known LCMS profile (internal standard) per well. LCMS analysis of each sample is carried out and the amount of unmetabolized test compound is measured as AUC, compound concentration vs time is plotted, and the t_{1/2} value of the test compound is extrapolated.

Preferred compounds of the invention exhibit in vitro t_{1/2} values of greater than 10 minutes and less than 4 hours. Most preferred compounds of the invention exhibit in vitro t_{1/2} values of between 30 minutes and 1 hour in human liver microsomes.

### Example 57

### MDCK Toxicity Assay

Compounds causing acute cytotoxicity will decrease ATP production by Madin Darby canine kidney (MDCK) cells in the following assay.

MDCK cells, ATCC no. CCL-34 (American Type Culture Collection, Manassas, Va.) are maintained in sterile conditions following the instructions in the ATCC production information sheet. The PACKARD, (Meriden, Conn.) ATP-LITE-M Luminescent ATP detection kit, product no. 6016941, allows measurement ATP production in MDCK cells.

Prior to assay 1 ul of test compound or control sample is pipetted into PACKARD (Meriden, Conn.) clear bottom 96-well plates. Test compounds and control samples are diluted in DMSO to give final concentration in the assay of 10 micromolar, 100 micromolar, or 200 micromolar. Control samples are drug or other compounds having known toxicity properties.

Confluent MDCK cells are trypsinized, harvested, and diluted to a concentration of 0.1×10⁶ cells/ml with warm (37° C.) VITACELL Minimum Essential Medium Eagle (ATCC catalog #30-2003). 100 ul of cells in medium is pipetted into each of all but five wells of each 96-well plate. Warm medium without cells (100 ul) is pipetted in the remaining five wells of each plate to provide standard curve control wells. These wells, to which no cells are added, are used to determine the standard curve. The plates are then incubated at 37° C. under 95% O₂, 5% CO₂ for 2 hours with constant shaking. After incubation, 50 ul of mammalian cell lysis solution is added per well, the wells are covered with PACKARD TOPSEAL stickers, and plates are shaken at approximately 700 rpm on a suitable shaker for 2 minutes.

During the incubation, PACKARD ATP LITE-M reagents are allowed to equilibrate to room temperature. Once equilibrated the lyophilized substrate solution is reconstituted in 5.5 mls of substrate buffer solution (from kit). Lyophilized ATP standard solution is reconstituted in deionized water to give a 10 mM stock. For the five control wells, 10 ul of serially diluted PACKARD standard is added to each of the five standard curve control wells to yield a final concentration in each subsequent well of 200 nM, 100 nM, 50 nM, 25 nM, and 12.5 nM.

PACKARD substrate solution (50 ul) is added to all wells. Wells are covered with PACKARD TOPSEAL stickers, and plates are shaken at approximately 700 rpm on a suitable shaker for 2 minutes. A white PACKARD sticker is attached to the bottom of each plate and samples are dark adapted by wrapping plates in foil and placing in the dark for 10 minutes. Luminescence is then measured at 22° C. using a luminescence counter, e.g. PACKARD TOPCOUNT Microplate Scintillation and Luminescense Counter or TECAN SPECTRAFLUOR PLUS.

Luminescence values at each drug concentration are compared to the values computed from the standard curve for that concentration. Preferred test compounds exhibit luminescence values 80% or more of the standard, or preferably 90% or more of the standard, when a 10 micromolar (uM) concentration of the test compound is used. When a 100 uM concentration of the test compound is used, preferred test compounds exhibit luminescence values 50% or more of the standard, or more preferably 80% or more of the standard.

1. A compound of the Formula I-a: 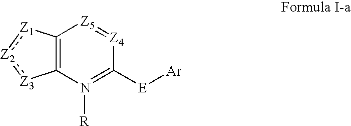
or a pharmaceutically acceptable salt thereof, wherein:
E is a single bond, O, S(O)ₘ, NR₁₀ or CR₁₀R₁₁;
R₁₀ and R₁₁ are independently hydrogen or C₁-C₄ alkyl;
m is 0, 1, or 2;
Ar is chosen from:
phenyl which is mono-, di-, or tri-substituted, 1-naphthyl and 2-naphthyl, each of which is optionally mono-, di-, or tri-substituted, and optionally mono-, di-, or tri-substituted heteroaryl, said heteroaryl having from 1 to 3 rings, 5 to 7 ring members in each ring and, in at least one of said rings, from 1 to about 3 heteroatoms selected from the group consisting of N, O, and S;
R is oxygen or absent;
the group: 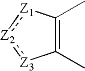
represents a saturated, unsaturated or aromatic 5-membered ring system containing 0 or 1 heteroatoms, wherein:
Z₁ is CR₁ or CR₁R₁′;
Z₂ is nitrogen, oxygen, sulfur, CR₂, CR₂R₂′ or NR₂″,
Z₃ is nitrogen, oxygen, sulfur, sulfoxide, sulfone, CR₃, or CR₃R₃′;
R₁ is chosen from hydrogen, halogen, hydroxy, cyano, amino, optionally substituted alkyl, optionally substituted alkenyl, optionally substituted alkynyl, optionally substituted alkoxy, optionally substituted mono or dialkylamino, optionally substituted cycloalkyl, optionally substituted (cycloalkyl)alkyl, optionally substituted alkylthio, optionally substituted alkylsulfinyl, optionally substituted alkylsulfonyl, optionally substituted mono- or dialkylcarboxamide, optionally substituted carbocyclic aryl, optionally substituted heterocycle and optionally substituted heteroaryl, said optionally substituted heterocycle or heteroaryl having from 1 to 3 rings, 5 to 7 ring members in each ring and, in at least one of said rings, from 1 to about 3 heteroatoms selected from the group consisting of N, O, and S;
R₂ and R₃ are independently chosen from hydrogen, halogen, hydroxy, amino, cyano, nitro, alkyl, haloalkyl, alkoxy, aminoalkyl, hydroxyalkyl and mono and dialkylamino, wherein when R₁ or R₁″ is optionally substituted alkyl, then R₃ is optionally substituted C₁₋₃alkyl;
R₁′, R₂′ and R₃′ are independently chosen from hydrogen, halogen, alkyl, haloalkyl, and aminoalkyl;
R₂″ is chosen from hydrogen, optionally substituted alkyl, optionally substituted haloalkyl, and optionally substituted aminoalkyl;
Z₄ is NR or CR₄;
Z₅ is NR or CR₅;
R₄ and R₅ are independently chosen from hydrogen, halogen, hydroxy, amino, cyano, nitro, optionally substituted alkyl, optionally substituted alkenyl, optionally substituted alkynyl, optionally substituted alkoxy, optionally substituted mono or dialkylamino, optionally substituted (cycloalkyl)alkyl, optionally substituted alkylthio, optionally substituted alkylsulfinyl, optionally substituted alkylsulfonyl, optionally substituted mono- or dialkylcarboxamide, optionally substituted carbocyclic aryl, and optionally substituted heteroaryl, said optionally substituted heteroaryl having from 1 to 3 rings, 5 to 7 ring members in each ring and, in at least one of said rings, from 1 to about 3 heteroatoms selected from the group consisting of N, O, and S.

2. A compound of the Formula I-a: 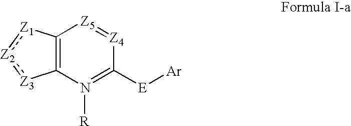
or a pharmaceutically acceptable salt thereof, wherein:
E is a single bond, O, S(O)ₘ, NR₁₀ or CR₁₀R₁₁;
R₁₀ and R₁₁ are independently hydrogen or C₁-C₄ alkyl;
m is 0, 1, or 2;
R is oxygen or absent;
Ar is chosen from:
phenyl which is mono-, di-, or tri-substituted with R_{A}, and 1-naphthyl, 2-naphthyl, pyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
the group: 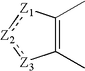
represents a saturated, unsaturated or aromatic ring system comprising 0 or 1 heteroatoms, wherein:
Z₁ is CR₁ or CR₁R₁′;
Z₂ is nitrogen, oxygen, sulfur, CR₂, CR₂R₂′ or NR₂″,
Z₃ is nitrogen, oxygen, sulfur, sulfoxide, sulfone, CR₃, or CR₃R₃′;
R₁ is chosen from
i) hydrogen, halogen, hydroxy, cyano, amino, C₁-C₁₀alkyl, —O(C₁-C₆alkyl), mono or di(C₁-C₆alkyl)amino, (C₃-C₇cycloalkyl)C₁-C₄alkyl, halo(C₁-C₆)alkyl, —O(halo(C₁-C₆)alkyl) and S(O)ₙ(C₁-C₆alkyl), —O(C₃-C₇cycloalkyl)C₁-C₄alkyl, and S(O)ₙ(C₁-C₆alkyl),
where each alkyl is independently straight, branched, or cyclic, contains zero or 1 or more double or triple bonds, and is optionally substituted with one or more substituents independently chosen from halogen, hydroxy, amino, oxo, cyano, C₁₋C₁-C₄alkoxy, and mono- or di(C₁-C₄)alkylamino,
and
where each C₃-C₇cycloalkyl is optionally substituted by one or more substituents independently chosen from halogen, amino, hydroxy, oxo, cyano, C₁-C₄alkoxy, and mono- or di(C₁-C₄)alkylamino, and
ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₂ and R₃ are independently chosen from hydrogen, halogen, hydroxy, amino, cyano, nitro, C₁-C₃alkyl, halo(C₁-C₃)alkyl, C₁-C₃alkoxy, amino(C₁-C₃)alkyl, and mono and di(C₁-C₆)alkylamino;
R₁′, R₂′ and R₃′ are independently chosen from hydrogen, halogen, C₁-C₆alkyl, halo(C₁-C₆)alkyl, and amino(C₁-C₆)alkyl;
R₂″ is chosen from hydrogen, C₁-C₆alkyl, halo(C₁-C₆)alkyl, and amino(C₁-C₆)alkyl;
Z₄ is NR or CR₄;
Z₅ is NR or CR₅;
wherein Z₄ and Z₅ are not both NR;
R₄ and R₅ are independently chosen from hydrogen, halogen, cyano, nitro, amino, mono or di(C₁-C₆carbhydryl)amino, C₁-C₆carbhydryl, (C₃-C₇cyclocarbhydryl)C₀-C₄carbhydryl, —O(C₃-C₇cyclocarbhydryl), halo(C₁-C₆)carbhydryl, —O(halo(C₁-C₆)carbhydryl), —O(C₁-C₆carbhydryl), and S(O)ₙ(C₁-C₆carbhydryl),
where each carbhydryl is independently straight, branched, or cyclic, contains zero or 1 or more double or triple bonds, and is optionally substituted with one or more substituents independently chosen from halogen, hydroxy, amino, oxo, cyano, C₁-C₄alkoxy, and mono- and di(C₁-C₄)alkylamino,
and
where each C₃-C₇carbhydryl is optionally substituted by one or more substituents independently chosen from halogen, amino, hydroxy, oxo, cyano, C₁-C₄alkoxy, and mono- and di(C₁-C₄)alkylamino;
R_{A} is independently selected at each occurrence from halogen, cyano, nitro, halo(C₁-C₆)alkyl, halo(C₁-C₆)alkoxy, hydroxy, amino, C₁-C₆alkyl substituted with 0-2 R_{B}, C₂-C₆alkenyl substituted with 0-2 R_{B}, C₂-C₆alkynyl substituted with 0-2 R_{B}, C₃-C₇cycloalkyl substituted with 0-2 R_{B}, (C₃-C₇cycloalkyl)C₁-C₄alkyl substituted with 0-2 R_{B}, C₁-C₆alkoxy substituted with 0-2 R_{B}, —NH(C₁-C₆alkyl) substituted with 0-2 R_{B}, —N(C₁-C₆alkyl)(C₁-C₆alkyl) each C₁-C₆alkyl independently substituted with 0-2 R_{B}, X XR_{C}, and Y;
R_{B} is independently selected at each occurrence from the group consisting of halogen, hydroxy, cyano, amino, C₁-C₄alkyl, —O(C₁-C₄alkyl), —NH(C₁-C₄alkyl), —N(C₁-C₄alkyl)(C₁-C₄alkyl), —S(O)ₙ(alkyl), halo(C₁-C₄)alkyl, halo(C₁-C₄)alkoxy, CO(C₁-C₄alkyl), CONH(C₁-C₄alkyl), CON(C₁-C₄alkyl)(C₁-C₄alkyl), —XR_{C}, and Y;
R_{C} and R_{D}, which may be the same or different, are independently selected at each occurrence from:
hydrogen, and straight, branched, or cyclic alkyl groups, including (cycloalkyl)alkyl groups consisting of 1 to 8 carbon atoms, which straight, branched, or cyclic alkyl groups contain zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
X is independently selected at each occurrence from the group consisting of —CH₂—, —CHR_{D}—, —O—, —C(═O)—, —C(═O)O—, —S(O)ₙ—, —NH—, —NR_{D}—, —C(═O)NH—, —C(═O)NR_{D}—, —S(O)ₙNH—, —S(O)ₙNR_{D}—, —OC(═S)S—, —NHC(═O)—, —NR_{D}C(═O)—, —NHS(O)ₙ—, —OSiH₂—, —OSiH(C₁-C₄alkyl)-, —OSi(C₁-C₄alkyl)(C₁-C₄alkyl)-, and —NR_{D}S(O)ₙ—;
Y and Z are independently selected at each occurrence from: 3- to 7-membered carbocyclic or heterocyclic groups which are saturated, unsaturated, or aromatic, which may be further substituted with one or more substituents independently selected from halogen, oxo, hydroxy, amino, cyano, C₁-C₄alkyl, —O(C₁-C₄alkyl), —NH(C₁-C₄alkyl), —N(C₁-C₄alkyl)(C₁-C₄alkyl), and —S(O)ₙ(alkyl),
wherein said 3- to 7-membered heterocyclic groups contain one or more heteroatom(s) independently selected from N, O, and S, with the point of attachment being either carbon or nitrogen; and
n is independently selected at each occurrence from 0, 1, and 2.

3. A compound of the Formula I-b: 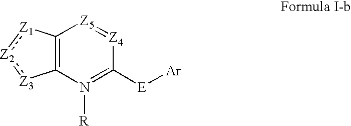
or a pharmaceutically acceptable salt thereof, wherein:
E is a single bond, O, S(O)ₘ, NR₁₀ or CR₁₀R₁₁;
R₁₀ and R₁₁ are independently hydrogen or C₁-C₄ alkyl;
m is 0, 1, or 2;
R is oxygen or absent;
Ar is chosen from:
phenyl which is mono-, di-, or tri-substituted, 1-naphthyl and 2-naphthyl, each of which is optionally mono-, di-, or tri-substituted, and optionally mono-, di-, or tri-substituted heteroaryl, said heteroaryl having from 1 to 3 rings, 5 to 7 ring members in each ring and, in at least one of said rings, from 1 to about 3 heteroatoms selected from the group consisting of N, O, and S;
the group: 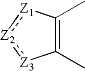
represents a saturated, unsaturated or aromatic ring system comprising 0 or 1 heteroatoms, wherein:
Z₁ is CR₁, CR₁R₁′, or NR₁″;
Z₂ is CR₂ or CR₂R₂′;
Z₃ is CR₃, CR₃R₃′, or NR₃″;
R₁ and R₁″ are chosen from hydrogen, C₁-C₁₀alkyl, C₂-C₁₀alkenyl, C₂-C₁₀alkynyl, C₃-C₇cycloalkyl, (benzo)C₃-C₇cycloalkyl, (C₃-C₇cycloalkyl)C₁-C₄alkyl, C₃₋₉ heterocycloalkyl, (C₃₋₉ heterocycloalkyl)C₁-C₄alkyl, (benzo)C₃₋₉ heterocycloalkyl, ((benzo)C₃₋₉ heterocycloalkyl)C₁-C₄alkyl and halo(C₁-C₆)alkyl, each of which is substituted with 0 or more substituents independently chosen from halogen, hydroxy, amino, oxo, cyano, C₁-C₆alkyl, C₁-C₆alkoxy, haloC₁-C₆alkoxy,C₁-C₆alkanoyl, C₁-C₆alkanoyloxy, C₁-C₆alkoxycarbonyl, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, C₁-C₆alkylsulfonamide, C₁-C₆alkylsulfonyl, C₁-C₆alkylsulfonyloxy, C₁-C₆ hydroxyalkyl, C₁-C₆alkoxyC₁-C₆alkyl, C₁-C₆ haloalkoxy, 5 to 7 membered heteroaryl, 5 to 7 membered heterocycloalkyl, mono- and di-(C₁-C₆)alkylamino, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, mono- and di-(C₁-C₆)alkylcarbamoyl, —XR_{C} and X-Z, with the proviso that R₁ and R₁″ is not aryl or heteroaryl substituted alkyl;
R₂ is chosen from hydrogen, halogen, hydroxy, amino, cyano, nitro, C₁-C₃alkyl, halo(C₁-C₃)alkyl, C₁-C₃alkoxy, amino(C₁-C₃)alkyl, and mono and di(C₁-C₆)alkylamino;
R₃ is chosen from hydrogen, hydroxy, amino, halogen, cyano, nitro, C₁-C₃alkyl, halo(C₁-C₃)alkyl, C₁-C₃alkoxy, amino(C₁-C₃)alkyl, hydroxy(C₁-C₃)alkyl, cyano(C₁-C₃)alkyl, and mono and di(C₁-C₃)alkylamino;
R₃″ is chosen from hydrogen, hydroxy, amino, C₁-C₃alkyl, halo(C₁-C₃)alkyl, C₁-C₃alkoxy, amino(C₁-C₃)alkyl, hydroxy(C₁-C₃)alkyl, cyano(C₁-C₃)alkyl, and mono and di(C₁-C₃)alkylamino;
R₁′, R₂′ and R₃′ are independently chosen from hydrogen, halogen, C₁-C₆alkyl, halo(C₁-C₆)alkyl, and amino(C₁-C₆)alkyl;
Z₄ is NR or CR₄;
Z₅ is NR or CR₅;
R₄ and R₅ are independently chosen from hydrogen, halogen, cyano, nitro, amino, mono or di(C₁-C₆carbhydryl)amino, C₁-C₆carbhydryl, (C₃-C₇cyclocarbhydryl)C₀-C₄carbhydryl, —O(C₃-C₇cyclocarbhydryl), halo(C₁-C₆)carbhydryl, —O(halo(C₁-C₆)carbhydryl), —O(C₁-C₆carbhydryl), S(O)ₙ(C₁-C₆carbhydryl), and 4 to 7 membered heterocycloalkyl,
where each carbhydryl is independently straight, branched, or cyclic, contains zero or 1 or more double or triple bonds, and is optionally substituted with one or more substituents independently chosen from halogen, hydroxy, amino, oxo, cyano, C₁-C₄alkoxy, and mono- and di(C₁-C₄)alkylamino,
and
where each C₃-C₇carbhydryl heterocycloalkyl is optionally substituted by one or more substituents independently chosen from halogen, amino, hydroxy, oxo, cyano, C₁-C₄alkoxy, and mono- and di(C₁-C₄)alkylamino; or
R₅, taken in combination with R₁ or R₁″, forms a 5-9 membered heterocycle;
R_{A} is independently selected at each occurrence from halogen, cyano, nitro, halo(C₁-C₆)alkyl, halo(C₁-C₆)alkoxy, hydroxy, amino, C₁-C₆alkyl substituted with 0-2 R_{B}, C₂-C₆alkenyl substituted with 0-2 R_{B}, C₂-C₆alkynyl substituted with 0-2 R_{B}, C₃-C₇cycloalkyl substituted with 0-2 R_{B}, (C₃-C₇cycloalkyl)C₁-C₄alkyl substituted with 0-2 R_{B}, C₁-C₆alkoxy substituted with 0-2 R_{B}, —NH(C₁-C₆alkyl) substituted with 0-2 R_{B}, —N(C₁-C₆alkyl)(C₁-C₆alkyl) each C₁-C₆alkyl independently substituted with 0-2 R_{B}, —XR_{C}, and Y;
R_{B} is independently selected at each occurrence from the group consisting of halogen, hydroxy, cyano, amino, C₁-C₄alkyl, —O(C₁-C₄alkyl), —NH(C₁-C₄alkyl), —N(C₁-C₄alkyl)(C₁-C₄alkyl), —S(O)ₙ(alkyl), halo(C₁-C₄)alkyl, halo(C₁-C₄)alkoxy, CO(C₁-C₄alkyl), CONH(C₁-C₄alkyl), CON(C₁-C₄alkyl)(C₁-C₄alkyl), —XR_{C}, and Y;
R_{C} and R_{D}, which may be the same or different, are independently selected at each occurrence from:
hydrogen, and
straight, branched, or cyclic alkyl groups, including (cycloalkyl)alkyl groups consisting of 1 to 8 carbon atoms, which straight, branched, or cyclic alkyl groups contain zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
X is independently selected at each occurrence from the group consisting of —O—, —C(═O)O—, —S(O)ₙ—, —NH—, —NR_{D}—, —C(═O)NH—, —C(═O)NR_{D}—, —S(O)ₙNH—, —S(O)ₙNR_{D}—, —OC(═S)S—, —NHC(═O)—, —NR_{D}C(═O)—, —NHS(O)ₙ—, —OSiH₂—, —OSiH(C₁-C₄alkyl)-, —OSi(C₁-C₄alkyl)(C₁-C₄alkyl)-, and —NR_{D}S(O)ₙ—;
Y and Z are independently selected at each occurrence from: 3- to 7-membered carbocyclic or heterocyclic groups which are saturated, unsaturated, or aromatic, which may be further substituted with one or more substituents independently selected from halogen, oxo, hydroxy, amino, cyano, C₁-C₄alkyl, —O(C₁-C₄alkyl), —NH(C₁-C₄alkyl), —N(C₁-C₄alkyl)(C₁-C₄alkyl), —C(O)(C₁-C₄alkyl), and —S(O)ₙ(alkyl), wherein said 3- to 7-membered heterocyclic groups contain one or more heteroatom(s) independently selected from N, O, and S, with the point of attachment being either carbon or nitrogen; and
n is independently selected at each occurrence from 0, 1, and 2.

4. A compound or salt according to claim 3, wherein
Ar is chosen from:
phenyl which is mono-, di-, or tri-substituted with R_{A}, and 1-naphthyl, 2-naphthyl, pyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A}; and
R₁ and R₁″ are chosen from hydrogen, C₁-C₁₀alkyl, C₂-C₁₀alkenyl, C₂-C₁₀alkynyl, C₃-C₇cycloalkyl, (C₃-C₇cycloalkyl)C₁-C₄alkyl, C₃₋₉ heterocycloalkyl, (C₃₋₉ heterocycloalkyl)C₁-C₄alkyl and halo(C₁-C₆)alkyl, each of which is substituted with 0, 1, 2, or 3 substituents independently chosen from halogen, hydroxy, amino, oxo, cyano, C₁-C₆alkyl, C₁-C₆alkoxy, haloC₁-C₆alkoxy,C₁-C₆alkanoyl, C₁-C₆alkanoyloxy, C₁-C₆alkoxycarbonyl, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, C₁-C₆alkylsulfonamide, C₁-C₆alkylsulfonyl, C₁-C₆alkylsulfonyloxy, C₁-C₆ hydroxyalkyl, C₁-C₆alkoxyC₁-C₆alkyl, C₁-C₆ haloalkoxy, 5 to 7 membered heteroaryl, 5 to 7 membered heterocycloalkyl, mono- and di-(C₁-C₆)alkylamino, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, mono- and di-(C₁-C₆)alkylcarbamoyl, —XR_{C} and X-Z.

5. A compound or salt according to claim 3 of Formula II 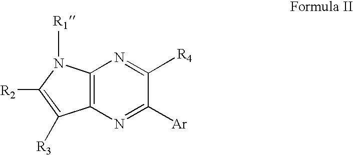
wherein R₁″, R₂, R₃, R₄ and Ar are as defined in claim 3.

6. A compound or salt according to claim 5, wherein:
R₁″ is as defined for claim 3;
R₂ is selected from hydrogen, methyl, and ethyl;
R₃ is hydrogen or C₁-C₃alkyl;
R₄ is selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula II is substituted.

7. A compound or salt according to claim 5, wherein:
R₁″ is selected from C₁-C₁₀alkyl and (C₃-C₇cycloalkyl)C₀-C₄alkyl, each of which is substituted with 0 or more substituents independently chosen from halogen, hydroxy, amino, oxo, cyano, C₁-C₄alkoxy, and mono- and di-(C₁-C₄)alkylamino.

8. A compound or salt according to claim 5, wherein:
R₁″ is selected from C₃₋₉ heterocycloalkyl and (C₃₋₉ heterocycloalkyl)C₁₋₄alkyl, each of which is substituted with 0-4 substitutents selected from halogen, amino, hydroxy, nitro, cyano, C₁-C₆alkyl, C₁-C₆alkoxy, C₁-C₆ hydroxyalkyl, C₁-C₆alkoxyC₁-C₆alkyl, (C₁-C₆)haloalkyl, (C₁-C₆)haloalkoxy, mono- and di-(C₁-C₆)alkylamino, —XR_{C}.

9. A compound or salt according to claim 8, wherein:
R₁″ is chosen from tetrahydrofuranyl, tetrahydropyranyl, morpholinyl, pyrrolidinyl, piperidinyl, piperazinyl [2.2.1]-azabicyclic rings, [2.2.2]-azabicyclic rings, [3.3.1]-azabicyclic rings, quinuclidinyl, azetidinyl, azetidinonyl, oxindolyl, dihydroimidazolyl, and pyrrolidinonyl, each of which is substituted with from 0 to 2 substituents independently chosen from:
(i) halogen, hydroxy, amino, cyano, or
(ii) C₁-C₄alkyl, C₁-C₄alkoxy, and mono- and di-(C₁-C₄)alkylamino, each of which is substituted with 0 or 1 substituents selected from halogen, hydroxy, amino, C₁₋₂alkoxy, or C₃₋₉ heterocycloalkyl.

10. A compound or salt according to claim 3 of Formula VI 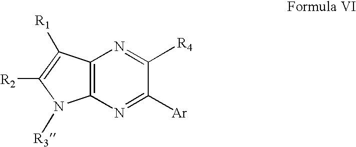
wherein R₁, R₂, R₃″, R₄, and Ar are as defined in claim 3.

11. A compound or salt according to claim 10, wherein:
R₁ is as defined for claim 3;
R₂ is selected from hydrogen, methyl, and ethyl;
R₃″ is hydrogen or C₁-C₃alkyl;
R₄ is selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula VI is substituted.

12. A compound or salt according to claim 11, wherein:
R₁″ is selected from C₁-C₁₀alkyl and (C₃-C₇cycloalkyl)C₀-C₄alkyl, each of which is substituted with 0 or more substituents independently chosen from halogen, hydroxy, amino, oxo, cyano, C₁-C₄alkoxy, and mono- and di-(C₁-C₄)alkylamino.

13. A compound or salt according to claim 11, wherein:
R₁″ is selected from C₃₋₉ heterocycloalkyl and (C₃₋₉ heterocycloalkyl)C₁₋₄alkyl, each of which is substituted with 0-4 substitutents selected from halogen, amino, hydroxy, nitro, cyano, C₁-C₆alkyl, C₁-C₆alkoxy, C₁-C₆ hydroxyalkyl, C₁-C₆alkoxyC₁-C₆alkyl, (C₁-C₆)haloalkyl, (C₁-C₆)haloalkoxy, mono- and di-(C₁-C₆)alkylamino, —XR_{C}.

14. A compound or salt according to claim 13, wherein:
R₁″ is chosen from tetrahydrofuranyl, tetrahydropyranyl, morpholinyl, pyrrolidinyl, piperidinyl, piperazinyl [2.2.1]-azabicyclic rings, [2.2.2]-azabicyclic rings, [3.3.1]-azabicyclic rings, quinuclidinyl, azetidinyl, azetidinonyl, oxindolyl, dihydroimidazolyl, and pyrrolidinonyl, each of which is substituted with from 0 to 2 substituents independently chosen from:
(i) halogen, hydroxy, amino, cyano, or
(ii) C₁-C₄alkyl, C₁-C₄alkoxy, and mono- and di-(C₁-C₄)alkylamino, each of which is substituted with 0 or 1 substituents selected from halogen, hydroxy, amino, C₁₋₂alkoxy, or C₃₋₉ heterocycloalkyl.

15. A compound or salt according to claim 3 of Formula VIII 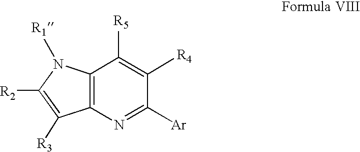
wherein R₁″, R₂, R₃, R₄, R₅, and Ar are as defined in claim 3.

16. A compound or salt according to claim 15, wherein:
R₁″ is as defined for claim 3;
R₂ is selected from hydrogen, methyl, and ethyl;
R₃ is C₁-C₃alkyl;
R₄ and R₅ are independently selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula VIII is substituted.

17. A compound or salt according to claim 15, wherein:
R₁″ is selected from C₁-C₁₀alkyl and (C₃-C₇cycloalkyl)C₀-C₄alkyl, each of which is substituted with 0 or more substituents independently chosen from halogen, hydroxy, amino, oxo, cyano, C₁-C₄alkoxy, and mono- and di-(C₁-C₄)alkylamino.

18. A compound or salt according to claim 15, wherein:
R₁″ is selected from C₃₋₉ heterocycloalkyl and (C₃₋₉ heterocycloalkyl)C₁₋₄alkyl, each of which is substituted with 0-4 substitutents selected from halogen, amino, hydroxy, nitro, cyano, C₁-C₆alkyl, C₁-C₆alkoxy, C₁-C₆ hydroxyalkyl, C₁-C₆alkoxyC₁-C₆alkyl, (C₁-C₆)haloalkyl, (C₁-C₆)haloalkoxy, mono- and di-(C₁-C₆)alkylamino, —XR_{C}.

19. A compound or salt according to claim 18, wherein:
R₁″ is chosen from tetrahydrofuranyl, tetrahydropyranyl, morpholinyl, pyrrolidinyl, piperidinyl, piperazinyl [2.2.1]-azabicyclic rings, [2.2.2-azabicyclic rings, [3.3.1]-azabicyclic rings, quinuclidinyl, azetidinyl, azetidinonyl, oxindolyl, dihydroimidazolyl, and pyrrolidinonyl, each of which is substituted with from 0 to 2 substituents independently chosen from:
(i) halogen, hydroxy, amino, cyano, or
(ii) C₁-C₄alkyl, C₁-C₄alkoxy, and mono- and di-(C₁-C₄)alkylamino, each of which is substituted with 0 or 1 substituents selected from halogen, hydroxy, amino, C₁₋₂alkoxy, or C₃₋₉ heterocycloalkyl.

20. A compound or salt according to claim 3 of Formula X 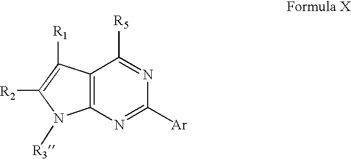
wherein R₁, R₂, R₃″, R₅, and Ar are as defined in claim 3.

21. A compound or salt according to claim 20, wherein
R₁ is as defined for claim 3;
R₂ is selected from hydrogen, methyl, and ethyl;
R₃″ is selected from hydrogen and C₁-C₆alkyl;
R₅ is selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula X is substituted.

22. A compound or salt according to claim 20, wherein:
R₁ is selected from C₁-C₁₀alkyl and (C₃-C₇cycloalkyl)C₀-C₄alkyl, each of which is substituted with 0 or more substituents independently chosen from halogen, hydroxy, amino, oxo, cyano, C₁-C₄alkoxy, and mono- and di-(C₁-C₄)alkylamino.

23. A compound or salt according to claim 20, wherein:
R₁ is selected from C₃₋₉ heterocycloalkyl and (C₃₋₉ heterocycloalkyl)C₁₋₄alkyl, each of which is substituted with 0-4 substitutents selected from halogen, amino, hydroxy, nitro, cyano, C₁-C₆alkyl, C₁-C₆alkoxy, C₁-C₆ hydroxyalkyl, C₁-C₆alkoxyC₁-C₆alkyl, (C₁-C₆)haloalkyl, (C₁-C₆)haloalkoxy, mono- and di-(C₁-C₆)alkylamino, —XR_{C}.

24. A compound or salt according to claim 23, wherein:
R₁ is chosen from tetrahydrofuranyl, tetrahydropyranyl, morpholinyl, pyrrolidinyl, piperidinyl, piperazinyl [2.2.1]-azabicyclic rings, [2.2.2]-azabicyclic rings, [3.3.1]-azabicyclic rings, quinuclidinyl, azetidinyl, azetidinonyl, oxindolyl, dihydroimidazolyl, and pyrrolidinonyl, each of which is substituted with from 0 to 2 substituents independently chosen from:
(i) halogen, hydroxy, amino, cyano, or
(ii) C₁-C₄alkyl, C₁-C₄alkoxy, and mono- and di-(C₁-C₄)alkylamino, each of which is substituted with 0 or 1 substituents selected from halogen, hydroxy, amino, C₁₋₂alkoxy, or C₃₋₉ heterocycloalkyl.

25. A compound or salt according to claim 3 of Formula XII 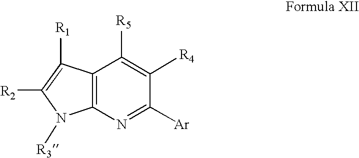
wherein R₁, R₂, R₃″, R₄, R₅, and Ar are as defined in claim 3.

26. A compound or salt according to claim 25, wherein
R₁ is as defined for claim 3;
R₂ is selected from hydrogen, methyl, and ethyl;
R₃″ is selected from hydrogen and C₁-C₆alkyl;
R₄ and R₅ are independently selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XII is substituted.

27. A compound or salt according to claim 25, wherein:
R₁ is selected from C₁-C₁₀alkyl and (C₃-C₇cycloalkyl)C₀-C₄alkyl, each of which is substituted with 0 or more substituents independently chosen from halogen, hydroxy, amino, oxo, cyano, C₁-C₄alkoxy, and mono- and di-(C₁-C₄)alkylamino.

28. A compound or salt according to claim 25, wherein:
R₁ is selected from C₃₋₉ heterocycloalkyl and (C₃₋₉ heterocycloalkyl)C₁₋₄alkyl, each of which is substituted with 0-4 substitutents selected from halogen, amino, hydroxy, nitro, cyano, C₁-C₆alkyl, C₁-C₆alkoxy, C₁-C₆ hydroxyalkyl, C₁-C₆alkoxyC₁-C₆alkyl, (C₁-C₆)haloalkyl, (C₁-C₆)haloalkoxy, mono- and di-(C₁-C₆)alkylamino, —XR_{C}.

29. A compound or salt according to claim 28, wherein:
R₁ is chosen from tetrahydrofuranyl, tetrahydropyranyl, morpholinyl, pyrrolidinyl, piperidinyl, piperazinyl [2.2.1]-azabicyclic rings, [2.2.2]-azabicyclic rings, [3.3.1]-azabicyclic rings, quinuclidinyl, azetidinyl, azetidinonyl, oxindolyl, dihydroimidazolyl, and pyrrolidinonyl, each of which is substituted with from 0 to 2 substituents independently chosen from:
(i) halogen, hydroxy, amino, cyano, or
(ii) C₁-C₄alkyl, C₁-C₄alkoxy, and mono- and di-(C₁-C₄)alkylamino, each of which is substituted with 0 or 1 substituents selected from halogen, hydroxy, amino, C₁₋₂alkoxy, or C₃₋₉ heterocycloalkyl.

30. A compound or salt according to claim 3 of Formula XIV 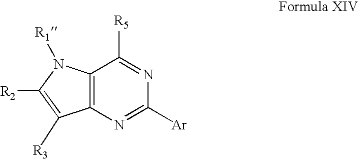
wherein R₁″, R₂, R₃, R₅, and Ar are as defined in claim 3.

31. A compound or salt according to claim 30, wherein
R₁″ is as defined for claim 3;
R₂ is selected from hydrogen, methyl, and ethyl;
R₃ is C₁-C₃alkyl;
R₅ is selected from hydrogen, halogen, cyano, amino, C₁-C₆alkyl, C₁-C₆alkoxy, C₃-C₇cycloalkyl, (C₃-C₇cycloalkyl)C₁-C₄alkyl, (C₃-C₇cycloalkyl)C₁-C₄alkoxy, mono and di(C₁-C₆alkyl)amino, amino(C₁-C₆)alkyl, mono and di(C₁-C₆alkyl)amino(C₁-C₆)alkyl, halo(C₁-C₆)alkyl, and halo(C₁-C₆)alkoxy; and
Ar is selected from the group consisting of phenyl, pyridyl and pyrimidinyl each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XIV is substituted.

32. A compound or salt according to claim 30, wherein:
R₁″ is selected from C₁-C₁₀alkyl and (C₃-C₇cycloalkyl)C₀-C₄alkyl, each of which is substituted with 0 or more substituents independently chosen from halogen, hydroxy, amino, oxo, cyano, C₁-C₄alkoxy, and mono- and di-(C₁-C₄)alkylamino.

33. A compound or salt according to claim 30, wherein:
R₁″ is selected from C₃₋₉ heterocycloalkyl and (C₃₋₉ heterocycloalkyl)C₁₋₄alkyl, each of which is substituted with 0-4 substitutents selected from halogen, amino, hydroxy, nitro, cyano, C₁-C₆alkyl, C₁-C₆alkoxy, C₁-C₆ hydroxyalkyl, C₁-C₆alkoxyC₁-C₆alkyl, (C₁-C₆)haloalkyl, (C₁-C₆)haloalkoxy, mono- and di-(C₁-C₆)alkylamino, —XR_{C}.

34. A compound or salt according to claim 33, wherein:
R₁″ is chosen from tetrahydrofuranyl, tetrahydropyranyl, morpholinyl, pyrrolidinyl, piperidinyl, piperazinyl [2.2.1]-azabicyclic rings, [2.2.2]-azabicyclic rings, [3.3.1]-azabicyclic rings, quinuclidinyl, azetidinyl, azetidinonyl, oxindolyl, dihydroimidazolyl, and pyrrolidinonyl, each of which is substituted with from 0 to 2 substituents independently chosen from:
(i) halogen, hydroxy, amino, cyano, or
(ii) C₁-C₄alkyl, C₁-C₄alkoxy, and mono- and di-(C₁-C₄)alkylamino, each of which is substituted with 0 or 1 substituents selected from halogen, hydroxy, amino, C₁₋₂alkoxy, or C₃₋₉ heterocycloalkyl.

35. A compound of the Formula XX: 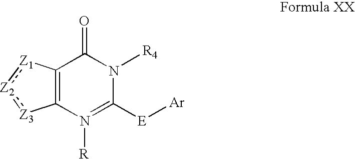
or a pharmaceutically acceptable salt thereof, wherein:
E is a single bond, O, S(O)ₘ, NR₁₀ or CR₁₀R₁₁;
R₁₀ and R₁₁ are independently hydrogen or C₁-C₄ alkyl;
m is 0, 1, or 2;
Ar is chosen from:
phenyl which is mono-, di-, or tri-substituted, 1-naphthyl and 2-naphthyl, each of which is optionally mono-, di-, or tri-substituted, and optionally mono-, di-, or tri-substituted heteroaryl, said heteroaryl having from 1 to 3 rings, 5 to 7 ring members in each ring and, in at least one of said rings, from 1 to about 3 heteroatoms selected from the group consisting of N, O, and S;
R is oxygen or absent;
the group: 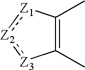
represents a saturated, unsaturated or aromatic 5-membered ring system containing 0 or 1 heteroatoms, wherein:
Z₁ is CR₁CR₁R₁′, or NR₁″;
Z₂ is nitrogen, oxygen, sulfur, CR₂, CR₂R₂′ or NR₂″,
Z₃ is nitrogen, oxygen, sulfur, sulfoxide, sulfone, CR₃, CR₃R₃′, or NR₃″;
R₁ is chosen from halogen, hydroxy, cyano, amino, optionally substituted alkyl, optionally substituted alkenyl, optionally substituted alkynyl, optionally substituted alkoxy, optionally substituted mono or dialkylamino, optionally substituted (cycloalkyl)alkyl, optionally substituted cycloalkyl, optionally substituted heterocycloalkyl, optionally substituted alkylthio, optionally substituted alkylsulfinyl, optionally substituted alkylsulfonyl, optionally substituted mono- or dialkylcarboxamide, optionally substituted carbocyclic aryl, and optionally substituted heteroaryl, said optionally substituted heteroaryl having from 1 to 3 rings, 5 to 7 ring members in each ring and, in at least one of said rings, from 1 to about 3 heteroatoms selected from the group consisting of N, O, and S;
R₁″ is chosen from optionally substituted alkyl, optionally substituted alkenyl, optionally substituted alkynyl, optionally substituted (cycloalkyl)alkyl, optionally substituted cycloalkyl, optionally substituted heterocycloalkyl, optionally substituted (heterocycloalkyl)alkyl, optionally substituted carbocyclic aryl, and optionally substituted heteroaryl, said optionally substituted heteroaryl having from 1 to 3 rings, 5 to 7 ring members in each ring and, in at least one of said rings, from 1 to about 3 heteroatoms selected from the group consisting of N, O, and S;
R₂ and R₃ are independently chosen from hydrogen, halogen, hydroxy, amino, cyano, nitro, alkyl, haloalkyl, alkoxy, aminoalkyl, and mono and dialkylamino;
R₁′, R₂′ and R₃′ are independently chosen from hydrogen, halogen, alkyl, haloalkyl, and aminoalkyl;
R₂″ and R₃″ are independently chosen from hydrogen, alkyl, haloalkyl, and aminoalkyl; and
R₄ is hydrogen, alkyl, aminoalkyl, and haloalkyl

36. A compound of the formula: 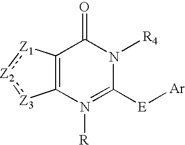
or a pharmaceutically acceptable salt thereof, wherein:
E is a single bond, O, S(O)ₘ, NR₁₀ or CR₁₀R₁₁;
R₁₀ and R₁₁ are independently hydrogen or C₁-C₄ alkyl;
m is 0, 1, or 2;
R is oxygen or absent;
Ar is chosen from:
phenyl which is mono-, di-, or tri-substituted, 1-naphthyl and 2-naphthyl, each of which is optionally mono-, di-, or tri-substituted, and optionally mono-, di-, or tri-substituted heteroaryl, said heteroaryl having from 1 to 3 rings, 5 to 7 ring members in each ring and, in at least one of said rings, from 1 to about 3 heteroatoms selected from the group consisting of N, O, and S;
the group: 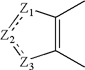
represents a saturated, unsaturated or aromatic ring system comprising 0 or 1 heteroatoms, wherein:
Z is CR₁, CR₁R₁′, or NR₁″;
Z₂ is nitrogen, oxygen, sulfur, CR₂, CR₂R₂′ or NR₂″,
Z₃ is nitrogen, oxygen, sulfur, sulfoxide, sulfone, CR₃, CR₃R₃′ or NR₃″;
R₁ is chosen from
i) halogen, hydroxy, cyano, amino, C₁-C₁₀carbhydryl, —O(C₁-C₆carbhydryl), mono or di(C₁-C₆carbhydryl)amino, (C₃-C₇cyclocarbhydryl)C₁-C₄carbhydryl, halo(C₁-C₆)carbhydryl, —O(halo(C₁-C₆)carbhydryl) and S(O)ₙ(C₁-C₆carbhydryl), —O(C₃-C₇cyclocarbhydryl)C₁-C₄carbhydryl, C₃₋₉ heterocycloalkyl, (C₃₋₉ heterocycloalkyl)C₁-C₄alkyl, and S(O)ₙ(C₁-C₆carbhydryl),
where each carbhydryl is independently straight, branched, or cyclic, contains zero or 1 or more double or triple bonds,
where each heterocycloalkyl has 1 or 2 ring heteroatoms selected from N, O, or S and
the point of attachment is carbon or nitrogen; and
where each carbhydryl, heterocycloalkyl, or cyclocarbhydryl is optionally substituted by one or more substituents independently chosen from halogen, hydroxy, amino, oxo, cyano, C₁-C₆alkyl, C₁-C₆alkoxy, haloC₁-C₆alkoxy,C₁-C₆alkanoyl, C₁-C₆alkanoyloxy, C₁-C₆alkoxycarbonyl, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, C₁-C₆alkylsulfonamide, C₁-C₆alkylsulfonyl, C₁-C₆alkylsulfonyloxy, C₁-C₆ hydroxyalkyl, C₁-C₆alkoxyC₁-C₆alkyl, C₁-C₆ haloalkoxy, 5 to 7 membered heteroaryl, 5 to 7 membered heterocycloalkyl, mono- and di-(C₁-C₆)alkylamino, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, mono- and di-(C₁-C₆)alkylcarbamoyl, —XR_{C} and X-Z, and
ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₁″ is chosen from C₁-C₁₀alkyl, C₂-C₁₀alkenyl, C₂-C₁₀alkynyl, C₃-C₇cycloalkyl, (C₃-C₇cycloalkyl)C₁-C₄alkyl, C₃₋₉ heterocycloalkyl, (C₃₋₉ heterocycloalkyl)C₁-C₄alkyl and halo(C₁-C₆)alkyl, each of which is substituted with 0 or more substituents independently chosen from halogen, hydroxy, amino, oxo, cyano, C₁-C₆alkyl, C₁-C₆alkoxy, haloC₁-C₆alkoxy,C₁-C₆alkanoyl, C₁-C₆alkanoyloxy, C₁-C₆alkoxycarbonyl, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, C₁-C₆alkylsulfonamide, C₁-C₆alkylsulfonyl, C₁-C₆alkylsulfonyloxy, C₁-C₆ hydroxyalkyl, C₁-C₆alkoxyC₁-C₆alkyl, C₁-C₆ haloalkoxy, 5 to 7 membered heteroaryl, 5 to 7 membered heterocycloalkyl, mono- and di-(C₁-C₆)alkylamino, N-(C₁-C₆alkanoyl)-N-(C₀-C₆alkyl)amino, N-(C₁-C₆alkanoyloxy)-N-(C₀-C₆alkyl)amino, N-(C₁-C₆alkoxycarbonyl)-N-(C₀-C₆alkyl)amino, mono- and di-(C₁-C₆)alkylcarbamoyl, —XR_{C} and X-Z;
R₂ and R₃ are independently chosen from hydrogen, halogen, hydroxy, amino, cyano, nitro, C₁-C₆alkyl, halo(C₁-C₆)alkyl, C₁-C₆alkoxy, amino(C₁-C₆)alkyl, and mono and di(C₁-C₆)alkylamino;
R₂′ and R₃′ are independently chosen from hydrogen, halogen, C₁-C₆alkyl, halo(C₁-C₆)alkyl, and amino(C₁-C₆)alkyl;
R₂″ and R₃″ are independently chosen from hydrogen, C₁-C₆alkyl, halo(C₁-C₆)alkyl, and amino(C₁-C₆)alkyl;
R₄ is hydrogen, C₁-C₆alkyl, C₁-C₆aminoalkyl, and C₁-C₆ haloalkyl
R_{A} is independently selected at each occurrence from halogen, cyano, nitro, halo(C₁-C₆)alkyl, halo(C₁-C₆)alkoxy, hydroxy, amino, C₁-C₆alkyl substituted with 0-2 R_{B}, C₂-C₆alkenyl substituted with 0-2 R_{B}, C₂-C₆alkynyl substituted with 0-2 R_{B}, C₃-C₇cycloalkyl substituted with 0-2 R_{B}, (C₃-C₇cycloalkyl)C₁-C₄alkyl substituted with 0-2 R_{B}, C₁-C₆alkoxy substituted with 0-2 R_{B}, —NH(C₁-C₆alkyl) substituted with 0-2 R_{B}, —N(C₁-C₆alkyl)(C₁-C₆alkyl) each C₁-C₆alkyl independently substituted with 0-2 R_{B}, —XR_{C}, and Y;
R_{B} is independently selected at each occurrence from the group consisting of halogen, hydroxy, cyano, amino, C₁-C₄alkyl, —O(C₁-C₄alkyl), —NH(C₁-C₄alkyl), —N(C₁-C₄alkyl)(C₁-C₄alkyl), —S(O)ₙ(alkyl), halo(C₁-C₄)alkyl, halo(C₁-C₄)alkoxy, CO(C₁-C₄alkyl), CONH(C₁-C₄alkyl), CON(C₁-C₄alkyl)(C₁-C₄alkyl), —XR_{C}, and Y;
R_{C} and R_{D}, which may be the same or different, are independently selected at each occurrence from:
hydrogen, and
straight, branched, or cyclic alkyl groups, including (cycloalkyl)alkyl groups consisting of 1 to 8 carbon atoms, which straight, branched, or cyclic alkyl groups contain zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
X is independently selected at each occurrence from the group consisting of —CH₂—, —CHR_{D}—, —O—, —C(═O)—, —C(═O)O—, —S(O)ₙ—, —NH—, —NR_{D}—, —C(═O)NH—, —C(═O)NR_{D}—, —S(O)ₙNH—, —S(O)ₙNR_{D}—, —OC(═S)S—, —NHC(═O)—, —NR_{D}C(═O)—, —NH S(O)ₙ—, —OSiH₂—, —OSiH(C₁-C₄alkyl)-, —OSi(C₁-C₄alkyl)(C₁-C₄alkyl)-, and —NR_{D}S(O)ₙ—;
Y and Z are independently selected at each occurrence from: 3- to 7-membered carbocyclic or heterocyclic groups which are saturated, unsaturated, or aromatic, which may be further substituted with one or more substituents independently selected from halogen, oxo, hydroxy, amino, cyano, C₁-C₄alkyl, —O(C₁-C₄alkyl), —C(O)(C₁-C₄alkyl), —NH(C₁-C₄alkyl), —N(C₁-C₄alkyl)(C₁-C₄alkyl), and —S(O)ₙ(alkyl),
wherein said 3- to 7-membered heterocyclic groups contain one or more heteroatom(s) independently selected from N, O, and S, with the point of attachment being either carbon or nitrogen; and
n is independently selected at each occurrence from 0, 1, and 2.

37. A compound or salt according to claim 36 of Formula XXI 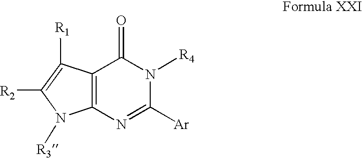
wherein,
R₁, R₂, R₃″, R₄ are as defined in claim 37; and
Ar is chosen from:
phenyl which is mono-, di-, or tri-substituted with R_{A}, and 1-naphthyl, 2-naphthyl, pyridyl and pyrimidinyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A}.

38. A compound or salt according to claim 37, wherein
R₁ is as defined in claim 36;
R₂ and R₄ are independently selected from hydrogen, methyl, and ethyl;
R₃″ is selected from hydrogen and C₁-C₆alkyl; and
Ar is selected from the group consisting of phenyl, pyridyl and pyrimidinyl each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XXI is substituted.

39. A compound or salt according to claim 36 of Formula XXII 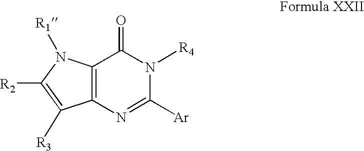
wherein R₁″, R₂, R₃, R₄, and R₅ are as defined in claim 36; and
Ar is chosen from:
phenyl which is mono-, di-, or tri-substituted with R_{A}, and 1-naphthyl, 2-naphthyl, pyridyl and pyrimidinyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A}.

40. A compound or salt according to claim 39, wherein
R₁″ is as defined in claim 39;
R₂ and R₄ are independently selected from hydrogen, methyl, and ethyl;
R₃ is selected from hydrogen and C₁-C₆alkyl; and
Ar is selected from the group consisting of phenyl, pyridyl and pyrimidinyl each of which is mono- di- or trisubstituted with substituents independently chosen from halogen, cyano, nitro, halo(C₁-C₆)alkyl, halo(C₁-C₆)alkoxy, hydroxy, amino, C₁-C₆alkyl, C₂-C₆alkenyl, C₂-C₆alkynyl, C₃-C₇cycloalkyl, (C₃-C₇cycloalkyl)C₁-C₄alkyl, C₁-C₆alkoxy, mono- and di(C₁-C₆alkyl)amino, amino(C₁-C₆)alkyl, and mono- and di(C₁-C₆alkyl)amino, wherein, in Ar, at least one of the positions ortho to the point of attachment of Ar shown in Formula XXII is substituted.

41. A compound or pharmaceutically acceptable salt thereof, wherein the compound is selected from the group consisting of:
5-(1-Ethyl-propyl)-2-(2-methoxy-4-trifluoromethoxy-phenyl)-3,7-dimethyl-5H-pyrrolo[2,3-b]pyrazine;
{4-Ethyl-5-[5-(1-ethyl-propyl)-3,7-dimethyl-5H-pyrrolo[2,3-b]pyrazin-2-yl]-pyridin-2-yl}-dimethyl-amine;
{3-Bromo-4-ethyl-5-[5-(1-ethyl-propyl)-3,7-dimethyl-5H-pyrrolo[2,3-b]pyrazin-2-yl]-pyridin-2-yl}-ethyl-methyl-amine;
Ethyl-{4-ethyl-5-[5-(1-ethyl-propyl)-3,7-dimethyl-5H-pyrrolo[2,3-b]pyrazin-2-yl]-pyridin-2-yl}-methyl-amine;
{5-[5-(1-Ethyl-propyl)-3,7-dimethyl-5H-pyrrolo[2,3-b]pyrazin-2-y]-4-methoxy-pyridin-2-yl}-dimethyl-amine;
2-[2-Ethoxy-5-methanesulfonyl-6-(1-methyl-but-3-enyl)-pyridin-3-yl]-5-(1-ethyl-propyl)-3,7-dimethyl-5H-pyrrolo[2,3-b]pyrazine;
2-(2-Ethoxy-6-ethyl-5-methanesulfonyl-pyridin-3-yl)-5-(1-ethyl-propyl)-3,7-dimethyl-5H-pyrrolo[2,3-b]pyrazine;
{5-[3-Chloro-5-(1-ethyl-propyl)-7-methyl-5H-pyrrolo[2,3-b]pyrazin-2-yl]-4-ethyl-pyridin-2-yl}-ethyl-methyl-amine;
{5-[3-Chloro-5-(1-ethyl-propyl)-7-methyl-5H-pyrrolo[2,3-b]pyrazin-2-yl]-4-ethyl-pyridin-2-yl}-dimethyl-amine;
{5-[3-Chloro-5-(1-ethyl-propyl)-7-methyl-5H-pyrrolo[2,3-b]pyrazin-2-yl]-4-ethyl-pyridin-2-yl}-diethyl-amine;
3-Chloro-5-(1-ethyl-propyl)-2-(3-isopropyl-5-methoxy-2,3-dihydro-furo[3,2-b]pyridin-6-yl)-7-methyl-5H-pyrrolo[2,3-b]pyrazine;
3-Chloro-5-(1-ethyl-propyl)-2-(3-isopropyl-5-methoxy-furo[3,2-b]pyridin-6-yl)-7-methyl-5H-pyrrolo[2,3-b]pyrazine;
(R)-2-[2-(6-Isopropyl-2-methoxy-pyridin-3-yl)-3,7-dimethyl-pyrrolo[2,3-b]pyrazin-5-yl]-3-methoxy-propan-1-ol;
5-(1-Ethyl-propyl)-2-(6-isopropyl-2-methoxy-pyridin-3-yl)-3,7-dimethyl-5H-pyrrolo[2,3-b]pyrazine;
2-(2-Ethyl-6-isopropyl-pyridin-3-yl)-5-(1-ethyl-propyl)-3,7-dimethyl-5H-pyrrolo[2,3-b]pyrazine;
2-[(S)-2-(6-Isopropyl-2-methoxy-pyridin-3-yl)-3,7-dimethyl-pyrrolo[2,3-b]pyrazin-5-yl]-butan-1-ol;
Methanesulfonic acid 2-[(S)-2-(6-isopropyl-2-methoxy-pyridin-3-yl)-3,7-dimethyl-pyrrolo[2,3-b]pyrazin-5-yl]-butyl ester;
3-{2-[(S)-2-(6-Isopropyl-2-methoxy-pyridin-3-y)-3,7-dimethyl-pyrrolo[2,3-b]pyrazin-5-yl]-butyl}-oxazolidin-2-one;
(S)-2-(6-Isopropyl-2-methoxy-pyridin-3-yl)-5-(1-methoxymethyl-propyl)-3,7-dimethyl-5H-pyrrolo[2,3-b]pyrazine;
Ethyl-{2-[(S)-2-(6-isopropyl-2-methoxy-pyridin-3-yl)-3,7-dimethyl-pyrrolo[2,3-b]pyrazin-5-yl]-butyl}-methyl-amine;
{2-[(S)-2-(6-Isopropyl-2-methoxy-pyridin-3-yl)-3,7-dimethyl-pyrrolo[2,3-b]pyrazin-5-yl]-butyl}-methyl-amine;
N-{2-[(S)-2-(6-Isopropyl-2-methoxy-pyridin-3-y)-3,7-dimethyl-pyrrolo[2,3-b]pyrazin-5-yl]-butyl}-N-methyl-methanesulfonamide;
N-{2-[(S)-2-(6-Isopropyl-2-methoxy-pyridin-3-yl)-3,7-dimethyl-pyrrolo[2,3-b]pyrazin-5-yl]-butyl}-N-methyl-acetamide;
{2-[(S)-2-(6-Isopropyl-2-methoxy-pyridin-3-yl)-3,7-dimethyl-pyrrolo[2,3-b]pyrazin-5-yl]-butyl}-methyl-carbamic acid methyl ester;
(R)-2-(6-Isopropyl-2-methoxy-pyridin-3-yl)-5-(1-methoxymethyl-propyl)-3,7-dimethyl-5H-pyrrolo[2,3-b]pyrazine;
Acetic acid 2-[(R)-2-(6-isopropyl-2-methoxy-pyridin-3-yl)-3,7-dimethyl-pyrrolo[2,3-b]pyrazin-5-yl]-butyl ester;
2-[(R)-2-(6-Isopropyl-2-methoxy-pyridin-3-yl)-3,7-dimethyl-pyrrolo[2,3-b]pyrazin-5-yl]-butan-1-ol;
(R)-2-(2-Ethyl-6-isopropyl-pyridin-3-yl)-5-(1-methoxymethyl-propyl)-3,7-dimethyl-5H-pyrrolo[2,3-b]pyrazine;
{6-Isopropyl-3-[(R)-5-(1-methoxymethyl-propyl)-3,7-dimethyl-5H-pyrrolo[2,3-b]pyrazin-2-yl]-pyridin-2-yl}-methyl-amine;
{2-[(R)-2-(6-Isopropyl-2-methoxy-pyridin-3-yl)-3,7-dimethyl-pyrrolo[2,3-b]pyrazin-5-yl]-butyl}-dimethyl-amine;
(R)-2-(6-Isopropyl-2-methoxy-pyridin-3-yl)-3,7-dimethyl-5-(1-pyrrolidin-1-ylmethyl-propyl)-5H-pyrrolo[2,3-b]pyrazine;
Diethyl-2-[(R)-2-(6-isopropyl-2-methoxy-pyridin-3-yl)-3,7-dimethyl-pyrrolo[2,3-b]pyrazin-5-yl]-butyl}-amine;
Isopropyl-{2-[(R)-2-(6-isopropyl-2-methoxy-pyridin-3-yl)-3,7-dimethyl-pyrrolo[2,3-b]pyrazin-5-yl]-butyl}-methyl-amine;
(R)-2-(6-Isopropyl-2-methoxy-pyridin-3-yl)-3,7-dimethyl-5-(-morpholin-4-ylmethyl-propyl)-5H-pyrrolo[2,3-b]pyrazine;
Cyclobutyl-{2-[(R)-2-(6-isopropyl-2-methoxy-pyridin-3-yl)-3,7-dimethyl-pyrrolo[2,3-b]pyrazin-5-yl]-butyl}-amine;
{2-[(R)-2-(6-Isopropyl-2-methoxy-pyridin-3-y)-3,7-dimethyl-pyrrolo[2,3-b]pyrazin-5-yl]-butyl}-(2-methoxy-ethyl)-methyl-amine;
2-(6-Isopropyl-2-methoxy-pyridin-3-yl)-3,7-dimethyl-5-(1-methylene-propyl)-5H-pyrrolo[2,3-b]pyrazine;
Butyl-ethyl-{2-[(R)-2-(6-isopropyl-2-methoxy-pyridin-3-yl)-3,7-dimethyl-pyrrolo[2,3-b]pyrazin-5-yl]-butyl}-amine;
5-sec-Butyl-2-(6-isopropyl-2-methoxy-pyridin-3-yl)-3,7-dimethyl-5H-pyrrolo[2,3-b]pyrazine;
Dimethyl-carbamic acid 2-[(R)-2-(6-isopropyl-2-methoxy-pyridin-3-yl)-3,7-dimethyl-pyrrolo[2,3-b]pyrazin-5-yl]-butyl ester;
{2-[(R)-2-(6-Isopropyl2-methoxy-pyridin-3-yl)-3,7-dimethyl-pyrrolo[2,3-b]pyrazin-5-yl]-butyl}-dipropyl-amine;
2-(6-Isopropyl-2-methoxy-pyridin-3-y)-3,7-dimethyl-5-[(R)-1-(4-methyl-piperazin-1-ylmethyl)-propyl]-5H-pyrrolo[2,3-b]pyrazine;
1-(4-{(R)-2-[2-(6-Isopropyl-2-methoxy-pyridin-3-yl)-3,7-dimethyl-pyrrolo[2,3-b]pyrazin-5-yl]-butyl}-piperazin-1-yl)-ethanone;
2-(2-Ethyl-6-isopropyl-pyridin-3-yl)-3,7-dimethyl-5-((R)-1-morpholin-4-ylmethyl-propyl)-5H-pyrrolo[2,3-b]pyrazine;
{3-[3,7-Dimethyl-5-((R)-1-morpholin-4-ylmethyl-propyl)-5H-pyrrolo[2,3-b]pyrazin-2-yl]-6-isopropyl-pyridin-2-yl}-methyl-amine;
{(R)-2-[2-(4-Difluoromethoxy-2-methoxy-phenyl)-3,7-dimethyl-pyrrolo[2,3-b]pyrazin-5-yl]-butyl}-ethyl-methyl-amine;
{(R)-2-[2-(2-Chloro-4-methoxy-phenyl)-3,7-dimethyl-pyrrolo[2,3-b]pyrazin-5-yl]-butyl}-ethyl-methyl-amine;
5-Isopropyl-2-(6-isopropyl-2-methoxy-pyridin-3-yl)-3,7-dimethyl-5H-pyrrolo[2,3-b]pyrazine;
[6-Isopropyl-3-(5-isopropyl-3,7-dimethyl-5H-pyrrolo[2,3-b]pyrazin-2-yl)-pyridin-2-yl]-methyl-amine;
2-(2-Ethyl-6-isopropyl-pyridin-3-yl)-5-isopropyl-3,7-dimethyl-5H-pyrrolo[2,3-b]pyrazine;
2-(4-Difluoromethoxy-2-methoxy-phenyl)-5-isopropyl-3,7-dimethyl-5H-pyrrolo[2,3-b]pyrazine;
5-Isopropyl-2-(2-methoxy-4-trifluoromethyl-phenyl)-3,7-dimethyl-5H-pyrrolo[2,3-b]pyrazine;
[3-(3,7-Dimethyl-5-propyl-5H-pyrrolo[2,3-b]pyrazin-2-yl)-6-isopropyl-pyridin-2-yl]-methyl-amine;
2-(6-Isopropyl-2-methoxy-pyridin-3-yl)-3,7-dimethyl-5-propyl-5H-pyrrolo[2,3-b]pyrazine;
5-Isopropyl-2-(2-methoxy-4-trifluoromethoxy-phenyl)-3,7-dimethyl-5H-pyrrolo[2,3-b]pyrazine;
2-(2-Ethyl-6-isopropyl-pyridin-3-yl)-3,7-dimethyl-5-propyl-5H-pyrrolo[2,3-b]pyrazine;
(R)-2-(6-Isopropyl-pyridin-3-yl)-5-(1-methoxymethyl-propyl)-3,7-dimethyl-5H-pyrrolo[2,3-b]pyrazine;
(S)-2-(2-Ethyl-6-isopropyl-pyridin-3-yl)-5-(1-methoxymethyl-propyl)-3,7-dimethyl-5H-pyrrolo[2,3-b]pyrazine;
{6-Isopropyl-3-[(S)-5-(1-methoxymethyl-propyl)-3,7-dimethyl-5H-pyrrolo[2,3-b]pyrazin-2-yl]-pyridin-2-yl}-methyl-amine;
(S)-3-Chloro-2-(6-isopropyl-2-methoxy-pyridin-3-yl)-5-(2-methoxy-1-methyl-ethyl)-7-methyl-5H-pyrrolo[2,3-b]pyrazine;
(S)-3-Ethyl-2-(6-isopropyl-2-methoxy-pyridin-3-yl)-5-(2-methoxy-1-methyl-ethyl)-7-methyl-5H-pyrrolo[2,3-b]pyrazine;
{3-[3-Ethyl-5-((S)-2-methoxy-1-methyl-ethyl)-7-methyl-5H-pyrrolo[2,3-b]pyrazin-2-yl]-6-isopropyl-pyridin-2-yl}-methyl-amine;
{3-[3-Chloro-5-((S)-2-methoxy-1-methyl-ethyl)-7-methyl-5H-pyrrolo[2,3-b]pyrazin-2-yl]-6-isopropyl-pyridin-2-yl}-methyl-amine;
{6-Isopropyl-3-[5-((R)-1-methoxymethyl-propyl)-3,7-dimethyl-5H-pyrrolo[2,3-b]pyrazin-2-yl]-pyridin-2-yl}-dimethyl-amine;
3-Chloro-2-(6-isopropyl-2-methyl-pyridin-3-yl)-5-((S)-2-methoxy-1-methyl-ethyl)-7-methyl-5H-pyrrolo[2,3-b]pyrazine;
5-((R)-1-Ethoxymethyl-propyl)-2-(6-isopropyl-2-methoxy-pyridin-3-yl)-3,7-dimethyl-5H-pyrrolo[2,3-b]pyrazine;
2-(6-Isopropyl-2-methyl-pyridin-3-yl)-5-((R)-1-methoxymethyl-propyl)-3,7-dimethyl-5H-pyrrolo[2,3-b]pyrazine;
{3-[5-((R)-1-Ethoxymethyl-propyl)-3,7-dimethyl-5H-pyrrolo[2,3-b]pyrazin-2-yl]-6-isopropyl-pyridin-2-yl}-methyl-amine;
Ethyl-{6-isopropyl-3-[5-((R)-1-methoxymethyl-propyl)-3,7-dimethyl-5H-pyrrolo[2,3-b]pyrazin-2-yl]-pyridin-2-yl}-amine;
1-(1-Ethyl-propyl)-5-(2-methoxy-4-trifluoromethoxy-phenyl)-3,6-dimethyl-1H-pyrrolo[3,2-b]pyridine;
Ethyl-{4-ethyl-5-[1-(1-ethyl-propyl)-3,6-dimethyl-1H-pyrrolo[3,2-b]pyridin-5-yl]-pyridin-2-yl}-methyl-amine;
1-(1-Ethyl-propyl)-5-(6-isopropyl-2-methoxy-pyridin-3-yl)-3,6-dimethyl-1H-pyrrolo[3,2-b]pyridine;
5-(2-Ethyl-6-isopropyl-pyridin-3-yl)-1-(1-ethyl-propyl)-3,6-dimethyl-1H-pyrrolo[3,2-b]pyridine;
{4-Ethyl-5-[1-(1-ethyl-propyl)-3,6-dimethyl-1H-pyrrolo[3,2-b]pyridin-5-yl]-pyridin-2-yl}-dimethyl-amine;
{3-[1-(1-Ethyl-propyl)-3,6-dimethyl-1H-pyrrolo[3,2-b]pyridin-5-yl]-6-isopropyl-pyridin-2-yl}-methyl-amine;
1-sec-Butyl-5-(2-methoxy-4-trifluoromethoxy-phenyl)-3,6-dimethyl-1H-pyrrolo[3,2-b]pyridine;
1-sec-Butyl-5-(6-isopropyl-2-methoxy-pyridin-3-yl)-3,6-dimethyl-1H-pyrrolo[3,2-b]pyridine;
1-(2-Methoxy-1-methyl-ethyl)-5-(2-methoxy-4-trifluoromethoxy-phenyl)-3,6-dimethyl-1H-pyrrolo[3,2-b]pyridine;
5-(6-Isopropyl-2-methoxy-pyridin-3-yl)-1-(2-methoxy-1-methyl-ethyl)-3,6-dimethyl-1H-pyrrolo[3,2-b]pyridine;
1-sec-Butyl-5-(2-ethyl-6-isopropyl-pyridin-3-yl)-3,6-dimethyl-1H-pyrrolo[3,2-b]pyridine;
[3-(1-sec-Butyl-3,6-dimethyl-1H-pyrrolo[3,2-b]pyridin-5-yl)-6-isopropyl-pyridin-2-yl]-methyl-amine;
5-(2-Ethyl-6-isopropyl-pyridin-3-yl)-1-(2-methoxy-1-methyl-ethyl)-3,6-dimethyl-1H-pyrrolo[3,2-b]pyridine;
{6-Isopropyl-3-[1-(2-methoxy-1-methyl-ethyl)-3,6-dimethyl-1H-pyrrolo[3,2-b]pyridin-5-yl]-pyridin-2-yl}-methyl-amine;
1-Isopropyl-5-(2-methoxy-4-trifluoromethoxy-phenyl)-3,6-dimethyl-1H-pyrrolo[3,2-b]pyridine;
1-Isopropyl-5-(6-isopropyl-2-methoxy-pyridin-3-yl)-3,6-dimethyl-1H-pyrrolo[3,2-b]pyridine;
[6-Isopropyl-3-(1-isopropyl-3,6-dimethyl-1H-pyrrolo[3,2-b]pyridin-5-yl)-pyridin-2-yl]-methyl-amine;
5-(2-Ethyl-6-isopropyl-pyridin-3-yl)-1-isopropyl-3,6-dimethyl-1H-pyrrolo[3,2-b]pyridine;
1-sec-Butyl-6-ethyl-5-(6-isopropyl-2-methoxy-pyridin-3-yl)-3-methyl-1H-pyrrolo[3,2-b]pyridine;
1-(2-Fluoro-ethyl)-5-(6-isopropyl-2-methoxy-pyridin-3-yl)-3,6-dimethyl-1H-pyrrolo[3,2-b]pyridine;
1-[5-(6-Isopropyl-2-methoxy-pyridin-3-y)-3,6-dimethyl-pyrrolo[3,2-b]pyridin-1-yl]-ethanone;
[5-(6-Isopropyl-2-methoxy-pyridin-3-yl)-3,6-dimethyl-pyrrolo[3,2-b]pyridin-1-yl]-acetic acid ethyl ester;
1-Ethyl-5-(6-isopropyl-2-methoxy-pyridin-3-yl)-3,6-dimethyl-1H-pyrrolo[3,2-b]pyridine;
2-[5-(6-Isopropyl-2-methoxy-pyridin-3-yl)-3,6-dimethyl-pyrrolo[3,2-b]pyridin-1-yl]-propionic acid ethyl ester;
5-(6-Isopropyl-2-methoxy-pyridin-3-yl)-1,3,6-trimethyl-1H-pyrrolo[3,2-b]pyridine;
5-(6-Isopropyl-2-methoxy-pyridin-3-yl)-1-(2-methoxy-ethyl)-3,6-dimethyl-1H-pyrrolo[3,2-b]pyridine;
2-[5-(6-Isopropyl-2-methoxy-pyridin-3-yl)-3,6-dimethyl-pyrrolo[3,2-b]pyridin-1-yl]-propionic acid tert-butyl ester;
1-Ethyl-5-(2-ethyl-6-isopropyl-pyridin-3-yl)-3,6-dimethyl-1H-pyrrolo[3,2-b]pyridine;
[3-(1-Ethyl-3,6-dimethyl-1H-pyrrolo[3,2-b]pyridin-5-yl)-6-isopropyl-pyridin-2-yl]-methyl-amine;
5-(6-Isopropyl-2-methoxy-pyridin-3-yl)-3,6-dimethyl-1-propyl-1H-pyrrolo[3,2-b]pyridine;
1-(2-Ethoxy-ethyl)-5-(6-isopropyl-2-methoxy-pyridin-3-yl)-3,6-dimethyl-1H-pyrrolo[3,2-b]pyridine;
5-(2-Ethyl-6-isopropyl-pyridin-3-yl)-1-(2-fluoro-ethyl)-3,6-dimethyl-1H-pyrrolo[3,2-b]pyridine;
{3-[1-(2-Fluoro-ethyl)-3,6-dimethyl-1H-pyrrolo[3,2-b]pyridin-5-yl]-6-isopropyl-pyridin-2-yl}-methyl-amine;
2-[5-(6-Isopropyl-2-methoxy-pyridin-3-yl)-3,6-dimethyl-pyrrolo[3,2-b]pyridin-1-yl]-ethanol;
2-[5-(6-Isopropyl-2-methoxy-pyridin-3-yl)-3,6-dimethyl-pyrrolo[3,2-b]pyridin-1-yl]-N-methyl-propionamide;
5-(2-Ethyl-6-isopropyl-pyridin-3-yl)-3,6-dimethyl-1-propyl-1H-pyrrolo[3,2-b]pyridine;
1-Isobutyl-5-(6-isopropyl-2-methoxy-pyridin-3-yl)-3,6-dimethyl-1H-pyrrolo[3,2-b]pyridine;
1-Cyclopropylmethyl-5-(6-isopropyl-2-methoxy-pyridin-3-y)-3,6-dimethyl-1H-pyrrolo[3,2-b]pyridine;
Ethyl-[6-isopropyl-3-(1-isopropyl-3,6-dimethyl-1H-pyrrolo[3,2-b]pyridin-5-yl)-pyridin-2-yl]-amine;
[3-(3,6-Dimethyl-1-propyl-1H-pyrrolo[3,2-b]pyridin-5-yl)-6-isopropyl-pyridin-2-yl]-methyl-amine;
[3-(3,6-Dimethyl-1-propyl-1H-pyrrolo[3,2-b]pyridin-5-yl)-6-isopropyl-pyridin-2-yl]-ethyl-amine;
1-(3-Fluoro-propyl)-5-(6-isopropyl-2-methoxy-pyridin-3-yl)-3,6-dimethyl-1H-pyrrolo[3,2-b]pyridine;
1-[2-(2-Fluoro-ethoxy)-ethyl]-5-(6-isopropyl-2-methoxy-pyridin-3-y)-3,6-dimethyl-1H-pyrrolo[3,2-b]pyridine;
5-(2-Ethyl-6-isopropyl-pyridin-3-yl)-1-(3-fluoro-propyl)-3,6-dimethyl-1H-pyrrolo[3,2-b]pyridine;
{3-[1-(3-Fluoro-propyl)-3,6-dimethyl-1H-pyrrolo[3,2-b]pyridin-5-yl]-6-isopropyl-pyridin-2-yl}-methyl-amine;
5-(6-Isopropyl-2-methoxy-pyridin-3-yl)-1-(3-methoxy-propyl)-3,6-dimethyl-1H-pyrrolo[3,2-b]pyridine;
{6-Isopropyl-3-[1-((S)-2-methoxy-1-methyl-ethyl)-3,6-dimethyl-1H-pyrrolo[3,2-b]pyridin-5-yl]-pyridin-2-yl}-methyl-amine;
5-(2-Ethyl-6-isopropyl-pyridin-3-yl)-1-((S)-2-methoxy-1-methyl-ethyl)-3,6-dimethyl-1H-pyrrolo[3,2-b]pyridine;
[3-(1-Isobutyl-3,6-dimethyl-1H-pyrrolo[3,2-b]pyridin-5-yl)-6-isopropyl-pyridin-2-yl]-methyl-amine;
5-(2-Ethyl-6-isopropyl-pyridin-3-yl)-1-isobutyl-3,6-dimethyl-1H-pyrrolo[3,2-b]pyridine;
1-Butyl-5-(6-isopropyl-2-methoxy-pyridin-3-yl)-3,6-dimethyl-1H-pyrrolo[3,2-b]pyridine;
5-(6-Isopropyl-2-methoxy-pyridin-3-yl)-3,6-dimethyl-1-(2-morpholin-4-yl-ethyl)-1H-pyrrolo[3,2-b]pyridine;
1-Allyl-5-(6-isopropyl-2-methoxy-pyridin-3-yl)-3,6-dimethyl-1H-pyrrolo[3,2-b]pyridine;
[3-(1-Butyl-3,6-dimethyl-1H-pyrrolo[3,2-b]pyridin-5-yl)-6-isopropyl-pyridin-2-yl]-methyl-amine;
1-Butyl-5-(2-ethyl-6-isopropyl-pyridin-3-yl)-3,6-dimethyl-1H-pyrrolo[3,2-b]pyridine;
(R)-2-[5-(6-Isopropyl-2-methoxy-pyridin-3-y)-3,6-dimethyl-pyrrolo[3,2-b]pyridin-1-yl]-propan-1-ol;
{6-Isopropyl-3-[1-((R)-2-methoxy-1-methyl-ethyl)-3,6-dimethyl-1H-pyrrolo[3,2-b]pyridin-5-yl]-pyridin-2-yl}-methyl-amine;
5-(2-Ethyl-6-isopropyl-pyridin-3-yl)-1-((R)-2-methoxy-1-methyl-ethyl)-3,6-dimethyl-1H-pyrrolo[3,2-b]pyridine;
5-(6-Isopropyl-2-methoxy-pyridin-3-yl)-1-((R)-2-methoxy-1-methyl-ethyl)-3,6-dimethyl-1H-pyrrolo[3,2-b]pyridine;
1-((R)-2-Fluoro-1-methyl-ethyl)-5-(6-isopropyl-2-methoxy-pyridin-3-yl)-3,6-dimethyl-1H-pyrrolo[3,2-b]pyridine;
5-(6-Isopropyl-2-methoxy-pyridin-3-yl)-3,6-dimethyl-1-(2-methyl-allyl)-1H-pyrrolo[3,2-b]pyridine;
[3-(1-Allyl-3,6-dimethyl-1H-pyrrolo[3,2-b]pyridin-5-yl)-6-isopropyl-pyridin-2-yl]-methyl-amine;
1-Allyl-5-(2-ethyl-6-isopropyl-pyridin-3-yl)-3,6-dimethyl-1H-pyrrolo[3,2-b]pyridine;
5-(6-Isopropyl-pyridin-3-yl)-3,6-dimethyl-1-propyl-1H-pyrrolo[3,2-b]pyridine;
(S)-2-[5-(6-Isopropyl-2-methoxy-pyridin-3-yl)-3,6-dimethyl-pyrrolo[3,2-b]pyridin-1-yl]-3-methoxy-propan-1-ol;
1-((R)-1-Fluoromethyl-2-methoxy-ethyl)-5-(6-isopropyl-2-methoxy-pyridin-3-yl)-3,6-dimethyl-1H-pyrrolo[3,2-b]pyridine;
{3-[1-((R)-1-Fluoromethyl-2-methoxy-ethyl)-3,6-dimethyl-1H-pyrrolo[3,2-b]pyridin-5-yl]-6-isopropyl-pyridin-2-yl}-methyl-amine;
5-(2-Ethyl-6-isopropyl-pyridin-3-yl)-1-((R)-1-fluoromethyl-2-methoxy-ethyl)-3,6-dimethyl-1H-pyrrolo[3,2-b]pyridine;
1-((R)-1-Fluoromethyl-2-methoxy-ethyl)-5-(6-isopropyl-pyridin-3-y)-3,6-dimethyl-1H-pyrrolo[3,2-b]pyridine;
5-(6-Isopropyl-2-methoxy-pyridin-3-yl)-1-(1-methoxymethyl-butyl)-3,6-dimethyl-1H-pyrrolo[3,2-b]pyridine;
{5-Bromo-6-isopropyl-3-[1-((S)-2-methoxy-1-methyl-ethyl)-3,6-dimethyl-1H-pyrrolo[3,2-b]pyridin-5-yl]-pyridin-2-yl}-methyl-amine;
{5-Ethyl-6-isopropyl-3-[1-((S)-2-methoxy-1-methyl-ethyl)-3,6-dimethyl-1H-pyrrolo[3,2-b]pyridin-5-yl]-pyridin-2-yl}-methyl-amine;
1-((S)-1-Fluoromethyl-propyl)-5-(6-isopropyl-2-methoxy-pyridin-3-yl)-3,6-dimethyl-1H-pyrrolo[3,2-b]pyridine;
1-((R)-1-Fluoromethyl-propyl)-5-(6-isopropyl-2-methoxy-pyridin-3-yl)-3,6-dimethyl-1H-pyrrolo[3,2-b]pyridine;
{3-[1-((S)-1-Fluoromethyl-propyl)-3,6-dimethyl-1H-pyrrolo[3,2-b]pyridin-5-yl]-6-isopropyl-pyridin-2-yl}-methyl-amine;
(S)-3-[5-(6-Isopropyl-2-methoxy-pyridin-3-yl)-3,6-dimethyl-pyrrolo[3,2-b]pyridin-1-yl]-4-methoxy-butyronitrile;
(R)-2-[5-(6-Isopropyl-2-methoxy-pyridin-3-yl)-3,6-dimethyl-pyrrolo[3,2-b]pyridin-1-yl]-pentan-1-ol;
5-(6-Isopropyl-2-methoxy-pyridin-3-yl)-1-((R)-1-methoxymethyl-butyl)-3,6-dimethyl-1H-pyrrolo[3,2-b]pyridine;
1-((R)-1-Fluoromethyl-butyl)-5-(6-isopropyl-2-methoxy-pyridin-3-yl)-3,6-dimethyl-1H-pyrrolo[3,2-b]pyridine;
5-(6-Isopropyl-2-methoxy-pyridin-3-yl)-1-(1-methoxymethyl-vinyl)-3,6-dimethyl-1H-pyrrolo[3,2-b]pyridine;
{6-Isopropyl-3-[1-((R)-1-methoxymethyl-butyl)-3,6-dimethyl-1H-pyrrolo[3,2-b]pyridin-5-yl]-pyridin-2-yl}-methyl-amine;
5-(2-Ethyl-6-isopropyl-pyridin-3-yl)-1-((R)-1-methoxymethyl-butyl)-3,6-dimethyl-1H-pyrrolo[3,2-b]pyridine;
(S)-2-[6-Ethyl-5-(6-isopropyl-2-methoxy-pyridin-3-yl)-3-methyl-pyrrolo[3,2-b]pyridin-1-yl]-3-methoxy-propan-1-ol;
6-Ethyl-1-((R)-1-fluoromethyl-2-methoxy-ethyl)-5-(6-isopropyl-2-methoxy-pyridin-3-yl)-3-methyl-1H-pyrrolo[3,2-b]pyridine;
5-(6-Isopropyl-2-methoxy-pyridin-3-yl)-1-(2-methoxy-1-methoxymethyl-ethyl)-3,6-dimethyl-1H-pyrrolo[3,2-b]pyridine;
5-(2-Ethyl-6-isopropyl-pyridin-3-yl)-1-((S)-1-fluoromethyl-propyl)-3,6-dimethyl-1H-pyrrolo[3,2-b]pyridine;
1-((S)-1-Fluoromethyl-propyl)-5-(6-isopropyl-pyridin-3-yl)-3,6-dimethyl-1H-pyrrolo[3,2-b]pyridine;
{6-Isopropyl-3-[1-(2-methoxy-1-methoxymethyl-ethyl)-3,6-dimethyl-1H-pyrrolo[3,2-b]pyridin-5-yl]-pyridin-2-yl}-methyl-amine;
1-((S)-1-Ethoxymethyl-propyl)-5-(6-isopropyl-2-methoxy-pyridin-3-yl)-3,6-dimethyl-1H-pyrrolo[3,2-b]pyridine;
(R)-2-[5-(6-Isopropyl-2-methoxy-pyridin-3-yl)-3,6-dimethyl-pyrrolo[3,2-b]pyridin-1-yl]-3-methoxy-propan-1-ol;
1-((S)-1-Fluoromethyl-2-methoxy-ethyl)-5-(6-isopropyl-2-methoxy-pyridin-3-yl)-3,6-dimethyl-1H-pyrrolo[3,2-b]pyridine;
{3-[1-((S)-1-Fluoromethyl-2-methoxy-ethyl)-3,6-dimethyl-1H-pyrrolo[3,2-b]pyridin-5-yl]-6-isopropyl-pyridin-2-yl}-methyl-amine;
6-Ethyl-1-isopropyl-5-(6-isopropyl-2-methoxy-pyridin-3-yl)-3-methyl-1H-pyrrolo[3,2-b]pyridine;
[3-(6-Ethyl-1-isopropyl-3-methyl-1H-pyrrolo[3,2-b]pyridin-5-yl)-6-isopropyl-pyridin-2-yl]-methyl-amine;
6-Ethyl-5-(2-ethyl-6-isopropyl-pyridin-3-yl)-1-isopropyl-3-methyl-1H-pyrrolo[3,2-b]pyridine;
5-(2-Ethyl-6-isopropyl-pyridin-3-yl)-1-(2-methoxy-1-methoxymethyl-ethyl)-3,6-dimethyl-1H-pyrrolo[3,2-b]pyridine;
{3-[1-((S)-1-Ethoxymethyl-propyl)-3,6-dimethyl-1H-pyrrolo[3,2-b]pyridin-5-yl]-6-isopropyl-pyridin-2-yl}-methyl-amine;
2,5-Diethyl-6-[1-(1-ethyl-propyl)-3,6-dimethyl-1H-pyrrolo[3,2-b]pyridin-5-yl]-3-isopropyl-3H-imidazo[4,5-b]pyridine;
(S)-2-[5-(2-Methoxy-4-trifluoromethoxy-phenyl)-3,6-dimethyl-pyrrolo[3,2-b]pyridin-1-yl]-butan-1-ol;
1-((S)-1-Methoxymethyl-propyl)-5-(2-methoxy-4-trifluoromethoxy-phenyl)-3,6-dimethyl-1H-pyrrolo[3,2-b]pyridine;
1-((S)-1-Chloromethyl-propyl)-5-(2-methoxy-4-trifluoromethoxy-phenyl)-3,6-dimethyl-1H-pyrrolo[3,2-b]pyridine;
1-((S)-2-Methoxy-1-methyl-ethyl)-5-(2-methoxy-4-trifluoromethoxy-phenyl)-3,6-dimethyl-1H-pyrrolo[3,2-b]pyridine;
5-(2-Methoxy-4-trifluoromethoxy-phenyl)-3,6-dimethyl-1H-pyrrolo[3,2-b]pyridine;
5-(2-Methoxy-4-trifluoromethoxy-phenyl)-3,6-dimethyl-1-((S)-1-morpholin-4-ylmethyl-propyl)-1H-pyrrolo[3,2-b]pyridine;
{(S)-2-[5-(2-Methoxy-4-trifluoromethoxy-phenyl)-3,6-dimethyl-pyrrolo[3,2-b]pyridin-1-yl]-butyl}-dimethyl-amine;
5-(2-Methoxy-4-trifluoromethoxy-phenyl)-3,6-dimethyl-1-((S)-1-pyrrolidin-1-ylmethyl-propyl)-1H-pyrrolo[3,2-b]pyridine;
(S)-2-[5-(6-Isopropyl-2-methoxy-pyridin-3-yl)-3,6-dimethyl-pyrrolo[3,2-b]pyridin-1-yl]-butan-1-ol;
5-(6-Isopropyl-2-methoxy-pyridin-3-yl)-1-((S)-1-methoxymethyl-propyl)-3,6-dimethyl-1H-pyrrolo[3,2-b]pyridine;
Methanesulfonic acid (S)-2-[5-(6-isopropyl-2-methoxy-pyridin-3-yl)-3,6-dimethyl-pyrrolo[3,2-b]pyridin-1-yl]-butyl ester;
{(S)-2-[5-(6-Isopropyl-2-methoxy-pyridin-3-yl)-3,6-dimethyl-pyrrolo[3,2-b]pyridin-1-yl]-butyl}-dimethyl-amine;
(2R,6S)-2,6-Dimethyl-morpholine-4-carboxylic acid 2-[5-(2-methoxy-4-trifluoromethoxy-phenyl)-3,6-dimethyl-pyrrolo[3,2-b]pyridin-1-yl]-butylester;
Piperidine-1-carboxylic acid (S)-2-[5-(2-methoxy-4-trifluoromethoxy-phenyl)-3,6-dimethyl-pyrrolo[3,2-b]pyridin-1-yl]-butyl ester;
4-Methyl-piperazine-1-carboxylic acid (S)-2-[5-(2-methoxy-4-trifluoromethoxy-phenyl)-3,6-dimethyl-pyrrolo[3,2-b]pyridin-1-yl]-butyl ester;
Azepane-1-carboxylic acid (S)-2-[5-(2-methoxy-4-trifluoromethoxy-phenyl)-3,6-dimethyl-pyrrolo[3,2-b]pyridin-1-yl]-butyl ester;
4-Acetyl-piperazine-1-carboxylic acid (S)-2-[5-(2-methoxy-4-trifluoromethoxy-phenyl)-3,6-dimethyl-pyrrolo[3,2-b]pyridin-1-yl]-butyl ester;
Ethyl-methyl-carbamic acid (S)-2-[5-(2-methoxy-4-trifluoromethoxy-phenyl)-3,6-dimethyl-pyrrolo[3,2-b]pyridin-1-yl]-butyl ester;
Diethyl-carbamic acid (S)-2-[5-(2-methoxy-4-trifluoromethoxy-phenyl)-3,6-dimethyl-pyrrolo[3,2-b]pyridin-1-yl]-butyl ester;
Ethyl-(2-methoxy-ethyl)-carbamic acid (S)-2-[5-(2-methoxy-4-trifluoromethoxy-phenyl)-3,6-dimethyl-pyrrolo[3,2-b]pyridin-1-yl]-butyl ester;
(2-Methoxy-ethyl)-carbamic acid (S)-2-[5-(2-methoxy-4-trifluoromethoxy-phenyl)-3,6-dimethyl-pyrrolo[3,2-b]pyridin-1-yl]-butyl ester;
Cyclopentyl-carbamic acid (S)-2-[5-(2-methoxy-4-trifluoromethoxy-phenyl)-3,6-dimethyl-pyrrolo[3,2-b]pyridin-1-yl]-butyl ester;
1-[(S)-1-((2S,6R)-2,6-Dimethyl-morpholin-4-ylmethyl)-propyl]-5-(2-methoxy-4-trifluoromethoxy-phenyl)-3,6-dimethyl-1H-pyrrolo[3,2-b]pyridine;
5-(2-Methoxy-4-trifluoromethoxy-phenyl)-3,6-dimethyl-1-((S)-1-piperidin-1-ylmethyl-propyl)-1H-pyrrolo[3,2-b]pyridine;
1-((S)-1-Methanesulfonylmethyl-propyl)-5-(2-methoxy-4-trifluoromethoxy-phenyl)-3,6-dimethyl-1H-pyrrolo[3,2-b]pyridine;
5-(2-Methoxy-4-trifluoromethoxy-phenyl)-3,6-dimethyl-1-[(S)-1-(4-methyl-piperazin-1-ylmethyl)-propyl]-1H-pyrrolo[3,2-b]pyridine;
1-((S)-1-Azepan-1-ylmethyl-propyl)-5-(2-methoxy-4-trifluoromethoxy-phenyl)-3,6-dimethyl-1H-pyrrolo[3,2-b]pyridine;
Methanesulfonic acid (S)-2-[5-(2-methoxy-4-trifluoromethoxy-phenyl)-3,6-dimethyl-pyrrolo[3,2-b]pyridin-1-yl]-butyl ester;
5-(6-Isopropyl-2-methoxy-pyridin-3-yl)-3,6-dimethyl-1-((S)-1-morpholin-4-ylmethyl-propyl)-1H-pyrrolo[3,2-b]pyridine;
{3-[3,6-Dimethyl-1-((S)-1-morpholin-4-ylmethyl-propyl)-1H-pyrrolo[3,2-b]pyridin-5-yl]-6-isopropyl-pyridin-2-yl}-methyl-amine;
5-(2-Ethyl-6-isopropyl-pyridin-3-yl)-3,6-dimethyl-1-((S)-1-morpholin-4-ylmethyl-propyl)-1H-pyrrolo[3,2-b]pyridine;
1-[(S)-1-(3,3-Dimethyl-piperidin-1-ylmethyl)-propyl]-5-(2-methoxy-4-trifluoromethoxy-phenyl)-3,6-dimethyl-1H-pyrrolo[3,2-b]pyridine;
5-(2-Methoxy-4-trifluoromethoxy-phenyl)-3,6-dimethyl-1-((S)-1-thiomorpholin-4-ylmethyl-propyl)-1H-pyrrolo[3,2-b]pyridine;
1-[(S)-1-(4,4-Difluoro-piperidin-1-ylmethyl)-propyl]-5-(2-methoxy-4-trifluoromethoxy-phenyl)-3,6-dimethyl-1H-pyrrolo[3,2-b]pyridine;
(R)-2-[5-(6-Isopropyl-2-methoxy-pyridin-3-yl)-3,6-dimethyl-pyrrolo[3,2-b]pyridin-1-yl]-butan-1-ol;
5-(6-Isopropyl-2-methoxy-pyridin-3-yl)-3,6-dimethyl-1-((R)-1-morpholin-4-ylmethyl-propyl)-1H-pyrrolo[3,2-b]pyridine;
5-(6-Isopropyl-2-methoxy-pyridin-3-yl)-1-((R)-1-methoxymethyl-propyl)-3,6-dimethyl-1H-pyrrolo[3,2-b]pyridine;
{3-[3,6-Dimethyl-1-((R)-1-morpholin-4-ylmethyl-propyl)-1H-pyrrolo[3,2-b]pyridin-5-yl]-6-isopropyl-pyridin-2-yl}-methyl-amine;
5-(2-Ethyl-6-isopropyl-pyridin-3-yl)-3,6-dimethyl-1-((R)-1-morpholin-4-ylmethyl-propyl)-1H-pyrrolo[3,2-b]pyridine;
5-(2-Ethyl-6-isopropyl-pyridin-3-yl)-1-((R)-1-methoxymethyl-propyl)-3,6-dimethyl-1H-pyrrolo[3,2-b]pyridine;
{6-Isopropyl-3-[1-((R)-1-methoxymethyl-propyl)-3,6-dimethyl-1H-pyrrolo[3,2-b]pyridin-5-yl]-pyridin-2-yl}-methyl-amine;
5-(6-Isopropyl-pyridin-3-yl)-1-((R)-1-methoxymethyl-propyl)-3,6-dimethyl-1H-pyrrolo[3,2-b]pyridine;
6-Ethyl-5-(6-isopropyl-2-methoxy-pyridin-3-yl)-1-((S)-2-methoxy-1-methyl-ethyl)-3-methyl-1H-pyrrolo[3,2-b]pyridine;
(S)-2-[6-Ethyl-5-(6-isopropyl-2-methoxy-pyridin-3-yl)-3-methyl-pyrrolo[3,2-b]pyridin-1-yl]-propan-1-ol;
6-Ethyl-5-(6-isopropyl-pyridin-3-yl)-1-((S)-2-methoxy-1-methyl-ethyl)-3-methyl-1H-pyrrolo[3,2-b]pyridine;
6-Ethyl-5-(2-ethyl-6-isopropyl-pyridin-3-yl)-1-((S)-2-methoxy-1-methyl-ethyl)-3-methyl-1H-pyrrolo[3,2-b]pyridine;
{3-[6-Ethyl-1-((S)-2-methoxy-1-methyl-ethyl)-3-methyl-1H-pyrrolo[3,2-b]pyridin-5-yl]-6-isopropyl-pyridin-2-yl}-methyl-amine;
(R)-1-[5-(6-Isopropyl-2-methoxy-pyridin-3-yl)-3,6-dimethyl-pyrrolo[3,2-b]pyridin-1-yl]-butan-2-ol;
1-[5-(6-Isopropyl-2-methoxy-pyridin-3-yl)-3,6-dimethyl-pyrrolo[3,2-b]pyridin-1-yl]-2-methyl-propan-2-ol;
5-(6-Isopropyl-2-methoxy-pyridin-3-yl)-1-((R)-2-methoxy-butyl)-3,6-dimethyl-1H-pyrrolo[3,2-b]pyridine;
(R)-2-[6-Ethyl-5-(6-isopropyl-2-methoxy-pyridin-3-yl)-3-methyl-pyrrolo[3,2-b]pyridin-1-yl]-propan-1-ol;
(R)-2-[5-(2-Ethoxy-6-ethyl-5-methanesulfonyl-pyridin-3-yl)-6-ethyl-3-methyl-pyrrolo[3,2-b]pyridin-1-yl]-propan-1-ol;
5-(2-Ethoxy-6-ethyl-5-methanesulfonyl-pyridin-3-yl)-6-ethyl-1-((R)-2-methoxy-1-methyl-ethyl)-3-methyl-1H-pyrrolo[3,2-b]pyridine;
(R)-2-[6-Ethyl-5-(6-isopropyl-2-methoxy-pyridin-3-yl)-3-methyl-pyrrolo[3,2-b]pyridin-1-yl]-butan-1-ol;
(R)-2-[5-(2-Ethoxy-6-ethyl-5-methanesulfonyl-pyridin-3-yl)-6-ethyl-3-methyl-pyrrolo[3,2-b]pyridin-1-yl]-butan-1-ol;
5-(2-Ethoxy-6-ethyl-5-methanesulfonyl-pyridin-3-yl)-6-ethyl-1-((R)-1-methoxymethyl-propyl)-3-methyl-1H-pyrrolo[3,2-b]pyridine;
6-Ethyl-5-(6-isopropyl-2-methoxy-pyridin-3-yl)-1-((R)-1-methoxymethyl-propyl)-3-methyl-1H-pyrrolo[3,2-b]pyridine;
6-Ethyl-5-(6-isopropyl-2-methoxy-pyridin-3-yl)-1-((R)-2-methoxy-1-methyl-ethyl)-3-methyl-1H-pyrrolo[3,2-b]pyridine;
5-(2-Azetidin-1-yl-6-isopropyl-pyridin-3-yl)-1-((R)-1-methoxymethyl-propyl)-3,6-dimethyl-1H-pyrrolo[3,2-b]pyridine;
5-(2-Azetidin-1-yl-6-isopropyl-pyridin-3-yl)-6-ethyl-1-((R)-1-methoxymethyl-propyl)-3-methyl-1H-pyrrolo[3,2-b]pyridine;
{3-[6-Ethyl-1-((R)-1-methoxymethyl-propyl)-3-methyl-1H-pyrrolo[3,2-b]pyridin-5-yl]-6-isopropyl-pyridin-2-yl}-methyl-amine;
6-Ethyl-5-(2-ethyl-6-isopropyl-pyridin-3-yl)-1-((R)-1-methoxymethyl-propyl)-3-methyl-1H-pyrrolo[3,2-b]pyridine;
6-Ethyl-5-(2-ethyl-6-isopropyl-pyridin-3-yl)-1-((R)-2-methoxy-1-methyl-ethyl)-3-methyl-1H-pyrrolo[3,2-b]pyridine;
{3-[6-Ethyl-1-((R)-2-methoxy-1-methyl-ethyl)-3-methyl-1H-pyrrolo[3,2-b]pyridin-5-yl]-6-isopropyl-pyridin-2-yl}-methyl-amine;
6-Ethyl-5-(4-isopropyl-2-methoxy-phenyl)-1-((R)-2-methoxy-1-methyl-ethyl)-3-methyl-1H-pyrrolo[3,2-b]pyridine;
6-Ethyl-1-((R)-2-fluoro-1-methyl-ethyl)-5-(4-isopropyl-2-methoxy-phenyl)-3-methyl-1H-pyrrolo[3,2-b]pyridine;
6-Ethyl-1-((R)-2-fluoro-1-methyl-ethyl)-5-(6-isopropyl-2-methoxy-pyridin-3-y1)-3-methyl-1H-pyrrolo[3,2-b]pyridine;
{5-Chloro-3-[7-chloro-6-ethyl-1-((R)-2-methoxy-1-methyl-ethyl)-3-methyl-1H-pyrrolo[3,2-b]pyridin-5-yl]-6-isopropyl-pyridin-2-yl}-methyl-amine;
{5-Chloro-3-[6-ethyl-1-((R)-2-methoxy-1-methyl-ethyl)-3-methyl-1H-pyrrolo[3,2-b]pyridin-5-yl]-6-isopropyl-pyridin-2-yl}-methyl-amine;
{5-Bromo-3-[6-ethyl-1-((R)-2-methoxy-1-methyl-ethyl)-3-methyl-1H-pyrrolo[3,2-b]pyridin-5-yl]-6-isopropyl-pyridin-2-yl}-methyl-amine;
{5-Cyclopropyl-3-[6-ethyl-1-((R)-2-methoxy-1-methyl-ethyl)-3-methyl-1H-pyrrolo[3,2-b]pyridin-5-yl]-6-isopropyl-pyridin-2-yl}-methyl-amine;
{5-Ethyl-3-[6-ethyl-1-((R)-2-methoxy-1-methyl-ethyl)-3-methyl-1H-pyrrolo[3,2-b]pyridin-5-yl]-6-isopropyl-pyridin-2-yl}-methyl-amine;
(S)-2-[6-Bromo-5-(6-isopropyl-2-methoxy-pyridin-3-yl)-3-methyl-pyrrolo[3,2-b]pyridin-1-yl]-butan-1-ol;
6-Bromo-5-(6-isopropyl-2-methoxy-pyridin-3-yl)-1-((S)-1-methoxymethyl-propyl)-3-methyl-1H-pyrrolo[3,2-b]pyridine;
5-(6-Isopropyl-2-methoxy-pyridin-3-yl)-1-((S)-1-methoxymethyl-propyl)-3-methyl-1H-pyrrolo[3,2-b]pyridine;
5-Ethyl-6-[1-(1-ethyl-propyl)-3,6-dimethyl-1H-pyrrolo[3,2-b]pyridin-5-yl]-3-isopropyl-3H-imidazo[4,5-b]pyridine;
(3S,4R)-3-(2-Fluoro-ethoxy)-4-[5-(2-methoxy-4-trifluoromethoxy-phenyl)-3,6-dimethyl-pyrrolo[3,2-b]pyridin-1-yl]-pyrrolidine-1-carboxylic acid benzyl ester;
(3S,4R)-3-(2-Fluoro-ethoxy)-4-[5-(2-methoxy-4-trifluoromethoxy-phenyl)-3,6-dimethyl-pyrrolo[3,2-b]pyridin-1-yl]-pyrrolidine-1-carboxylic acid methyl ester;
(3S,4R)-3-(2-Fluoro-ethoxy)-4-[5-(6-isopropyl-2-methoxy-pyridin-3-yl)-3,6-dimethyl-pyrrolo[3,2-b]pyridin-1-yl]-pyrrolidine-1-carboxylic acid benzyl ester;
(3S,4R)-3-(2-Fluoro-ethoxy)-4-[5-(6-isopropyl-2-methoxy-pyridin-3-yl)-3,6-dimethyl-pyrrolo[3,2-b]pyridin-1-yl]-pyrrolidine-1-carboxylic acid methyl ester;
1-[(3R,4S)-4-(2-Fluoro-ethoxy)-1-methanesulfonyl-pyrrolidin-3-yl]-5-(6-isopropyl-2-methoxy-pyridin-3-yl)-3,6-dimethyl-1H-pyrrolo[3,2-b]pyridine;
1-[(3R,4S)-4-(2-Fluoro-ethoxy)-1-methyl-pyrrolidin-3-yl]-5-(6-isopropyl-2-methoxy-pyridin-3-yl)-3,6-dimethyl-1H-pyrrolo[3,2-b]pyridine;
(3S,4R)-3-(2-Fluoro-ethoxy)-4-[5-(6-isopropyl-2-methoxy-pyridin-3-y)-3,6-dimethyl-pyrrolo[3,2-b]pyridin-1-yl]-pyrrolidine-1-carboxylic acid 2-morpholin-4-yl-ethyl ester;
3-Chloro-1-isopropyl-5-(6-isopropyl-2-methoxy-pyridin-3-yl)-6-methyl-1H-pyrrolo[3,2-b]pyridine;
3-Ethyl-5-(6-isopropyl-2-methoxy-pyridin-3-yl)-1-((S)-2-methoxy-1-methyl-ethyl)-6-methyl-1H-pyrrolo[3,2-b]pyridine;
3-Chloro-1-((S)-2-methoxy-1-methyl-ethyl)-5-(2-methoxy-4-trifluoromethoxy-phenyl)-6-methyl-1H-pyrrolo[3,2-b]pyridine;
3-Bromo-1-((S)-2-methoxy-1-methyl-ethyl)-5-(2-methoxy-4-trifluoromethoxy-phenyl)-6-methyl-1H-pyrrolo[3,2-b]pyridine;
1-((S)-2-Methoxy-1-methyl-ethyl)-5-(2-methoxy-4-trifluoromethoxy-phenyl)-6-methyl-1H-pyrrolo[3,2-b]pyridine;
3-Fluoro-1-((S)-2-methoxy-1-methyl-ethyl)-5-(2-methoxy-4-trifluoromethoxy-phenyl)-6-methyl-1H-pyrrolo[3,2-b]pyridine;
5-(6-Isopropyl-2-methoxy-pyridin-3-yl)-1-((S)-2-methoxy-1-methyl-ethyl)-6-methyl-1H-pyrrolo[3,2-b]pyridine;
3-Chloro-5-(6-isopropyl-2-methoxy-pyridin-3-yl)-1-((S)-2-methoxy-1-methyl-ethyl)-6-methyl-1H-pyrrolo[3,2-b]pyridine;
(R)-2-[5-(6-Isopropyl-2-methoxy-pyridin-3-yl)-6-methyl-pyrrolo[3,2-b]pyridin-1-yl]-butan-1-ol;
3-Bromo-5-(6-isopropyl-2-methoxy-pyridin-3-yl)-1-((S)-2-methoxy-1-methyl-ethyl)-6-methyl-1H-pyrrolo[3,2-b]pyridine;
(R)-2-[3-Chloro-5-(6-isopropyl-2-methoxy-pyridin-3-yl)-6-methyl-pyrrolo[3,2-b]pyridin-1-yl]-butan-1-ol;
5-(6-Isopropyl-2-methoxy-pyridin-3-yl)-1-((R)-1-methoxymethyl-propyl)-6-methyl-1H-pyrrolo[3,2-b]pyridine;
3-Chloro-5-(6-isopropyl-2-methoxy-pyridin-3-yl)-1-((R)-1-methoxymethyl-propyl)-6-methyl-1H-pyrrolo[3,2-b]pyridine;
1-[1-((S)-2-Methoxy-1-methyl-ethyl)-5-(2-methoxy-4-trifluoromethoxy-phenyl)-6-methyl-1H-pyrrolo[3,2-b]pyridin-7-yl]-pyrrolidine-2,5-dione;
1-[5-(6-Isopropyl-2-methoxy-pyridin-3-yl)-1-((R)-1-methoxymethyl-propyl)-6-methyl-1H-pyrrolo[3,2-b]pyridin-7-yl]-pyrrolidine-2,5-dione;
{3-[3-Chloro-1-((S)-2-methoxy-1-methyl-ethyl)-6-methyl-1H-pyrrolo[3,2-b]pyridin-5-yl]-6-isopropyl-pyridin-2-yl}-methyl-amine;
{3-[3-Chloro-1-((R)-1-methoxymethyl-propyl)-6-methyl-1H-pyrrolo[3,2-b]pyridin-5-yl]-6-isopropyl-pyridin-2-yl}-methyl-amine;
3-Chloro-5-(2-ethyl-6-isopropyl-pyridin-3-yl)-1-((S)-2-methoxy-1-methyl-ethyl)-6-methyl-1H-pyrrolo[3,2-b]pyridine;
3-Chloro-5-(6-isopropyl-pyridin-3-yl)-1-((S)-2-methoxy-1-methyl-ethyl)-6-methyl-1H-pyrrolo[3,2-b]pyridine;
6-Ethyl-7-[1-((R)-1-hydroxymethyl-propyl)-3,6-dimethyl-1H-pyrrolo[3,2-b]pyridin-5-yl]-4-isopropyl-2-methyl-4H-pyrido[2,3-b]pyrazin-3-one;
6-Ethyl-2,4-diisopropyl-7-[1-((R)-1-methoxymethyl-propyl)-3,6-dimethyl-1H-pyrrolo[3,2-b]pyridin-5-yl]-4H-pyrido[2,3-b]pyrazin-3-one;
2,6-Diethyl-4-isopropyl-7-[1-((R)-1-methoxymethyl-propyl)-3,6-dimethyl-1H-pyrrolo[3,2-b]pyridin-5-yl]-4H-pyrido[2,3-b]pyrazin-3-one;
5-(6-Isopropyl-2-methoxy-pyridin-3-yl)-1-((S)-2-methoxy-1-methyl-ethyl)-6-methyl-1H-pyrrolo[3,2-b]pyridine-3-carbonitrile;
5-(6-Isopropyl-2-methylamino-pyridin-3-yl)-1-((S)-2-methoxy-1-methyl-ethyl)-6-methyl-1H-pyrrolo[3,2-b]pyridine-3-carbonitrile;
6-Ethyl-7-[6-ethyl-1-((S)-2-methoxy-1-methyl-ethyl)-3-methyl-1H-pyrrolo[3,2-b]pyridin-5-yl]-4-isopropyl-2-methyl-4H-pyrido[2,3-b]pyrazin-3-one;
5-(2-Ethyl-6-methoxy-pyridin-3-yl)-1-isopropyl-3,6-dimethyl-1H-pyrrolo[3,2-b]pyridine;
5-(2-Ethyl-6-methoxy-pyridin-3-yl)-1-((S)-2-methoxy-1-methyl-ethyl)-3,6-dimethyl-1H-pyrrolo[3,2-b]pyridine;
5-(2-Ethyl-6-isopropoxy-pyridin-3-yl)-1-((S)-2-methoxy-1-methyl-ethyl)-3,6-dimethyl-1H-pyrrolo[3,2-b]pyridine;
{6-Ethyl-5-[1-((S)-2-methoxy-1-methyl-ethyl)-3,6-dimethyl-1H-pyrrolo[3,2-b]pyridin-5-yl]-pyridin-2-yl}-dimethyl-amine;
5-(2,6-Diethyl-pyridin-3-yl)-1-((S)-2-methoxy-1-methyl-ethyl)-3,6-dimethyl-1H-pyrrolo[3,2-b]pyridine;
5-(2,6-Diethyl-pyridin-3-yl)-1-isopropyl-3,6-dimethyl-1H-pyrrolo[3,2-b]pyridine;
5-(2-Ethyl-6-isopropoxy-pyridin-3-yl)-1-isopropyl-3,6-dimethyl-1H-pyrrolo[3,2-b]pyridine;
[6-Ethyl-5-(1-isopropyl-3,6-dimethyl-1H-pyrrolo[3,2-b]pyridin-5-yl)-pyridin-2-yl]-dimethyl-amine;
5-(6-Cyclopropylmethoxy-2-ethyl-pyridin-3-yl)-1-((S)-2-methoxy-1-methyl-ethyl)-3,6-dimethyl-1H-pyrrolo[3,2-b]pyridine;
2-[5-(6-Isopropyl-2-methoxy-pyridin-3-yl)-3,6-dimethyl-pyrrolo[3,2-b]pyridin-1-yl]-2-methyl-propan-1-ol;
5-(6-Cyclopropyl-2-ethyl-pyridin-3-yl)-1-((S)-2-methoxy-1-methyl-ethyl)-3,6-dimethyl-1H-pyrrolo[3,2-b]pyridine;
5-(6-Ethoxy-2-ethyl-pyridin-3-yl)-1-((S)-2-methoxy-1-methyl-ethyl)-3,6-dimethyl-1H-pyrrolo[3,2-b]pyridine;
5-(6-Isopropyl-2-methoxy-pyridin-3-yl)-1-(2-methoxy-1,1-dimethyl-ethyl)-3,6-dimethyl-1H-pyrrolo[3,2-b]pyridine;
(R)-2-[5-(2-Ethyl-6-methoxy-pyridin-3-yl)-3,6-dimethyl-pyrrolo[3,2-b]pyridin-1-yl]-butan-1-ol;
6-Ethyl-2-methoxy-5-[1-((S)-2-methoxy-1-methyl-ethyl)-3,6-dimethyl-1H-pyrrolo[3,2-b]pyridin-5-yl]-N-methyl-nicotinamide;
5-(2-Ethyl-6-methoxy-pyridin-3-yl)-1-((R)-1-methoxymethyl-propyl)-3,6-dimethyl-1H-pyrrolo[3,2-b]pyridine;
1-{6-Ethyl-2-methoxy-5-[1-((S)-2-methoxy-1-methyl-ethyl)-3,6-dimethyl-1H-pyrrolo[3,2-b]pyridin-5-yl]-pyridin-3-yl}-ethanone;
5-(2-Ethyl-6-isopropyl-pyridin-3-yl)-1-(2-methoxy-1,1-dimethyl-ethyl)-3,6-dimethyl-1H-pyrrolo[3,2-b]pyridine;
{6-Isopropyl-3-[1-(2-methoxy-1,1-dimethyl-ethyl)-3,6-dimethyl-1H-pyrrolo[3,2-b]pyridin-5-yl]-pyridin-2-yl}-methyl-amine;
5-(6-Ethoxy-2-ethyl-pyridin-3-yl)-1-((R)-1-methoxymethyl-propyl)-3,6-dimethyl-1H-pyrrolo[3,2-b]pyridine;
5-(6-Cyclopropylmethoxy-2-ethyl-pyridin-3-yl)-1-((R)-1-methoxymethyl-propyl)-3,6-dimethyl-1H-pyrrolo[3,2-b]pyridine;
5-(2-Ethyl-6-isopropoxy-pyridin-3-yl)-1-((R)-1-methoxymethyl-propyl)-3,6-dimethyl-1H-pyrrolo[3,2-b]pyridine;
6-Ethyl-5-(2-ethyl-6-methoxy-pyridin-3-yl)-1-((R)-2-fluoro-1-methoxymethyl-ethyl)-3-methyl-1H-pyrrolo[3,2-b]pyridine;
5-[2-Ethyl-6-(2-methoxy-ethoxy)-pyridin-3-yl]-1-((S)-2-methoxy-1-methyl-ethyl)-3,6-dimethyl-1H-pyrrolo[3,2-b]pyridine;
5-(6-Isopropyl-2-methoxy-pyridin-3-yl)-1-((S)-2-methoxy-1-methyl-ethyl)-3,6,7-trimethyl-1H-pyrrolo[3,2-b]pyridine;
{6-Isopropyl-3-[1-((S)-2-methoxy-1-methyl-ethyl)-3,6,7-trimethyl-1H-pyrrolo[3,2-b]pyridin-5-yl]-pyridin-2-yl}-methyl-amine;
5-(2-Ethyl-6-isopropyl-pyridin-3-yl)-1-((S)-2-methoxy-1-methyl-ethyl)-3,6,7-trimethyl-1H-pyrrolo[3,2-b]pyridine;
{6-Isopropyl-3-[1-((S)-2-methoxy-1-methyl-ethyl)-3,6,7-trimethyl-1H-pyrrolo[3,2-b]pyridin-5-yl]-pyridin-2-yl}-dimethyl-amine;
5-(2-Azetidin-1-yl-6-isopropyl-pyridin-3-yl)-1-((S)-2-methoxy-1-methyl-ethyl)-3,6,7-trimethyl-1H-pyrrolo[3,2-b]pyridine;
[3-(3,6-Dimethyl-1-propyl-1H-pyrrolo[3,2-b]pyridin-5-yl)-6-isopropyl-pyridin-2-yl]-(2-methoxy-ethyl)-amine;
6-Isopropyl-3-(1-isopropyl-3,6-dimethyl-1H-pyrrolo[3,2-b]pyridin-5-yl)-pyridin-2-yl]-(2-methoxy-ethyl)-amine;
5-(6-Isopropyl-2-methoxy-pyridin-3-yl)-1-((S)-2-methoxy-1-methyl-ethyl)-3,6-dimethyl-1H-pyrrolo[3,2-b]pyridine;
{6-Isopropyl-3-[1-((S)-2-methoxy-1-methyl-ethyl)-3,6-dimethyl-1H-pyrrolo[3,2-b]pyridin-5-yl]-pyridin-2-yl}-dimethyl-amine;
[6-Isopropyl-3-(1-isopropyl-3,6-dimethyl-1H-pyrrolo[3,2-b]pyridin-5-yl)-pyridin-2-yl]-dimethyl-amine;
[3-(3,6-Dimethyl-1-propyl-1H-pyrrolo[3,2-b]pyridin-5-yl)-6-isopropyl-pyridin-2-yl]-dimethyl-amine;
5-(2-Azetidin-1-yl-6-isopropyl-pyridin-3-yl)-3,6-dimethyl-1-propyl-1H-pyrrolo[3,2-b]pyridine;
5-(2-Azetidin-1-yl-6-isopropyl-pyridin-3-yl)-1-isopropyl-3,6-dimethyl-1H-pyrrolo[3,2-b]pyridine;
5-(2-Azetidin-1-yl-6-isopropyl-pyridin-3-yl)-1-((S)-2-methoxy-1-methyl-ethyl)-3,6-dimethyl-1H-pyrrolo[3,2-b]pyridine;
5-[2-(3,3-Difluoro-azetidin-1-yl)-6-isopropyl-pyridin-3-yl]-1-((S)-2-methoxy-1-methyl-ethyl)-3,6-dimethyl-1H-pyrrolo[3,2-b]pyridine;
5-(2-Ethoxy-6-isopropyl-pyridin-3-yl)-1-((S)-2-methoxy-1-methyl-ethyl)-3,6-dimethyl-1H-pyrrolo[3,2-b]pyridine;
5-(2-Isopropoxy-6-isopropyl-pyridin-3-yl)-1-((S)-2-methoxy-1-methyl-ethyl)-3,6-dimethyl-1H-pyrrolo[3,2-b]pyridine;
5-(6-Isopropyl-2-methyl-pyridin-3-yl)-1-((S)-2-methoxy-1-methyl-ethyl)-3,6-dimethyl-1H-pyrrolo[3,2-b]pyridine;
[3-(3,6-Dimethyl-1-propyl-1H-pyrrolo[3,2-b]pyridin-5-yl)-6-isopropyl-pyridin-2-yl]-isopropyl-amine;
Isopropyl-[6-isopropyl-3-(1-isopropyl-3,6-dimethyl-1H-pyrrolo[3,2-b]pyridin-5-yl)-pyridin-2-yl]-amine;
{6-Isopropyl-3-[1-(2-methoxy-1-methyl-ethyl)-3,6-dimethyl-1H-pyrrolo[3,2-b]pyridin-5-yl]-pyridin-2-yl}-(2-methoxy-ethyl)-amine; Isopropyl-{6-isopropyl-3-[1-((S)-2-methoxy-1-methyl-ethyl)-3,6-dimethyl-1H-pyrrolo[3,2-b]pyridin-5-yl]-pyridin-2-yl}-amine;
Ethyl-{6-isopropyl-3-[1-((S)-2-methoxy-1-methyl-ethyl)-3,6-dimethyl-1H-pyrrolo[3,2-b]pyridin-5-yl]-pyridin-2-yl}-amine;
1-Isopropyl-5-[6-isopropyl-2-(4-methyl-piperazin-1-yl)-pyridin-3-yl]-3,6-dimethyl-1H-pyrrolo[3,2-b]pyridine;
[5-Chloro-6-isopropyl-3-(1-isopropyl-3,6-dimethyl-1H-pyrrolo[3,2-b]pyridin-5-yl)-pyridin-2-yl]-ethyl-amine;
1-Isopropyl-5-(6-isopropyl-2-methyl-pyridin-3-yl)-3,6-dimethyl-1H-pyrrolo[3,2-b]pyridine;
5-(6-Isopropyl-2-methyl-pyridin-3-yl)-3,6-dimethyl-1-propyl-1H-pyrrolo[3,2-b]pyridine;
1-Isopropyl-5-(6-isopropyl-pyridin-3-yl)-3,6-dimethyl-1H-pyrrolo[3,2-b]pyridine;
5-(6-Isopropyl-2-methoxy-pyridin-3-yl)-6-methoxy-1-((S)-2-methoxy-1-methyl-ethyl)-3-methyl-1H-pyrrolo[3,2-b]pyridine;
Cyclopropyl-{6-isopropyl-3-[1-((S)-2-methoxy-1-methyl-ethyl)-3,6-dimethyl-1H-pyrrolo[3,2-b]pyridin-5-yl]-pyridin-2-yl}-amine;
5-(6-Isopropyl-pyridin-3-yl)-6-methoxy-1-((S)-2-methoxy-1-methyl-ethyl)-3-methyl-1H-pyrrolo[3,2-b]pyridine;
5-(2-Ethyl-6-isopropyl-pyridin-3-yl)-6-methoxy-1-((S)-2-methoxy-1-methyl-ethyl)-3-methyl-1H-pyrrolo[3,2-b]pyridine;
Ethyl-{6-isopropyl-3-[6-methoxy-1-((S)-2-methoxy-1-methyl-ethyl)-3-methyl-1H-pyrrolo[3,2-b]pyridin-5-yl]-pyridin-2-yl}-amine;
{6-Isopropyl-3-[6-methoxy-1-((S)-2-methoxy-1-methyl-ethyl)-3-methyl-1H-pyrrolo[3,2-b]pyridin-5-yl]-pyridin-2-yl}-dimethyl-amine;
{6-Isopropyl-3-[6-methoxy-1-((S)-2-methoxy-1-methyl-ethyl)-3-methyl-1H-pyrrolo[3,2-b]pyridin-5-yl]-pyridin-2-yl}-methyl-amine;
6-Chloro-5-(6-isopropyl-2-methoxy-pyridin-3-y1)-1-((S)-2-methoxy-1-methyl-ethyl)-3-methyl-1H-pyrrolo[3,2-b]pyridine;
6-Chloro-5-(2-ethyl-6-isopropyl-pyridin-3-yl)-1-((S)-2-methoxy-1-methyl-ethyl)-3-methyl-1H-pyrrolo[3,2-b]pyridine;
6-Chloro-5-(6-isopropyl-pyridin-3-yl)-1-((S)-2-methoxy-1-methyl-ethyl)-3-methyl-1H-pyrrolo[3,2-b]pyridine;
{3-[6-Chloro-1-((S)-2-methoxy-1-methyl-ethyl)-3-methyl-1H-pyrrolo[3,2-b]pyridin-5-yl]-6-isopropyl-pyridin-2-yl}-dimethyl-amine;
{3-[6-Chloro-1-((S)-2-methoxy-1-methyl-ethyl)-3-methyl-1H-pyrrolo[3,2-b]pyridin-5-yl]-6-isopropyl-pyridin-2-yl}-methyl-amine;
{3-[6-Chloro-1-((S)-2-methoxy-1-methyl-ethyl)-3-methyl-1H-pyrrolo[3,2-b]pyridin-5yl]-6-isopropyl-pyridin-2-yl}-ethyl-amine;
{3-[6-Chloro-1-((R)-1-fluoromethyl-2-methoxy-ethyl)-3-methyl-1H-pyrrolo[3,2-b]pyridin-5-yl]-6-isopropyl-pyridin-2-yl}-methyl-amine;
6-Chloro-5-(2-ethyl-6-isopropyl-pyridin-3-yl)-1-((R)-1-fluoromethyl-2-methoxy-ethyl)-3-methyl-1H-pyrrolo[3,2-b]pyridine;
6-Chloro-1-((R)-1-fluoro-methyl-2-methoxy-ethyl)-5-(6-isopropyl-2-methyl-pyridin-3-yl)-3-methyl-1H-pyrrolo[3,2-b]pyridine;
{5-Chloro-3-[1-((R)-1-fluoromethyl-2-methoxy-ethyl)-3,6-dimethyl-1H-pyrrolo[3,2-b]pyridin-5-yl]-6-isopropyl-pyridin-2-yl}-methyl-amine;
1-(R)-1-Fluoromethyl-2-methoxy-ethyl)-5-(6-isopropyl-2-methyl-pyridin-3-yl)-3,6-dimethyl-1H-pyrrolo[3,2-b]pyridine;
Ethyl-{3-[1-((R)-1-fluoromethyl-2-methoxy-ethyl)-3,6-dimethyl-1H-pyrrolo[3,2-b]pyridin-5-yl]-6-isopropyl-pyridin-2-yl}-amine;
2-Bromo-7-(1-ethyl-propyl)-3-(2-methoxy-4-trifluoromethoxy-phenyl)-5-methyl-5H-pyrrolo[2,3-b]pyrazine;
7-(1-Ethyl-propyl)-3-(2-methoxy-4-trifluoromethoxy-phenyl)-5-methyl-5H-pyrrolo[2,3-b]pyrazine;
2-Ethyl-7-(1-ethyl-propyl)-3-(2-methoxy-4-trifluoromethoxy-phenyl)-5-methyl-5H-pyrrolo[2,3-b]pyrazine;
7-(1-Ethyl-propyl)-3-(2-methoxy-4-trifluoromethoxy-phenyl)-2,5-dimethyl-5H-pyrrolo[2,3-b]pyrazine;
2-Ethyl-7-(1-ethyl-propyl)-3-(6-isopropyl-2-methoxy-pyridin-3-yl)-5-methyl-5H-pyrrolo[2,3-b]pyrazine;
2-Ethyl-3-(2-ethyl-6-isopropyl-pyridin-3-yl)-7-(1-ethyl-propyl)-5-methyl-5H-pyrrolo[2,3-b]pyrazine;
{3-[2-Ethyl-7-(1-ethyl-propyl)-5-methyl-5H-pyrrolo[2,3-b]pyrazin-3-yl]-6-isopropyl-pyridin-2-yl}-methyl-amine;
Diethyl-{4-ethyl-5-[2-ethyl-7-(1-ethyl-propyl)-5-methyl-5H-pyrrolo[2,3-b]pyrazin-3-yl]-pyridin-2-yl}-amine;
2-Ethyl-7-(1-ethyl-propyl)-3-(3-isopropyl-5-methoxy-2,3-dihydro-furo[3,2-b]pyridin-6-yl)-5-methyl-5H-pyrrolo[2,3-b]pyrazine;
2-[3-(2-Methoxy-4-trifluoromethoxy-phenyl)-2,5-dimethyl-5H-pyrrolo[2,3-b]pyrazin-7-yl]-propan-1-ol;
7-(2-Methoxy-1-methyl-ethyl)-3-(2-methoxy-4-trifluoromethoxy-phenyl)-2,5-dimethyl-5H-pyrrolo[2,3-b]pyrazine;
2-[3-(2-Methoxy-4-trifluoromethoxy-phenyl)-2,5-dimethyl-5H-pyrrolo[2,3-b]pyrazin-7-yl]-propionic acid methyl ester;
2-[3-(6-Isopropyl-2-methoxy-pyridin-3-yl)-2,5-dimethyl-5H-pyrrolo[2,3-b]pyrazin-7-yl]-propan-1-ol;
Methanesulfonic acid 2-[3-(6-isopropyl-2-methoxy-pyridin-3-yl)-2,5-dimethyl-5H-pyrrolo[2,3-b]pyrazin-7-yl]-propyl ester;
3-(6-Isopropyl-2-methoxy-pyridin-3-yl)-7-(2-methoxy-1-methyl-ethyl)-2,5-dimethyl-5H-pyrrolo[2,3-b]pyrazine;
3-(6-Isopropyl-2-methoxy-pyridin-3-yl)-2,5-dimethyl-7-(1-methyl-2-morpholin-4-yl-ethyl)-5H-pyrrolo[2,3-b]pyrazine;
7-sec-Butyl-3-(6-isopropyl-2-methoxy-pyridin-3-yl)-2,5-dimethyl-5H-pyrrolo[2,3-b]pyrazine;
7-Isopropyl-3-(6-isopropyl-2-methoxy-pyridin-3-yl)-2,5-dimethyl-5H-pyrrolo[2,3-b]pyrazine;
2-[3-(6-Isopropyl-2-methoxy-pyridin-3-yl)-2,5-dimethyl-5H-pyrrolo[2,3-b]pyrazin-7-yl]-butan-1-ol;
3-(6-Isopropyl-2-methoxy-pyridin-3-yl)-7-(1-methoxymethyl-propyl)-2,5-dimethyl-5H-pyrrolo[2,3-b]pyrazine;
Methanesulfonic acid 2-[3-(6-isopropyl-2-methoxy-pyridin-3-yl)-2,5-dimethyl-5H-pyrrolo[2,3-b]pyrazin-7-yl]-butyl ester;
2-Ethyl-7-isopropyl-3-(6-isopropyl-2-methoxy-pyridin-3-yl)-5-methyl-5H-pyrrolo[2,3-b]pyrazine;
3-(2-Ethyl-6-isopropyl-pyridin-3-yl)-7-(2-methoxy-1-methyl-ethyl)-2,5-dimethyl-5H-pyrrolo[2,3-b]pyrazine;
2-Ethyl-3-(2-ethyl-6-isopropyl-pyridin-3-y)-7-isopropyl-5-methyl-5H-pyrrolo[2,3-b]pyrazine;
[3-(2-Ethyl-7-isopropyl-5-methyl-5H-pyrrolo[2,3-b]pyrazin-3-yl)-6-isopropyl-pyridin-2-yl]-methyl-amine;
{6-Isopropyl-3-[7-(2-methoxy-1-methyl-ethyl)-2,5-dimethyl-5H-pyrrolo[2,3-b]pyrazin-3-yl]-pyridin-2-yl}-methyl-amine;
{4-Ethyl-5-[2-ethyl-7-(1-ethyl-propyl)-5-methyl-5H-pyrrolo[2,3-b]pyrazin-3-yl]-pyridin-2-yl}-dimethyl-amine;
Ethyl-{4-ethyl-5-[2-ethyl-7-(1-ethyl-propyl)-5-methyl-5H-pyrrolo[2,3-b]pyrazin-3-yl]-pyridin-2-yl}-methyl-amine;
2,2′-Diethyl-7,7′-bis-(1-ethyl-propyl)-5,5′-dimethyl-5H,5¹H-[3,3′]bi[pyrrolo[2,3-b]pyrazinyl];
5-Ethyl-6-[2-ethyl-7-(1-ethyl-propyl)-5-methyl-5H-pyrrolo[2,3-b]pyrazin-3-yl]-3-isopropyl-3H-imidazo[4,5-b]pyridine;
2-Ethyl-7-(2-methoxy-ethyl)-3-(2-methoxy-4-trifluoromethoxy-phenyl)-5-methyl-5H-pyrrolo[2,3-b]pyrazine;
3-[2-Ethyl-3-(2-methoxy-4-trifluoromethoxy-phenyl)-5-methyl-5H-pyrrolo[2,3-b]pyrazin-7-yl]-propionitrile;
5-Bromo-3-(1-ethyl-propyl)-6-(2-methoxy-4-trifluoromethoxy-phenyl)-1-methyl-1H-pyrrolo[2,3-b]pyridine;
3-(1-Ethyl-propyl)-6-(2-methoxy-4-trifluoromethoxy-phenyl)-1,5-dimethyl-1H-pyrrolo[2,3-b]pyridine;
5-Chloro-3-(1-ethyl-propyl)-6-(2-methoxy-4-trifluoromethoxy-phenyl)-1-methyl-1H-pyrrolo[2,3-b]pyridine;
3-(1-Ethyl-propyl)-6-(2-methoxy-4-trifluoromethoxy-phenyl)-1,5-dimethyl-1H-pyrrolo[2,3-b]pyridine;
3-sec-Butyl-6-(6-isopropyl-2-methoxy-pyridin-3-yl)-1,5-dimethyl-1H-pyrrolo[2,3-b]pyridine;
3-sec-Butyl-6-(2-ethyl-6-isopropyl-pyridin-3-yl)-1,5-dimethyl-1H-pyrrolo[2,3-b]pyridine;
[3-(3-sec-Butyl-1,5-dimethyl-1H-pyrrolo[2,3-b]pyridin-6-yl)-6-isopropyl-pyridin-2-yl]-methyl-amine;
2-[6-(6-Isopropyl-2-methoxy-pyridin-3-yl)-1,5-dimethyl-1H-pyrrolo[2,3-b]pyridin-3-yl]-butan-1-ol;
6-(6-Isopropyl-2-methoxy-pyridin-3-yl)-3-(1-methoxymethyl-propyl)-1,5-dimethyl-1H-pyrrolo[2,3-b]pyridine;
5-Bromo-3-isopropyl-6-(6-isopropyl-2-methoxy-pyridin-3-yl)-1-methyl-1H-pyrrolo[2,3-b]pyridine;
3-Isopropyl-6-(6-isopropyl-2-methoxy-pyridin-3-yl)-1,5-dimethyl-1H-pyrrolo[2,3-b]pyridine;
3-(1-Ethoxymethyl-propyl)-6-(6-isopropyl-2-methoxy-pyridin-3-yl)-1,5-dimethyl-1H-pyrrolo[2,3-b]pyridine; and
5-Ethyl-3-isopropyl-6-(6-isopropyl-2-methoxy-pyridin-3-yl)-1-methyl-1H-pyrrolo[2,3-b]pyridine.

42. A compound or salt according to any of claims 1-41 wherein, in a standard in vitro CRF receptor binding assay the compound exhibits an IC₅₀ value for CRF receptors of less than or equal to 1 micromolar.

43. A compound or salt according to any of claims 1-41 wherein, in a standard in vitro CRF receptor binding assay the compound exhibits an IC₅₀ value for CRF receptors of less than or equal to 100 nanomolar.

44. A compound or salt according to any of claims 1-41 wherein, in a standard in vitro CRF receptor binding assay, the compound exhibits an IC₅₀ value for CRF receptors of less than or equal to 10 nanomolar.

45. A method for treating an anxiety disorder, a stress-related disorder, or an eating disorder, comprising administering to a patient in need of such treatment a therapeutically effective amount of a compound or salt according to any of claims 1-41.

46. A method for treating an depression or bipolar disorder, comprising administering to a patient in need of such treatment a therapeutically effective amount of a compound or salt according to any of claims 1-41.

47. A method for treating anorexia nervosa, bulimia nervosa, or obesity, comprising administering to a patient in need of such treatment a therapeutically effective amount of a compound or salt according to any of claims 1-41.

48. A compound or salt according to any of claims 1-41, wherein in a standard in vitro Na channel functional assay the compound does not show any statistically significant detectable Na channel modulatory activity at the p<0.05 level of significance in a standard parametric test of statistical significance.

49. A method for demonstrating the presence of CRF receptors in cell or tissue samples, said method comprising:
preparing a plurality of matched cell or tissue samples,
preparing at least one control sample by contacting (under conditions that permit binding of CRF to CRF receptors within cell and tissue samples) at least one of the matched cell or tissue samples (that has not previously been contacted with any compound or salt of any of claims 1-41) with a control solution comprising a detectably-labeled preparation of a selected compound or salt of any of claims 1-41 at a first measured molar concentration, said control solution further comprising an unlabelled preparation of the selected compound or salt at a second measured molar concentration, which second measured concentration is greater than said first measured concentration,
preparing at least one experimental sample by contacting (under conditions that permit binding of CRF to CRF receptors within cell and tissue samples) at least one of the matched cell or tissue samples (that has not previously been contacted with any compound or salt of any of claims 1-41) with an experimental solution comprising the detectably-labeled preparation of the selected compound or salt at the first measured molar concentration, said experimental solution not further comprising an unlabelled preparation of any compound or salt of any of claims 1-41 at a concentration greater than or equal to said first measured concentration;
washing the at least one control sample to remove unbound selected compound or salt to produce at least one washed control sample;
washing the at least one experimental sample to remove unbound selected compound or salt to produce at least one washed experimental sample;
measuring the amount of detectable label of any remaining bound detectably-labeled selected compound or salt in the at least one washed control sample;
measuring the amount detectable label of any remaining bound detectably-labeled selected compound or salt in the at least one washed experimental sample;
comparing the amount of detectable label measured in each of the at least one washed experimental sample to the amount of detectable label measured in each of the at least one washed control sample
wherein, a comparison that indicates the detection of a greater amount of detectable label in the at least one washed experimental sample than is detected in any of the at least one washed control samples demonstrates the presence of CRF receptors in that experimental sample.

50. A method of inhibiting the binding of CRF to a CRF1 Receptor, which method comprises:
contacting a solution comprising CRF and a compound or salt of any of claims 1 to 41 with a cell expressing the CRF receptor, wherein the compound or salt is present in the solution at a concentration sufficient to inhibit in vitro CRF binding to IMR32 cells.

51. The method of claim 50 wherein the cell expressing the CRF receptor is a neuronal cell that is contacted in vivo in an animal, and wherein the solution is a body fluid of said animal.

52. The method of claim 51 wherein the animal is a human patient.

53. A pharmaceutical composition comprising a pharmaceutically acceptable carrier and a compound or salt of any of claims 1-41.

54. A package comprising a pharmaceutical composition of claim 53 in a container and further comprising indicia comprising at least one of:
instructions for using the composition to treat a patient suffering from an anxiety disorder, or
instructions for using the composition to treat a patient suffering from a stress-related disorder, or
instructions for using the composition to treat a patient suffering from an eating disorder.

55. A package comprising a pharmaceutical composition of claim 53 in a container and further comprising indicia comprising at least one of: instructions for using the composition to treat a patient suffering from depression or instructions for using the composition to treat a patient suffering from a bipolar disorder.
